(19) United States
(12) Patent Application Publication
(10) Pub. No.: US 2024/0197846 A1
(43) Pub. Date: Jun. 20, 2024

(54) Antigen-Presenting Polypeptides with Chemical Conjugation Sites and Methods of Use Thereof

(71) Applicant: Cue Biopharma, Inc., Boston, MA (US)

(72) Inventors: Ronald D. SEIDEL, III, Natick, MA (US); John F. ROSS, Arlington, MA (US); Chee Meng LOW, Cambridge, MA (US)

(21) Appl. No.: 18/287,739

(22) PCT Filed: Apr. 20, 2022
(86) PCT No.: PCT/US2022/025532

§ 371 Date: Oct. 20, 2023

Related U.S. Application Data
(60) Provisional application No. 63/178,500, filed on Apr. 22, 2021.

(51) Int. Cl. A61K 39/00; C07K 14/47; C07K 14/495; C07K 14/525; C07K 14/55; C07K 14/62; C07K 14/705; C07K 14/74; C12N 9/16; C12N 9/88

(52) U.S. Cl. CPC A61K 39/0008; C07K 14/47; C07K 14/495; C07K 14/525; C07K 14/55; C07K 14/62; C07K 14/70532; C07K 14/70539; C12N 9/16; C12N 9/88; C12Y 301/03009; C12Y 301/03048; C12Y 401/01015

(57) ABSTRACT

The present disclosure provides antigen presenting polypeptide comprising a TGF-β MOD that is reversibly masked and acts as a TGF-β receptor agonist. The antigen presenting polypeptides comprising one or more chemical conjugation sites for incorporation of, for example, Type 1 Diabetes (T1D) associated epitope containing polypeptides. The antigen-presenting polypeptides and their T1D-associated epitope conjugates are useful for modulating the activity of a T-cell, and accordingly, the present disclosure provides methods of modulating activity of a T-cell in vitro and in vivo as a method of treatment of T1D.

This application contains a sequence listing submitted electronically via EFS-web, which serves as both the paper copy and the computer readable form (CRF) and consists of a file entitled “2910_27PCT_seqlist_ST25.txt”, which was created on Apr. 20, 2022, is 275,983 bytes in size, and which is herein incorporated by reference in its entirety.

### I. INTRODUCTION

An adaptive immune response involves the engagement of the T cell receptor (TCR), present on the surface of a T cell, with a small antigenic molecule non-covalently presented on the surface of an antigen presenting cell (APC) by a major histocompatibility complex (MHC; also referred to in humans as a human leukocyte antigen (“HLA”) complex). This engagement represents the immune system's targeting mechanism and is a requisite molecular interaction for T cell modulation (activation or inhibition) and effector function. In addition to epitope-specific cell targeting, the targeted T cells are activated through engagement of costimulatory proteins found, for example, on the APC with counterpart costimulatory proteins (e.g., receptors) on the T cells. Both signals—epitope/TCR binding and engagement of APC costimulatory proteins with T cell costimulatory proteins—are required to drive T cell specificity and activation or inhibition. The TCR is specific for a given epitope; however, costimulatory proteins are not epitope specific, and instead are generally expressed on all T cells or on subsets of T cells.

APCs generally serve to capture and break the proteins from foreign organisms, or abnormal proteins (e.g., from genetic mutation in cancer cells), into smaller fragments suitable as signals for scrutiny by the larger immune system, including T cells. In particular, APCs break down proteins into small peptide fragments, which are then paired with proteins of the major histocompatibility complex (“MHC”) and displayed on the cell surface. Cell surface display of an MHC together with a peptide fragment, also known as a T cell epitope, provides the underlying scaffold surveilled by T cells, allowing for specific recognition. The peptide fragments can be pathogen-derived (infectious agent-derived), tumor-derived, or derived from natural host proteins (self-proteins). Moreover, APCs can recognize other foreign components, such as bacterial toxins, viral proteins, viral DNA, viral RNA, etc., whose presence denotes an escalated threat level. The APCs relay this information to T cells through additional costimulatory signals in order to generate a more effective response.

T cells recognize peptide-major histocompatibility complex (“pMHC”) complexes through a specialized cell surface receptor, the T cell receptor (“TCR”). The TCR is unique to each T cell; as a consequence, each T cell is highly specific for a particular pMHC target. In order to adequately address the universe of potential threats, a very large number (˜10,000,000) of distinct T cells with distinct TCRs exist in the human body. Further, any given T cell, specific for a particular T cell peptide, is initially a very small fraction of the total T cell population. Although normally dormant and in limited numbers, T cells bearing specific TCRs can be readily activated and amplified by APCs to generate highly potent T cell responses that involve many millions of T cells. Such activated T cell responses are capable of attacking and clearing viral infections, bacterial infections, and other cellular threats including tumors. Conversely, the broad, non-specific activation of overly active T cell responses against self-antigens or shared antigens can give rise to T cells that inappropriately attack and destroy healthy tissues or cells.

MHC proteins are referred to as human leukocyte antigens (HLA) in humans. HLA proteins are divided into two major classes, class I and class II proteins, which are encoded by separate loci. Unless expressly stated otherwise, for the purpose of this disclosure, references to MHC or HLA proteins are directed to class II MHC or HLA proteins. HLA class II proteins each comprise alpha and beta polypeptide chains encoded by separate loci. HLA class II gene loci include HLA-DM (HLA-DMA and HLA-DMB that encode HLA-DM α chain and HLA-DM β chain, respectively), HLA-DO (HLA-DOA and HLA-DOB that encode HLA-DO α chain and HLA-DO β chain, respectively), HLA-DP (HLA-DPA and HLA-DPB that encode HLA-DP α chain and HLA-DP β chain, respectively), HLA-DQ (HLA-DQA and HLA-DQB that encode HLA-DQ α chain and HLA-DQ β chain, respectively), and HLA-DR (HLA-DRA and HLA-DRB that encode HLA-DR α chain and HLA-DR β chain, respectively).

Although the immune system is designed to avoid the development of immune responses to proteins and other potentially antigenic materials of the body, in some instances the immune system develops T cells with specificity for an epitope of an autoantigen (self-antigen) leading to autoimmune diseases.

Transforming growth factor beta (TGF-β) is a cytokine belonging to the transforming growth factor superfamily that includes three mammalian (human) isoforms, TGF-β1, TGF-β2, and TGF-β3. TGF-βs are synthesized as precursor molecules containing a propeptide region in addition to the TGF-β sequences that homodimerize as an active form of TGF-β. TGF-β is secreted by macrophages and other cell types in a latent complex in which it is combined with two other polypeptides-latent TGF-β binding protein (LTBP) and latency-associated peptide (LAP). The latent TGF-β complex is stored in the extra cellular matrix (ECM), for example, bound to the surface of cells by CD36 via thrombospondin-1 (where it can be activated by plasmin) or to latent transforming growth factor beta binding proteins 1, 2, 3, and/or 4 (LTBP1-4).

The biological functions of TGF-β are seen after latent TGF-β activation, which is tightly regulated in response to ECM perturbations. TGF-β may be activated by a variety of cell or tissue specific pathways, or pathways observed in multiple cell or tissue types; however, the full mechanisms behind such activation pathways are not fully known. Activators include, but are not limited to, proteases, integrins, pH, and reactive oxygen species (ROS). In effect, the cell/tissue bound latent TGF-β complex functions, senses and responds to environmental perturbations releasing active TGF-β in a spatial and/or temporal manner. The released TGF-β acts to promote or inhibit cell proliferation depending on the context of its release. It also recruits stem/progenitor cells to participate in the tissue regeneration/remodeling process. Aberrations in TGF-β ligand expression, bioavailability, activation, receptor function, or post-transcriptional modifications disturb the normal function, and can lead to pathological consequences associated with many diseases, such as through the recruitment of excessive progenitors (e.g., in osteoarthritis or Camurati-Engelmann disease), or by the trans-differentiation of resident cells to unfavorable lineages (e.g., in epithelial to mesenchymal transition during cancer metastasis or tissue/organ fibrosis). Xu et al Bone Research, 6 (Article No. 2) (2018).

A number of approaches to regulate TGF-β action at the level of the protein by sequestering it to effectively neutralize its action have been described in the literature and are sometimes referred to as “TGF-β traps.” For example, monoclonal antibodies such as Metelimumab (CAT192) that is directed against TGF-β1, and Fresolimumab directed against multiple isoforms of TGF-β have been developed to bind, sequester, and neutralize TGF-β in vivo. In addition, receptor traps that tightly bind and sequester TGF-β thereby sequestering and neutralizing it have also been developed (see, e.g., Swaagrtra, et al., Mol Cancer Ther; 11(7): 1477-87 (2012) and U.S. Pat. Pub. No. 2018/0327477).

### II. SUMMARY

The present disclosure provides T-cell modulatory antigen-presenting polypeptide(s) referred to as a “TMAPP” comprising at least one TGF-β sequence, a masking sequence that binds to a TGF-β sequence thereby reversibly masking it, or both (the combination together forming a “masked TGF-β MOD”), and having at least one chemical conjugation site where an epitope presenting molecule and/or a payload (e.g., a therapeutic) may be covalently attached. The TMAPPs may also comprise one or more additional wild-type (wt.) and/or variant MODs (e.g., IL-2) other than masked TGF-β MODs.). Individual TMAPPs may further comprise a scaffold polypeptide that permits, among other things, the formation of higher order complexes of two or more TMAPPs (e.g., duplex TMAPPs comprising two TMAPPs). Epitope presentation by a TMAPP to a target T cell is accomplished via a moiety that comprises MHC Class II polypeptides and the covalently conjugated epitope. Where TMAPPs have been conjugated to an epitope presenting molecule that becomes covalently bound at a chemical conjugation site of a TMAPP they are termed a “TMAPP-epitope conjugate.” Such moieties may be either (i) a single polypeptide chain, or (ii) a complex comprising two or more polypeptide chains. TMAPPs that include the MHC elements necessary for epitope presentation to a T cell receptor (“TCR”), for example the α1, α2, β1 and β2 domain sequences, in a single polypeptide chain (termed a “presenting sequence”) are denoted single-chain (“sc-TMAPP”). Examples of sc-TMAPPs are depicted in FIGS. 10C and 10D. TMAPPs that include the MHC elements necessary for epitope presentation to a T cell receptor (“TCR”) in complex comprising two or more polypeptide chains (termed a presenting complex) are denoted multimeric TMAPPs (“m-TMAPPs”) depicted in, for example, FIGS. 10A and 10B.

The terms “TMAPP” and “TMAPPs” as used herein will be understood to refer in different contexts to sc-TMAPPs and m-TMAPPs that are unconjugated to an epitope (unconjugated TMAPPs) or are in the form of an epitope conjugate. The corresponding epitope conjugates are termed “TMAPP-epitope conjugates” and are referred to in some instances more specifically as sc-TMAPP-epitope conjugates and m-TMAPP-epitope conjugates. TMAPP and TMAPPs also refer to higher order complexes of TMAPPs including their duplexes. Where reference to both a TMAPP and higher order TMAPP complex is made it is done for the purpose of emphasis. It will be clear to the skilled artisan when specific reference to only higher order structures are intended (e.g., by reference to duplex TMAPPs etc.).

By providing a chemical conjugation site for the incorporation of an epitope in the TMAPPs may be used as a T-cell receptor (“TCR”) presentation platform into which various epitopes (e.g., peptide antigens) may be covalently bound, and the resulting epitope conjugate used for modulating the activity of a T-cell bearing a TCR specific to the epitope. The effect of TMAPP-epitope conjugates on such T cells depends on their response to the TGF-β, and any other MODs that may be present in the TMAPP.

The masked TGF-β MOD of TMAPPs includes both a TGF-β amino acid sequence that is reversibly masked by a peptide with affinity for the TGF-β sequence (a “masking sequence”). Individual TMAPPs may comprise a complete masked TGF-β MOD where both the TGF-β sequence and masking sequence are present on the same polypeptide (i.e., placed in “cis,” see, e.g., FIG. 1C at (c) and (d)). Alternatively, an m-TMAPP may comprise a complete masked TGF-β MOD where the TGF-β sequence and masking sequence are present in “trans” located on separate polypeptides of the m-TMAPP. The masking sequence and TGF-β sequence may be placed in trans on peptides of two TMAPPs so that they form a complete masked TGF-β MOD when those TMAPP are brought together in a duplex TMAPP, see e.g., FIG. 1C at (a) or a higher order TMAPP complex. Control of pairing between a TGF-β sequence and a masking sequence on separate TMAPPs can be obtained by using scaffold that comprise interspecific binding sequences (see, e.g., FIG. 1C at (a) and (b)).

Unlike the molecules TGF-β traps and related discussed above designed to bind and sequester the TGF-β and that act as antagonists to TGF-β action, masked TGF-β MODs provide active TGF-β polypeptides (e.g., TGF-β signaling pathway agonists). The TGF-β polypeptides and a masking polypeptide (e.g., a TGF-β receptor fragment) of masked TGF-β MODs interact with each other to reversibly mask the TGF-β polypeptide sequence permitting the TGF-β polypeptide sequence to interact with its cellular receptor. In addition, the masking sequence competes with cellular receptors that can scavenge TGF-β, such as the non-signaling TβRIII, thereby permitting the TMAPP to effectively deliver active TGF-β agonist to target cells. While the TMAPP construct permits epitope-specific/selective presentation of a reversibly masked TGF-β to a target T cell, it also provides sites for the presentation of one or more additional MODs. The ability of the TMAPP construct to include one or more additional MODs thereby permits the combined presentation of TGF-β and the additional MOD(s) to direct a target T cell's response in a substantially epitope-specific/selective manner.

Accordingly, the TMAPP described herein function as a platform for the incorporation of epitopes into MHC constructs for presentation to a TCR of a target T cell selective for the epitope, in the presence of a TGF-β agonist in the form of a masked TGF-β MOD. The TMAPPs also provide a structure for the presentation of one or more additional MODs that can further influence response of the target T cell. As such, the TMAPPs, duplex TMAPPs, and TMAPPs of higher described herein provide a means by which various epitope may be readily presented in the context of a Class II MHC (e.g., Class II HLA) to a target T cell displaying a TCR specific for the epitope, while at the same time permitting for the flexible presentation of at least one masked TGF-§ MOD, and optionally one or more additional MODs. The TMAPPs and higher order TMAPP complexes thereby permit delivery of one or more masked TGF-β MODs in a substantially epitope-specific/selective manner that permits (i) formation of an active immune synapse with a target T cell, such as a CD4+ cell selective for the epitope, and (ii) modulation (e.g., control/regulation) of the target T cell's response to the epitope. The TMAPPs and their epitope conjugates described herein find use in, among other things, the delivery of TGF-β and any additional MOD present in a TMAPP to T cells, the modulation of T cell responses in vitro and in vivo, and in the treatment of various disorders including autoimmune disease, graft vs. host disease (GVHD), host vs. graft disease (HVGD), allergic reactions.

### III. BRIEF DESCRIPTION OF THE DRAWINGS

FIG. 1 provides a schematic depiction of exemplary unconjugated TMAPPs bearing a masked TGF-β MOD. The presenting sequence or complexes, which comprise the MHC Class II domains sufficient to present an epitope to a TCR (e.g., the α1, α2, β1 and β2 domain sequences) are show generically and appear in FIG. 19A to FIG. 19J. The constructs in the figures are oriented from left to right as N-terminus to C terminus (with the masked TGF-β MOD appearing on the C-terminal end of the molecules). A TMAPP lacking a scaffold sequence is shown with the masked TGF-β MOD the closed position at (a) and the open position at (b). At (c) and (d) TMAPPs with scaffold sequences are shown in the closed and open positions. At (e) a first TMAPP and a second TMAPP are shown in a higher order complex (a duplex TMAPP) formed by interactions between their scaffolds, which may be non-covalent or covalent. At (f) the duplex TMAPP shown in (e) is depicted with the masked TGF-β MOD in the open position. At (g) and (h) duplex TMAPPs are shown in which the masking sequence is present on a first TMAPP of the duplex and the TGF-β sequences is present on a second TMAPP of the duplex (i.e., the masking sequence and the TGF-β sequence are placed in trans). Pairing of the TMAPPs in (g) and (h) to form a duplex occurs through interspecific scaffold sequences indicated by a knob-in-hole structure, although any other interspecific sequence pair could be used. Lines between the elements in FIG. 1, and other drawings depicting TMAPPs or their elements represent optional aa linker sequences. The locations of chemical conjugation sites are not indicated in the drawings, but chemical conjugation sites for epitopes are typically in one of the MHC α1, α2, β1 and β2 domain sequences, or linkers attached to them. Payloads conjugation sites are typically in the scaffold sequences.

FIGS. 2A-2H provide amino acid sequences of immunoglobulin polypeptides including their heavy chain constant regions (“Ig Fc” or “Fc”, e.g., the CH2-CH3 domain of IgG1) (SEQ ID NOs:1-13).

FIG. 2I provides the sequence of an Ig CH1 domain (SEQ ID NO:14).

FIG. 2J provides the sequence of a human Ig-J chain (SEQ ID NO:62).

FIG. 3A provides the sequence of an Ig α chain (kappa chain) constant region (SEQ ID NO:15).

FIG. 3B provides the sequence of an Ig λ chain (lambda chain) constant region (SEQ ID NO:16).

FIG. 4 provides an amino acid sequence of an HLA Class II DRA (sometimes referred to as DRA1) α chain (SEQ ID NO:17).

FIG. 5 provides amino acid sequences of HLA Class II DRB1 β chains (SEQ ID NOs:18-54).

FIG. 6 provides amino acid sequences of HLA Class II DRB3 β chains (SEQ ID NOs:55-58).

FIG. 7 provides an amino acid sequence of a HLA Class II DRB4 β chain (SEQ ID NOs:59-60).

FIG. 8 provides an amino acid sequence of a HLA Class II DRB5 β chain (SEQ ID NO:61). [0027].

FIG. 9A-9J provided in FIGS. 9A to 9D are exemplary structures of presenting complexes whose incorporation into a TMAPP structure (e.g., as in FIG. 1) gives rise to a m-TMAPP. The present complex first sequence is the sequence terminating in the symbol “−//−”, which indicates the point of attachment between the presenting sequences or complexes and the scaffold or masked TGF-β MOD structures. The presenting complex second sequence is joined covalently (e.g., by disulfide bonds) or non-covalently to the presenting complex first sequence. FIGS. 9E to 9J provide the structures of exemplary presenting sequences whose incorporation into a TMAPP structure gives rise to a sc-TMAPP. Elements labeled “*MOD” represent the location where one or more (wt. or variant) MODs, including MODs in tandem (e.g., two IL-2 sequences with at most an intervening linker), may be incorporated into presenting sequences or presenting complexes.

FIGS. 10A-10D. FIG. 10A shows a duplex of unconjugated m-TMAPPs (each TMAPP bearing a presenting complex as in FIG. 9A) with the individual TMAPPs having the masking and TGF-β sequences placed in cis and brought into proximity by the interspecific scaffold sequence pair (illustrated by a knob-in-hole sequence pair with disulfide bonds). The covalent attachment by one or more disulfide bonds is optional. The structure in FIG. 10B parallels that in FIG. 10A, however, there are two TGF-β MODs with the masking and TGF-β sequences located in cis, and the scaffolds are not an interspecific pair. Examples of a presenting complex first sequence and second sequences are indicated in FIG. 10B. FIG. 10C and FIG. 10D show duplexed unconjugated sc-TMAPPs that parallel the structures in FIGS. 10A and 10B respectively (each TMAPP bearing a presenting sequence as in FIG. 9G). In each of FIGS. 10A-10D the masked TGF-β MODs are shown in the closed position. An example of a presenting sequence is indicated in FIG. 10D.

FIG. 11 shows a schematic of hydrazinyl indoles reacting with an aldehyde containing polypeptide adapted from U.S. Pat. No. 9,310,374.

FIG. 12 provides a table showing examples of HLA Class II alleles, MODs, and T1D epitopes that may be incorporated into a TMAPP for T1D therapy.

FIG. 13 show the formation of a duplex TMAPP epitope conjugate using a pair of interspecific scaffold sequences. At A a pair of TMAPPs with interspecific scaffolds Ig Fc scaffolds (“Fc”) are provided. Both TMAPPs have a presenting sequence as illustrated in FIG. 9G, with the first bearing two IL-2 MODs in tandem and the second bearing a PD-L1 MOD. At B the TMAPPs are shown in duplex form, that is contacted with the epitope bound to a maleimide group via a linker provided at C. The resulting duplex TMAPP-epitope conjugate is shown at D.

FIG. 14 provides the sequences of three different isoforms of TGF-β as preproproteins and the mature form of TGF-β3 along with the C77S mutant of the mature protein.

FIG. 15 provides an alignment of TGF-β isoforms 1-3 with the residues corresponding to the mature form of TGF-β2 bolded, except aa residues Lys 25, Cys 77, Ile 92, and Lys 94 of TGF-β2 and their corresponding residues in the other forms of TGF-β isoforms 1 and 3 that are underlined and italicized but not bolded. TGF-β1 (NP_000651.3 and P01137), TGF-β2 (AAA50405.1), and TGF-β-3 isoform 1 (NP_0013168.1).

FIG. 16A provides the sequences of a type 1 TGF-β receptor (TβRI) and its ectodomain.

FIG. 16B provides the sequences of a type 2 TGF-β receptor (TβRII), its ectodomain, and fragments of the ectodomain. The locations indicated in bold and underlining in the isoform B are aas F30, D32, S52, E55 and D118 of the mature polypeptide, any of which may be substituted with an as other than the naturally occurring aa. The ectodomain fragments are based upon NCBI Ref. Seq. NP_003233.4, and UniProtKB Ref. P37173; with the ectodomain sequence corresponding to aas 49 to 159 of those sequences. The substitution at aspartic acid “D119” of the mature protein with an alanine “A” (bolded, italicized, and underlined) is marked as a “D118A” substitution for consistency with the literature describing that substitution when signal peptide is understood to be 23 aas in length as opposed to 22 aas in the NCBI record. The as D119 numbering assignment is based on the mature protein, and accordingly, it is D141 of the precursor protein when the 22 as signal sequence is included. The location of D32, sometimes substituted with asparagine (D32N), corresponds to D55 in the precursor protein. The corresponding aas in mature isoform A lacking its signal sequence are F55, D57, S77, E80, and D143 (see e.g., SEQ ID NO:215).

FIG. 16C provides the sequences of a type 3 TGF-β receptor (TβRIII).

### IV. DEFINITIONS

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The terms “polypeptide,” and “protein” are used interchangeably herein, and refer to a polymeric form of amino acids, which unless stated otherwise are the naturally occurring proteinogenic L-amino acids that are incorporated biosynthetically into proteins during translation in a mammalian cell. Furthermore, as used herein, a “polypeptide” and “protein” include modifications, such as deletions, additions, and substitutions (generally conservative in nature as would be known to a person in the art) to the native sequence, as long as the protein maintains the desired activity. These modifications can be deliberate, as through site-directed mutagenesis, or can be accidental, such as through mutations of hosts that produce the proteins, or errors due to polymerase chain reaction (PCR) amplification or other recombinant DNA methods. References to a specific residue or residue number in a known polypeptide, e.g., position 72 or 75 of human DRA MHC class II polypeptide, are understood to refer to the amino acid at that position in the wild-type polypeptide (i.e. I72 or K75). To the extent that the sequence of the wild-type polypeptide is altered, either by addition or deletion of one or more amino acids, the specific residue or residue number will refer to the same specific amino acid in the altered polypeptide (e.g., in the addition of one amino acid at the N-terminus of a peptide reference as position I72, will be understood to indicate the amino acid, Ile, that is now position 73). Substitution of an amino acid at a specific position is denoted by an abbreviation comprising, in order, the original amino acid, the position number, and the substituted amino acid, e.g., substituting the Ile at position 72 with a cysteine is denoted as I72C.

A nucleic acid or polypeptide has a certain percent “sequence identity” to another polynucleotide or polypeptide, meaning that, when aligned, that percentage of bases or amino acids are the same, and in the same relative position, when comparing the two sequences. Sequence identity can be determined in a number of different ways. To determine sequence identity, sequences can be aligned using various convenient methods and computer programs (e.g., BLAST, T-COFFEE, MUSCLE, MAFFT, etc.), available over the world wide web at sites including blast.ncbi.nlm.nih.gov/Blast.cgi for BLAST+2.10.0, ebi.ac.uk/Tools/msa/tcoffee/, ebi.ac.uk/Tools/msa/muscle/, and mafft.cbrc.jp/alignment/software/. See, e.g., Altschul et al. (1990), J. Mol. Biol. 215:403-10. Unless otherwise indicated, the percent sequence identities described herein are those determined using the BLAST program.

As used herein amino acid (“aa” singular or “aas” plural) means the naturally occurring proteogenic amino acids incorporated into polypeptides and proteins in mammalian cell translation. Unless stated otherwise: L (Leu, leucine), A (Ala, alanine), G (Gly, glycine), S (Ser, serine), V (Val, valine), F (Phe, phenylalanine), Y (Tyr, tyrosine), H (His, histidine), R (Arg, arginine), N (Asn, asparagine), E (Glu, glutamic acid), D (Asp, asparagine), C (Cys, cysteine), Q (Gln, glutamine), I (Ile, isoleucine), M (Met, methionine), P (Pro, proline), T (Thr, threonine), K (Lys, lysine), and W (Trp, tryptophan). Amino acid also includes the amino acids hydroxyproline and selenocysteine, which appear in some proteins found in mammalian cells, however, unless their presence is expressly indicated they are not understood to be included.

As used herein the term “in vivo” refers to any process or procedure occurring inside of the body, e.g., of a patient.

As used herein, “in vitro” refers to any process or procedure occurring outside of the body.

The term “conservative amino acid substitution” refers to the interchangeability in proteins of as residues having similar side chains. For example, a group of aas having aliphatic side chains consists of glycine, alanine, valine, leucine, and isoleucine; a group of aas having aliphatic-hydroxyl side chains consists of serine and threonine; a group of aas having amide containing side chains consists of asparagine and glutamine; a group of aas having aromatic side chains consists of phenylalanine, tyrosine, and tryptophan; a group of aas having basic side chains consists of lysine, arginine, and histidine; a group of aas having acidic side chains consists of glutamate and aspartate; and a group of aas having sulfur containing side chains consists of cysteine and methionine. Exemplary conservative as substitution groups are: valine-leucine-isoleucine, phenylalanine-tyrosine, lysine-arginine, alanine-valine-glycine, and asparagine-glutamine.

The term “binding” refers to a direct association between molecules and/or atoms, due to, for example, covalent, electrostatic, hydrophobic, and ionic and/or hydrogen-bond interactions, including interactions such as salt bridges and water bridges. “Covalent bonding,” or “covalent binding” as used herein, refers to the formation of one or more covalent chemical bonds between two different molecules. The term “binding,” as used with reference to the interaction between a TMAPP and a T cell receptor (TCR) on a T cell, refers to a non-covalent interaction between the TMAPP and TCR.

“Affinity” as used herein generally refers to the strength of non-covalent binding, increased binding affinity being correlated with a lower K_{D}. As used herein, the term “affinity” may be described by the dissociation constant (K_{D}) for the reversible binding of two agents (e.g., an antibody and an antigen. As used herein, the term “avidity” refers to the resistance of a complex of two or more agents to dissociation after dilution.

“T cell” includes all types of immune cells expressing CD3, including T-helper cells (CD4⁺ T-helper cells), cytotoxic T cells (CD8⁺ cells), T-regulatory cells (Treg), and NK-T cells.

The term “immunomodulatory polypeptide” (also referred to as a “costimulatory polypeptide” or, as noted above, “MOD”), as used herein includes a wild-type or variant of a polypeptide or portion thereof that can specifically bind a cognate co-immunomodulatory polypeptide (“co-MOD”) present on a T cell, and provide a modulatory signal to the T cell when the TCR of the T cell is engaged with an MHC-epitope moiety that is specific for the TCR. Unless stated otherwise the term “MOD” includes wild-type and/or variant MODs, and statements including reference to both wild-type and variant MODs are made to emphasize that one, the other, or both are being referenced. The signal provided by the MOD engaging its co-MOD mediates (e.g., directs) a T cell response. Such responses include, but are not limited to, proliferation, activation, differentiation, suppression/inhibition of proliferation, activation and/or differentiation, and the like.

“Heterologous,” as used herein, means a nucleotide or polypeptide that is not found in the native nucleic acid or protein, respectively.

“Recombinant,” as used herein, means that a particular nucleic acid (DNA or RNA) is the product of various combinations of cloning, restriction, polymerase chain reaction (PCR) and/or ligation steps resulting in a construct having a structural coding or non-coding sequence distinguishable from endogenous nucleic acids found in natural systems. DNA sequences encoding polypeptides can be assembled from cDNA fragments or from a series of synthetic oligonucleotides, to provide a synthetic nucleic acid which is capable of being expressed from a recombinant transcriptional unit contained in a cell or in a cell-free transcription and translation system.

The terms “recombinant expression vector,” or “DNA construct” are used interchangeably herein to refer to a DNA molecule comprising a vector and at least one insert. Recombinant expression vectors are usually generated for the purpose of expressing and/or propagating the insert(s), or for the construction of other recombinant nucleotide sequences. The insert(s) may or may not be operably linked to a promoter sequence and may or may not be operably linked to DNA regulatory sequences.

The terms “treatment,” “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease or symptom in a mammal, and includes: (a) preventing the disease or symptom from occurring in a subject which may be predisposed to acquiring the disease or symptom but has not yet been diagnosed as having it; (b) inhibiting the disease or symptom, i.e., arresting its development; and/or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy will desirably be administered during the symptomatic stage of the disease, and in some cases after the symptomatic stage of the disease.

The terms “individual,” “subject,” “host,” and “patient” are used interchangeably herein and refer to any mammalian subject for whom diagnosis, treatment, or therapy is desired. Mammals include humans and non-human primates, and in addition include rodents (e.g., rats; mice), lagomorphs (e.g., rabbits), ungulates (e.g., cows, sheep, pigs, horses, goats, and the like), felines, canines, etc.

Unless indicated otherwise, the term “substantially” is intended to encompass both “wholly” and “largely but not wholly”. For example, an Ig Fc that “substantially does not induce cell lysis” means an Ig Fc that induces no cell lysis at all or that largely but not wholly induces no cell lysis.

As used herein, the term “about” used in connection with an amount indicates that the amount can vary by 10%. For example, “about 100” means an amount of from 90-110. Where about is used in the context of a range, the “about” used in reference to the lower amount of the range means that the lower amount includes an amount that is 10% lower than the lower amount of the range, and “about” used in reference to the higher amount of the range means that the higher amount includes an amount 10% higher than the higher amount of the range. For example, from about 100 to about 1000 means that the range extends from 90 to 1100.

The terms “purifying”, “isolating”, and the like, refer to the removal of a desired substance, e.g., a TMAPP, from a solution containing undesired substances, e.g., contaminates, or the removal of undesired substances from a solution containing a desired substance, leaving behind essentially only the desired substance. In some instances, a purified substance may be essentially free of other substances, e.g., contaminates. As will be understood by those of skill in the art, generally, components of the solution itself, e.g., water or buffer, or salts are not considered when determining the purity of a substance.

Where a range of values is provided, it is understood that each intervening value between the upper and lower limit of that range to a tenth of the lower limit of the range is encompassed within the disclosure along with any other stated or intervening value in the range. The upper and lower limits of these smaller ranges may independently be included in smaller ranges, that are also encompassed within the disclosure subject to any specifically excluded limit in the stated range. Where the stated range a value (e.g., an upper or lower limit), ranges excluding those values are also included.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a Treg” includes a plurality of such Tregs and reference to “the MHC Class II alpha chain” includes reference to one or more MHC Class II alpha chains and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

### V. DETAILED DESCRIPTION

The present disclosure provides T-cell modulatory antigen-presenting polypeptide(s) referred to as a “TMAPP” bearing at least one masked TGF-β MOD, and prior to chemical conjugation with an epitope or payload (e.g., a therapeutic) having at least one chemical conjugation for their attachment. TMAPPs (singular) or “TMAPPs” (plural) include the MHC elements necessary for epitope presentation to a T cell receptor (“TCR”) (e.g., the MHC α1, α2, β1, and β2 domain sequences). Chemical conjugation sites for the conjugation of epitopes are typically located in the MHC, or in linkers attached to those domains. Chemical conjugation sites for the conjugation of payload molecules, when present, are typically located on scaffold sequences. By providing a chemical conjugation site for the incorporation of an epitope in TMAPPs, unconjugated TMAPPs may be used as a T-cell receptor (“TCR”) presentation platform into which various epitopes (e.g., peptide antigens) may be covalently bound, and the resulting epitope conjugate used for modulating the activity of a T-cell bearing a TCR specific to the epitope. The effect of TMAPP-epitope conjugates on T-cells with TCRs specific to the epitope conjugate depends on which, if any, MODs in addition to the masked TGF-β MOD are present in the TMAPP-epitope conjugate. TMAPPs may comprise MODs with reduced affinity for their cognate receptors (co-MODs) on T cells. The combination of the reduced affinity of the MOD(s) for their Co-MOD(s), and the affinity of the epitope for a TCR, provides for enhanced selectivity of a TMAPP-epitope conjugates while retaining the activity of the MODs. Accordingly, the present disclosure provides methods of modulating the activity of T-cells selective for the epitope presented by the TMAPP in vitro and in vivo, and the use of TMAPPs as therapeutics for, among other things, methods of treatment of disease and disorders including autoimmune diseases, GVHD, HVGD, and allergies, as well as metabolic disorders.

TMAPPs may comprise a scaffold sequences that permits two or more TMAPPs to associate, thereby forming a higher order structure (e.g., a duplex). The scaffolds do not comprise an MHC (e.g., HLA) class II α chain or β chain polypeptide sequence; and as such, interaction brought about by those sequences are not considered higher order TMAPP complex formation. TMAPPs provide a structure upon which other polypeptides can be organized for presentation to cellular TCRs.

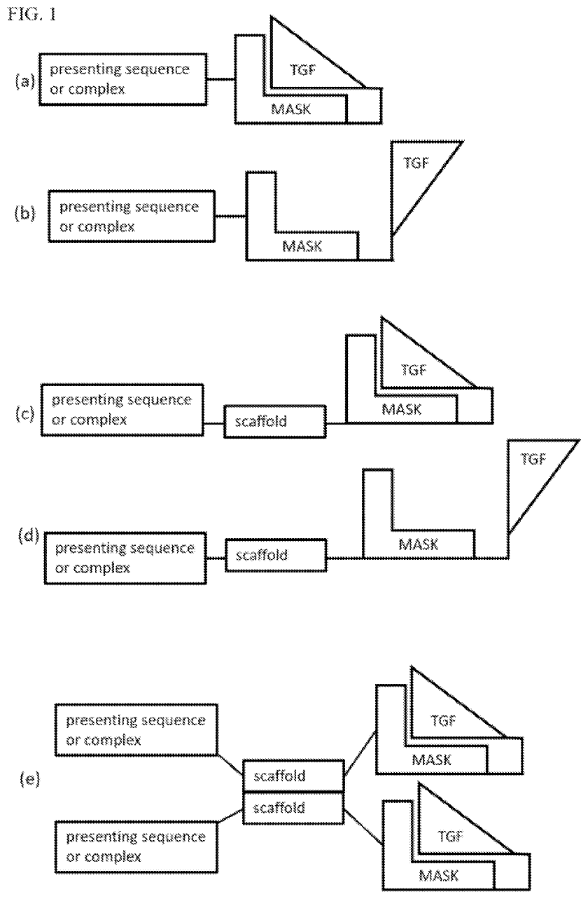

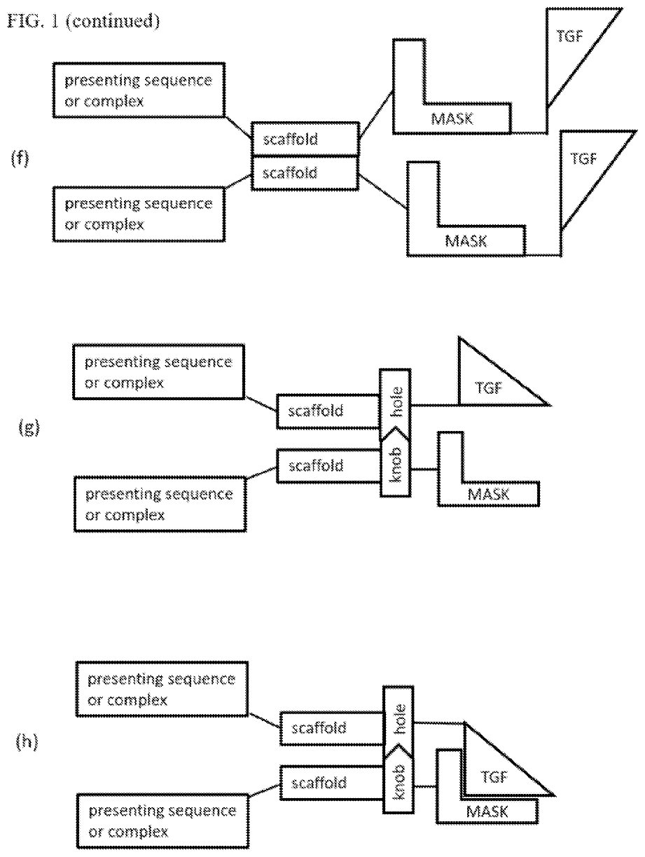

FIGS. 1E to 1H show as examples a series of duplex TMAPPs with masked TGF-β MODs located on the carboxyl end of the scaffold sequences in the close or open positions.

Epitope conjugates of TMAPPs (e.g., duplex TMAPP-epitope conjugates) provide a means by which peptide epitopes may be delivered in the context of an MHC (e.g., HLA) along with one or more masked TGF-β MOD(s) to a target T cell displaying a TCR specific for the epitope, while at the same time permitting for the flexible presentation of one or more MODs in addition to the masked TGF-β MOD(s). The TMAPP-epitope conjugates, and their higher complexes thereby permit deliver of one or more MODs in an epitope selective (e.g., dependent/specific) manner that permits formation of an active immune synapse with a target T cell selective for the epitope, and control/regulation of the target T cell's response to the epitope. The target T cell's response to the TMAPP-epitope conjugate depend on the MODs and epitope presented by the TMAPP-epitope conjugate. Accordingly, where TMAPP-epitope conjugates comprise stimulatory or activating MODs (e.g., IL-2, CD80, CD86, and/or 4-1BBL) that increase T cell proliferation and/or effector functions in an epitope selective manner. In contrast, where TMAPP-epitope conjugates comprise suppressive/inhibitory MODs (e.g., FasL and/or PD-Li) they generally decrease T cell activation, proliferation, and/or effector functions in an epitope selective manner. The TMAPP-epitope conjugates, particularly when comprising one or more masked TGF-β MOD and one or more IL-2 MOD polypeptide sequences may function to increase the induction or proliferation of Tregs in an epitope selective manner. TMAPP-epitope conjugates, which bear at least one masked TGF-β MOD alone or in combination with one or more IL-2 MOD polypeptide sequence may also be combined with additional MOD such as PD-L1 or 4-1BBL to provide additional modulatory signals.

TMAPPs and their higher order structures may also be understood to provide flexibility in locating MODs. Acceptable combinations of properties may be obtained when the MOD are positioned at the N-terminus of a presenting sequence or presenting complex polypeptide, respectively. In some cases, acceptable combinations of properties may be obtained when a MOD is located at the C-terminus of a presenting complex second sequence or positioned at the C-terminus of a scaffold.

TMAPPs and accordingly their higher order complexes (duplexes, triplexes etc.) comprise MHC Class II polypeptide sequences that bind an epitope for presentation to a TCR, and accordingly may present peptides to T cells (e.g., CD4⁺ T cells). The effect of TMAPP-epitope conjugates on T cells with TCRs specific to the epitope depends on which, if any, MODs in addition to the masked TGF-β MOD(s) that are present in the TMAPP-epitope conjugate. As noted above, TMAPP-epitope conjugates, (e.g., duplex TMAPP-epitope conjugates) permit MOD delivery to T cells in an epitope selective manner and the MODs principally dictate the effect of TMAPP-epitope conjugate-T cell engagement in light of the specific cell type stimulated and the environment. While not wishing to be bound by any particular theory, the effect of TMAPP-epitope conjugate presentation of MOD(s) and epitope to a T cells in some cases may be enhanced relative to the situation encountered in antigen presenting cells (APC) where epitope can diffuse away from the MHC (e.g., HLA) complex and any MODs the APC is presenting. This cannot occur with a TMAPP-epitope conjugate where the epitope and MOD(s) are part of the TMAPP polypeptide(s) and cannot diffuse away even if the epitope's affinity for the MHC complex would normally permit it to leave the comparable cell complex. The inability of epitope to diffuse away from MHC and MOD components of a TMAPP-epitope conjugate may be further limited where the polypeptide(s) of the TMAPP-epitope conjugate (e.g., presenting complex first and second sequences) are covalently attached to each other (e.g., by disulfide bonds). Consequently, TMAPP-epitope conjugates and their higher order structures may be able to prolong delivery of MOD(s) to T cells in an epitope selective manner relative to systems where epitopes can diffuse away from the presenting MHC.

Incorporation of one or more MODs with affinity for their cognate receptor on T cells (“co-MOD”) can reduce the specificity of TMAPP-epitope conjugates (e.g., duplex TMAPP-epitope conjugates) for epitope selective/specific T cells. The reduction in epitope selectivity/specificity of the TMAPP-epitope conjugates becomes more pronounced where MOD/co-MOD binding interactions increase in strength (binding energy) and significantly compete with MHC/epitope binding to target cell TCR. The inclusion of variant MODs, including TGF-β MODs, with reduced affinity for their co-MOD(s) thus may provide a lower contribution of MOD binding energy, thereby permitting MHC-epitope interactions in which the TCR dominates the binding and provides epitope selective interactions with T cells while retaining the activity of the MODs. Variant MODs with one or more substitutions (or deletions or insertions) that reduced the affinity of the MOD for their co-MOD may be incorporated into TMAPPs and their higher order complexes alone or in combination with wild-type MODs polypeptide sequences. Wild-type and variant MODs are described further below. Inclusion of masking sequences that bind tightly to the TGF-β polypeptide sequence effectively reduces the apparent affinity of the TGF-β polypeptide sequence for the cellular receptors, thereby decreasing the contribution of TGF-β polypeptide to cellular TβR bind in TMAPP association with a T cells, which permits MHC-epitope interactions with the TCR to dominate the T-cell binding interactions and effect epitope specific/selective T cell interactions and epitope specific/selective delivery of the masked TGF-β MOD and any other MODs on the TMAPP-epitope conjugate to the target T cell.

The ability of TMAPP-epitope conjugates to modulate T cells in an epitope selective/specific manner thus provides methods of modulating activity of a T cell in vitro and in vivo, and accordingly, methods of treating disease such as GVHD, HVGD, and disorders related to immune dysregulation/disfunction, including allergies and autoimmune diseases, as well as metabolic disorders.

The present disclosure provides nucleic acids comprising nucleotide sequences encoding TMAPP polypeptides, cells genetically modified with the nucleic acids and capable of producing the TMAPP, and methods of producing TMAPPs and their higher order complexes utilizing such cells.

Each presenting sequence or presenting complex present in a TMAPP comprises MHC class II alpha and beta chain polypeptide sequences (e.g., human MHC class II sequences) sufficient to bind a peptide epitope and present it to a TCR. MHC Class II peptides, may include sequence variations that are designed to stabilize the MHC, stabilize the MHC peptide epitope complex, and/or stabilize the TMAPP. Sequence variations may also serve to enhance cellular expression of TMAPPs prepared in cell-based systems as well as the stability (e.g., thermal stability) of TMAPPs and their higher order complexes such as duplex TMAPPs. Some MHC class II sequences suitable for use in TMAPPs are described below.

As indicated in the description of the drawings, TMAPPs may comprise one or more independently selected peptide sequences or (one or more “linker” or “linkers”) between any two or more components of the TMAPP, which in the figures may be shown as a line between peptide and/or polypeptide elements of the TMAPPs. The same sequences used as linkers may also be located at the N- and/or C-termini of the TMAPP peptides to prevent, for example, proteolytic degradation. Linker sequences include but are not limited to polypeptides comprising: glycine; glycine and serine; glycine and alanine; alanine and serine; and glycine, alanine and serine; any one which may comprise a cysteine for formation of an intrapolypeptide or interpolypeptide disulfide bond. Various linkers are described in more detail below.

### V.A. Structure and Organization of TMAPPs

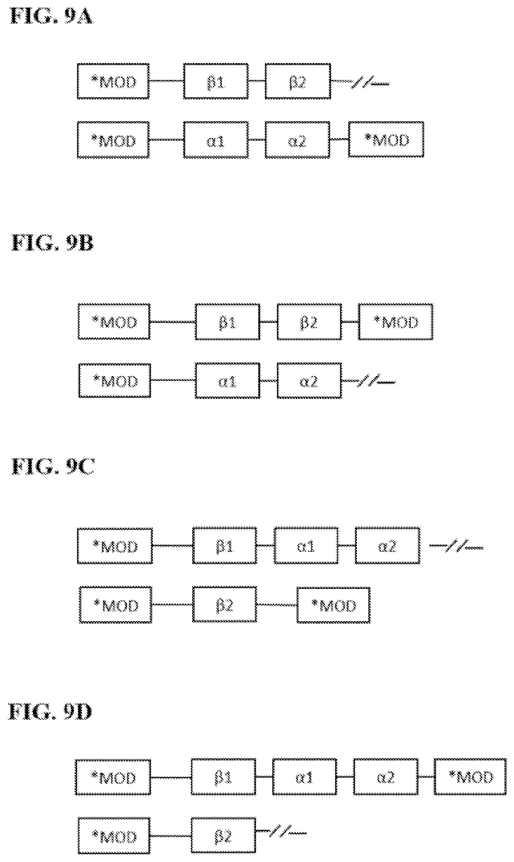

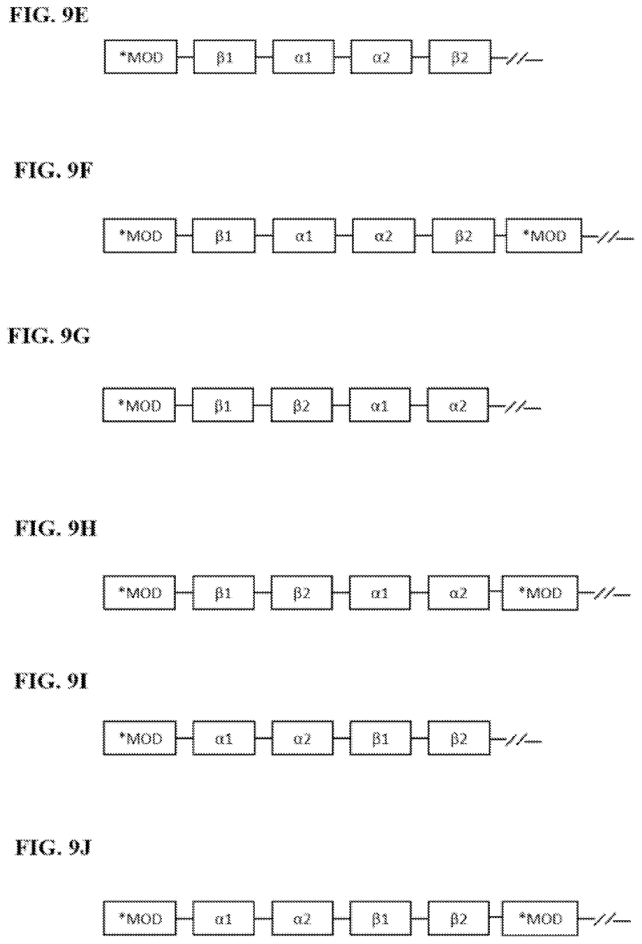

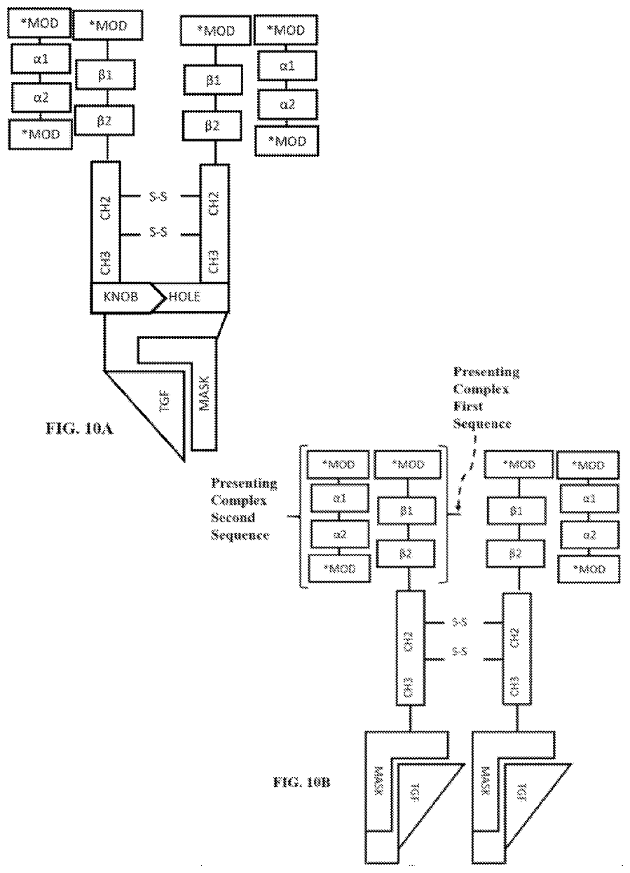

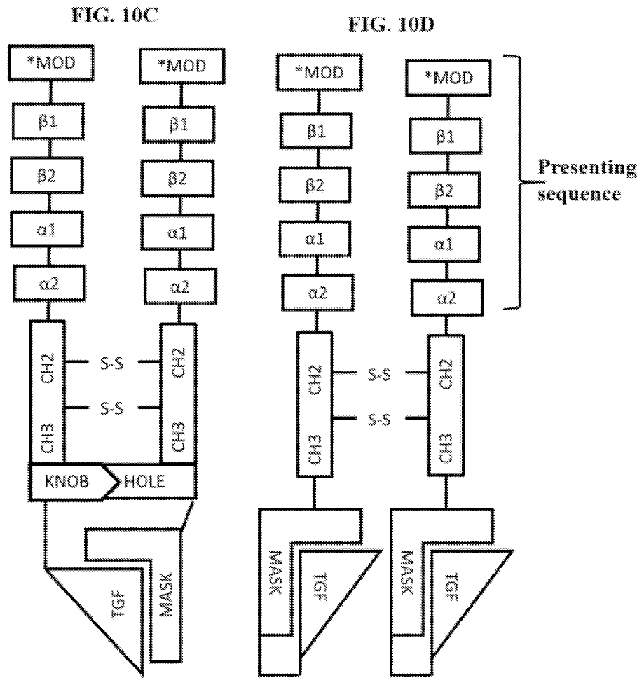

As discussed above, epitope presentation by a TMAPP to a target T cell is accomplished via a moiety that comprises MHC Class II polypeptides. Such moieties may be either (i) a single polypeptide chain, or (ii) a complex comprising two or more polypeptide chains. Where the MHC Class II polypeptides, and optionally one or more MODs are provided in a single polypeptide chain, it is termed a “presenting sequence” See, e.g., FIG. 9E to 9J. Presenting sequences may be integrated into a TMAPP to form a sc-TMAPP by direct or indirect (via a linker) linkage to a masked TGF-β MOD (see, e.g., FIG. 1 at (a) and (b)) or by direct or indirect linkage to a scaffold sequence (see, e.g., FIG. 1 at (c) and (d) and FIGS. 10 C and D).

As an alternative to utilizing a single polypeptide to present an epitope, the MHC components (e.g., α1, α2, β1, and β2 domain sequences) may be divided among two separate polypeptide sequences; (i) the presenting complex first sequence, and (ii) the presenting complex second sequence, which together are denoted herein as a “presenting complex.” See, e.g., FIGS. 9A to 9D. Presenting complex may be integrated into a TMAPP to form an m-TMAPP by direct or indirect (via a linker) linkage of the presenting complex first sequence to a masked TGF-β MOD (see, e.g., FIG. 1 at (a) and (b)), or by direct or indirect linkage to a scaffold sequence (see, e.g., FIG. 1 at (c) and (d) and FIGS. 10 A and B). The presenting complex first sequence and presenting complex second sequence generally associate through non-covalent interactions between the α chain and β chain polypeptide sequences and may be stabilized by disulfide bonds between either the MHC sequences (e.g., a disulfide bond between α and β chains), or between an MHC α or β chain amino acid and a peptide/polypeptide linkers attached to the MHC α or β chain (e.g., a linker at the N- or C-t terminus of the MHC sequences).

Where TMAPPs (including both sc-TMAPPs and m-TMAPPs) have been conjugated to an epitope presenting molecule that becomes bound at a chemical conjugation site of a TMAPP they are denoted as epitope conjugates (e.g., TMAPP-epitope conjugates, or more specifically, sc-TMAPP-epitope conjugates, or m-TMAPP-epitope conjugates). Where a TMAPP is conjugated to a payload at a chemical conjugation site it is denote as a TMAPP payload-conjugate.

The scaffold of a TMAPP has several function including, but not limited to, serving as a structure upon which to organize TMAPP components including the masked TGF-β MOD, the presenting sequences or complexes, and any additional MODs. In addition, the scaffold may contain sequences for organizing TMAPPs into higher order structures containing two or more TMAPPs as exemplified in FIG. 1 at (e) to (h). The interaction between TMAPPs in duplexes or other higher order TMAPP structures can be controlled by the use of interspecific binding sequence pairs that allow two different TMAPPs to be formed into a heterodimeric duplex. Where the masking sequence and masked TGF-β sequence of the masked TGF-β MOD are located in trans on different TMAPPs, interspecific sequences can be utilized to organize TMAPP structures and allow a functional masked TGF-β MOD to be formed (see e.g., FIG. 1 at (g) and (h). Scaffold sequences may also serve as the location for payload conjugation, and as a location for the incorporation of additional peptides.

While the TMAPP presenting sequences and presenting complexes comprise the MHC elements required for presenting an epitope to a TCR (e.g., α1, α2, β1, and β2 domain sequences), those elements may be ordered in more than one fashion. Exemplary organizations (from the N-terminus on the left to C terminus on the right) for presenting complexes are provided in FIG. 9A-9D, and presenting sequences are provided in FIGS. 9E to 9J. In those figures: the lines joining elements are optional linkers (e.g., polypeptide linkers). Exemplary locations where MODs, including masked TGF-β MODs with the mask and TGF-β sequences in cis, can be placed in presenting sequences and presenting complexes are indicated by *MOD. Although not shown in the figures, a masking sequence and a TGF-β sequence may be placed in trans at the N-termini of first and second peptides of the presenting complexes in any of FIGS. 9A to 9D, such placement results in the formation of a masked TGF-β MOD when presenting complex first and second sequence combine.

In addition to MODs being located in the presenting sequences or presenting complexes, MODs may be associated with the scaffold. Typically, a masked TGF-β MOD will occupy the carboxyl terminus of a TMAPP scaffold but other MODs may also be located there. For example, in duplex or higher order TMAPPs where at least one carboxyl terminus of a scaffold is not occupied by a MOD (e.g., by a masked TGF-β MOD with its elements in cis), an additional MOD (a MOD other than a masked TGF-β MOD) can be located the carboxyl terminus of one of the scaffolds. Where the duplex is formed with a pair of interspecific binding sequence in their scaffolds the carboxy termini of the scaffolds may be occupied by different MODs (e.g., a masked TGF-β MOD with its elements in cis on one scaffold, and an IL-2, PD-L1, or 4-1BBL on the other scaffold of the duplex).

Additional MODs (MODs other than a masked TGF-β MOD) may also be located on (linked to) the either the TGF-β sequence or the masking sequence of a masked TGF-β MOD regardless of whether those sequence are placed in cis or trans. Although the masking sequence is depicted as being N-terminal to the TGF-β sequence when placed in cis, the order may be reversed such that the TGF-β sequence is N-terminal relative to the masking sequence (e.g., the location of TGF-β sequence and the masking sequence are reversed in FIG. 1 at (a) to (f).

Linker sequences may be used between any of the elements of a TMAPP to provide appropriate spacing and positioning of those elements in the TMAPP and its higher order complexes. In some cases, the linker will be flexible, and in other instances the linker may be rigid.

As discussed in more detail below, chemical conjugation sites for the attachment of epitopes and payloads (e.g., solvent accessible cysteines that have been engineered into a peptide of a TMAPP) may be located on any of the elements of the TMAPP. Typically, chemical conjugation sites for the incorporation of epitope presenting molecules (e.g., peptide epitopes) will be located in one of the MHC α1, α2, β1, or β2 domain sequences or on a linker attached directly to one of the those domain sequences.

An unconjugated TMAPP of the present disclosure may comprise:
(i) a presenting sequence or a presenting complex,
(ii) optionally at least one scaffold polypeptide sequence, and
(iii) a TGF-β sequence, a masking sequence that binds to a TGF-β sequence reversibly masking it, or at least one masked TGF-β MOD;
wherein
   (a) each presenting sequence comprises MHC Class II α1, α2, β1, and β2 domain polypeptide sequences;
   (b) each presenting complex comprises a presenting complex first sequence and a presenting complex second sequence, wherein
   the presenting complex first sequence and presenting complex second sequence comprises at least one of the α1, α2, β1, and β2 polypeptide sequences, and
   the presenting complex first sequence and presenting complex second sequence together comprise the MHC Class II α1, α2, β1, and β2 domain polypeptide sequences, and (c) each masked TGF-β MOD comprises a masking sequence and TGF-β sequence;
wherein the unconjugated TMAPP optionally comprises one or more independently selected additional MODs (wt. and/or variant) or pairs of additional MODs (e.g., in tandem, both wt, both variant, or one wt. and one variant) (e.g., located at the N-terminus of a presenting sequence, presenting complex first and/or second sequences);
wherein a the unconjugated TMAPP comprises a chemical conjugation site for conjugation of an epitope presenting molecule, and optionally comprises an additional chemical conjugation site for the conjugation of a payload; and
wherein the unconjugated TMAPP optionally comprise one or more linker sequences that are selected independently. (see, e.g., FIGS. 1A to 1H and FIGS. 10A to 10D).
   The TMAPPs of the present disclosure may be subject to the proviso that the amino acid sequences of any component (e.g., MHC-Class II polypeptide sequences) do not include an amino acid sequence that will anchor the TMAPP in a mammalian cell (e.g., a COS cell) membrane (e.g., the TMAPP does not comprise an MHC transmembrane domain, or a portion thereof, that will anchor the TMAPP in a cell membrane). The above components may or may not be arranged in the stated order from N-terminus to C-terminus in the TMAPP.

Where such TMAPPs comprise a scaffold sequence that can multimerize (e.g., dimerize) two or more of such unconjugated TMAPPs may be complexed to for higher order complexes such as dimers. For example, a pair of such unconjugated dimers each comprising non-interspecific scaffold and a masked TGF-β MOD with its components in cis, may form a duplex as in FIG. 1 at (e) and (f). Where two TMAPPs comprise scaffolds with a pair of interspecific binding sequences, a duplex comprising two masked TGF-β MOD with the mask and TGF-β sequences in cis. Alternatively, where two TMAPPs comprise scaffolds with a pair of interspecific binding sequences with the masking and TGF-β sequence in trans a duplex unconjugated TMAPP as in FIG. 1 at (g) and (h).

While the term TMAPP(s) as used in the present disclosure refers to singular uncomplexed TMAPPs, the term TMAPP, and its plural TMAPPs, also refer to their higher order complexes comprising two or more singular uncomplexed TMAPPs in both their conjugated form and their conjugate forms. Where specific forms of higher order complexes are being referred to, e.g., duplexes of singular complexed TMAPPs, they are specified as duplex, triplex, etc. Accordingly, unless specified otherwise, where the term terms TMAPP or TMAPPs are used, the terms include unconjugated TMAPPs, TMAPP-epitope conjugates, and higher order complexes thereof, such as duplexes.

Following conjugation with an epitope presenting molecule, such as a peptide epitope of an auto antigen, any of the unconjugated TMAPPs or their higher order complexes described above may be converted to the corresponding TMAPP-epitope conjugates.

TMAPP-epitope, and accordingly their higher order complexes (duplexes, triplexes etc.), comprise MHC Class II polypeptide sequences, and their epitope conjugates (TMAPP-epitope conjugates) further comprise a conjugate epitope presenting molecules (e.g., a peptide epitope) for presentation to a TCR. Accordingly, they may present the epitope to T cells (e.g., CD4+ T cells) that have a TCR specific for the epitope. Once engaged with the TCR of a T cell, the effect of a TGF-β MOD-containing TMAPP-epitope conjugate on the T cell depends on the T cell's response to the TGF-β and any additional MODs (e.g., IL-2 MOD polypeptides) that are present as part of the TMAPP-epitope conjugate.

The masked TGF-β MOD-containing TMAPPs of the present disclosure can function as a means of producing TGF-β driven T cell responses. For example, TGF-β by itself can inhibit the development of effector cell functions of T cells, activate macrophages, and/or promote tissue the repair after local immune and inflammatory action subside. Accordingly, the TGF-β MOD-containing TMAPPs may be employed in vitro or in vivo, including as a therapeutic to induce any of those functions. TGF-β also regulates the differentiation of functional distinct subsets of T cell. TGF-β in the presence of IL-1 and/or IL-6 promotes the development of cells of the Th17 lineage, particularly in the absence of either IL-2 or an IL-2 agonist (e.g., an antibody binding to and acting as an agonist of the IL-2 receptor).

The TGF-β MOD-containing TMAPP-epitope conjugates, and particularly those comprising one or more IL-2 MODs (e.g., variant MODs) or co-administered with an IL-2 or an IL-2 agonist, can bring about the induction and/or proliferation and/or maintenance (survival) of CD4⁺ FOXP3⁺ T reg cells specific/selective for the epitope presented by the TMAPP. Contacting T cells with a combination of a TMAPP-epitope conjugate and IL-2 (either as an IL-2 MOD, an IL-2R agonist or IL-2) in vitro or in vivo inhibits effector T helper (Th) cell differentiation into cells of the Th1, Th2, and/or Th17 lineages. Accordingly, the masked TGF-β MOD-containing TMAPP-epitope conjugates (e.g., those bearing an IL-2 MOD) are capable of suppressing the immune response to the TMAPP-epitope conjugate-included epitope through, for example, the induction, proliferation, and/or maintenance of T reg cells induced/produced in response to the TMAPP-epitope conjugates, and any downstream effects of those T reg cells, including suppression of CD8+ T cells (activity and/or proliferation) and/or suppression B cells (e.g., antibody production and/or proliferation). TMAPP-epitope conjugates (e.g., their duplexes) therefore may provide methods of suppressing T cell and B cell activity in vitro and in vivo, and the use of TMAPP-epitope conjugates (e.g., duplex of TMAPP-epitope conjugates) as therapeutics for in vivo or in vitro methods of treatment. Thus, the present disclosure provides methods of modulating activity of T cells and/or B cells in vitro and in vivo, in disorders related to immune dysregulation/disfunction including allergies and autoimmune diseases, as well as metabolic disorders. The TMAPP-epitope conjugates also find use in the prophylaxis and/or treatment of graft rejection, in the context of either host vs graft rejection/disease (“HVGD”) or graft vs host rejection/disease (“GVHD”).

In addition to the foregoing, TMAPPs, can function as a means of selectively delivering the MODs, including masked TGF-β MODs, to T cells with a TCR specific for the epitope conjugated to and presented by a TMAPP-epitope conjugate, thereby resulting in MOD-driven responses to those TMAPPs (e.g., the reduction in number and/or suppression of CD4+ effector T cells reactive with TMAPP-associated epitopes). Depending on the chosen MOD, the incorporation of one or more MODs with increased affinity for their cognate receptor on T cells (“co-MOD”) may reduce the specificity of TMAPP-epitope conjugates and duplex TMAPP-epitope conjugates for epitope specific T cells where MOD-co-MOD binding interactions significantly compete with MHC/epitope binding to target cell TCR. Conversely, and again depending on the chosen MOD, the inclusion of MODs with reduced affinity for their co-MOD(s), and the affinity of the epitope for a TCR, MOD selection may provide for enhanced selectivity of TMAPP-epitope conjugates and duplex TMAPP-epitope conjugates, while retaining the desired activity of the MODs. Where a MOD already possesses a relatively low affinity for its cognate receptor, mutations that reduce the affinity may be unnecessary and/or undesirable for their incorporation into a TMAPP.

The ability of TMAPP-epitope conjugates (e.g., as duplexes) to modulate T cells provides methods of modulating T cell activity in vitro and in vivo, and accordingly TMAPP-epitope conjugates are useful as therapeutics in methods of treating a variety of diseases and conditions including autoimmune diseases, GVHD, HVGD, and allergies, as well as metabolic disorders.

The present disclosure provides nucleic acids comprising nucleotide sequences encoding individual TMAPP polypeptides and TMAPPs (e.g., all polypeptides of a TMAPP), as well as cells genetically modified with the nucleic acids and vectors for and producing TMAPP polypeptides and/or TMAPP proteins (e.g., duplex TMAPPs). The present disclosure also provides methods of producing TMAPPs, duplex TMAPPs, and higher order TMAPPs utilizing such cells. The disclosure also includes and provides for methods of conjugating epitopes and payload molecules to chemical conditions sites of unconjugated TMAPPs forming their TMAPP-epitope conjugates, TMAPP-payload conjugates, and where both are conjugated to an unconjugated TMAPP, TMAPP-epitope and payload conjugates.

### V.A(i). Class II MHC Polypeptides

As noted above, TMAPPs may include MHC Class II polypeptides of various species, including human MHC polypeptides (HLA polypeptides), rodent (e.g., mouse, rat, etc.) MHC polypeptides, and MHC polypeptides' of other mammalian species (e.g., lagomorphs, non-human primates, canines, felines, ungulates (e.g., equines, bovines, ovines, caprines, etc.)), and the like. For the purpose of this disclosure the term “MHC polypeptide” is meant to include Class II MHC polypeptides, including the α- and β-chains or portions thereof. More specifically, MHC Class II polypeptides include the α1 and α2 domains of Class II MHC α chains, and the β1 and β2 domains of Class II MHC β chains, which represent all or most of the extracellular Class II protein required for presentation of an epitope to a TCR. In an embodiment, both the α and β Class II MHC polypeptide sequences in a TMAPP are of human origin.

TMAPPs (e.g., duplex TMAPPs) are intended to be soluble in aqueous media under physiological conditions (e.g., soluble in human blood plasma at therapeutic levels). Unless expressly stated otherwise, as noted above, the TMAPPs described herein are not intended to include membrane anchoring domains (such as transmembrane regions of MHC Class II α and β chains) or a part thereof sufficient to anchor TMAPP molecules (e.g., more than 50% of the TMAPP molecules), or a peptide thereof, in the membrane of a cell (e.g., a eukaryotic cell such as a mammalian cell, for example, a Chinese Hamster Ovary or “CHO” cell) in which the TMAPP is expressed. Similarly, unless expressly stated otherwise, the TMAPPs described herein do not include the leader and/or intracellular portions (e.g., cytoplasmic tails) that may be present in some naturally-occurring MHC Class II proteins or other components of a TMAPP such as the scaffold.

### 1. T1D and its Risk-Associated Alleles and Haplotypes

Certain alleles and haplotypes of MHC Class II have been associated with disease, e.g., increased risk of developing T1D. See, e.g., Erlich et al. (2008) Diabetes 57:1084; Gough and Simmonds (2007) Curr. Genomics 8:453; Mitchell et al. (2007) Robbins Basic Pathology Philadelphia: Saunders, 8th ed.; Margaritte-Jeannin et al. (2004) Tissue Antigens 63:562; and Kurko et al. (2013) Clin. Rev. Allergy Immunol. 45:170.

a. MHC Class II Polypeptides in Type 1 Diabetes Mellitus (T1D)

Alleles/isoforms showing increased association with T1D represent suitable sources of MHC II α1, α2, β1, and β2 polypeptide sequences for incorporation into TMAPPs directed to the treatment of T1D. T1D is associated with alleles belonging to the HLA-DR3 and HLA-DR4 haplotypes/serotypes, with the strongest risk associated with the HLA-DQ8, (e.g., HLA-DQB1*03:02) and alleles of the HLA-DQ2 serotype. Some high and moderate risk haplotypes and their association with various DR serotypes are shown in Table 2 adopted from Kantárová and Buc, Physiol. Res. 56: 255-266 (2007).

TABLE 2

| | | | | |
|---|---|---|---|---|
| DR serotype | DRB allele | DQ serotype | DQA allele | DQB allele |

High risk T1D haplotypes

| | | | | |
|---|---|---|---|---|
| DR3 | DRB1*0301 | DQ 2.5 | DQA1*0501 | DQB1*0201 |
| DR4 | DRB1*0401 | DQ 8.1 | DQA1*0301 | DQB1*0302 |
| DR4 | DRB1*0402 | DQ 8.1 | DQA1*0301 | DQB1*0302 |
| DR4 | DRB1*0405 | DQ 8.1 | DQA1*0301 | DQB1*0302 |

Moderate risk T1D haplotypes

| | | | | |
|---|---|---|---|---|
| DR1 | DRB1*01 | DQ 5 | DQA1*0101 | DQB1*0501 |
| DR8 | DRB1*0801 | | DQA1*0401 | DQB1*0402 |
| DR9 | DRB1*0901 | | DQA1*0301 | DQB1*0303 |

The serotypically defined DR3 and DR4 protein isoforms/haplotypes of the DRB1 gene are associated with increased risk that an individual expressing such alleles will develop T1D. The DR3 serotype includes the alleles encoding the DRB1*03:01, *03:02, *03:03, and *03:04 proteins, with the HLA-DRB1*0301 allele often found associated with a predisposition to T1D. The DR4 serotype includes the alleles encoding the DRB1*04:01, *04:02, *04:03, *04:04, *04:05, *04:06, *04:07, *04:08, *04:09, *04:10, *04:11, *04:12, and *04:13 proteins. Certain HLA-DR4 (e.g., HLA-DRB1*0401 and HLA-DRB1*0405) predispose individuals to T1D, whereas HLA-DRB1*04:03 allele/isoform may afford protection. DRB1*16:01 also show an increased frequency in diabetic children relative to healthy controls (Deja, et al., Mediators of Inflammation 2006:1-7 (2006)). Alleles/isoforms showing increased association with T1D represent suitable sources of MHC II α1, α2, β1, and β2 polypeptide sequences. The above-mentioned alleles associated with an increased risk of T1D represent suitable candidates from which the α1, α2, β1, and/or β2 polypeptide sequences present in a TMAPP may be taken.

### 2. MHC Class II α and β Polypeptides

TMAPPs of the present disclosure comprise Class II MHC polypeptides. Naturally occurring Class II MHC polypeptides comprise an α chain and a β chain (e.g., HLA α- and β-chains). While MHC Class II polypeptides include MHC Class II DP α and β polypeptides, DM α and β polypeptides, DO α and β polypeptides, DQ α and β polypeptides, and DR α and β polypeptides. The polypeptides of central interest for the treatment of T1D are DQ α and β polypeptides and DR α and β polypeptides. As used herein, the term “Class II MHC polypeptide” refers to a Class II MHC α chain polypeptide, a Class II MHC β chain polypeptide, or only a portion of a Class II MHC α and/or β chain polypeptide, or combinations of the foregoing. For example, the term “Class II MHC polypeptide” as used herein can be a polypeptide that includes: i) only the α1 domain of a Class II MHC α chain; ii) only the α2 domain of a Class II MHC α chain; iii) only the α1 domain and an α2 domain of a Class II MHC α chain; iv) only the β1 domain of a Class II MHC D chain; v) only the β2 domain of a Class II MHC β chain; vi) only the β1 domain and the β2 domain of a Class II MHC β chain; vii) the α1 domain of a Class II MHC α chain, the β1 domain of a Class II MHC D chain, and the β2 domain of a Class II MHC; and the like.

The human MHC or HLA locus is highly polymorphic in nature, and thus as used herein, the term “Class II MHC polypeptide” includes allelic forms of any known Class II MHC polypeptide. See, e.g., the HLA Nomenclature site run by the Anthony Nolan Research Institute, available on the world wide web at hla.alleles.org/nomenclature/index.html, which indicates that there are numerous DRA alleles, DRB1 alleles, DRB3 alleles, DRB4 alleles, DRB5 alleles, DRB6 alleles, DRB7 alleles, DRB9 alleles, DQA1 alleles, DQB1 alleles, DPA1, DPB1 alleles, DMA alleles, DMB alleles, DOA alleles and DOB alleles.

In some cases, a TMAPP comprises a Class II MHC α chain, without the leader, transmembrane, and intracellular portions (e.g., cytoplasmic tails) that may be present in a naturally-occurring Class II MHC α chain. Thus, in some cases, a TMAPP comprises only the α1 and α2 portions of a Class II MHC α chain; and does not include the leader, transmembrane, and intracellular portions (e.g., cytoplasmic tails) that may be present in a naturally-occurring Class II MHC α chain.

In some cases, a TMAPP comprises a Class II MHC β chain, without the leader, transmembrane, and intracellular portions (e.g., cytoplasmic tails) that may be present in a naturally-occurring Class II MHC β chain. Thus, in some cases, a TMAPP comprises only the β1 and β2 portions of a Class II MHC β chain; and does not include the leader, transmembrane, and intracellular portions (e.g., cytoplasmic tails) that may be present in a naturally-occurring Class II MHC β chain.

a. MHC Class II Alpha Chains

MHC Class II alpha chains comprise an α1 domain and an α2 domain. In some cases, the α1 and α2 domains present in an antigen-presenting cell are from the same MHC Class II α chain polypeptide. In some cases, the α1 and α2 domains present in an antigen-presenting cell are from two different MHC Class II α chain polypeptides.

MHC Class II alpha chains suitable for inclusion in a presenting sequence or complex of a TMAPP may lack a signal peptide. An MHC Class II alpha chain suitable for inclusion in a TMAPP can have a length of from about 60 aas to about 200 aas; for example, an MHC Class II alpha chain suitable for inclusion in a TMAPP can have a length of from about from about 60 amino acids to about 80 amino acids, 80 aas to about 100 aas, from about 100 aas to about 140 aas, from about 140 aas to about 170 aas, from about 170 aas to about 200 aas. An MHC Class II α1 domain suitable for inclusion in a TMAPP can have a length of from about 30 aas to about 95 aas; for example, an MHC Class II al domain suitable for inclusion in a TMAPP can have a length of from about 30 aas to about 50 aas, from about 50 aas to about 70 aas, or from about 70 aas to about 95 aas. In an embodiment an MHC Class II α1 domain of a TMAPP is from about 70 aas to about 95 aas. An MHC Class II α2 domain suitable for inclusion in a TMAPP can have a length of from about 30 aas to about 95 aas; for example, an MHC Class II α2 domain suitable for inclusion in a TMAPP can have a length of from about 30 aas to about 50 aas, from about 50 aas to about 70 aas, or from about 70 aas to about 95 aas. In an embodiment, an MHC Class II α2 domain of a TMAPP is from about 70 aas to about 95 aas.

### (i) DRA Polypeptides

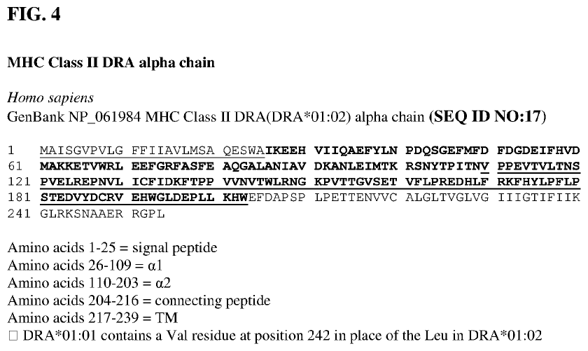

A suitable MHC Class II DRA polypeptide for inclusion in a TMAPP may have at least 60%, at least 70%, at least 80%, at least 90%, at least 95%, at least 98%, at least 99%, or 100% aa sequence identity with at least 150, at least 160, or at least 170 contiguous amino acids of the aa sequence from aa 26 to aa 203 (the α1 and α2 domain region) of the DRA au sequence depicted in FIG. 4 or a-naturally occurring allelic variant thereof. In some cases, the DRA polypeptide has a length of about 178 aas (e.g., 175, 176, 177, 178, 179, or 180 aas).

As used herein, the term “DRA polypeptide” includes allelic variants, e.g., naturally occurring allelic variants. Thus, in some cases, a suitable DRA polypeptide comprises aas 26-203 of DRA*01:02:01 (see FIG. 4), or an allelic variant thereof. In some cases, the allelic variant is the DRA*01:01 polypeptide (e.g., from the DRA*01:01:01:01 allele) that differs from DRA*01:02 by having a valine in place of the leucine at position 242 (see FIG. 4).

A suitable DRA for inclusion in a TMAPP polypeptide can have at least 70%, at least 80%, at least 90%, at least 95%, at least 98%, at least 99%, or 100%, aa sequence identity with at least 160, at least 170, or at least 180 contiguous aas of the sequence from aa 26 to aa 216 of the DRA*01:02 sequence depicted in FIG. 4. A “DRA polypeptide” includes allelic variants, e.g., naturally occurring allelic variants.

Thus, in some cases, a suitable DRA polypeptide comprises the following amino acid sequence: IKEEH VIIQAEFYLN PDQSGEFMFD FDGDEIFHVD MAKKETVWRL EEFGRFASFE AQGALANIAV DKANLEIMTK RSNYTPITNV PPEVTVLTNS PVELREPNVL ICFIDKFTPP VVNVTWLRNG KPVTTGVSET VFLPREDHLF RKFHYLPFLP STEDVYDCRV EHWGLDEPLL KHW (SEQ ID NO:63, amino acids 26-203 of DRA*01:02, see FIG. 4), or an allelic variant thereof. In some cases, the allelic variant is the DRA*01:01 allelic variant that differs from DRA*01:02 polypeptide by having a valine in place of the leucine at position 242 of the sequence in FIG. 4. In some cases, a DRA polypeptide suitable for inclusion in a TMAPP comprises an amino acid substitution, relative to a wild-type DRA polypeptide, where the amino acid substitution replaces an amino acid (other than a Cys) with a Cys (e.g., for forming a disulfide bond that stabilizes the TMAPP).

In some cases, a TMAPP comprises a variant DRA polypeptide that comprises a non-naturally occurring Cys residue (e.g., for forming a disulfide bond that stabilizes the TMAPP). For example, in some cases, a TMAPP comprises a variant DRA polypeptide that comprises at least one aa substitution selected from E3C, E4C, F12C, G28C, D29C, I72C, K75C, T80C, P81C, I82C, T93C, N94C, and S95C (see, e.g., FIG. 4 SEQ ID NO: 17).

A suitable DRA α1 domain for inclusion in a TMAPP polypeptide, including naturally occurring allelic variants thereof, may comprise an aa sequence having at least 70%, at least 80%, at least 90%, at least 95%, at least 98%, at least 99%, or 100% aa sequence identity to the following aa sequence: VIIQAEFYLN PDQSGEFMFD FDGDEIFHVD MAKKETVWRL EEFGRFASFE AQGALANIAV DKANLEIMTK RSNYTPITN (SEQ ID NO:64); and can have a length of about 84 aas (e.g., 80, 81, 82, 83, 84, 85, or 86 aas).

A suitable DRA α2 domain for inclusion in a TMAPP polypeptide, including naturally occurring allelic variants thereof, may comprise an aa sequence having at least 70%, at least 80%, at least 90%, at least 95%, at least 98%, at least 99%, or 100% aa sequence identity to the following aa sequence: V PPEVTVLTNSPVELREPNVL ICFIDKFTPP VVNVTWLRNG KPVTTGVSET VFLPREDHLF RKFHYLPFLP STEDVYDCRV EHWGLDEPLL KHW (SEQ ID NO:65); and can have a length of about 94 aas (e.g., 90, 91, 92, 93, 94, 95, 96, 97, or 98 aas).

### (ii) DQA Polypeptides

A suitable MHC Class II α DQA polypeptide for inclusion in a TMAPP may have at least 60%, at least 70%, at least 80%, at least 90%, at least 95%, at least 98%, at least 99%, or 100% aa sequence identity with the α1, α2, β1, and/or the β2 domain(s) of DQA1*0101, DQA1*0301, or DQA1*0401. In some cases, the DQA polypeptide has a length of about 178 aas (e.g., 175, 176, 177, 178, 179, or 180 aas). The sequences of those HLA polypeptides are available, for example on the world wide web at hla.alleles.org/nomenclature/index.html, which is run by the Anthony Nolan Research Institute, and at www.ncbi.nlm.nih.gov (National Center for Biotechnical Information or “NCBI”) operated by the U.S. National library of Medicine.

b. MHC Class II Beta Chains

MHC Class II beta chains comprise a β1 domain and a β2 domain. In some cases, the β1 and β2 domains present in an antigen-presenting cell are from the same MHC Class II β chain polypeptide. In some cases, the β1 and β2 domains present in an antigen-presenting cell are from two different MHC Class II β chain polypeptides.

MHC Class II beta chains suitable for inclusion in a TMAPP (e.g., a higher order TMAPP construct such as a duplex TMAPP) lack a signal peptide. An MHC Class II beta chain suitable for inclusion in a TMAPP can have a length of from about 60 aas to about 210 aas; for example, an MHC Class II beta chain suitable for inclusion in a TMAPP can have a length of from about 60 aas to about 90 aas, from about 90 aas to about 120 aas, from about 120 aas to about 150 aas, from about 150 aas to about 180 aas, from about 180 aas to 210 aas. An MHC Class II β1 domain suitable for inclusion in a TMAPP can have a length of from about 30 aas to about 105 aas; for example, an MHC Class H1 β1 domain suitable for inclusion in a TMAPP can have a length of from about 30 aas to about 50 aas, from about 50 aas to about 70 aas, from about 70 aas to about 90 aas, from about 90 aas to about 105 aas. An MHC Class II β2 domain suitable for inclusion in a TMAPP can have a length of from about 30 aas to about 105 aas; for example, an MHC Class H1 β2 domain suitable for inclusion in a TMAPP can have a length of from about 30 aas to about 50 aas, from about 50 aas to about 70 aas, from about 70 aas to about 90 aas, from about 90 aas to about 105 aas.

An MHC Class H1 β chain polypeptide suitable for inclusion in a TMAPP may comprise an aa substitution, relative to a wild-type MHC Class H1 § chain polypeptide, where the aa substitution replaces an aa (other than a Cys) with a Cys (e.g., for forming a disulfide bond that stabilizes the TMAPP). For example, in some cases, the MHC Class H1 β chain polypeptide is a variant DRB1 MHC Class II polypeptide that comprises an aa substitution selected from the group consisting of P5C, F7C, Q10C, N19C, G20C, H33C, G151C, D152C, and W153C. In some cases, the MHC Class II β chain polypeptide is a variant DRB1 polypeptide comprising an aa sequence having at least 60%, at least 70%, at least 80%, at least 90%, at least 95%, at least 98%, or at least 99%, aa sequence identity to the following mature DRB1 aa sequence lacking the signal peptide: GDTRPRFLEQVKHECHFFNGTERVRFLDRYFYHQEEYVRFDSDVGEYRAVTELGRPDAEYWNS QKDLLEQKRAAVDTYCRHNYGVGESFTVQRRVYPEVTVYPAKTQPLQHHNLLVCSVNGFYPA SIEVRWFRNGQEEKTGVVSTGLIQNGDWTFQTLVMLETVPRSGEVYTCQVEHPSLTSPLTVEW R ARSESAQSKM (SEQ ID NO:66), and comprising an cysteine substitution at one or more (e.g., two or more) aas selected from the group consisting of P5C, F/C, Q10C, N19C, G20C, H33C, G151C, D152C, and W153C. In some cases, the MHC Class II β chain polypeptide is a variant of a mature DRB3 polypeptide, mature DRB4 polypeptide, or mature DRB5 polypeptide (lacking their signal sequences) comprising a cysteine substitution at one or more (e.g., two or more) of positions 5, 7, 10, 19, 20, 33, 151, 152, and 153 (e.g., P5C, F7C, Q10C, N19C, G20C, N33C, G151C, D152C, and/or W153C substitutions).

### (i) DRB Polypeptides

### (a) DRB1 Polypeptides

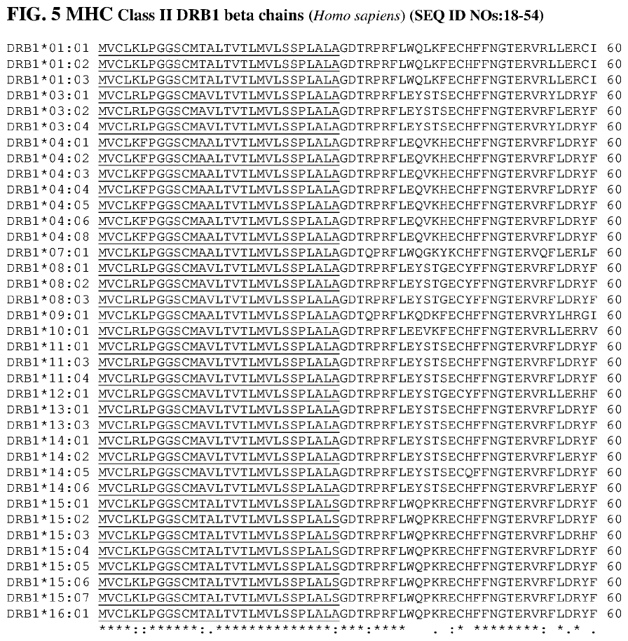

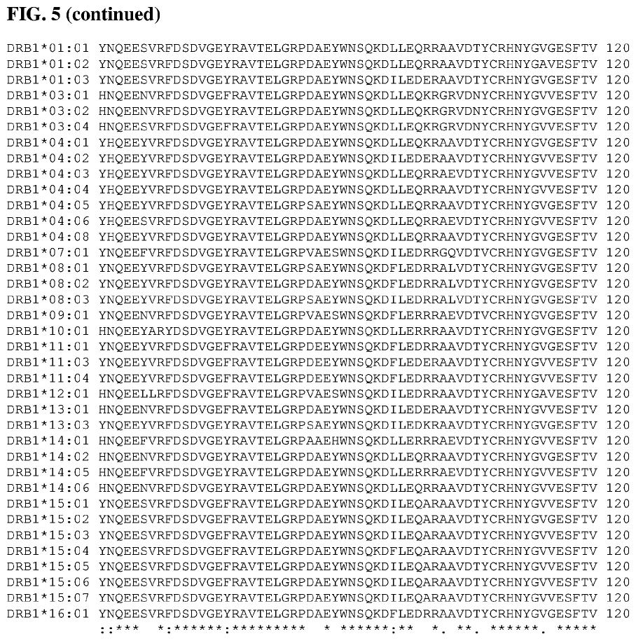

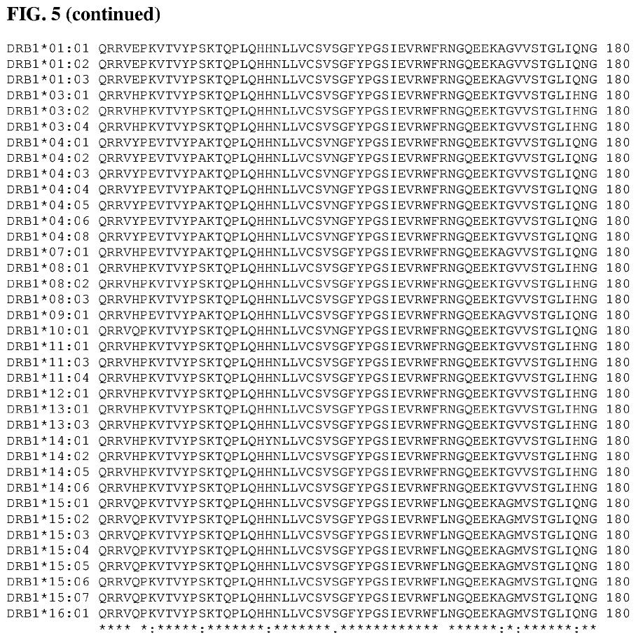

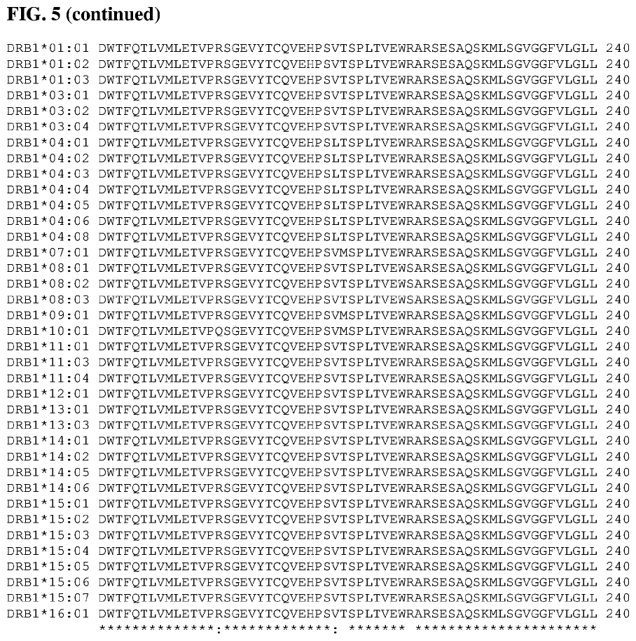

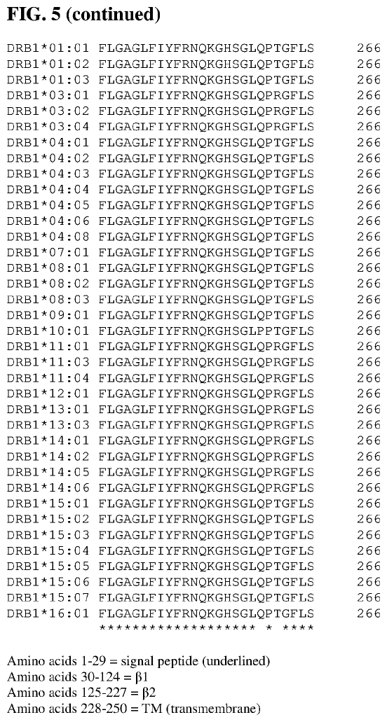

In some cases, a suitable MHC Class II β chain polypeptide is a DRB1 polypeptide. In an embodiment, a DRB1 polypeptide can have at least 60%, at least 70%, at least 80%, at least 90%, at least 95%, at least 98%, at least 99%, or 100%, aa sequence identity with at least 170, at least 180, or at least 190, contiguous aas of the sequence from aa 30 to aa 227 of any DRB1 aa sequence depicted in FIG. 5, including naturally occurring allelic variants. FIG. 5 displays the DRB1 precursor proteins in which aas 1-29 are the signal sequence (underlined), 30-124 the β1 region (bolded), 125-227 the β2 region (bolded and underlined), and 228-250 the transmembrane region. In some cases, a DRB1 polypeptide suitable for inclusion in a TMAPP comprises an aa substitution, relative to a wild-type DRB1 polypeptide, where the aa substitution replaces an an (other than a Cys) with a Cys.

A suitable MHC Class II β chain polypeptide suitable for incorporation into a TMAPP may be a DRB1 polypeptide, wherein the DRB1 polypeptide has at least 60%, at least 70%, at least 80%, at least 90%, at least 95%, at least 98%, at least 99%, or 100% an sequence identity with at least 170, at least 180, or at least 190, contiguous aas of the sequence from aa 30 to aa 227 (the β1 and β2 domain region) of a DRB1 sequence provided in FIG. 5, including one of the following DRB1 polypeptides:
(i) the DRB1-1 (DRB1*01:01) beta chain an sequence Swiss-Prot/UniProt reference (“sp”) P04229.2 in FIG. 5;
(ii) the DRB1-3 (DRB1*03:01) beta chain an sequence sp P01912.2 in FIG. 5;
(iii) the DRB1-4 (DRB1*04:01) beta chain an sequence sp P13760.1 in FIG. 5;
(iv) the DRB1-7 (DRB1*07:01) beta chain an sequence sp P13761.1 in FIG. 5;
(v) the DRB1-8 (DRB1*08:01) beta chain an sequence sp Q30134.2 in FIG. 5;
(vi) the DRB1-9 (DRB1*09:01) beta chain an sequence sp Q9TQE0.1 in FIG. 5;
(vii) the DRB1-10 (DRB1*10:01) beta chain an sequence sp Q30167.2 in FIG. 5;
(viii) the DRB1-11 (DRB1*11:01) beta chain an sequence sp P20039.1 in FIG. 5;
(ix) the DRB1-12 (DRB1*12:01) beta chain an sequence sp Q95IE3.1 in FIG. 5;
(x) the DRB1-13 (DRB1*13:01) beta chain an sequence sp Q5Y7A7.1 in FIG. 5;
(xi) the DRB1-14 (DRB1*14:01) beta chain an sequence sp Q9GIY3.1 in FIG. 5;
(xii) the DRB1-15 (DRB1*15:01) beta chain an sequence sp P01911 in FIG. 5; and
(xiii) the DRB1-16 (DRB1*16:01) beta chain an sequence sp Q29974.1 in FIG. 5.

As use herein “DRB1 polypeptide” includes allelic variants, e.g., naturally occurring allelic variants. Thus, in some cases, a suitable DRB1 polypeptide comprises aas 31-227 of DRB1*04:01 (DRB14) provided in FIG. 5 (SEQ ID NO:24) or an allelic variant thereof. Another suitable DRB1 polypeptide may comprise a sequence having at least 70%, at least 80%, at least 90%, at least 95%, at least 98%, at least 99%, or 100% aa sequence identity to at least 170, at least 180, or at least 190 contiguous aas of the following DRB1*04:01 an sequence: GDTRPRFLEQVKHECHFFNGTERVRFLDRYFYHQEEYVRFDSDVGEYRAVTELGRPDAEYWNS QKDLLEQKRAAVDTYCRHNYGVGESFTVQRRVYPEVTVYPAKTQPLQHHNLLVCSVNGFYPA SIEVRWFRNGQEEKTGVVSTGLIQNGDWTFQTLVMLETVPRSGEVYTCQVEHPSLTSPLTVEW RARSESAQSKM (SEQ ID NO:67), which may bear one or more cysteine substitutions. In an embodiment the cysteine substitution is a P5C substitution. In an embodiment the cysteine substitution is a G151C substitution. In an embodiment the cysteine substitution is a W153C substitution.

A suitable DRB1 β1 domain, including-naturally occurring allelic variants thereof, may comprise an aa sequence having at least 70%, at least 80%, at least 90%, at least 95%, at least 98%, at least 99%, or 100% an sequence identity to the following an sequence: DTRPRFLEQVKHECHFFNGTERVRFLDRYFYHQEEYVRFDSDVGEYRAVTELGRPDAEYWNSQ KDLLEQKRAAVDTYCRHNYGVGESFTVQRRV (SEQ ID NO:68); and can have a length of about 95 aas (including, e.g., 92, 93, 94, 95, 96, 97, or 98 aas). A suitable DRB1 β1 domain can comprise the following amino acid sequence: GDTRCRFLEQVKHECHFFNGTERVRFLDRYFYHQEEYVRFDSDVGEYRAVTELGRPDAEYWN SQKDLLEQKRAAVDTYCRHNYGVGESFTVQRRV (SEQ ID NO:69), where P5 is substituted with a Cys (shown in bold and italics text).

A suitable DRB1 β2 domain, including-naturally occurring allelic variants thereof, may comprise an aa sequence having at least 70%, at least 80%, at least 90%, at least 95%, at least 98%, at least 99%, or 100% aa sequence identity to the following aa sequence: YPEVTVYPAKTQPLQHHNLLVCSVNGFYPGSIEVRWFRNGQEEKTGVVSTGLIQNGDWTFQTL VMLETVPRSGEVYTCQVEHPSLTSPLTVEWRARSESAQSK (SEQ ID NO:70); and can have a length of about 103 aas, including, e.g., 100, 101, 102, 103, 104, 105, or 106 aas.

A suitable DRB1 β2 domain can comprise the following amino acid sequence: YPEVTVYPAKTQPLQHHNLLVCSVNGFYPASIEVRWFRNGQEEKTGVVSTGLIQNGDCTFQTL V MLETVPRSGEVYTCQVEHPSLTSPLTVEWRARSESAQSKM (SEQ ID NO:71), where W153 is substituted with a Cys (shown in bold and italics text).

### (b) DRB3 Polypeptides

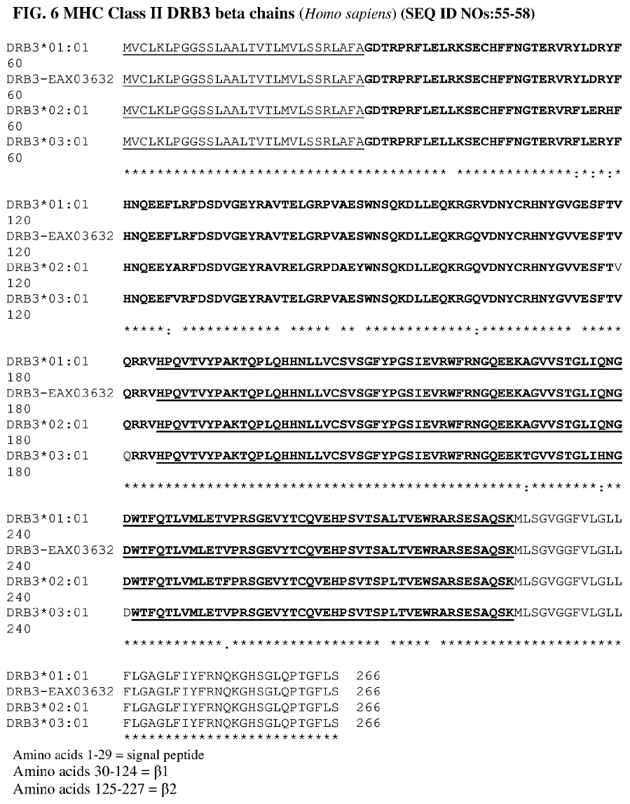

In some cases, a suitable MHC Class II β chain polypeptide is a DRB3 polypeptide. In an embodiment, a DRB3 polypeptide can have at least 60%, at least 70%, at least 80%, at least 90%, at least 95%, at least 98%, at least 99%, or 100% aa sequence identity with aas 30-227 of any DRB3 aa sequence depicted in FIG. 6, which displays the DRB3 precursor proteins in which aas 1-29 are the signal sequence (underlined), 30-124 form the β1 region (shown bolded), 125-227 form the β2 region, and 228-250, the transmembrane region. A DRB3 β chain polypeptide suitable for incorporation into a TMAPP may have at least 60%, at least 70%, at least 80%, at least 90%, at least 95%, at least 98%, at least 99%, or 100% aa sequence identity with aas 30-227 (the β1 and β2 domain region) of one of the following DRB3 polypeptides:
(i) the DRB1-3 (DRB3*01:01) beta chain aa sequence GenBank NP_072049.1 in FIG. 6;
(ii) the DRB1-3 beta chain an sequence in GenBank accession EAX03632.1 in FIG. 6;
(iii) the DRB1-3 (DRB3*02:01) beta chain aa sequence GenBank CAA23781.1 in FIG. 6; and
(iv) the DRB1-3 (DRB3*03:01) beta chain aa sequence GenBank AAN15205.1 in FIG. 6.

A DRB3 polypeptide suitable for inclusion in a TMAPP may comprise an aa substitution, relative to a wild-type DRB3 polypeptide, where the aa substitution replaces an aa (other than a Cys) with a Cys (e.g., for forming a disulfide bond that stabilizes the TMAPP).

As used herein, the term “DRB3 polypeptide” includes allelic variants, e.g., naturally occurring allelic variants. Thus, in some cases, a suitable DRB3 polypeptide comprises aas 30 to 227 of DRB3*01:01 provided in FIG. 6 (SEQ ID NO:55), or an allelic variant thereof. Thus, in some cases, a suitable DRB3 polypeptide comprises a sequence having at least 80%, at least 90%, at least 95%, at least 98%, at least 99%, or 100% sequence identity to at least 170, at least 180, or at least 190 contiguous aas of the following sequence: DTRPRFLELR KSECHFFNGT ERVRYLDRYF HNQEEFLRFD SDVGEYRAVT ELGRPVAESW NSQKDLLEQK RGRVDNYCRH NYGVGESFTV QRRVHPQVTV YPAKTQPLQH HNLLVCSVSG FYPGSIEVRW FRNGQEEKAG VVSTGLIQNG DWTFQTLVML ETVPRSGEVY TCQVEHPSVT SALTVEWRAR SESAQSK (SEQ ID NO:72), or an allelic variant thereof. In some cases, a DRB3 polypeptide suitable for inclusion in a TMAPP comprises an aa substitution, relative to a wild-type DRB3 polypeptide, where the aa substitution replaces an as (other than a Cys) with a Cys. Thus, e.g., in some cases, the MHC Class II β chain polypeptide is a variant DRB3 MHC Class II polypeptide that comprises a non-naturally occurring Cys at an aa selected from the group consisting of P5C, F/C, L10C, N19C, G20C, N33C, G151C, D152C, and W153C (of a mature DRB3 polypeptide (lacking the N-terminal signal peptide MVCLKLPGGSSLAALTVTLMVLSSRLAFA (SEQ ID NO:73) depicted in FIG. 6).

A suitable DRB3 β1 domain, including-naturally occurring allelic variants thereof, may comprise an aa sequence having at least 70%, at least 80%, at least 90%, at least 95%, at least 98%, at least 99%, or 100% aa sequence identity to the following an sequence: DTRPRFLELR KSECHFFNGT ERVRYLDRYF HNQEEFLRFD SDVGEYRAVT ELGRPVAESW NSQKDLLEQK RGRVDNYCRH NYGVGESFTV QRRV (SEQ ID NO:74); and can have a length of about 95 aas (e.g., 93, 94, 95, 96, 97, or 98 aas). A suitable DRB3 β1 domain can comprise the following an sequence: DTRPRFLELR KSECHFFNGT ERVRYLDRYF HNQEEFLRFD SDVGEYRAVT ELGRPVAESW NSQKDLLEQK RGRVDNYCRH NYGVGESFTV QRRV (SEQ ID NO:74), or a naturally-occurring allelic variant A suitable DRB3 β2 domain, including-naturally occurring allelic variants thereof, may comprise an aa sequence having at least 70%, at least 80%, at least 90%, at least 95%, at least 98%, at least 99%, or 100% an sequence identity to the following an sequence: HPQVTV YPAKTQPLQH HNLLVCSVSG FYPGSIEVRW FRNGQEEKAG VVSTGLIQNG DWTFQTLVML ETVPRSGEVY TCQVEHPSVT SALTVEWRAR SESAQSK (SEQ ID NO:75); and can have a length of about 103 aas (e.g., 100, 101, 102, 103, 104, or 105 aas). A suitable DRB3 β2 domain can comprise the following aa sequence: HPQVTV YPAKTQPLQH HNLLVCSVSG FYPGSIEVRW FRNGQEEKAG VVSTGLIQNG DWTFQTLVML ETVPRSGEVY TCQVEHPSVT SALTVEWRAR SESAQSK (SEQ ID NO:75), or a naturally-occurring allelic variant thereof.

### (c) DRB4 Polypeptides

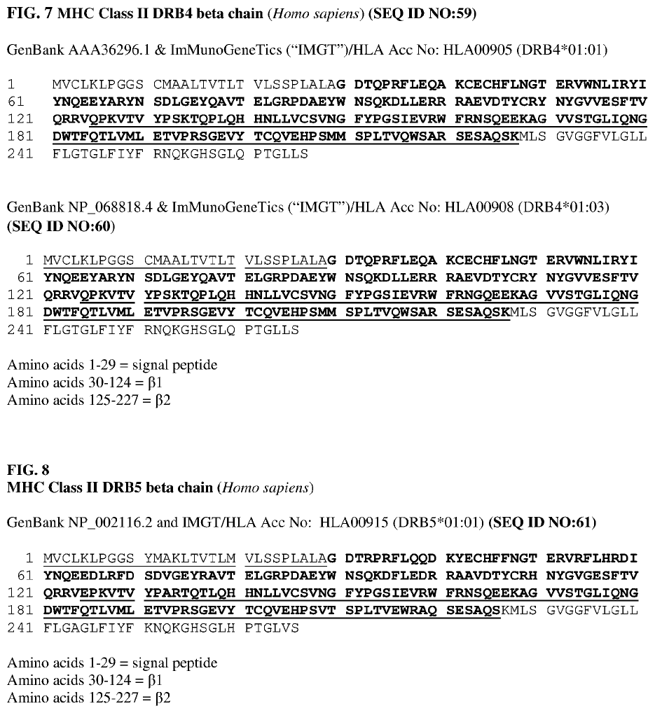

In some cases, a suitable MHC Class 1 chain polypeptide is a DRB4 polypeptide. A DRB4 polypeptide can have at least 60%, at least 70%, at least 80%, at least 90%, at least 95%, at least 98%, at least 99%, or 100% an sequence identity with aas 30-227 (the β1 and β2 domain region) of a DRB4 an sequence depicted in FIG. 7. In some cases, the DRB4 polypeptide has a length of about 198 aas (including, e.g., 195, 196, 197, 198, 199, 200, 201, or 202 aas). In some cases, a DRB4 polypeptide suitable for inclusion in a TMAPP comprises an amino acid substitution, relative to a wild-type DRB4 polypeptide, where the amino acid substitution replaces an amino acid (other than a Cys) with a Cys.

As used herein the term “DRB4 polypeptide” includes allelic variants, e.g., naturally occurring allelic variants. Thus, in some cases, a suitable DRB4 polypeptide comprises aas 30 to 227 of DRB4*01:03 (SEQ ID NO:60) provided in FIG. 7, or an allelic variant thereof. In some cases, a DRB4 polypeptide suitable for inclusion in a TMAPP comprises an amino acid substitution, relative to a wild-type DRB4 polypeptide, where the amino acid substitution replaces an amino acid (other than a Cys) with a Cys. Thus, e.g., in some cases, the MHC Class II β chain polypeptide is a variant DRB4 MHC Class II polypeptide that comprises a non-naturally occurring Cys residue; e.g., where the variant DRB4 MHC class II polypeptide comprises an amino acid substitution selected from the group consisting of P15C, F17C, Q20C, N29C, G30C, N43C, G161C, D162C, and W163C of a mature DRB4 polypeptide (lacking the N-terminal signal peptide MVCLKLPGGSCMAALTVTL (SEQ ID NO:76) depicted in FIG. 7).

A suitable DRB4 β1 domain, including-naturally occurring allelic variants thereof, may comprise an aa sequence having at least 70%, at least 80%, at least 90%, at least 95%, at least 98%, at least 99%, or 100% an sequence identity to the following aa sequence: T VLSSPLALAG DTQPRFLEQA KCECHFLNGT ERVWNLIRYI YNQEEYARYN SDLGEYQAVT ELGRPDAEYW NSQKDLLERR RAEVDTYCRY NYGVVESFTV QRRV (SEQ ID NO:77); and can have a length of about 95 aas (e.g., 93, 94, 95, 96, 97, or 98 aas).

A suitable DRB4 β2 domain, including-naturally occurring allelic variants thereof, may comprise an aa sequence having at least 70%, at least 80%, at least 90%, at least 95%, at least 98%, at least 99%, or 100% an sequence identity to the following an sequence: QPKVTV YPSKTQPLQH HNLLVCSVNG FYPGSIEVRW FRNGQEEKAG VVSTGLIQNG DWTFQTLVML ETVPRSGEVY TCQVEHPSMM SPLTVQWSAR SESAQSK (SEQ ID NO:78); and can have a length of about 103 aas (e.g., 100, 101, 102, 103, 104, or 105 aas).

### (d) DRB5 Polypeptides

A suitable MHC Class II β chain polypeptide is a DRB5 polypeptide. A DRB5 polypeptide can have at least 60%, at least 70%, at least 80%, at least 90%, at least 95%, at least 98%, at least 99%, or 100% an sequence identity with aas 30-227 (the β1 and β2 domain region) of the DRB5 an sequence depicted in FIG. 8. In some cases, the DRB5 polypeptide has a length of about 198 aas (including, e.g., 195, 196, 197, 198, 199, 200, 201, or 202 aas). In some cases, a DRB5 polypeptide suitable for inclusion in a TMAPP comprises an an substitution, relative to a wild-type DRB5 polypeptide, where the an substitution replaces an an (other than a Cys) with a Cys (e.g., for forming a disulfide bond that stabilizes the TMAPP).

As used herein, the term “DRB5 polypeptide” includes allelic variants, e.g., naturally occurring allelic variants. Thus, in some cases, a suitable DRB4 polypeptide comprises aas 30 to 227 of DRB5*01:01 (SEQ ID NO:61) provided in FIG. 8, or an allelic variant thereof.

A suitable DRB5 β1 domain, including-naturally occurring allelic variants thereof, may comprise an aa sequence having at least 70%, at least 80%, at least 90%, at least 95%, at least 98%, at least 99%, or 100% aa sequence identity to the following aa sequence: M VLSSPLALAG DTRPRFLQQD KYECHFFNGT ERVRFLHRDI YNQEEDLRFD SDVGEYRAVT ELGRPDAEYW NSQKDFLEDR RAAVDTYCRH NYGVGESFTV QRRV (SEQ ID NO:79); and can have a length of about 95 aas (e.g., 93, 94, 95, 96, 97, or 98 aas).

A suitable DRB5 β2 domain, including-naturally occurring allelic variants thereof, may comprise an aa sequence having at least 70%, at least 80%, at least 90%, at least 95%, at least 98%, at least 99%, or 100% an sequence identity to the following an sequence: EPKVTV YPARTQTLQH HNLLVCSVNG FYPGSIEVRW FRNSQEEKAG VVSTGLIQNG DWTFQTLVML ETVPRSGEVY TCQVEHPSVT SPLTVEWRAQ SESAQS (SEQ ID NO:80); and can have a length of about 103 aas (e.g., 100, 101, 102, 103, 104, or 105 aas).

### (ii) DQB Polypeptides

A suitable MHC Class II a DQB polypeptide may have at least 60%, at least 70%, at least 80%, at least 90%, at least 95%, at least 98%, at least 99%, or 100% aa sequence identity with the α1, α2, β1, and/or the β2 domain(s) of DQB1*0201, DQB1*0302, DQB1*0303, DQB1*0402, or DQB1*0501. In some cases, the DQA polypeptide has a length of about 178 aas (e.g., 175, 176, 177, 178, 179, or 180 aas). The sequences of those HLA polypeptides are available, for example on the world wide web at hla.alleles.org/nomenclature/index.html, which is run by the Anthony Nolan Research Institute, and at www.ncbi.nlm.nih.gov (National Center for Biotechnical Information or “NCBI”) operated by the U.S. National Library of Medicine.

### 3. Individual Disease Risk-Associated Alleles

a. DRB1

### (i) DRB1*01:01

A TMAPP may comprise a DRB1*01:01 polypeptide comprising an aa sequence having at least 70%, at least 80%, at least 90%, at least 95%, at least 98%, at least 99%, or 100%, an sequence identity to at least 170, at least 180, or at least 190, contiguous aas of the sequence from aa 30 to aa 227 of the DRB1*01:01 an sequence provided in FIG. 5. In some cases, a TMAPP comprises a DRB1*01:01 polypeptide comprising an an sequence having at least 70%, at least 80%, at least 90%, at least 95%, at least 98%, at least 99%, or 100%, aa sequence identity to aas 30-124 of the DRB1*01:01 aa sequence provided in FIG. 5. In some cases, a TMAPP comprises a DRB1*01:01 polypeptide comprising an aa sequence having at least 70%, at least 80%, at least 90%, at least 95%, at least 98%, at least 99%, or 100%, an sequence identity to aas 125-227 of the DRB1*01:01 aa sequence depicted in FIG. 5.

### (ii) DRB1*01:02

A TMAPP may comprise a DRB1*01:02 polypeptide comprising an aa sequence having at least 70%, at least 80%, at least 90%, at least 95%, at least 98%, at least 99%, or 100%, aa sequence identity to at least 170, at least 180, or at least 190, contiguous aas of the sequence from aa 30 to aa 227 of the DRB1*01:02 an sequence provided in FIG. 5. In some cases, a TMAPP comprises a DRB1*01:02 polypeptide comprising an an sequence having at least 70%, at least 80%, at least 90%, at least 95%, at least 98%, at least 99%, or 100%, aa sequence identity to aas 30-124 of the DRB1*01:02 aa sequence provided in FIG. 5. In some cases, a TMAPP comprises a DRB1*01:02 polypeptide comprising an as sequence having at least 70%, at least 80%, at least 90%, at least 95%, at least 98%, at least 99%, or 100%, as sequence identity to aas 125-227 of the DRB1*01:02 aa sequence depicted in FIG. 5.

(iii) DRB1*01:03

A TMAPP may comprise a DRB1*01:03 polypeptide comprising an as sequence having at least 70%, at least 80%, at least 90%, at least 95%, at least 98%, at least 99%, or 100%, aa sequence identity to at least 170, at least 180, or at least 190, contiguous aas of the sequence from aa 30 to aa 227 of the DRB1*01:03 as sequence provided in FIG. 5. In some cases, a TMAPP comprises a DRB1*01:03 polypeptide comprising an as sequence having at least 70%, at least 80%, at least 90%, at least 95%, at least 98%, at least 99%, or 100%, as sequence identity to aas 30-124 of the DRB1*01:03 aa sequence provided in FIG. 5. In some cases, a TMAPP comprises a DRB1*01:03 polypeptide comprising an as sequence having at least 70%, at least 80%, at least 90%, at least 95%, at least 98%, at least 99%, or 100%, as sequence identity to aas 125-227 of the DRB1*01:03 aa depicted in FIG. 5.

### (iv) DRB1*3:01

DRB1*0301 (“DRB1*03:01” in FIG. 5) is associated with increased risk of developing T1D. Thus, a TMAPP may comprise a DRB1*03:01 polypeptide comprising an as sequence having at least 70%, at least 80%, at least 90%, at least 95%, at least 98%, at least 99%, or 100%, as sequence identity to at least 170, at least 180, or at least 190, contiguous aas of the sequence from aa 30 to aa 227 of the DRB1*03:01 as sequence provided in FIG. 5. In some cases, a TMAPP comprises a DRB1*03:01 polypeptide comprising an as sequence having at least 70%, at least 80%, at least 90%, at least 95%, at least 98%, at least 99%, or 100%, as sequence identity to aas 30-124 of the DRB1*03:01 aa sequence depicted in FIG. 5. In some cases, a TMAPP comprises a DRB1*03:01 polypeptide comprising an as sequence having at least 70%, at least 80%, at least 90%, at least 95%, at least 98%, at least 99%, or 100%, as sequence identity to aas 125-227 of the DRB1*03:01 aa sequence depicted in FIG. 5.

### (v) DRB1*03:02

A TMAPP may comprise a DRB1*03:02 polypeptide comprising an as sequence having at least 70%, at least 80%, at least 90%, at least 95%, at least 98%, at least 99%, or 100%, as sequence identity to at least 170, at least 180, or at least 190, contiguous aas of the sequence from aa 30 to aa 227 of the DRB1*03:02 as sequence provided in FIG. 5. In some cases, a TMAPP comprises a DRB1*03:02 polypeptide comprising an as sequence having at least 70%, at least 80%, at least 90%, at least 95%, at least 98%, at least 99%, or 100%, as sequence identity to as 30-124 of the DRB1*03:02 aa sequence provided in FIG. 5. In some cases, a TMAPP comprises a DRB1*03:02 polypeptide comprising an as sequence having at least 70%, at least 80%, at least 90%, at least 95%, at least 98%, at least 99%, or 100%, as sequence identity to aas 125-227 of the DRB1*03:02 aa sequence depicted in FIG. 5.

### (vi) DRB1*03:04:

A TMAPP may comprise a DRB1*03:04 polypeptide comprising an aa sequence having at least 70%, at least 80% at least 90%, at least 95%, at least 98%, at least 99%, or 100%, aa sequence identity to at least 170, at least 180, or at least 190, contiguous aas of the sequence from aa 30 to aa 227 of the DRB1*03:04 as sequence provided in FIG. 5. In some cases, a TMAPP comprises a DRB1*03:04 polypeptide comprising an as sequence having at least 70%, at least 80%, at least 90%, at least 95%, at least 98%, at least 99%, or 100%, as sequence identity to aas 30-124 of the DRB1*03:04 aa sequence provided in FIG. 5. In some cases, a TMAPP comprises a DRB1*03:04 polypeptide comprising an as sequence having at least 70%, at least 80%, at least 90%, at least 95%, at least 98%, at least 99%, or 100%, as sequence identity to aas 125-227 of the DRB1*03:04 aa sequence depicted in FIG. 5.

(vii) DRB1*04:01

A TMAPP may comprise a DRB1*04:01 polypeptide comprising an as sequence having at least 70%, at least 80%, at least 90%, at least 95%, at least 98%, at least 99%, or 100%, as sequence identity to at least 170, at least 180, or at least 190, contiguous aas of the sequence from aa 30 to aa 227 of the DRB1*04:01 as sequence depicted in FIG. 5. In some cases, a TMAPP comprises a DRB1*04:01 polypeptide comprising an as sequence having at least 70%, at least 80%, at least 90%, at least 95%, at least 98%, at least 99%, or 100%, as sequence identity to aas 30-124 of the DRB1*04:01 aa sequence depicted in FIG. 5. In some cases, a TMAPP comprises a DRB1*04:01 polypeptide comprising an as sequence having at least 70%, at least 80%, at least 90%, at least 95%, at least 98%, at least 99%, or 100%, as sequence identity to aas 125-227 of the DRB1*04:01 aa sequence depicted in FIG. 5.

(viii) DRB1*04:02

A TMAPP may comprise a DRB1*04:02 polypeptide comprising an as sequence having at least 70%, at least 80%, at least 90%, at least 95%, at least 98%, at least 99%, or 100%, as sequence identity to at least 170, at least 180, or at least 190, contiguous aas of the sequence from aa 30 to aa 227 of the DRB1*04:02 as sequence provided in FIG. 5. In some cases, a TMAPP comprises a DRB1*04:02 polypeptide comprising an as sequence having at least 70%, at least 80%, at least 90%, at least 95%, at least 98%, at least 99%, or 100%, as sequence identity to aas 30-124 of the DRB1*04:02 aa sequence provided in FIG. 5. In some cases, a TMAPP comprises a DRB1*04:02 polypeptide comprising an as sequence having at least 70%, at least 80%, at least 90%, at least 95%, at least 98%, at least 99%, or 100%, as sequence identity to aas 125-227 of the DRB1*04:02 aa sequence provided in FIG. 5.

### (ix) DRB1*06:01

A TMAPP may comprise a DRB1*08:01 polypeptide comprising an as sequence having at least 70%, at least 80%, at least 90%, at least 95%, at least 98%, at least 99%, or 100%, as sequence identity to at least 170, at least 180, or at least 190, contiguous aas of the sequence from aa 30 to aa 227 of the DRB1*08:01 as sequence provided in FIG. 5 In some cases, a TMAPP comprises a DRB1*08:01 polypeptide comprising an as sequence having at least 70%, at least 80%, at least 90%, at least 95%, at least 98%, at least 99%, or 100%, as sequence identity to aas 30-124 of the DRB1*08:01 aa sequence provided in FIG. 5. In some cases, a TMAPP comprises a DRB1*08:01 polypeptide comprising an an sequence having at least 70%, at least 80%, at least 90%, at least 95%, at least 98%, at least 99%, or 100%, aa sequence identity to aas 125-227 of the DRB1*08:01 aa sequence depicted in FIG. 5.

### (x) DRB1*09:01

A TMAPP may comprise a DRB1*09:01 polypeptide comprising an an sequence having at least 70%, at least 80%, at least 90%, at least 95%, at least 98%, at least 99%, or 100%, an sequence identity to at least 170, at least 180, or at least 190, contiguous aas of the sequence from aa 30 to aa 227 of the DRB1*09:01 an sequence provided in FIG. 5. In some cases, a TMAPP comprises a DRB1*09:01 polypeptide comprising an an sequence having at least 70%, at least 80%, at least 90%, at least 95%, at least 98%, at least 99%, or 100%, an sequence identity to aas 30-124 of the DRB1*09:01 an sequence provided in FIG. 5. In some cases, a TMAPP comprises a DRB1*09:01 polypeptide comprising an an sequence having at least 70%, at least 80%, at least 90%, at least 95%, at least 98%, at least 99%, or 100%, an sequence identity to aas 125-227 of the DRB1*09:01 aa sequence depicted in FIG. 5.

b. DRB3

In some cases, a TMAPP comprises an MHC Class II β chain polypeptide of a DRB3 allele.

### (i) DRB3*03:01

A TMAPP may comprise a DRB3*03:01 polypeptide comprising an an sequence having at least 70%, at least 80%, at least 90%, at least 95%, at least 98%, at least 99%, or 100%, an sequence identity to at least 170, at least 180, or at least 190, contiguous aas of the sequence from aa 30 to an 227 of the DRB3*03:01 an sequence provided in FIG. 6. In some cases, a TMAPP comprises a DRB3*03:01 polypeptide comprising an an sequence having at least 70%, at least 80%, at least 90%, at least 95%, at least 98%, at least 99%, or 100%, an sequence identity to aas 30-124 of the DRB3*03:01 an sequence provided in FIG. 6. In some cases, a TMAPP comprises a DRB3*03:01 polypeptide comprising an an sequence having at least 70%, at least 80%, at least 90%, at least 95%, at least 98%, at least 99%, or 100%, an sequence identity to aas 125-227 of the DRB3*03:01 aa acid sequence depicted in FIG. 6.

c. DRB4

In some cases, a TMAPP comprises an MHC Class II β chain polypeptide of a DRB4 allele. A TMAPP may comprise a DRB4*01:01 polypeptide comprising an an sequence having at least 70%, at least 80%, at least 90%, at least 95%, at least 98%, at least 99%, or 100%, an sequence identity to at least 170, at least 180, or at least 190, contiguous aas of the sequence from an 30 to an 227 of the DRB4*01:01 an sequence provided in FIG. 7. In some cases, a TMAPP comprises a DRB4*01:01 polypeptide comprising an an sequence having at least 70%, at least 80%, at least 90%, at least 95%, at least 98%, at least 99%, or 100%, an sequence identity to aas 30-124 of the DRB4*01:01 an sequence provided in FIG. 7. In some cases, a TMAPP comprises a DRB3*03:01 polypeptide comprising an an sequence having at least 70%, at least 80%, at least 90%, at least 95%, at least 98%, at least 99%, or 100%, an sequence identity to aas 125-227 of the DRB4*01:01 aa sequence provided in FIG. 7.

d. DRB5

A TMAPP may comprise a MHC Class H β chain polypeptide of a DRB5 allele. In some cases, the DRB5 polypeptide has a length of about 198 aas (e.g., 195, 196, 197, 198, 199, 200, 201, or 202 aas). In some cases, a DRB5 polypeptide suitable for inclusion in a TMAPP comprises an as substitution, relative to a wild-type DRB5 polypeptide, where the aa substitution replaces an aa (other than a Cys) with a Cys.

### (i) DRB5*01:01

A TMAPP may comprise a DRB5*01:01 polypeptide comprising an as sequence having at least 70%, at least 80%, at least 90%, at least 95%, at least 98%, at least 99%, or 100%, as sequence identity to at least 170, at least 180, or at least 190, contiguous aas of the sequence from aa 30 to aa 227 of the DRB5*01:01 as sequence provided in FIG. 8. In some cases, a DRB5 polypeptide suitable for inclusion in a TMAPP comprises an aa substitution, relative to a wild-type DRB5 polypeptide, where the as substitution replaces an as (other than a Cys) with a Cys. Thus, e.g., in some cases, the MHC Class II chain polypeptide is a variant DRB5 MHC Class II polypeptide that comprises a non-naturally occurring Cys residue; e.g., where the variant DRB5 MHC Class II polypeptide comprises an aa substitution selected from the group consisting of P15C, F17C, Q20C, N29C, G30C, N43C, G161C, D162C, and W163C (of a mature DRB5 polypeptide (lacking the N-terminal signal peptide MVCLKLPGGSYMAKLTVTL (SEQ ID NO:81) depicted in FIG. 8), or a naturally-occurring allelic variant thereof.

A TMAPP may comprise a DRB5*01:01 β1 domain polypeptide comprising an as sequence having at least 70%, at least 80%, at least 90%, at least 95%, at least 98%, at least 99%, or 100%, as sequence identity to aas 30-124 of the DRB5*01:01 as sequence provided in FIG. 8. In some cases, a TMAPP comprises a DRB5*01:01 polypeptide comprising an as sequence having at least 70%, at least 80%, at least 90%, at least 95%, at least 98%, at least 99%, or 100%, as sequence identity to at least 170, at least 180, or at least 190, contiguous aas of the sequence from aa 30 to aa 227 of the DRB5*01:01 β2 domain an sequence depicted in FIG. 8, or a naturally-occurring allelic variant thereof.

### V.A(ii). The Structure and Organization of Presenting Sequences and Complexes

### 1. The Organization of MHC Peptides in Presenting Sequences and Complexes

As discussed above, the presenting sequences and presenting complexes comprise the MHC elements required for presenting an epitope to a TCR (e.g., α1, α2, β1, and β2 domain sequences), and those elements may be ordered in more than one fashion. While other arrangements are possible, presenting sequences are typically ordered in only a few fashions. For example, the presenting sequences may comprise, from N-terminus to C-terminus, MHC Class II: (i) β1, α1, α2, and β2 domain sequences; (ii) β1, β2, α1, and α2 domain sequences; or (iii) α1, α2 β1, and β2, domain sequences. See FIG. 9D to FIG. 9J. Those MHC Class II domain sequences may be joined by linker polypeptides (e.g., one or more GGGGS repeats) and may have one more wt. and/or variant MODs (e.g., two or more MODs, such as in tandem) located at their N- or C-termini. In addition, MODs may be located between the individual MHC α and β chain domain sequences.

Presenting complexes also have typically have the MHC elements required for presenting an epitope to a TCR ordered in their first and second presenting sequence in only a few fashions. While other arrangements are possible, presenting complexes typically comprise, from N-terminus to C-terminus, the MHC Class II: (i) α1 and α2 domains as part of one of the first or second presenting sequence, and the β1 and β2 domains as part of the other of the first or second presenting sequences (see e.g., FIGS. 9A and 9B.); or (ii) β1, α1, and α2 domains as part of one of the first or second presenting sequence, and the β2 domain as part of the other of the first or second presenting sequences (see e.g., FIGS. 9C and 9D). Those MHC Class H domain sequences may be joined by linker polypeptides (e.g., one or more GGGGS repeats) and may have one more wt. and/or variant MODs (e.g., two or more MODs, such as in tandem) located at their N- or C-termini. In addition, MODs may be located between the individual MHC α and β chain domain sequences.

### 2. Disulfide Bonding in Presenting Sequences and Presenting Complexes

Disulfide bonds involving an MHC peptide sequence may be included in a presenting sequence or complex of a TMAPP. The disulfide bonds may increase the stability of the TMAPP (e.g., thermal stability). The disulfide bonds may be between two MHC peptide sequences (e.g., a cysteine located in an α chain and a cysteine located in a β chain sequence). Disulfide bonds, and particularly disulfide bonds made to position a peptide epitope may be between two MHC peptide sequences or, alternatively, between an MHC peptide sequence and a linker attaching the peptide epitope and an MHC sequence (e.g., a linker between the epitope and chemical conjugation site in a β1 domain sequence in FIGS. 9-E to 9H). Disulfide bonds for stabilization of a TMAPP-epitope conjugate may be made using cysteines found within the MHC sequences and/or cysteines that have been provide in one or more MHC sequences using the techniques of molecular biology and protein engineering. The α chain may include, e.g., a cysteine at position 3, 4, 12, 28, 29, 72, 75, 80, 81, 82, 93, 94, or 95 of the mature α chain (lacking its signal sequence). For the DRA polypeptides cysteines substitutions include, e.g., those at E3C, E4C, F12C, G28C, D29C, 172C, K75C, T80C, P81C, 182C, T93C, N94C, and S95C (see FIG. 4). The β chain may include a cysteine, e.g., at position 5, 7, 10, 19, 20, 33, 151, 152, or 153 of the mature β chain (lacking its signal sequence). For the DB1 possible polypeptides cysteines substitutions include those at positions P5C, F7C, Q10C (may be Y10C or E10C for some DRB1 alleles), N19C, G20C, H33C (may be N33C for some DRB1alleles), G151C, D152C, and W153C.

Stabilizing disulfide bonds between α and α chain sequences in the body of the MHC complex (body disulfides) include those between the α and β chain positions set forth in Table 3, which also provides the specific cysteine substitutions for HLA DRA*01:02 and DRB*0401 sequences. The stabilizing disulfide bonds between the MHC (e.g., HLA) α and β chains may be incorporated into any of the TMAPP structures described herein. For example, such disulfide bonds may be incorporated into presenting sequences or complexes such as those shown in FIG. 9A to 9J.

TABLE 3

| α chain | β chain | DRA*01:02 α chain | DRB*0401 β chain |
|---|---|---|---|
| position | position | substitution | substitution |
| 3 | 19 | E3C | N19C |
| 3 | 20 | E3C | G20C |
| 4 | 19 | E4C | N19C |
| 4 | 20 | E4C | G20C |
| 28 | 151 | G28C | G151C |
| 28 | 152 | G28C | D152C |
| 28 | 153 | G28C | W153 |
| 29 | 151 | D29C | G151C |
| 29 | 152 | D29C | D152C |
| 29 | 153 | D29C | W153 |
| 80 | 33 | T80C | H33C |
| 81 | 33 | P81C | H33C |
| 82 | 33 | I82C | H33C |
| 93 | 156 | T93C | Q156C |
| 93 | 153 | T93C | W153C |
| 94 | 156 | N94C | Q156C |
| 94 | 120 | N94C | N120C |
| 95 | 156 | S95C | Q156C |
| 95 | 120 | S95C | N120C |

Disulfide bonds between the α a and β chain sequences that assist in stabilizing the TMAPP may be formed between a first as and second as of a TMAPP. The first as is either (i) an aa position proximate to the point where a peptide epitope (or a peptide epitope and linker) are conjugated to an MHC peptide sequence, or (ii) an aa (a cysteine) in a linker attached to the peptide epitope, the second as is position elsewhere in the MHC peptide sequence. By way of example, where a presenting sequence comprises from N-terminus to C-terminus a peptide epitope, β1 domain, β2 domain, al domain, and α2 domain aa sequences, a cysteine substituted within the first ten amino acids (e.g., aas 5-10) of the β1 domain can serve as a first aa and provide a point to anchor the peptide epitope and/or stabilize the TMAPP when bonded to a with second cysteine located in, for example, the α1 domain, or α2 domain of the presenting sequence Some examples of disulfide bonds between the MHC α and β chain sequences that assist in stabilizing the TMAPP include those set forth in Table 4.

TABLE 4

| α chain | β chain | DRA*01:02 α chain | DRB*0401 β chain |
|---|---|---|---|
| position | position | substitution | substitution |
| 12 | 7 | F12C | F7C |
| 12 | 10 | F12C | Q10C |
| 80 | 5 | T80C | P5C |
| 80 | 7 | T80C | F7C |
| 81 | 5 | P81C | P5C |
| 81 | 7 | P81C | F7C |
| 82 | 5 | I82C | P5C |
| 82 | 7 | I82C | F7C |

Thus, for example, when a presenting sequence of complex comprises in the N-terminal to C-terminal direction a peptide epitope bound to a β1 domain, then a disulfide bond between a cysteine substituted at one of position 5-7 of the β chain, and a cysteine at one of aa positions 80-82 of the α chain may be used for stabilizing the TMAPP. By way of example a disulfide bond between a β chain P5C substitution and an α chain P81C substitution may be used for stabilization of a TMAPP. The same type of disulfide bonding is applicable to presenting complexes, and both presenting complexes and presenting sequences may have additional disulfide bonds (e.g., as in Table 3) for stabilization.

Where a cysteine residue in a linker attached to the peptide epitope is employed to stabilize the TMAPP, the cysteine is typically located at an aa proximate to the point where the linker and peptide epitope meet. For example, where the TMAPP comprises an epitope place on the N-terminal side of a linker peptide sequence the cysteine may be within about 6 aas of the position were the linker and peptide epitope meet, that is to say at one of amino acids 1-5 (aa1, aa2, aa3, aa4, or aa5) of a TMAPP comprising the construct epitope-aa1-aa2-aa3-aa4-aa5-(remainder of the linker/TMAPP). Where the linker comprises repeats of the sequence GGGGS (SEQ ID NO:82), aa1 to aa5 are G1, G2, G3, G4, and S5, and the linker substitutions may be referred to as, for example a “G2C.” This is exemplified by SEQ ID NO:82, that has four repeats of GGGGS in which the aa at position 2 of the linker (aa2), is a glycine substituted by a cysteine: G [symbol] SGGGGSGGGGSGGGGS (SEQ ID NO:83). Examples of cysteine containing linkers suitable for forming disulfide bonds with a cysteine in an MHC peptide (e.g., an a chain peptide sequence such a DRA peptide) in a presenting sequence or complex comprising an epitope placed on the N-terminal side of a linker bound to an MHC β chain such as a DRB polypeptide (i.e., the TMAPP comprises the structure epitope-aa1-aa2-aa3-aa4-aa5-[remainder of linker if present]-MHC β1 domain, such as a DRB β1 domain) are set forth in Table 5. Also provided in Table 5 is the location for a cysteine substituted in a DRA polypeptide (see e.g., FIG. 4)

That will form the disulfide bond for stabilizing the TMAPP.

TABLE 5
Exemplary Linker disulfide bonds for TMAPPs comprising the structure epitope-aa1-aa2-
aa3-aa4-aa5-(remainder of linker if present)-MHC β1 domain e.g., of a DRB peptide

| Cysteine | Cysteine substitution in the | Cysteine substitution in a | α chain position, (DRA |
|---|---|---|---|
| position | general structure | GGGGS containing linker | Cysteine substitution) |
| aa1 | epitope-C-aa2-aa3-aa4-aa5- | epitope-CGGGS-(remainder | 72 (I72C) |
| | (remainder of linker)-DRB | of linker)-DRB | |
| | | (i.e., a linker G1C | |
| | | substitution) | |
| aa2 | epitope-aa1-C-aa3-aa4-aa5- | epitope-GCGGS-(remainder | 72 (I72C) |
| | (remainder of linker)-DRB | of linker)-DRB | |
| | | (i.e., a linker G2C | |
| | | substitution) | |
| aa3 | epitope-aa1-aa2-C-aa4-aa5- | epitope-GGCGS-(remainder | 72 (I72C) |
| | (remainder of linker)-DRB | of linker)-DRB | |
| | | (i.e., a linker G3C | |
| | | substitution) | |
| aa4 | epitope-aa1-aa2-aa3-C-aa5- | epitope-GGGCS-(remainder | 72 (I72C) |
| | (remainder of linker)-DRB | of linker)-DRB | |
| | | (i.e., a linker G4C | |
| | | substitution) | |
| aa5 | epitope-aa1-aa2-aa3-aa4-C- | epitope-GGGGC-(remainder | 72 (I72C) |
| | (remainder of linker)-DRB | of linker)-DRB | |
| | | (i.e., a linker S5C substitution) | |
| aa1 | epitope-C-aa2-aa3-aa4-aa5- | epitope-CGGGS-(remainder | 75 (K75C) |
| | (remainder of linker)-DRB | of linker)-DRB | |
| aa2 | epitope-aa1-C-aa3-aa4-aa5- | epitope-GCGGS-(remainder | 75 (K75C) |
| | (remainder of linker)-DRB | of linker)-DRB | |
| aa3 | epitope-aa1-aa2-C-aa4-aa5- | epitope-GGCGS-(remainder | 75 (K75C) |
| | (remainder of linker)-DRB | of linker)-DRB | |
| aa4 | epitope-aa1-aa2-aa3-C-aa5- | epitope-GGGCS-(remainder | 75 (K75C) |
| | (remainder of linker)-DRB | of linker)-DRB | |
| aa5 | epitope-aa1-aa2-aa3-aa4-C- | epitope-GGGGC-(remainder | 75 (K75C) |
| | (remainder of linker)-DRB | of linker)-DRB | |

TMAPPs with presenting sequences or complexes comprising an epitope-linker-DRB structure recited in Table 5 may have for example a disulfide bond for p stabilizing TMAPP. The disulfide may be formed between linker aa2 (e.g., a G2C) and a cysteine at DRA an 72 (e.g., I72C). The disulfide may be formed between linker aa2 (e.g., a G2C) and a cysteine at DRA an 72 (e.g., K75C).

Where a disulfide bond is formed between the linker and an MHC polypeptide of a presenting sequence or presenting complex, the presenting sequence or presenting complex may have additional disulfide bonds (e.g., as in Table 3) for stabilization.

### V.A(iii). Scaffold Polypeptides

TMAPPs and TMAPP-epitope conjugates may comprise an immunoglobulin heavy chain constant region (“Ig Fc” or “Fc”) polypeptide, or may comprise another suitable scaffold polypeptide. Where scaffold polypeptide sequences are identical and pair or multimerize (e.g., some Ig Fc sequences or leucine zipper sequences), they can form symmetrical pairs or multimers (e.g., homodimers, see e.g., FIGS. 10B and 10D with an Fc scaffold). In contrast, where an asymmetric pairing between two TMAPP molecules is desired (e.g., to produce a duplex TMAPP with each bearing one or more different MODs), the scaffold polypeptides present in the TMAPP may comprise interspecific binding sequences. Interspecific binding sequences are non-identical polypeptide sequences that selectively interact with their specific complementary counterpart sequence to form asymmetric pairs (heterodimers, see e.g., FIGS. 10A and 10C with an interspecific scaffold illustrated by a knob-in-hole Fc pair). Interspecific binding sequences may in some instances form an amount of homodimers, but preferentially dimerize by binding more strongly) with their counterpart interspecific binding sequence. Accordingly, specific heterodimers tend to be formed when an interspecific dimerization sequence and its counterpart interspecific binding sequence are incorporated into a pair of TMAPPs. By way of example, where an interspecific dimerization sequence and its counterpart are incorporated into a pair of TMAPPs they may selectively form greater than about 80%, 90%, 95%, 98% or 99% heterodimers when an equimolar mixture of the polypeptides are combined. The remainder of the polypeptides may be present as monomers or homodimers that may be separated from the heterodimer. Moreover, because interspecific sequences are selective for their counterpart sequence, they can limit the interaction with other proteins expressed by cells (e.g., in culture or in a subject) particularly where the interspecific sequences are not naturally occurring or are variants of naturally occurring protein sequences.

Scaffold polypeptide sequences generally may be less than 300 aa (e.g., about 100 to about 300 aa). Scaffold polypeptide sequences may be less than 250 an (e.g., about 75 to about 250 aa). Scaffold polypeptide sequences may be less than 200 an (e.g., about 60 to about 200 aa). Scaffold polypeptide sequences may be less than 150 aa (e.g., about 50 to about 150 aa).

Scaffold polypeptide sequences include, but are not limited to, interspecific and non-interspecific Ig Fc polypeptide sequences, however, polypeptide sequences other than Ig Fc polypeptide sequences (non-immunoglobulin sequences) may be used as scaffolds.

### 1. Non-Immunoglobulin Fc Scaffold Polypeptides

Non-immunoglobulin Fc scaffold polypeptides include, but are not limited to: albumin, XTEN (extended recombinant); transferrin; Fc receptor, elastin-like; albumin-binding; silk-like (see, e.g., Valluzzi et al. (2002) Philos Trans R Soc Lond B Biol Sci. 357:165); a silk-elastin-like (SELP; see, e.g., Megeed et al. (2002) Adv Drug Deliv Rev. 54:1075) polypeptides; and the like. Suitable XTEN polypeptides include, e.g., those disclosed in WO 2009/023270, WO 2010/091122, WO 2007/103515, US 2010/018%82, and US 2009/0092582; see, also, Schellenberger et al. (2009) Nat Biotechnol. 27:1186). Suitable albumin polypeptides include, e.g., human serum albumin. Suitable elastin-like polypeptides are described, for example, in Hassouneh et al. (2012) Methods Enzymol. 502:215.

Other non-immunoglobulin Fc scaffold polypeptide sequences include but are not limited to: polypeptides of the collectin family (e.g., ACRP30 or ACRP30-like proteins) that contain collagen domains consisting of collagen repeats Gly-Xaa-Yaa and/or Gly-Xaa-Pro (which may be repeated from 10-40 times); coiled-coil domains; leucine-zipper domains; Fos/Jun binding pairs; Ig CH1 and light chain constant region C_{L} sequences (Ig CH1/C_{L} pairs such as a Ig CH1 sequence paired with a Ig C_{L} κ or C_{L} λ light chain constant region sequence).

Non-immunoglobulin Fc scaffold polypeptides can be interspecific or non-interspecific in nature. For example, both Fos/Jun binding pairs and Ig CH1 polypeptide sequences and light chain constant region C_{L} sequences form interspecific binding pairs. Coiled-coil sequences, including leucine zipper sequences, can be either interspecific leucine zipper or non-interspecific leucine zipper sequences. See e.g., Zeng et al., (1997) PNAS (USA) 94:3673-3678; and Li et al., (2012), Nature Comms. 3:662.

The scaffold polypeptides of a duplex TMAPP may each comprise a leucine zipper polypeptide sequence. The leucine zipper polypeptides bind to one another to form a dimer. Non-limiting examples of leucine-zipper polypeptides include a peptide comprising any one of the following aa sequences: RMKQIEDKIEEILSKIYHIENEIARIKKLIGER (SEQ ID NO:84); LSSIEKKQEEQTSWLIWISN-ELTLIRNELAQS (SEQ ID NO:85); LSSIEKKLEEITSQLIQISNELTLIRNELAQ (SEQ ID NO:86); LSSIEKKLEEITSQLIQIRNELTLIRNELAQ (SEQ ID NO:87); LSSIEKKLEEITSQLQQIRNELTLI-RNELAQ (SEQ ID NO:88); LSSLEKKLEELTSQLIQLRNELTLLRNELAQ (SEQ ID NO:89); ISSLE-KKIEELTSQIQQLRNEITLLRNEIAQ (SEQ ID NO:90). In some cases, a leucine zipper polypeptide comprises the following aa sequence: LEIEAAFLERENTALETRVAELRQRVQRLRNRVSQYRT-RYGPLGGGK (SEQ ID NO:91). Additional leucine-zipper polypeptides are known in the art, a number of which are suitable for use as scaffold polypeptide sequences.

The scaffold polypeptide of a TMAPP may comprise a coiled-coil polypeptide sequence that forms a dimer. Non-limiting examples of coiled-coil polypeptides include, for example, a peptide of any one of the following aa sequences: LKSVENRLAVVENQLKTVIEELKTVKDLLSN (SEQ ID NO:92); LARIEEKLKTIKAQLSEIASTLNMIREQLAQ (SEQ ID NO:93); VSRLEEKVKTLKSQVTELAS-TVSLLREQVAQ (SEQ ID NO:94); IQSEKKIEDISSLIGQIQSEITLIRNEIAQ (SEQ ID NO:95); and LMSLEKKLEELTQTLMQLQNELSMLKNELAQ (SEQ ID NO:96).

The TMAPPs of a TMAPP duplex may comprise a pair of scaffold polypeptide sequences that each comprise at least one cysteine residue that can form a disulfide bond permitting homodimerization or heterodimerization of those polypeptides stabilized by an interchain disulfide bond between the cysteine residues. Examples of such aa sequences include: VDLEGSTSNGRQCAGIRL (SEQ ID NO:97); EDDVTITEELAPALVPPPKGTCAGWMA (SEQ ID NO:98); and GHDQEITrQGPGVLL-PLPKGACTGQMA (SEQ ID NO:99).

Some scaffold polypeptide sequences permit formation of TMAPP complexes of higher order than duplexes, such as triplexes, tetraplexes, pentaplexes or hexaplexes. Such aa sequences include, but are not limited to, IgM constant regions (discussed below). Collagen domains, which form trimers, can also be employed. Collagen domains may comprise the three aa sequence Gly-Xaa-Xaa and/or GlyXaaYaa, where Xaa and Yaa are independently any aa, with the sequence appear or are repeated multiple times (e.g., from 10 to 40 times). In Gly-Xaa-Yaa sequences, Xaa and Yaa are frequently proline and hydroxyproline respectively in greater than 25%, 50%, 75%, 80% 90% or 95% of the Gly-Xaa-Yaa occurrences, or in each of the Gly-Xaa-Yaa occurrences. In some cases, a collagen domain comprises the sequence Gly-Xaa-Pro repeated from 10 to 40 times. A collagen oligomerization peptide can comprise the following aa sequence:

(SEQ ID NO: 100)
VTAFSNMDDMLQKAHLVIEGTFIYLRDSTEFFI
RVRDGWKKLQLGELIPIPADSPPPPALSSNP.

### 2. Immunoglobulin Fc Scaffold Polypeptides

a. Non-Interspecific Immunoglobulin Fc Scaffold Polypeptides

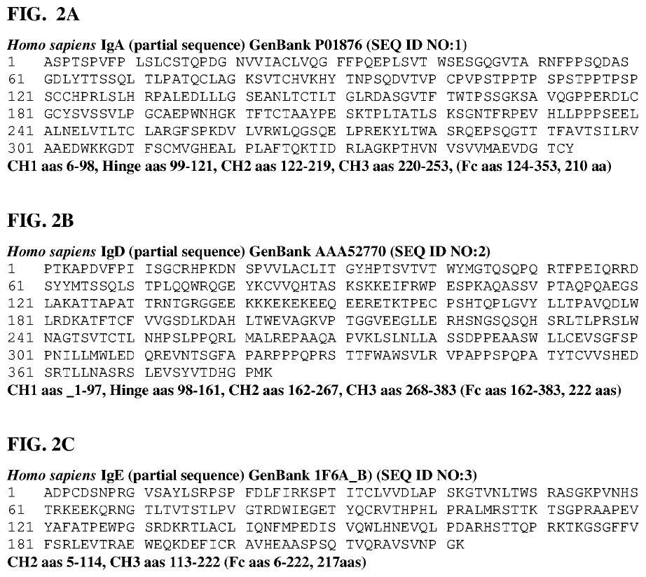

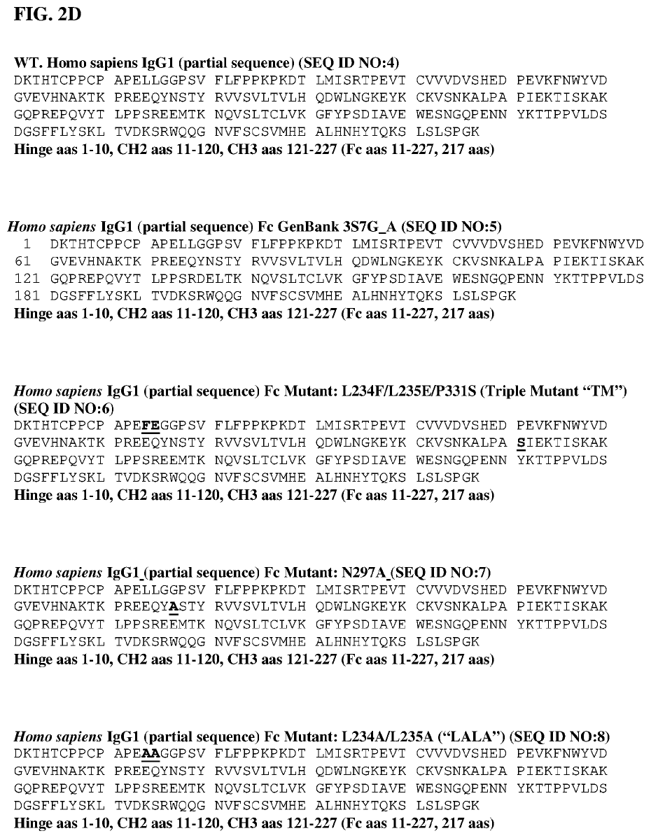

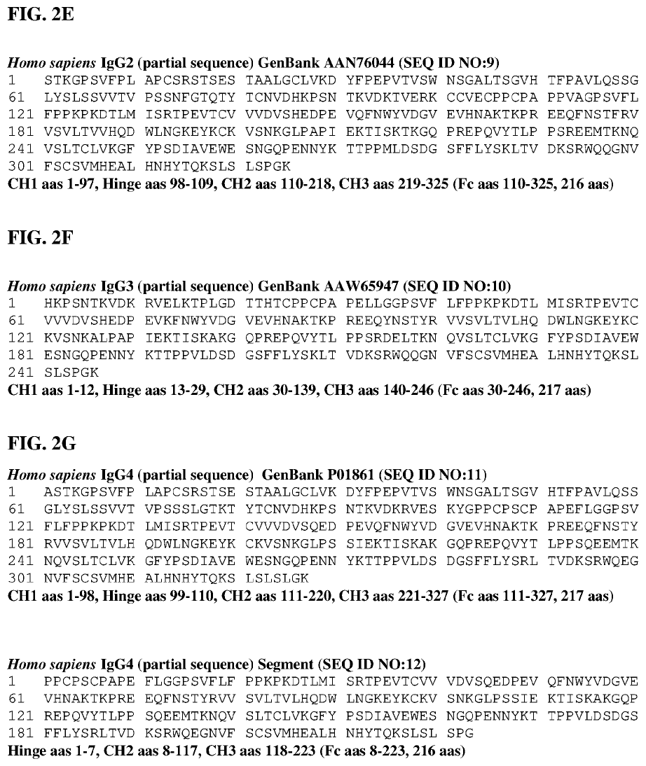

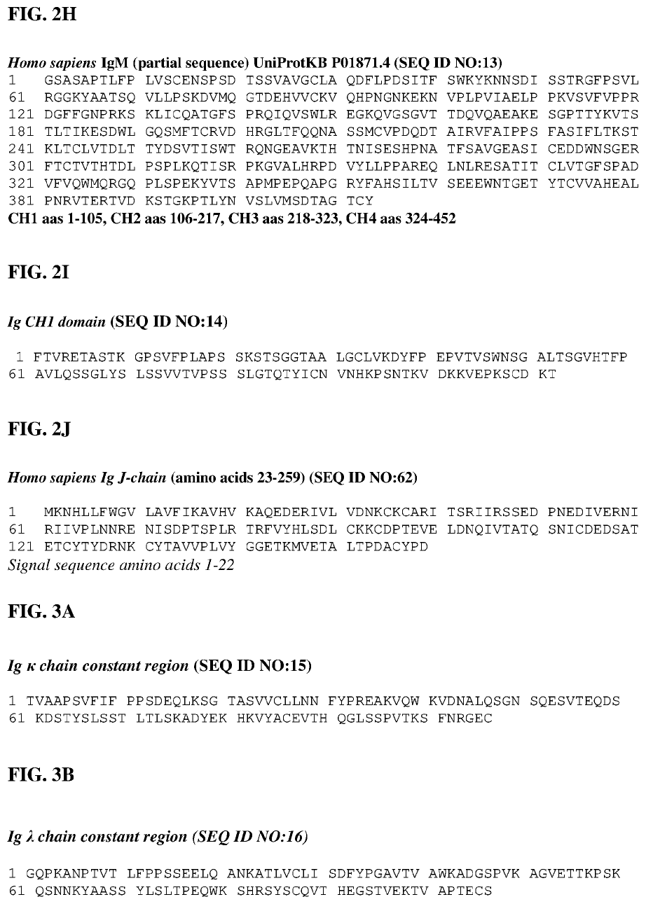

The scaffold polypeptide sequences of a TMAPP may comprise a Fc polypeptide from, for example, from an IgA, IgD, IgE, IgG, or IgM, any of which may be a human polypeptide sequence or a humanized polypeptide sequence. The Fc polypeptide can be from a human IgG1 Fc, a human IgG2 Fc, a human IgG3 Fc, a human IgG4 Fc, a human IgA Fc, a human IgD Fc, a human IgE Fc, a human IgM Fc, etc. In some cases, the Fc polypeptide comprises an as sequence having at least about 70% (e.g., at least about 75%, 80%, 85%, 90%, 95%, 98%, or 99%), or 100% aa sequence identity to at least 125 contiguous aas (e.g., at least 150, at least 175, at least 200, or at least 210 contiguous aas), or all aas of an as sequence of a Fc region depicted in FIGS. 2A-2H. The C-terminal lysine, in particular, provided in some of the sequences provided in FIGS. 2A-2H (e.g., the IgG sequences in FIGS. 2D, 2E, 2F, and 2G) may be removed during cellular processing of TMAPPs and may not be present on some or all of the TMAPP molecules as expressed. See, e.g., van den Bremer et al. (2015) mAbs 7:4; and Sissolak et al. (2019) J. Industrial Microbiol. & Biotechnol. 46:1167. The scaffold polypeptide sequences of a TMAPP may also comprise a Fc region polypeptide of a synthetic heavy chain constant region, or a consensus heavy chain constant region.

Such immunoglobulin sequences can interact forming a duplex or higher order structure from TMAPP molecules. In some instances, the Fc scaffold polypeptide sequences include naturally occurring cysteine residues (or non-naturally occurring cysteine residues provided by protein engineering) that are capable of forming interchain disulfide bonds covalently linking two TMAPP polypeptides together. As discussed below, the Ig Fc region can further contain substitutions that can reduce or substantially eliminate the ability of the Ig Fc to effect complement-dependent cytotoxicity (CDC) or antibody-dependent cell cytotoxicity (ADCC). Unless stated otherwise, the Fc polypeptides used in the TMAPPs and their epitope conjugates do not comprise a transmembrane anchoring domain or a portion thereof sufficient to anchor the TMAPP to a cell membrane.

Most immunoglobulin Fc scaffold polypeptides, particularly those comprising only or largely wt. sequences, may spontaneously link together via disulfide bonds to form homodimers resulting in duplex TMAPPs. In the case of IgM heavy chain constant regions, in the presences of a J-chains, higher order complexes may be formed.

Scaffold polypeptides may comprise an aa sequence having 100% aa sequence identity to the wt. human IgG1 Fc polypeptide depicted in FIG. 2D (SEQ ID NO: 4). A scaffold polypeptide may comprise an aa sequence having at least about 70% (e.g., at least about 80%, 90%, 95%, 98%, or 99%) or 100% aa sequence identity to at least 125 contiguous aas (e.g., at least 150, at least 175, at least 200, or at least 210 contiguous aas), or all aas, of the wt. human IgG1 Fc polypeptide depicted in FIG. 2D. Such scaffold sequences may include a substitution of N297 (N77 as numbered in FIG. 2D, SEQ ID NO:4) with an aa other than asparagine. In one case, N297 is substituted by alanine, (N297A). Substitutions at N297 lead to the removal of carbohydrate modifications and result antibody sequences with reduced complement component 1q (“C1q”) binding compared to the wt. protein, and accordingly a reduction in complement-dependent cytotoxicity (CDC). K322 (e.g., K322A) substitutions shows a substantial reduction in FcγR binding affinity and ADCC, with the C1q binding and CDC functions reduced or substantially eliminated. Hezareh et al., (2001) J. Virol. 75:12161-168.

Amino acid L234 and other aas in the lower hinge region (e.g., aas 234 to 239, such as L235, G236, G237, P238, S239) which correspond to aas 14-19 of SEQ ID NO:8) of IgG are involved in binding to the Fc gamma receptor (FcγR), and accordingly, mutations at that location reduce binding to the receptor (relative to the wt. protein) and resulting in a reduction in antibody-dependent cellular cytotoxicity (ADCC). Hezareh et al., (2001) have demonstrated that the double mutant (L234A, L235A) does not effectively bind either FcγR or C1q, and both ADCC and CDC functions were reduced or substantially eliminated. A scaffold polypeptide with a substitution in the lower hinge region may comprise an aa sequence having at least about 70% (e.g., at least about 80%, 90%, 95%, 98%, or 99%) aa sequence identity to at least 125 contiguous aas (e.g., at least 150, at least 175, at least 200, or at least 210 contiguous aas), or all aas, of the wt. human IgG1 Fc polypeptide depicted in FIG. 2D, that includes a substitution of L234 (L14 of the aa sequence depicted in FIG. 2D) with an aa other than leucine.

A scaffold polypeptide with a substitution in the lower hinge region may comprise an aa sequence having at least about 70% (e.g., at least about 80%, 90%, 95%, 98%, or 99%) aa sequence identity to at least 125 contiguous aas (e.g., at least 150, at least 175, at least 200, or at least 210 contiguous aas), or all aas, of the wt. human IgG1 Fc polypeptide depicted in FIG. 2D, that includes a substitution of L235 (L15 of the aa sequence depicted in FIG. 2D) with an aa other than leucine. In some cases, the scaffold polypeptide present in a TMAPP with substitutions in the lower hinge region includes L234A and L235A (“LALA”) substitutions (the positions corresponding to positions 14 and 15 of the wt. aa sequence depicted in FIG. 2D; see, e.g., SEQ ID NO:8).

A scaffold polypeptide with a substitution in the lower hinge region may comprise an aa sequence having at least about 70% (e.g., at least about 80%, 90%, 95%, 98%, or 99%) aa sequence identity to at least 125 contiguous aas (e.g., at least 150, at least 175, at least 200, or at least 210 contiguous aas), or all aas of the wt. human IgG1 Fc polypeptide depicted in FIG. 2D, that includes a substitution of P331 (P111 of the aa sequence depicted in FIG. 2D) with an aa other than proline. Substitutions at P331, like those at N297, lead to reduced binding to Clq relative to the wt. protein, and thus a reduction in complement dependent cytotoxicity. In one embodiment, the substitution is a P331S substitution. In another embodiment, the substitution is a P331A substitution.

A scaffold polypeptide may comprise an aa sequence having at least about 70% (e.g., at least about 80%, 90%, 95%, 98%, or 99%) aa sequence identity to at least 125 contiguous aas (e.g., at least 150, at least 175, at least 200, or at least 210 contiguous aas), or all aas, of the wt. human IgG1 Fc polypeptide depicted in FIG. 2D, and include substitutions of D270, K322, and/or P329 (corresponding to D50, K102, and P109 of SEQ ID NO:4 in FIG. 2D) that reduce binding to C1q protein relative to the wt. proteins.

A scaffold polypeptide may comprise an aa sequence having at least about 70% (e.g., at least about 80%, 90%, 95%, 98%, or 99%) aa sequence identity to at least 125 contiguous aas (e.g., at least 150, at least 175, at least 200, or at least 210 contiguous aas), or all aas, of the wt. human IgG1 Fc polypeptide depicted in FIG. 2D, including substitutions at L234 and/or L235 (L14 and/or L15 of the aa sequence depicted in FIG. 2D) with aas other than leucine (such as L234A and L235A substitutions), and a substitution of P331 (P111 of the aa sequence depicted in FIG. 2D) with an as other than proline such as P331S. In one instance, a scaffold polypeptide present in a TMAPP comprises the “Triple Mutant” aa sequence (SEQ ID NO:6) depicted in FIG. 2D (human IgG1 Fc) having L234F, L235E, and P331S substitutions (corresponding to aa positions 14, 15, and 111 of the as sequence depicted in FIG. 2D).

The scaffold Fc polypeptide of a TMAPP may comprise an aa sequence having at least about 70% (e.g., at least about 75%, 80%, 85%, 90%, 95%, 98%, or 99%), or 100% aa, sequence identity to at least 125 contiguous aas (e.g., at least 150, at least 175, at least 200, or at least 210 contiguous aas), or all aas, of a human IgG2 Fc polypeptide depicted in FIG. 2E. The scaffold Fc polypeptide of a TMAPP may comprise an as sequence having at least about 70% (e.g., at least about 75%, 80%, 85%, 90%, 95%, 98%, or 99%), or 100% aa, sequence identity to at least 125 contiguous aas (e.g., at least 150, at least 175, at least 200, or at least 210 contiguous aas), or all aas, of a human IgG3 Fc polypeptide depicted in FIG. 2F. The scaffold Fc polypeptide of a TMAPP may comprise an as sequence having at least about 70% (e.g., at least about 75%, 80%, 85%, 90%, 95%, 98%, or 99%), or 100% aa, sequence identity to at least 125 contiguous aas (e.g., at least 150, at least 175, at least 200, or at least 210 contiguous aas), or all aas, of a human IgG4 Fc polypeptide depicted in FIG. 2G. The scaffold Fc polypeptide of a TMAPP may comprise an as sequence having at least about 70% (e.g., at least about 75%, 80%, 85%, 90%, 95%, 98%, or 99%), or 100% aa sequence identity to at least 125 contiguous aas (e.g., at least 150, at least 175, at least 200, or at least 210 contiguous aas e.g., aas 99 to 327 or 111 to 327), or all of the GenBank P01861 human IgG4 Fc polypeptide depicted in FIG. 2G.

The scaffold Fc polypeptide of a TMAPP may comprise IgM heavy chain constant regions (see e.g., FIG. 2H), which forms hexamer, or pentamers (particularly when combined with a mature j-chain peptide lacking a signal sequence, such as that provided in FIG. 2J.

b. Interspecific Immunoglobulin Fc Scaffold Polypeptides

Where an asymmetric pairing between two TMAPP molecules is desired a scaffolds comprising an interspecific Ig Fc polypeptide pair may be employed to produce a heteroduplex TMAPP. Such TMAPP heteroduplexes may be desired when, for example, different MODs are to be located on each of the TMAPPs of the heteroduplex and/or when a masked TGF-β MOD with the masking sequence and TGF-β sequence in trans (on different TMAPPs of the duplex is being formed). Under such circumstances the scaffold polypeptide present in the two TMAPP forming the duplex may comprise, consist essentially of, or consist of an interspecific Ig Fc polypeptide pair. Such interspecific polypeptide sequences include, but are not limited to, knob-in-hole without (KiH) or with (KiHs-s) a stabilizing disulfide bond, HA-TF, ZW-1, 7.8.60, DD-KK, EW-RVT, EW-RVTs-s, and A107 sequences. One interspecific binding pair comprises a T366Y and Y407T mutant pair in the CH3 domain interface of IgG1, or the corresponding residues of other immunoglobulins. See Ridgway et al., Protein Engineering 9:7, 617-621 (1996). A second interspecific binding pair involves the formation of a knob by a T366W substitution, and a hole by the triple substitutions T366S, L368A and Y407V on the complementary Ig Fc sequence. See Xu et al. mAbs 7:1, 231-242 (2015). Another interspecific binding pair has a first Ig Fc polypeptide with Y349C, T366S, L368A, and Y407V substitutions and a second Ig Fc polypeptide with S354C, and T366W substitutions (disulfide bonds can form between the Y349C and the S354C). See e.g., Brinkmann and Konthermann, mAbs 9:2, 182-212 (2015). Ig Fc polypeptide sequences, either with or without knob-in-hole modifications, can be stabilized by the formation of disulfide bonds between the Ig Fc polypeptides (e.g., the hinge region disulfide bonds). Several interspecific binding sequences based upon immunoglobulin sequences are summarized in the table that follows, with cross reference to the numbering of the aa positions as they appear in the wt. IgG1 sequence (SEQ ID NO:4) set forth in FIG. 2D shown in brackets “{ }”.

TABLE 1
Interspecific immunoglobulin sequences and their cognate counterpart interspecific sequences

| | Substitutions in the first | Substitutions in the second | |
|---|---|---|---|
| Interspecific | interspecific polypeptide | (counterpart) interspecific | |
| Pair Name | sequence | polypeptide sequence | Comments |
| KiH | T366W | T366S/L368A/Y407V | Hydrophobic/steric |
| | {T146W} | {T146S/L148A/Y187V} | complementarity |
| KiHs-s | T366W/S354C* | T366S/L368A/Y407V/Y349C | KiH + inter-CH3 |
| | {T146W/S134C*} | {T146S/L148A/Y187V/Y129C} | domain S—S bond |
| HA-TF | S364H/F405A | Y349T/T394F | Hydrophobic/steric |
| | {S144H/F185A} | {Y129T/T174F} | complementarity |
| ZW1 | T350V/L351Y/F405A/Y407V | T350V/T366L/K392L/T394W | Hydrophobic/steric |
| | {T130V/L131Y/F185A/Y187V} | {T130V/T146L/K172L/T174W} | complementarity |
| 7.8.60 | K360D/D399M/Y407A | E345R/Q347R/T366V/K409V | Hydrophobic/steric |
| | {K140D/D179M/Y187A} | {E125R/Q127R/T146V/K189V} | complementarity + |
| | | | electrostatic |
| | | | complementarity |
| DD-KK | K409D/K392D | D399K/E356K | Electrostatic |
| | {K189D/K172D} | {D179K/E136K} | complementarity |
| EW-RVT | K360E/K409W | Q347R/D399V/F405T | Hydrophobic/steric |
| | {K140E/K189W} | {Q127R/D179V/F185T} | complementarity & |
| | | | long-range electro- |
| | | | static interaction |
| EW-RVTs-s | K360E/K409W/Y349C* | Q347R/D399V/F405T/S354C | EW-RVT + inter- |
| | {K140E/K189W/Y129C*} | {Q127R/D179V/F185T/S134C} | CH3 domain S—S |
| | | | bond |
| A107 | K370E/K409W | E357N/D399V/F405T | Hydrophobic/steric |
| | {K150E/K189W} | {E137N/D179V/F185T} | complementarity + |
| | | | hydrogen bonding |
| | | | complementarity |

Table 1 is modified from Ha et al., Frontiers in Immunol. 7: 1-16 (2016).
*aa forms a stabilizing disulfide bond.

In addition to the interspecific pairs of sequences in Table 1, scaffold polypeptides may include interspecific “SEED” sequences having 45 residues derived from IgA in an IgG1 CH3 domain of the interspecific sequence, and 57 residues derived from IgG1 in the IgA CH3 in its counterpart interspecific sequence. See Ha et al., Frontiers in Immunol. 7:1-16 (2016).

Interspecific immunoglobulin sequences may include the substitutions described above for non-interspecific immunoglobulin sequences that inhibit binding either or both of the FcγR or C1q binding, and reduce or substantially eliminate ADCC and/or CDC function.

In an embodiment, a scaffold polypeptide found in a TMAPP may comprise an interspecific binding sequence or its counterpart interspecific binding sequence selected from the group consisting of: knob-in-hole (KiH); knob-in-hole with a stabilizing disulfide (KiHs-s); HA-TF; ZW-1; 7.8.60; DD-KK; EW-RVT; EW-RVTs-s; A107; or SEED sequences.

In an embodiment, a TMAPP comprises a scaffold polypeptide comprising an IgG1 sequence with a T146W KiH sequence substitution, and its counterpart interspecific binding partner polypeptide comprises an IgG1 sequence having T146W, L148A, and Y187V KiH sequence substitutions, where the scaffold polypeptides comprises a sequence having at least 80%, at least 90%, at least 95%, or at least 97% sequence identity to at least 100 (e.g., at least 125, 150, 170, 180, 190, 200, 210, 220, or all 227) contiguous aas of the wt. IgG1 of FIG. 2D. Scaffold polypeptides optionally comprise substitutions at one of more of: L234 and L235 (e.g., L234A/L235A “LALA” or L234F/L235E); N297 (e.g., N297A); P331 (e.g., P331S); L351 (e.g., L351K); T366 (e.g., T366S); P395 (e.g., P395V); F405 (e.g., F405R); Y407 (e.g., Y407A); and K409 (e.g., K409Y). Those substitutions appear at: L14 and L15 (e.g., L14A/L15A “LALA” or L14F/L15E); N77 (e.g., N77A); P111 (e.g., P111S) L131 (e.g., L131K); T146 (e.g., T146S); P175 (e.g., P175V); F185 (e.g., F185R); Y187 (e.g., Y187A); and K189 (e.g., K189Y) in the wt. IgG1 sequence of FIG. 2D.

In an embodiment, a TMAPP or duplex TMAPP comprises a scaffold polypeptide comprising an IgG1 sequence with a T146W KiH sequence substitution, and its counterpart interspecific binding partner polypeptide comprises an IgG1 sequence having T146S, L148A, and Y187V KiH sequence substitutions, where the scaffold polypeptides comprise a sequence having at least 80%, at least 90%, at least 95%, or at least 97% sequence identity to at least 100 (e.g., at least 125, 150, 170, 180, 190, 200, 210, 220, or all 227) contiguous aas of the wt. IgG1 of FIG. 2D; where one or both (in the case of duplex TMAPP) scaffold polypeptide sequence(s) may comprise additional substitutions such as L14 and/or L15 substitutions (e.g., “LALA” substitutions L234A and L235A), and/or N77 (N297 e.g., N297A or N297G).

In an embodiment, a TMAPP or duplex TMAPP comprises a scaffold polypeptide comprising an IgG1 sequence with a T146W and S134C KiHs-s substitutions, and its counterpart interspecific binding partner polypeptide comprises an IgG1 sequence having T146S, L148A, Y187V and Y129C KiHs-s substitutions, where the scaffold polypeptides comprise a sequence having at least 80%, at least 90%, at least 95%, or at least 97% sequence identity to at least 100 (e.g., at least 125, 150, 170, 180, 190, 200, 210, 220, or all 227) contiguous aas of the wt. IgG1 of FIG. 2D; where one or both (in the case of duplex TMAPP) scaffold polypeptide sequence(s) sequences may comprise additional substitutions such as L14 and/or L15 substitutions (e.g., “LALA” substitutions L234A and L235A), and/or N77 (N297 e.g., N297A or N297G).

In an embodiment, a TMAPP comprises a scaffold polypeptide comprising an IgG1 sequence with a S144H and F185A HA-TF substitutions, and its counterpart interspecific binding partner polypeptide comprises an IgG1 sequence having Y129T and T174F HA-TF substitutions, where the scaffold polypeptides comprise a sequence having at least 80%, at least 90%, at least 95%, or at least 97% sequence identity to at least 100 (e.g., at least 125, 150, 170, 180, 190, 200, 210, 220, or all 227) contiguous aas of the wt. IgG1 of FIG. 2D; where one or both (in the case of duplex TMAPP) scaffold polypeptide sequence(s) may comprise additional substitutions such as L14 and/or L15 substitutions (e.g., “LALA” substitutions L234A and L235A), and/or N77 (N297 e.g., N297A or N297G).

In an embodiment, a TMAPP or duplex TMAPP comprises a scaffold polypeptide comprising an IgG1 sequence with a T130V, L131Y, F185A, and Y187V ZW1 substitutions, and its counterpart interspecific binding partner polypeptide comprises an IgG1 sequence having T130V, T146L, K172L, and T174W ZW1 substitutions, where the scaffold polypeptides comprise a sequence having at least 80%, at least 90%, at least 95%, or at least 97% sequence identity to at least 100 (e.g., at least 125, 150, 170, 180, 190, 200, 210, 220, or all 227) contiguous aas of the wt. IgG1 of FIG. 2D; where one or both (in the case of duplex TMAPP) scaffold polypeptide sequence(s) may comprise additional substitutions such as L14 and/or L15 substitutions (e.g., “LALA” substitutions L234A and L235A), and/or N77 (N297 e.g., N297A or N297G).

In an embodiment, a TMAPP or duplex TMAPP comprises a scaffold polypeptide comprising an IgG1 sequence with a K140D, D179M, and Y187A7.8.60 substitutions, and its counterpart interspecific binding partner polypeptide comprises an IgG1 sequence having T130V E125R, Q127R, T146V, and K189V 7.8.60 substitutions, where the scaffold polypeptides comprise a sequence having at least 80%, at least 90%, at least 95%, or at least 97% sequence identity to at least 100 (e.g., at least 125, 150, 170, 180, 190, 200, 210, 220, or all 227) contiguous aas of the wt. IgG1 of FIG. 2D; where one or both (in the case of duplex TMAPP) scaffold polypeptide sequence(s) may comprise additional substitutions such as L14 and/or L15 substitutions (e.g., “LALA” substitutions L234A and L235A), and/or N77 (N297 e.g., N297A or N297G).

In an embodiment, a TMAPP or duplex TMAPP comprises a scaffold polypeptide comprising an IgG1 sequence with a K189D, and K172D DD-KK substitutions, and its counterpart interspecific binding partner polypeptide comprises an IgG1 sequence having T130V D179K and E136K DD-KK substitutions, where the scaffold polypeptides comprise a sequence having at least 80%, at least 90%, at least 95%, or at least 97% sequence identity to at least 100 (e.g., at least 125, 150, 170, 180, 190, 200, 210, 220, or all 227) contiguous aas of the wt. IgG1 of FIG. 2D; where one or both (in the case of duplex TMAPP) scaffold polypeptide sequence(s) may comprise additional substitutions such as L14 and/or L15 substitutions (e.g., “LALA” substitutions L234A and L235A), and/or N77 (N297 e.g., N297A or N297G).

In an embodiment, a TMAPP or duplex TMAPP comprises a scaffold polypeptide comprising an IgG1 sequence with a K140E and K189W EW-RVT substitutions, its counterpart interspecific binding partner polypeptide comprises an IgG1 sequence having T130V Q127R, D179V, and F185T EW-RVT substitutions, where the scaffold polypeptides comprise a sequence having at least 80%, at least 90%, at least 95%, or at least 97% sequence identity to at least 100 (e.g., at least 125, 150, 170, 180, 190, 200, 210, 220, or all 227) contiguous aas of the wt. IgG1 of FIG. 2D; where one or both (in the case of duplex TMAPP) scaffold polypeptide sequence(s) may comprise additional substitutions such as L14 and/or L15 substitutions (e.g., “LALA” substitutions L234A and L235A), and/or N77 (N297 e.g., N297A or N297G).

In an embodiment, a TMAPP or duplex TMAPP comprises a scaffold polypeptide comprising an IgG1 sequence with a K140E, K189W, and Y129C EW-RVTs-s substitutions, its counterpart interspecific binding partner polypeptide comprises an IgG1 sequence having T130V Q127R, D179V, F185T, and S134C EW-RVTs-s substitutions, where the scaffold polypeptides comprise a sequence having at least 80%, at least 90%, at least 95%, or at least 97% sequence identity to at least 100 (e.g., at least 125, 150, 170, 180, 190, 200, 210, 220, or all 227) contiguous aas of the wt. IgG1 of FIG. 2D; where one or both (in the case of duplex TMAPP) scaffold polypeptide sequence(s) may comprise additional substitutions such as L14 and/or L15 substitutions (e.g., “LALA” substitutions L234A and L235A), and/or N77 (N297 e.g., N297A or N297G).

In an embodiment, a TMAPP or duplex TMAPP comprises a scaffold polypeptide comprising an IgG1 sequence with a K150E and K189W A107 substitutions, its counterpart interspecific binding partner polypeptide comprises an IgG1 sequence having T130V E137N, D179V, and F185T A107 substitutions, where the scaffold polypeptides comprise a sequence having at least 80%, at least 90%, at least 95%, or at least 97% sequence identity to at least 100 (e.g., at least 125, 150, 170, 180, 190, 200, 210, 220, or all 227) contiguous aas of the wt. IgG1 of FIG. 2D; where one or both (in the case of duplex TMAPP) scaffold polypeptide sequence(s) may comprise additional substitutions such as L14 and/or L15 substitutions (e.g., “LALA” substitutions L234A and L235A), and/or N77 (N297 e.g., N297A or N297G).

As an alternative to the use of immunoglobulin CH2 and CH3 heavy chain constant regions as scaffold sequences, immunoglobulin light chain constant regions (See FIGS. 3A and 3B) can be paired with Ig CH1 sequences (See, e.g., FIG. 2I) as interspecific scaffold sequences.

### 3. Immunoglobulin CH1 Domain Scaffolds

In an embodiment, a TMAPP scaffold polypeptide comprises an Ig CH1 domain (e.g., the polypeptide of FIG. 2I), and the scaffold sequence with which it will form a complex (its counterpart binding partner) comprises an Ig κ chain or Ig λ chain constant region sequence, where the scaffold polypeptide comprise a sequence having at least 80%, 85%, 90%, 95%, 98%, 99%, or 100% sequence identity to at least 70, at least 80, at least 90, at least 100, or at least 110 contiguous aas of SEQ ID NOs:15 or 16. See FIGS. 2I, 3A and 3B. The Ig CH1 and Ig κ sequences may be modified to increase their affinity for each other, and accordingly the stability of any heterodimer formed utilizing them. Among the substitutions that increase the stability of CH1-Ig κ heterodimers are those identified as the MD13 combination in Chen et al., MAbs, 8(4):761-774 (2016). In the MD13 combination two substitutions introduced into to each of the IgCH1 and Ig κ sequences. The Ig CH1 sequence is modified to contain S64E and S66V substitutions (S70E and S72V of the sequence shown in FIG. 2I). The Ig κ sequence is modified to contain S69L and T71S substitutions (S68L and T70S of the sequence shown in FIG. 3A).

In another embodiment, a scaffold polypeptide of a TMAPP comprises an Ig CH1 domain (e.g., the polypeptide of FIG. 2I SEQ ID NO:14), and its counterpart scaffold sequence comprises an Ig λ chain constant region sequence such as is shown in FIG. 3B (SEQ ID NO:16), where the scaffold polypeptide comprises a sequence having at least 80%, 85%, 90%, 95%, 98%, 99%, or 100% sequence identity to at least 70 (e.g., at least 80, at least 90, or at least 100) contiguous aas of the sequences shown in FIG. 3B.

### 4. Effects on Stability and Half-Life

Suitable scaffold polypeptides (e.g., those with an Ig Fc scaffold sequence) will in some cases extend the be half-life of TMAPP polypeptides and their higher order complexes. In some cases, a suitable scaffold polypeptide increases the in vivo half-life (e.g., the serum half-life) of the TMAPP or duplex TMAPP, compared to a control TMAPP or control duplex TMAPP lacking the scaffold polypeptide or comprising a control scaffold polypeptide. For example, in some cases, a scaffold polypeptide increases the in vivo half-life (e.g., serum half-life) of a conjugated or unconjugated TMAPP or duplex TMAPP, compared to an otherwise identical control TMAPP lacking the scaffold polypeptide, or having a control scaffold polypeptide, by at least about 10%, at least about 20%, at least about 30%, at least about 50%, at least about 2-fold, at least about 5-fold, at least about 10-fold, at least about 25-fold, at least about 50-fold, at least about 100-fold, or more than 100-fold.

### V.A(iv). Immunomodulatory Polypeptides (“MODs”)

A TMAPP may comprise one or more immunomodulatory polypeptides or “MODs”. MODs that are suitable for inclusion in a TMAPP include, but are not limited to, IL-1, IL-2, IL-4, IL-6, IL-7, IL-10, IL-12, IL-15, IL-17, IL-21, IL-23, CD7, CD30L, CD40, CD70, CD80, (B7-1), CD83, CD86 (B7-2), HVEM (CD270), ILT3 (immunoglobulin-like transcript 3), ILT4 (immunoglobulin-like transcript 4), Fas ligand (FasL), ICAM (intercellular adhesion molecule), ICOS-L (inducible costimulatory ligand), JAG1 (CD339), lymphotoxin beta receptor, 3/TR6, OX40L (CD252), PD-L1, PD-L2, TGF-β1, TGF-β2, TGF-β3, 4-1BBL, and fragments of any thereof, such as ectodomain fragments capable of engaging and signaling through their cognate receptor. Some MOD polypeptides suitable for inclusion in a TMAPP, and their “co-MODS (“co-immunomodulatory polypeptides” or cognate costimulatory receptors) include polypeptide sequences with T cell modulatory activity from the protein pairs recited in the following table:

### Exemplary Pairs of MODs and Co-MODs

a) 4-1BBL (MOD) and 4-1BB (Co-MOD);
b) PD-L1 (MOD) and PD1 (Co-MOD);
c) IL-2 (MOD) and IL-2 receptor (Co-MOD);
d) CD80 (MOD) and CD28 (Co-MOD);
e) CD86 (MOD) and CD28 (Co-MOD);
f) OX40L (CD252) (MOD) and OX40 (CD134) (Co-MOD);
g) Fas ligand (MOD) and Fas (Co-MOD);
h) ICOS-L (MOD) and ICOS (Co-MOD);
i) ICAM (MOD) and LFA-1 (Co-MOD);
j) CD30L (MOD) and CD30 (Co-MOD);
k) CD40 (MOD) and CD40L (Co-MOD);
l) CD83 (MOD) and CD83L (Co-MOD);
m) HVEM (CD270) (MOD) and CD160 (Co-MOD);
n) JAG1 (CD339) (MOD) and Notch (Co-MOD);
o) JAG1 (CD339) (MOD) and CD46 (Co-MOD);
p) CD70 (MOD) and CD27 (Co-MOD);
q) CD80 (MOD) and CTLA4 (Co-MOD);
r) CD86 (MOD) and CTLA4 (Co-MOD);
s) PD-L1(MOD) and CD-80 (Co-MOD); and
t) TGF-β1, TGF-β2, and/or TGF-β3 (MODs) and TGF-β Receptor
(e.g., TGFBR1 and/or TGFBR2) (Co-MOD)

In some cases, the MOD is selected from an IL-2 polypeptide, a 4-1BBL polypeptide, a B7-1 polypeptide; a B7-2 polypeptide, an ICOS-L polypeptide, an OX-40L polypeptide, a CD80 polypeptide, a CD86 polypeptide, a PD-L1 polypeptide, a FasL polypeptide, a TGFβ polypeptide, and a PD-L2 polypeptide. In some cases, the TMAPP or duplex TMAPP comprises two different MODs, such as an IL-2 MOD or IL-2 variant MOD polypeptide and either a CD80 or CD86 MOD polypeptide. In another instance, the TMAPP or duplex TMAPP comprises an IL-2 MOD or IL-2 variant MOD polypeptide and a PD-L1 MOD polypeptide. In some case MODs, which may be the same or different, are present in a TMAPP or duplex TMAPP in tandem. When MODs are presented in tandem, their sequences are immediately adjacent to each other on a single polypeptide, either without any intervening sequence or separated by only a linker polypeptide (e.g., no MHC sequences or epitope sequences intervene). The MOD polypeptide may comprise all or part of the extracellular portion of a full-length MOD. Thus, for example, the MOD can in some cases exclude one or more of a signal peptide, a transmembrane domain, and an intracellular domain normally found in a naturally-occurring MOD. Unless stated otherwise, a MOD present in a TMAPP or duplex TMAPP does not comprise the signal peptide, intracellular domain, or a sufficient portion of the transmembrane domain to anchor a substantial amount (e.g., more than 10% or more than 15%) of a TMAPP or duplex TMAPP into a mammalian cell (e.g., a COS cell) membrane.

In some cases, a MOD suitable for inclusion in a TMAPP comprises all or a portion of (e.g., an extracellular portion of) the aa sequence of a naturally-occurring MOD. In other instances, a MOD suitable for inclusion in a TMAPP is a variant MOD that comprises at least one aa substitution compared to the aa sequence of a naturally-occurring MOD. In some instances, a variant MOD exhibits a binding affinity for a co-MOD that is lower than the affinity of a corresponding naturally-occurring MOD (e.g., a MOD not comprising the aa substitution(s) present in the variant) for the co-MOD. Suitable variations in MOD polypeptide sequence that alter affinity may be identified by scanning (making aa substitution e.g., alanine substitutions or “alanine scanning” or charged residue changes) along the length of a peptide and testing its affinity. Once key an positions altering affinity are identified those positions can be subject to a vertical scan in which the effect of one or more aa substitutions other than alanine are tested.

V.A(v). MODs and Variant MODs with Reduced Affinity

A MOD can comprise a wild-type amino acid sequence, or can comprise one or more amino acid substitutions, insertions, and/or deletions relative to a wild-type amino acid sequence. The immunomodulatory polypeptide can comprise only the extracellular portion of a full-length immunomodulatory polypeptide. Alternatively, a MOD can comprise all or a portion of (e.g., an extracellular portion of) the amino acid sequence of a naturally-occurring MOD polypeptide.

Variant MODs comprise at least one amino acid substitution, addition and/or deletion as compared to the amino acid sequence of a naturally-occurring immunomodulatory polypeptide. As noted above, in some instances a variant MOD exhibits a binding affinity for a co-MOD that is lower than the affinity of a corresponding naturally-occurring MOD (e.g., an immunomodulatory polypeptide not comprising the amino acid substitution(s) present in the variant) for the co-MOD.

MOD polypeptides and variants, including reduced affinity variants, of proteins such as PD-L1, CD80, CD86, 4-1BBL and IL-2 are described in the published literature, e.g., published PCT application WO2020132138A1, the disclosure of which as it pertains to immunomodulatory polypeptides and specific variant immunomodulatory polypeptides of PD-L1, CD80, CD86, 4-1BBL, IL-2 are expressly incorporated herein by reference, including specifically paragraphs [00260]-[00455] of WO2020132138A1.

Suitable immunomodulatory domains that exhibit reduced affinity for a co-immunomodulatory domain can have from 1 as to 20 as differences from a wild-type immunomodulatory domain. For example, in some cases, a variant MOD present in a TMAPP may include a single as substitution compared to a corresponding reference (e.g., wild-type) MOD. A variant MOD present in a TMAPP may include 2 aa substitutions compared to a corresponding reference (e.g., wild-type) MOD. A variant MOD present in a TMAPP may include 3 or 4 as substitutions compared to a corresponding reference (e.g., wild-type) MOD. A variant MOD present in a TMAPP may include 5 or 6 as substitutions compared to a corresponding reference (e.g., wild-type) MOD. A variant MOD present in a TMAPP may include 7, 8, 9 or 10 as substitutions compared to a corresponding reference (e.g., wild-type) MOD. A variant MOD present in a TMAPP may include 11-15 or 15-20 as substitutions compared to a corresponding reference (e.g., wild-type) MOD.

As discussed above, a variant MOD suitable for inclusion in a TMAPP may exhibit reduced affinity for a cognate co-MOD, compared to the affinity of a corresponding wild-type MOD for the cognate co-MOD.

Binding affinity between a MOD polypeptide sequence and its cognate co-MOD polypeptide can be determined by bio-layer interferometry (BLI) using the purified MOD polypeptide sequence and purified cognate co-MOD polypeptide, following the procedure set forth in published PCT Application WO 2020˜132138 A1.

### 1. Masked TGF-β MODs

a. Masked TGF-β Peptides

As discussed above, a TMAPP of the present disclosure comprises at least one TGF-β polypeptide reversibly masked by a polypeptide (a “masking polypeptide”) that binds to the TGF-β polypeptide, which together form a masked TGF-β MOD. The masking polypeptide can be, for instance, a TGF-β receptor polypeptide or an antibody that functions to reversibly mask the TGF-β polypeptide present in the TMAPP or its epitope conjugate, where the TGF-β polypeptide is otherwise capable of acting as an agonist of a cellular TGF receptor. The masked TGF-β MODs provide active TGF-β polypeptides (e.g., TGF-β signaling pathway agonists). The TGF-β polypeptides and masking polypeptides (e.g., a TGF-β receptor fragment) interact with each other to reversibly mask the TGF-β polypeptide, thereby permitting the TGF-β polypeptide to interact with its cellular receptor. In addition, the masking sequence competes with cellular receptors that can scavenge TGF-β, such as the non-signaling TPRIII, thereby permitting the TGF-β MOD (and thus the TMAPP-epitope conjugate) to effectively deliver active TGF-β agonist to target cells. While the TMAPP-epitope conjugate constructs discussed herein permit epitope-specific presentation of a reversibly masked TGF-β to a target T cell, they also provide sites for the presentation of one or more additional MODs. The ability of the TMAPP construct to include one or more additional MODs thus permits the combined presentation of TGF-β and the additional MOD(s) to direct a target T cell's response in a substantially epitope-specific/selective manner in order to provide modulation of the target T cell. The TMAPP-epitope conjugate thereby permits delivery of one or more masked TGF-β MODs in an epitope-selective (e.g., dependent/specific) manner that permits (i) formation of an active immune synapse with a target T cell, such as a CD4+ cell selective for the epitope, and (ii) modulation (e.g., control/regulation) of the target T cell's response to the epitope. Once engaged with the TCR of a T cell, the effect of a masked TGF-β MOD-containing TMAPP-epitope conjugate on the T cell will depend on whether any additional MODs are present as part of the TMAPP and, if so, which additional MOD(s) is/are present.

Further, although the TMAPPs of this disclosure may comprise both one or more masked TGF-β MODs and one or more additional MODs such as a wt. or variant IL-2, PD-L1 and/or a 4-1BBL MOD (as discussed above), if desired, the TMAPPs of this disclosure may comprise only one or more masked TGF-β MODs. That is, the one or more additional MODs such as the wt. or variant IL-2, PD-L1 and/or a 4-1BBL MOD need not be included in a TMAPP of this disclosure. The masked TGF-β MOD-containing TMAPP-epitope conjugates of the present disclosure can function as a means of producing TGF-β-driven T cell responses. For example, TGF-β by itself can inhibit the development of effector cell functions of T cells, activate macrophages, and/or promote tissue the repair after local immune and inflammatory actions subside.

Although masked TGF-β MODs comprise a TGF-β polypeptide that is masked, the TGF-β polypeptide can still act as TβR agonist because the TGF-β polypeptide-mask complex is reversible and “breathes” between an open state where the TGF-beta polypeptide is available to cellular receptors, and a closed state where the mask engages the TGF-β polypeptide. Accordingly, the masking polypeptide functions to bind TGF-β polypeptide and prevent it from entering into tight complexes with, for example, ubiquitous non-signaling TβR3 molecules that can scavenge otherwise free TGF-β. Moreover, because the active forms of TGF-β are dimers that have higher affinity for TBR3, substitutions that limit dimerization (e.g., a C77Ssubstiitution of the cysteine at position 77 with a serine) can be incorporated into TGF-β sequences in order to avoid scavenging by that receptor.

One effect of the masking sequence is to reduce the effective affinity of TGF-β1, TGF-β2, and TGF-β3 polypeptides for TβRs. At the same time, the affinity of the masking polypeptide for the TGF-β polypeptide can be altered so that it dissociates more readily from the TGF-β polypeptide, making the TGF-β polypeptide more available to cellular TβR proteins. That is, where the affinity of a masking polypeptide for a TGF-β polypeptide is reduced, the masked TGF-β MOD will spend more time in the open state. Although in the open state with the TGF-β polypeptide available for binding to cellular receptors, because the TβRII protein is generally the first peptide of the heteromeric TβR1/TβR2 signaling complex to interact with TGF-β, control of the affinity of the TGF-β polypeptide for TβRII effectively controls entry of TGF-β into active signaling complexes. The incorporation of substitution at, for example, one or more, two or more, or all three of Lys 25, Ile 92, and/or Lys 94 of TGF-β2 (or the corresponding positions of TGF-β 1, TGF-β3) reduces affinity for TβRII polypeptides. The reduced affinity permits interactions between the target cell's TCR and the TMAPP-epitope conjugates MHC polypeptides and epitope to effectively control binding and allows for target cell-specific interactions.

When a TβRII polypeptide is used as the masking polypeptide, the possibility of direct interactions with cellular TβRI receptors and off-target signaling can be addressed by appropriate modifications of the masking sequence. Where it is desirable to block/limit signaling by the masked TGF-β polypeptide through TβRI and/or modify (e.g., reduce) the affinity of a masking TβRII polypeptide for TGF-β, it is possible to incorporate N-terminal deletions and/or aa substitutions in the masking TβRII polypeptide. Modifications that can be made include deletions of N-terminal amino acids (e.g., N-terminal Δ14 or Δ25 deletions), and/or substitutions at one or more of L27, F30, D32, S49, 150, T51, S52, 153, E55, V77, D118, and/or E119. Some specific TβRII modifications resulting in a reduction in TβRI association with TβRII and reduced affinity for TGF-β include any one or more of L27A, F30A, D32A, D32N, S49A, 150A, T51A, S52A, S52L, 153A, E55A, V77A, D118A, D118R, E119A, and/or E119Q.

The TGF-β polypeptide present in a TMAPP is in some cases a variant TGF-β polypeptide, including a variant TGF-β polypeptide that has a lower affinity for at least one class of TGF-β receptors, or is selective for at least one class of TGF-β receptors, compared to a wild-type TGF-β polypeptide.

While a TGF-β 1 polypeptide, a TGF-β2 polypeptide, or a TGF-β3 polypeptide can be incorporated into a TMAPP as part of a masked TGF-β polypeptide, a variety of factors may influence the choice of the specific TGF-β polypeptide, and the specific sequence and an substitutions that will be employed. For example, TGF-β0l and TGF-β3 polypeptides are subject to “clipping” of their amino acid sequences when expressed in a certain mammalian cell lines (e.g., CHO cells). In addition, dimerized TGF-β (e.g., TGF-β2) has a higher affinity for the TβR3 (beta glycan receptor) than for the TβR2 receptor, which could lead to off target binding and loss of biologically active masked protein to the large in vivo pool of non-signaling TβR3 molecules. To minimize high-affinity off target binding to TβR3, it may be desirable to substitute the residues leading to dimeric TGF-β molecules, which are joined by a disulfide bond. Accordingly, cysteine 77 (C77) may be substituted by an amino acid other than cysteine (e.g., a serine forming a C77S substitution).

Amino acid sequences of TGF-β polypeptides are known in the art. In some cases, the TGF-β polypeptide present in a masked TGF-β polypeptide is a TGF-β1 polypeptide. In some cases, the TGF-β polypeptide present in a masked TGF-β polypeptide is a TGF-β2 polypeptide. In some cases, the TGF-β polypeptide present in a masked TGF-β polypeptide is a TGF-β3 polypeptide.

A suitable TGF-β polypeptide can have a length from about 70 aas to about 125 aas; for example, a suitable TGF-β polypeptide can have a length from about 70 aas to about 80 aas from about 80 aas to about 90 aas; from about 90 aas to about 100 aas; from about 100 aas to about 105 aas, from about 105 aas to about 110 aas, from about 110 aas to about 112 aas, from about 113 aas to about 120 aas, or from about 120 aas to about 125 aas. A suitable TGF-β polypeptide can comprise an amino acid sequence having at least 60%, at least 70%, at least 80%, at least 80%, at least 85%, at least 90%, at least 95%, at least 98%, at least 99%, or 100%, aa sequence identity to at least 80, at least 90, at least 100, or at least 110 contiguous aas of the mature form of a human TGF-β0l polypeptide, a human TGF-β2 polypeptide, or a human TGF-β3 polypeptide.

### (i) TGF-β1 Polypeptides

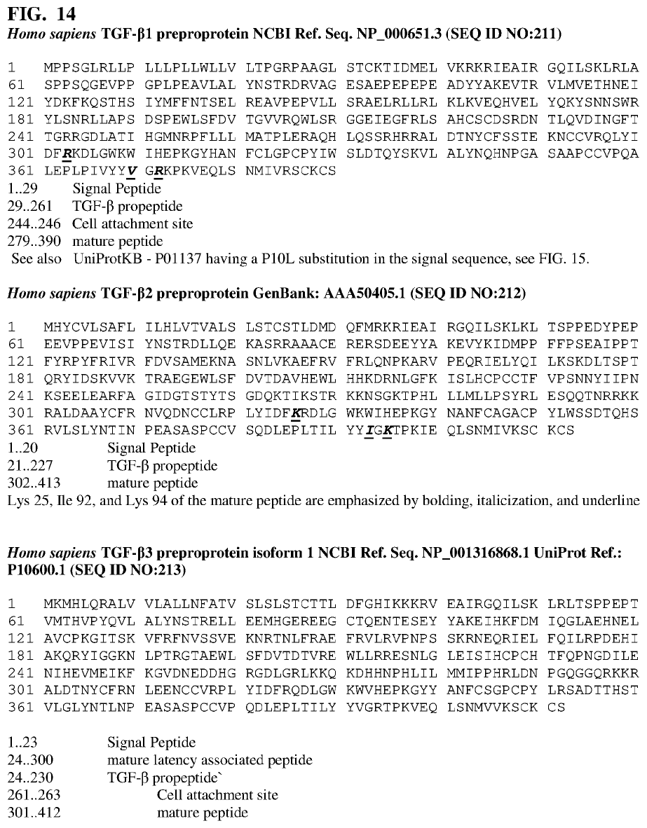

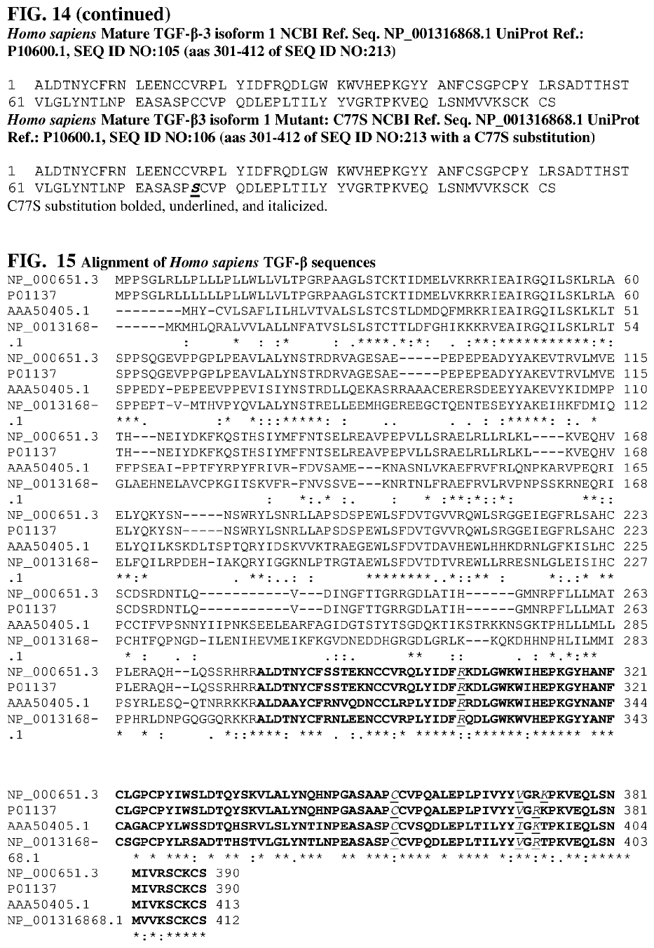

A suitable TGF-β1 polypeptide can comprise an amino acid sequence having at least 60%, at least 70%, at least 80%, at least 90%, at least 95%, at least 98%, at least 99%, or 100%, an sequence identity to at least 70, at least 80, at least 90, at least 100, at least 110, or 112 aas of the following TGF-β1 amino acid sequence: AL DTNYCFSSTE KNCCVRQLYI DF [symbol] KDLGWKW IHEPKGYHAN FCLGPCPYIW SLDTQYSKVL ALYNQHNPGA SAAP [symbol] CVPQA LEPLPIVYY [symbol] G [symbol] KPKVEQLS NMIVRSCKCS (SEQ ID NO:101, 112 aas in length); where the TGF-β1 polypeptide has a length of about 112 aas. A TGF-β1 preproprotein is provided in FIG. 14 as SEQ ID NO:211. Amino acids R25, C77, V92 and R94 are bolded and italicized. See FIG. 14.

A suitable TGF-β1 polypeptide may comprise a C77S substitution. Thus, in some cases, a suitable TGF-β1 polypeptide comprises an amino acid sequence having at least 60%, at least 70%, at least 80%, at least 90%, at least 95%, at least 98%, at least 99%, or 100%, an sequence identity to at least 70, at least 80, at least 90, at least 100, at least 110, or 112 aas of the following TGF-β1 amino acid sequence: AL DTNYCFSSTE KNCCVRQLYI DF [symbol] KDLIWKW IHEPKGYHAN FCLGPCPYIW SLDTQYSKVL ALYNQHNPGA SAAP [symbol] CVPOA LEPLPIVYY [symbol] G [symbol] RKPKVEQLS NMIVRSCKCS (SEQ ID NO:102), where amino acid 77 is Ser. Positions 25, 77, 92 and 94 are bolded and italicized.

### (ii) TGF-β2 Polypeptides

A suitable TGF-β2 polypeptide can comprise an amino acid sequence having at least 60%, at least 70%, at least 80%, at least 90%, at least 95%, at least 98%, at least 99%, or 100%, aa sequence identity to at least 70, at least 80, at least 90, at least 100, at least 110, or 112 aas of the following TGF-β2 amino acid sequence: ALDAAYCFR NVQDNCCLRP LYIDF [symbol] RDLG WKWIHEPKGY NANFCAGACP YLWSSDTQHS RVLSLYNTIN PEASASPCCV SQDLEPLTIL YY [symbol] G [symbol] TPKIE QLSNMIVKSC KCS (SEQ ID NO:103), where the TGF-β2 polypeptide has a length of about 112 aas. A TGF-β2 preproprotein is provided in FIG. 14 as SEQ ID NO:212. Residues Lys 25, Cys 77, Ile 92, and Lys 94 are bolded and italicized.

A suitable TGF-β2 polypeptide may comprise a C77S substitution. Thus, in some cases, a suitable TGF-β2 polypeptide comprises an amino acid sequence having at least 60%, at least 70%, at least 80%, at least 90%, at least 95%, at least 98%, at least 99%, or 100%, an sequence identity to at least 70, at least 80, at least 90, at least 100, at least 110, or 112 aas of the following TGF-β2 amino acid sequence: ALDAAYCFR NVQDNCCLRP LYIDFKRDLG WKWIHEPKGY NANFCAGACP YLWSSDTQHS RVLSLYNTIN PEASASPSCV SQDLEPLTIL YYIGKTPKIE QLSNMIVKSC KCS (SEQ ID NO:104), where amino acid 77 is substituted by a Ser that is bolded and italicized.

(iii) TGF-β3 Polypeptides

A suitable TGF-β3 polypeptide can comprise an amino acid sequence having at least 60%, at least 70%, at least 80%, at least 90%, at least 95%, at least 98%, at least 99%, or 100%, aa sequence identity to at least 70, at least 80, at least 90, at least 100, at least 110, or 112 aas of the following TGF-β3 amino acid sequence: ALDTNYCFRN LEENCCVRPL YIDF [symbol] QDLGW KWVHEPKGYY ANFCSGPCPY LRSADTTHST VLGLYNTLNP EASASPCCVP QDLEPLTILY Y [symbol] G [symbol] TPKVEQ LSNMVVKSCK CS (SEQ ID NO:105), where the TGF-β3 polypeptide has a length of about 112 aas. A TGF-β3 isoform 1 preproprotein is provided in FIG. 14 as SEQ ID NO:213. Positions 25, 92 and 94 are bolded and italicized.

A suitable TGF-β3 polypeptide may comprise a C77S substitution. In some cases, a suitable TGF-β3 polypeptide comprises an amino acid sequence having at least 60%, at least 70%, at least 80%, at least 90%, at least 95%, at least 98%, at least 99%, or 100%, aa sequence identity to at least 70, at least 80, at least 90, at least 100, at least 110, or 112 aas of the following TGF-β3 amino acid sequence: ALDTNYCFRN LEENCCVRPL YIDFRQDLGW KWVHEPKGYY ANFCSGPCPY LRSADTTHST VLGLYNTLNP EASASPSCVP QDLEPLTILY YVGRTPKVEQ LSNMVVKSCK CS (SEQ ID NO: 106), where amino acid 77 is Ser. Positions 25, 92 and 94 are bolded and italicized.

### (iv) Additional TGF-β Polypeptide Sequence Variations

In addition to sequence variations that alter TGF-β molecule dimerization (e.g., cysteine 77 substitutions such as C77S), TGF-β1, TGF-β2, and TGF-β3 polypeptides having sequence variations that affect affinity and other properties may be incorporated into a masked TGF-β MOD. When a variant TGF-β with reduced affinity for the masking polypeptide (e.g., a TβR polypeptide such as a TβRII polypeptide) is present in the masked TGF-β MOD those components dissociate more readily, making the TGF-β polypeptide more available to cellular TβR proteins. Because the TβRII protein is generally the first peptide of the heteromeric TβR signaling complex to interact with TGF-β, interactions with TβRII effectively controls entry of TGF-β into active signaling complexes. Accordingly, variants controlling the affinity of TGF-β for TβRII may effectively control entry of masked TGF-β MODs into active signaling complexes.

The present disclosure includes and provides for masked TGF-β MODs comprising a variant masking TβR (e.g., TβRII) polypeptide sequence and/or a variant TGF-β polypeptide having altered (e.g., reduced) affinity for each other (relative to an otherwise identical masked TGF-β MOD without the sequence variation(s)). Affinity between a TGF-β polypeptide and a TβR (e.g., TβRII) polypeptide may be determined using (BLI) as described above for MODs and their co-MODs.

### (a) Additional TGF-β2 Sequence Variants

The present disclosure includes and provides for masked TGF-β2 MODs comprising a masking TβR (e.g., TβRII) polypeptide sequence and either a wt. or a variant TGF-β2 polypeptide; where the variant polypeptide has a reduced affinity for the masking TβR (relative to an otherwise identical wt. TGF-β polypeptide sequence without the sequence variations).

The disclosure provides for a masked TGF-β MODs that comprise a masking TβRII receptor sequence and a variant TGF-β2 polypeptide having greater than 85% (e.g., greater than 90%, 95%, 98% or 99%) sequence identity to at least 100 contiguous aa of SEQ ID NO:103, and comprising a substitution reducing the affinity of the variant TGF-β2 polypeptide for the TβRII receptor sequence.

In some cases, a masked TGF-β MOD comprises a masking TβRII polypeptide and a variant TGF-β (e.g., TGF-β2) polypeptide comprising a substitution at one or more, two or more, or all three of Lys 25, Ile 92, and/or Lys 94 (see SEQ ID NO:103 for the location of the residues, and FIG. 15 for the corresponding residues in TGF-β 1 and TGF-β3). Those aa residues have been shown to affect the affinity of TGF-β2 for TβRII polypeptides (see Crescenzo et al., J. Mol. Biol. 355: 47-62 (2006)). The TMAPP optionally comprises one or more independently selected MODs such as IL-2 or a variant thereof. In one instance, the masked TGF-β MOD comprises a masking TβRII polypeptide and a TGF-β2 polypeptide having an as other than Lys or Arg at position 25 of SEQ ID NO:103; with the TMAPP optionally comprising one or more additional independently selected MODs (e.g., one or more IL-2 MOD polypeptide or reduced affinity variant thereof). A masked TGF-β MOD with a masking TβRII polypeptide may comprises a TGF-β2 polypeptide having an aa other than Ile or Val at position 92 of SEQ ID NO:103 (or an as other than Ile, Val, or Leu at position 92); with the TMAPP optionally comprising one or more additional independently selected MODs (e.g., one or more IL-2 MOD polypeptide or reduced affinity variant thereof). A masked TGF-β MOD with a masking TβRII polypeptide may comprise a TGF-β2 polypeptide having an aa other than Lys or Arg at position 94 of SEQ ID NO:103); with the TMAPP optionally comprising one or more additional independently selected MODs (e.g., one or more IL-2 MOD polypeptide or reduced affinity variant thereof). A masked TGF-β MOD with a masking TβRII polypeptide may comprise a TGF-β2 polypeptide comprising a substitution at one or more, two or more or all three of Lys 25, Ile 92, and/or Lys 94); with the TMAPP optionally comprising one or more additional independently selected MODs. A masked TGF-β MOD with a masking TβRII polypeptide may comprise a TGF-β2 polypeptide comprising a substitution at one or more, two or more or all three of Lys 25, Ile 92, and/or Lys 94); with the TMAPP optionally comprising one or more additional independently selected IL-2 MODs or reduced affinity variants thereof.

### (b) Additional TGF-β1 and TGF-β3 Sequence Variants

In some cases, a masked TGF-β MOD comprises a masking TβRH polypeptide and a variant TGF-β1 or TGF-β3 polypeptide comprising a substitution at one or more, two or more or all three aa positions corresponding to Lys 25, Ile 92, and/or Lys 94 in TGF-β2 SEQ ID NO:103. In TGF-β01 or TGF-β3, the as that corresponds to: Lys 25 is an Arg 25, le 92 is Val 92, and Lys 94 is Arg 94, each of which is a conservative substitution. See e.g., SEQ ID NOs:211 and 102 for TGF-β1 and SEQ ID NOs:213 and 106 for TGF-β3.

As noted above, the masked TGF-β MOD optionally comprises one or more independently selected MODs such as IL-2 or a variant thereof. In one instance, the masked TGF-β MOD with a masking TβRII polypeptide comprises a TGF-β1 or 03 polypeptide having an aa other than Arg or Lys at position 25; and optionally comprises one or more independently selected MODs (e.g., one or more IL-2 MOD polypeptide or reduced affinity variant thereof). In one instance, the masked TGF-§ MOD with a masking TβRII polypeptide comprises a TGF-β1 or β3 polypeptide having an aa other than Val or Ile at position 92 (or an aa other than Ile, Val, or Leu at position 92); and optionally comprises one or more independently selected MODs (e.g., one or more IL-2 MOD polypeptide or reduced affinity variant thereof). In another instance, the masked TGF-β MOD with a masking TβRII polypeptide comprises a TGF-β2 polypeptide having an aa other than Arg or Lys; and optionally comprises one or more independently selected MODs (e.g., one or more IL-2 MOD polypeptide or reduced affinity variant thereof). In one specific instance, a masked TGF-β MOD with a masking TβRII polypeptide comprises a TGF-β1 or 03 polypeptide comprising a substitution at one or more, two or more or all three of Arg 25, Val 92, and/or Arg 94, and further comprises one or more independently selected MODs (e.g., IL-2 or variant IL-2 MODs). In another specific instance, a masked TGF-β MOD with a masking TβRII polypeptide comprises a TGF-β1 or 03 polypeptide comprising a substitution at one or more, two or more or all three of Arg 25, Val 92, and/or Arg 94, and further comprises one or more independently selected IL-2 MODs, or reduced affinity variants thereof.

b. TGF-β Receptor Polypeptides and Other Polypeptides that Bind and Mask TGF-β

In any of the above-mentioned TGF-β polypeptides or polypeptide complexes the polypeptide that binds to and masks the TGF-β polypeptide (the “masking polypeptide”) can take a variety of forms, including fragments of TβRI, TβRII, TβRIII and anti-TGF-β antibodies or antibody-related molecules (e.g., antigen binding fragment of an antibody, Fab, Fab′, single chain antibody, scFv, peptide aptamer, or nanobody).

### (i) TGF-β Receptor Polypeptides

The masking of TGF-β in masked TGF-β MODs may be accomplished by utilizing a TGF-β receptor fragment (e.g., the ectodomain sequences of TβRI, TβRII or TβRIII) that comprises polypeptide sequences sufficient to bind a TGF-β polypeptide (e.g., TGF-β 1, TGF-β2 or TGF-β3). In an embodiment, the masking sequence comprises all or part of the TβRI, TβRII, or TβRIII ectodomain.

### (a) TGF-β Receptor I (TβRI)

The polypeptide sequence masking TGF-β in a masked TGF-β MODs may be derived from a TβRI (e.g., isoform 1 SEQ ID NO:214) and may comprises all or part of the TβRI ectodomain (aas 34-126). A suitable TβRI polypeptide for masking TGF-β may comprise an amino acid sequence having at least 60%, at least 70%, at least 80%, at least 90%, at least 95%, at least 98%, at least 99%, or 100%, aa sequence identity to at least 70, at least 80, at least 90, at least 100, or 103 aas of the following TβRI ectodomain aa sequence: LQCFCHL CTKDNFTCVT DGLCFVSVTE TTDKVIHNSM CIAEIDLIPR DRPFVCAPSS KTGSVTITYC CNQDHCNKIE LPTTVKSSPG LGPVEL (SEQ ID NO:107).

### (b) TGF-β Receptor II (TβRII)

A polypeptide sequence masking TGF-β in a masked TGF-β MOD may be derived from a TβRII (e.g., isoform A SEQ ID NO:215), and may comprises all or part of the TβRII ectodomain sequence (aas 24 to 177). A suitable TβRII isoform A polypeptide for masking TGF-β may comprise an amino acid sequence having at least 60%, at least 70%, at least 80%, at least 90%, at least 95%, at least 98%, at least 99%, or 100%, aa sequence identity to at least 70, at least 80, at least 90, at least 100, at least 110, at least 120, at least 130, at least 140, at least 150 or at least 154 aas of the following TβRII isoform A ectodomain aa sequence: IPPHVQK SDVEMEAQKD EIICPSCNRT AHPLRHINND MIVTDNNGAV KFPQLCKFCD VRFSTCDNQK SCMSNCSITS ICEKPQEVCV AVWRKNDENI TLETVCHDPK LPYHDFILED AASPKCIMKE KKKPGETFFM CSCSS [symbol] ECND NIIFSEE (SEQ ID NO:108). The location of the aspartic acid residue corresponding to D118 in the B isoform is bolded and italicized.

A polypeptide sequence masking TGF-β in a masked TGF-β MOD may be derived from TRII isoform B SEQ ID NO:216) and may comprises all or part of the TβRII ectodomain sequence (aas 24 to 166). A suitable TβRII isoform B polypeptide for masking TGF-β may comprise an amino acid sequence having at least 60%, at least 70%, at least 80%, at least 90%, at least 95%, at least 98%, at least 99%, or 100%, aa sequence identity to at least 70, at least 80, at least 90, at least 100, at least 110, at least 120, at least 130, at least 140, or 143 aas of the RII isoform B ectodomain aa sequence: IPPHVQKSVN NDMIVTDNNG AVKFPQLCK [symbol] C [symbol] VRFSTCDN QKSCMSNCSI TSIC [symbol] KPQEV CVAVWRKNDE NITLETVCHD PKLPYHDFIL EDA APiM KEKKKPGETF FMCSCSS [symbol] EC NDNIIFSEEY NTSNPDLLLV IFQ (SEQ ID NO:109). As discussed below, any one or more of F30, D32, S52, E55, or D118 (italicized and bolded) may be substituted by an amino acid other than the naturally occurring aa at those positions (e.g., alanine). A polypeptide sequence masking TGF-β may comprise the polypeptide of SEQ ID NO:109 bearing a D118A or D118R substitution. A sequence masking TGF-β may comprise the peptide of SEQ ID NO:109 bearing a D118A or D118R substitution and one or more of a F30A, D32N, S52L and/or E55A substitution.

Although TβRII's ectodomain may be utilized as a masking polypeptide, that region of the protein has charged and hydrophobic patches that can lead to an unfavorable pI and can be toxic to cells expressing the polypeptide. In addition, combining a TβRII ectodomain with the an active TGF-β polypeptide can result in a complex that could combine with cell surface TβRI and cause activation of that signaling receptor (e.g., signaling through the Smad pathway). Modifying TβRII ectodomain sequences used to mask TGF-β by removing or altering sequences involved in TβRI association can avoid the unintentional stimulation of cells by the masked TGF-β except through their own cell surface heterodimeric TβRI/TβRII complex. Modifications of TβRII may also alter (e.g., reduce) the affinity of the TβRII for TGF-β (e.g., TGF-β3), thereby permitting control of TGF-β unmasking and its availability as a signaling molecule. Masked TGF-β MODs comprising TBR (e.g., TβRII) peptides with the highest affinity for TGF-β (e.g., TGF-β3) most tightly mask the TGF-β sequence and require higher doses to achieve the same effect. In contrast, aa substitutions in TβRII that lower the affinity unmask the TGF-β polypeptide and are biologically effective at lower doses.

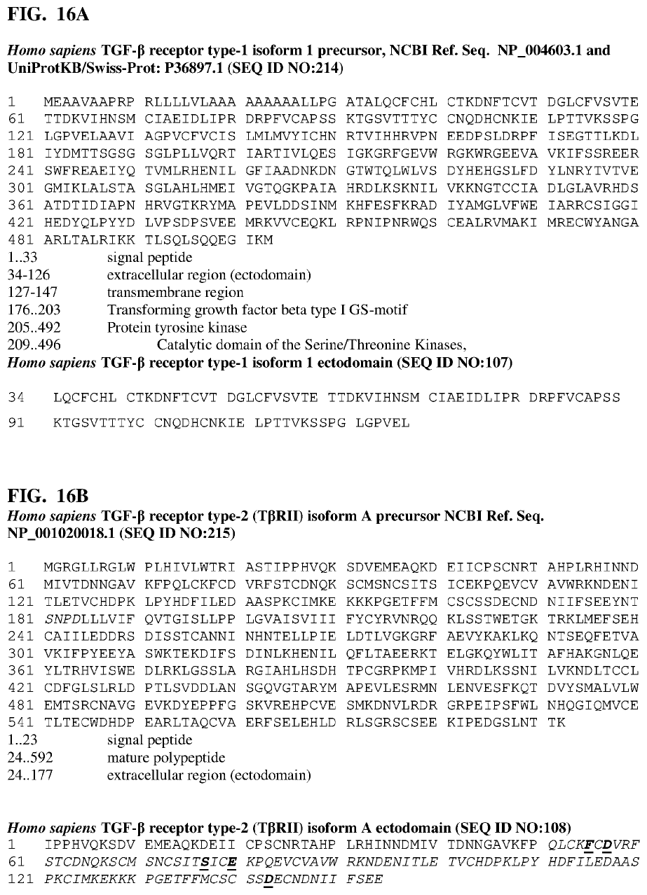

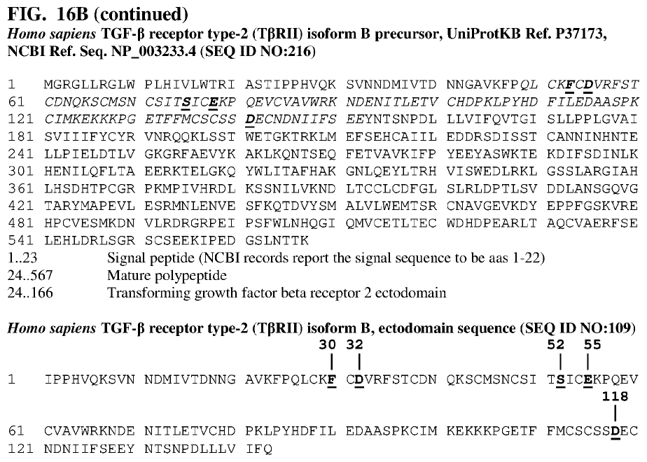

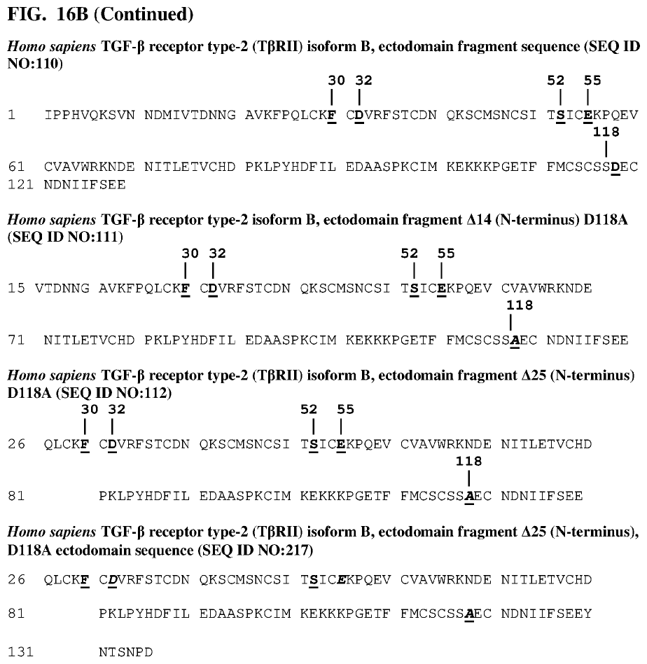

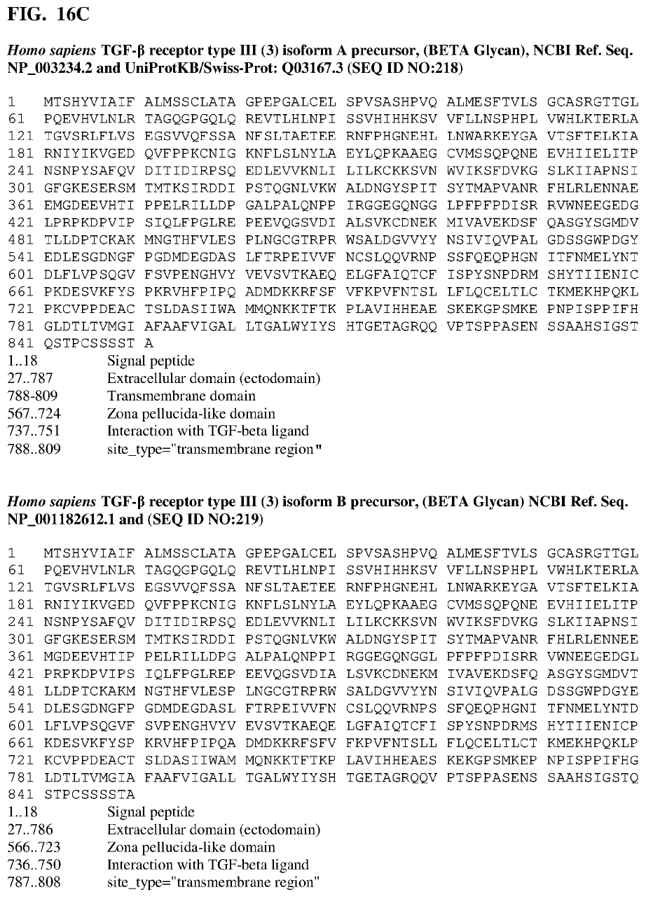

Accordingly, where it is desirable to block/limit signaling by the masked TGF-β polypeptide through TβRI and/or modify (e.g., reduce) the affinity of a masking TβRII polypeptide for TGF-β a number of alterations to TβRII may be incorporated into the TβRII polypeptide sequence. Modifications that can be made include the above-mentioned deletions of N-terminal amino acids, such as 14 or 25 N-terminal amino acids (from 1 to 14aas or from 1 to 25 aas; Δ14, Δ25 modifications), and/or substitutions at one or more of L27, F30, D32, S49, 150, T51, S52, I53, E55, V77, D118, and/or E119. Some specific TβRII modifications resulting in a reduction in TβRI association with TβRII and reduced affinity for TGF-β include any one or more of L27A, F30A, D32A, D32N, S49A, I50A, T51A, S52A, S52L, I53A, E55A, V77A, D118A, D118R, E119A, and/or E119Q based on SEQ ID NO:109. See e.g., J. Groppe et al. Mol Cell 29, 157-168, (2008) and De Crescenzo et al. JMB 355, 47-62 (2006) for the effects of those substitutions on TGF-β3-TβRII and TβRI-TβRII complexes. Modifications of TβRII the including an N-terminal Δ25 deletion and/or substitutions at F24 (e.g., an F24A substitution) substantially or completely block signal through the canonical SMAD signaling pathway). In one aspect, the aspartic acid at position I18 (D118) of the mature TβRII B isoform (SEQ ID NO:109) is replaced by an amino acid other than Asp or Glu, such as Ala giving rise to a “D118A” substitution or by an Arg giving rise to a D118R substitution. The Asp residues corresponding D118 are indicated SEQ ID NOs:108, 216, 109, 109, 111, 112, and 217 (with bold and underlining in FIG. 16B). N-terminal deletions of from 1 to 25 aa in length (e.g., a A25 deletions) and/or substitutions at F24 (e.g., an F24A substitution) may be combined with D118 substitutions (e.g., D118A or D118R). N-terminal deletions of from 1 to 25 aa in length (e.g., a A25 deletions) and/or substitutions at F24 (e.g., an F24A substitution) may also be combined with substitutions at any of L27, F30, D32, S49, 150, T51, S52, 153, E55, V77, D118, and/or E119 (e.g., D118A) substitutions, and particularly any of the specific substitutions recited for those locations in SEQ ID NO:109 described above to alter the affinity.

Deletions of the N-terminus of the TβRII polypeptides may also result in loss of TβRI interactions and prevent masked TGF-β MODs comprising a TβRII polypeptide from acting as a constitutively active complex that engages and activates TβRI signaling. A14 aa deletion (Δ14) of the TβRII polypeptide substantively reduces the interaction of the protein with TβRI, and a Δ25 aa deletion of TβRII appears to completely abrogate the interaction with TβRI. N-terminal deletions also substantially alter the pI of the protein, with the Δ14 TβRII ectodomain mutant displaying a pI of about 4.5-5.0 (e.g., about 4.74). Accordingly, TGF-β MODs may comprise TβRII ectodomain polypeptides (e.g., polypeptides of SEQ ID NOs:108 or 217) with N-terminal deletions, such as from 14 to 25 aas (e.g., 14, 15, 16, 17, 18, 19, 20, 21, 22, 23, 24, or 25 aa). Modified ectodomain sequences, including those that limit interactions with TβRI, that may be utilized to mask TGF-β polypeptides in a masked TGF-β MOD are described in the paragraphs that follow.

In an embodiment, the sequence masking TGF-β in a masked TGF-β MOD comprises sequence having at least 60%, at least 70%, at least 80%, at least 90%, at least 95%, at least 98%, at least 99%, or 100%, aa sequence identity to at least 70, at least 80, at least 90, at least 100, at least 110, at least 120, at least 130, at least 140, or 142 aas of the T RII isoform B ectodomain sequence: IPPHVQKSVN NDMIVTDNNG AVKFPQLCK [symbol] C [symbol] VRFSTCDN QKSCMSNCSI TSIC [symbol] KPQEV CVAVWRKNDE NITLETVCHD PKLPYHDFIL EDAASPKCIM KEKKKPGETF FMCSCSS [symbol] EC NDNIIFSEE (SEQ ID NO:110). Any one or more of F30, D32, S52, E55, or D118 (italicized and bolded) may be substituted by an amino acid other than the naturally occurring aa at those positions (e.g., alanine). In an embodiment, the sequence masking TGF-β comprises the peptide of SEQ ID NO:110 bearing a D118A substitution. In an embodiment, the sequence masking TGF-β comprises the polypeptide of SEQ ID NO:110 bearing a Dl18A substitution and one or more of a F30A, D32N, S52L and/or E55A substitution.

Combinations of N-terminal deletions of TβRII, such as from 14 to 25 aas (e.g., 14, 15, 16, 17, 18, 19, 20, 21, 22, 23, 24, or 25 aa), that block inadvertent cell signaling due to the masked TGF-β/TβRII complex interacting with TβRI may be combined with other TβRII ectodomain substitutions, including those at any one or more of F30, D32, S52, E55, and/or D118. The combination of deletions and substitutions ensures the masked TGF-β MOD does not cause cell signaling except through the cell's membrane bound TβRI & TβRII receptors.

In an embodiment, the sequence masking TGF-β in a masked TGF-β MOD comprises sequence having at least 60%, at least 70%, at least 80%, at least 90%, at least 95%, at least 98%, at least 99%, or 100%, aa sequence identity to at least 70, at least 80, at least 90, at least 100, at 1 t 1Q r 114 aas of the TβRII isoform B ectodomain sequence: VTDNNG AVKFPQLCK [symbol] C [symbol] VRFSTCDN QKSCMSNCSI T [symbol] IC [symbol] KPWEV CVAVWRKNDE NITLETVCHD PKLPYHDFIL EDAASPKCIM KEKKKPGETF FMCSCSS [symbol] EC NDNIIFSEE (SEQ ID NO:111), which has aas 1-14 (Δ14) deleted. Any one or more of F30, D32, S52, E55, or D118 (italicized and bolded) may be substituted by an amino acid other than the naturally occurring aa at those positions (e.g., alanine). In an embodiment, the sequence masking TGF-β comprises the peptide of SEQ ID NO:111 bearing a D118A substitution. In an embodiment, the sequence masking TGF-β comprises the polypeptide of SEQ ID NO:111 bearing a D118A substitution and one or more of a F30A, D32N, S52L and/or E55A substitution.

In an embodiment, the sequence masking TGF-β in a masked TGF-β MOD comprises sequence having at least 60%, at least 70%, at least 80%, at least 90%, at least 95%, at least 98%, at least 99%, or 100%, aa sequence identity to at least 70, at least 80, at least 90, at least 100, or 104 aas of the TARII isoform B ectodomain sequence: QLCK [symbol] CDVRFSTCDN QKSCMSNCSI T [symbol] IC [symbol] KPQEV CVAVWRKNDE NITLETVCHD PKLPYHDFIL EDAASPKCIM KEKKKPGETF FMCSCSS [symbol] EC NDNIIFSEE (SEQ ID NO:112), which has aas 1-25 (Δ25) deleted. Any one or more of F30, D32, S52, E55, or D118 (italicized and bolded) may be substituted by an amino acid other than the naturally occurring as at those positions (e.g., alanine). In an embodiment, the sequence masking TGF-β comprises the polypeptide of SEQ ID NO:112 bearing a D118A substitution (shown as SEQ ID NO:217 in FIG. 16B). In an embodiment, the sequence masking TGF-β in a masked TGF-β MOD comprises the peptide of SEQ ID NO:112 bearing a D118A substitution and one or more of a F30A, D32N, S52L and/or E55A substitution. In an embodiment, the sequence masking TGF-β in a masked TGF-β MOD comprises the peptide of SEQ ID NO:112 (see FIG. 16B) bearing D118A and F30A substitutions. In an embodiment, the sequence masking TGF-β in a masked TGF-β MOD comprises the peptide of SEQ ID NO:112 (see FIG. 16B) bearing D118A and D32N substitutions. In an embodiment, the sequence masking TGF-β in a masked TGF-β MOD comprises the peptide of SEQ ID NO:112 (see FIG. 16B) bearing D118A and S52L substitutions. In an embodiment, the sequence masking TGF-β in a masked TGF-β MOD comprises the peptide of SEQ ID NO:112 (see FIG. 16B) bearing D118A and E55A.

### (c) TGF-β Receptor III (TβRIII)

In an embodiment, the polypeptide sequence masking TGF-β in a masked TGF-β MOD may be derived from a TβRIII (e.g., isoform A SEQ ID NO:218 and isoform B 125), and may comprises all or part of a TβRIII ectodomain (aas 27-787 of the A isoform or 27-786 of the B isoform). In some cases, a suitable TβRIII polypeptide for masking TGF-β comprises an amino acid sequence having at least 60%, at least 70%, at least 80%, at least 90%, at least 95%, at least 98%, at least 99%, or 100%, aa sequence identity to at least 70, at least 80, at least 90, at least 100, or 120 aas of a TβRIII A isoform or B isoform ectodomain sequences (e.g., provided in FIG. 16C as SEQ ID NO:218 or SEQ ID NO:219).

### (ii) Antibodies

Although TGF-β receptor polypeptides (e.g., ectodomain sequences) can function to bind and mask TGF-β polypeptides in masked TGF-β MODs, other polypeptide sequences (protein sequences) that bind to TGF-β sequences can also be employed as masking polypeptides. Among the suitable polypeptide or protein sequences that can be used to mask TGF-β are antibodies with affinity for TGF-β (e.g., antibodies specific for an one or more of TGF-β1, TGF-β2, or TGF-β3) or antibody-related molecules such as anti-TGF-β antibody fragments, nanobodies with affinity for TGF-β polypeptides, and particularly single chain anti-TGF-β antibodies (e.g., any of which may be humanized). Some antibodies, including scFV antibodies, that bind and neutralize TGF-β have been described. See e.g., U.S. Pat. No. 9,090,685. Throughout the embodiments and/or aspects of the invention described in this disclosure, TβR (e.g., TβRII) sequences used to mask TGF-β polypeptides may be replaced with masking antibody sequences (e.g., a scFV or a nanobody) with affinity for the TGF-β polypeptide. For instance, in each of the masked TGF-β MODs in FIG. 1 where a TGF-β receptor sequence is used to mask a TGF-β polypeptide, the receptor polypeptide may be replaced with a masking antibody polypeptide (e.g., scFV or a nanobody) with affinity for the TGF-β polypeptide.

One potential advantage of using an antibody (e.g., a single chain antibody) as a masking polypeptide is the ability to limit it to the isoform of the TGF-β polypeptide(s) to be masked. By way of example, single chain antibody sequences based on Metelimumab (CAT192) directed against TGF-β1 (e.g., Lord et al., mAbs 10(3): 444-452 (2018)) can be used to mask that TGF-β isoform when present in TGF-β MODs. In another embodiment, a single chain antibody sequence specific for TGF-β2 is used to mask that TGF-β isoform when present in TGF-β MODs. In another embodiment, a single chain antibody sequence specific for TGF-β3 is used to mask that TGF-β isoform when present in TGF-β MODs. Single chain antibodies can also be specific for a combination of TGF-β isoforms (e.g., ectodomain sequences appearing in masked TGF-β MODs selected from the group consisting of: TGF-β1 & TGF-β2; TGF-β1 & TGF-β3; and TGF-β2 & TGF-β3. The single chain antibodies may also be pan-specific for TGF-β1, TGF-β2, and TGF-β3 ectodomain sequences appearing in masked TGF-β MODs See e.g., WO 2014/164709. Antibodies and single chain antibodies that have the desired specificity and affinity for TGF-β isoforms can be prepared by a variety of methods, including screening hybridomas and/or modification (e.g., combinatorial modification) to the variable region sequence of antibodies that have affinity for a target TGF-β polypeptide sequence.

In an embodiment, a masked TGF-β MOD comprises a single chain antibody to mask a TGF-β sequence (e.g., a TGF-β3 sequence). In one such embodiment the single chain amino acid sequence is specific for the TGF-β3 set forth in SEQ ID NO:105 comprising a C77S substitution (see SEQ ID NO:213).

c. Placement of TGF-β and TGF-β Masking Sequence in TMAPPs

The masking sequence (e.g., a TGF-β receptor sequence) of a masked TGF-β MOD may either be part of the same polypeptide as the TGF-β sequence, that is both the masking and TGF-β sequences are present in “cis.” Alternatively, the masking sequence (e.g., a TGF-β receptor sequence) and the TGF-β sequence may be part of a different polypeptides, that is to say they are present in “trans.”

When the masking sequence and the TGF-β sequence of a masked TGF-β MOD are present in a single aa sequence (single polypeptide) of a TMAPP (placed in cis), the aa sequence may be arranged in the N-terminal to C-terminal direction as either: a) TGF-β receptor sequence(s) followed by TGF-β sequence(s), or b) TGF-β sequence(s) followed by TGF-β receptor sequence(s). Regardless of the order from N-terminus to C-terminus, the polypeptide sequence of a masked TGF-β MOD may be linked to any other TMAPP polypeptide at its N-terminus or C-terminus. Independently selected linker polypeptide (e.g., Gly₄Ser repeats) may be used to join the masking sequence (e.g., a TGF-β receptor sequence) and the TGF-β sequence, and also to join the TGF-β MOD to a polypeptide of the TMAPP. As an example, a cis-masked TGF-β MOD may be linked to the C terminus of a TMAPP scaffold polypeptide and have the order from N-terminus to C-terminus a) TGF-β receptor sequence (e.g., a TβRII sequence) followed by TGF-β sequence (e.g., TGF-β3). To further that example, the cis-masked TGF-β MOD may be linked to the scaffold polypeptide (e.g., at its C-terminus), and the cis-masked TGF-β MOD may optionally be followed by another MOD such as IL-2.

One example of a masked TGF-β MOD with the TβR and TGF-β in cis (a cis-masked TGF-β MOD) is the sequence: QLCKFCDVRFSTCDNQKSCMSNCSITSICEKPQEVCVAVWRKNDENITLETVCHDPKLPYHDFI LEDAASPKCIMKEKKKPGETFFMCSCSSAECNDNIIFSEEYNTSNPDGGGGSGGGGSGGGGSGG GGSGGGGSALDTNYCFRNLEENCCVRPLYIDFRQDLGWKWVHEPKGYYANFCSGPCPYLRSA DTTHSTVLGLYNTLNPEASASPSCVPQDLEPLTILYYVGRTPKVEQLSNMVVKSCKCS (SEQ ID NO:220), where: aas 1-111 are a human TβRII masking sequence with the N-terminal 25 aas removed (Δ25) and a D118A substitution; aas 112-136 are a linker (five Gly4Ser repeats); and 137-248 is a human TGF-β3 sequence with a C77S substitution. Such a sequence may be attached, for example, by its N-terminus, directly or indirectly, via an independently selected linker to the C-terminus of a TMAPP polypeptide (e.g., a scaffold polypeptide). In addition, the cis masked TGF-β MOD sequence may have appended to it another MOD sequence (e.g., a human IL-2 or variant IL-2 MOD polypeptide sequence).

When the masking sequence (e.g., TGF-β receptor sequence) and the TGF-β sequence of a masked TGF-β MOD are present as part of different TMAPP polypeptides (placed in trans), those polypeptide sequences are attached to different (separate) TMAPP polypeptides that interact, thereby pairing the TGF-β sequences with masking polypeptide (e.g., a TGF-β receptor sequence). The TGF-β sequence and masking sequence may be located at the N-terminus or C-terminus of TMAPP polypeptides. In one example the TGF-β and masking sequences when placed in trans may be located at the C-terminus of the scaffold sequences of duplex TMAPPs (see e.g., FIGS. 1G and 1H). In another example the TGF-β and masking sequences may be located at the N-terminus of first and second presentation sequences. Independently selected linker polypeptides (e.g., Gly₄Ser repeats) may be used to join the masking sequence (e.g., TGF-β receptor sequence) or the TGF-β sequence to other TMAPP polypeptides. As an example, in a duplex TMAPP with a trans-masked TGF-β MOD a masking TGF-β receptor sequence (e.g., TβRII) may be part of a first scaffold polypeptide and the TGF-β sequence (e.g., a TGF-β3 sequence) part of a second scaffold polypeptide, where the first and second scaffold polypeptides associate through interspecific interactions. To further that example, the TGF-β sequence and TGF-β receptor sequence may be located at the C-terminus of the scaffold polypeptides and may optionally be followed by another MOD such as IL-2. In any of the foregoing examples, the masking TβR sequence may, for example, be a TβRII sequence lacking its N-terminal 25 aas (Δ25) and bearing a D118A substitution: SQLCKFCDVRFSTCDNQKSCMSNCSITSICEKPQEVCVAVWRKNDENITLETVCHDPKLPYHDFI LEDAASPKCIMKEKKKPGETFFMCSCSSAECNDNIIFSEEYNTSNPD (SEQ ID NO:113). The TGF-β polypeptide may be a human TGF-β3 polypeptide bearing a C77S substitution:

(SEQ ID NO: 114)
ALDTNYCFRNLEENCCVRPLYIDFRQDLGW
KWVHEPKGYYANFCSGPCPYLRSADTTHST
VLGLYNTLNPEASASPSCVPQDLEPLTILY
YVGRTPKVEQLSNMVVKSCKCS

Linkers that are selected independently may be used to join the TGF-β and TβR sequences to a TMAPP polypeptide.

### 2. IL-2 and its Variants

As one non-limiting example, a MOD or variant MOD present in a TMAPP is an IL-2 or variant IL-2 polypeptide. In some cases, a variant MOD present in a TMAPP is a variant IL-2 polypeptide. Wild-type IL-2 binds to an IL-2 receptor (IL-2R). A wild-type IL-2 aa sequence can be as follows:

APTSSSTKKT QLQLEHLLLD LQMILNGINN
YKNPKLTRML TFKFYMPKKA TELKHLQCLE
EELKPLEEVL NLAQSKNFHL RPRDLISNIN
VIVLELKGSE TTFMCEYADE TATIVEFLNR
WITFCQSIIS TLT
(aa 21-153 of UniProt P60568, SEQ ID NO: 115).

Wild-type IL2 binds to an I12 receptor (IL2R) on the surface of a cell. An IL2 receptor is in some cases a heterotrimeric polypeptide comprising an alpha chain (IL-2Rα; also referred to as CD25), a beta chain (IL-2Rβ; also referred to as CD122) and a gamma chain (IL-2Rγ; also referred to as CD132). Amino acid sequences of human IL-2Rα, IL2Rβ, and IL-2Rγ are provided in the accompanying sequence listing as SEQ ID NO:116, SEQ ID NO:117 and SEQ ID NO:118 respectively, and are also provided in, for example, U.S. Patent Pub. No. 20200407416.

In some cases, a variant IL-2 polypeptide exhibits reduced binding affinity to one or more of the IL-2Rα, IL2Rβ, and/or IL-2Rγ chains of human IL-2R, compared to the binding affinity of an IL-2 polypeptide comprising the aa sequence set forth in SEQ ID NO:115. For example, in some cases, a variant IL-2 polypeptide binds to one or more of the IL-2Rα, IL2Rβ, and/or IL-2Rγ chains of human IL-2R with a binding affinity that is at least 10% less, at least 20% less, at least 30% less, at least 40% less, at least 50% less, at least 60% less, at least 70% less, at least 80% less, at least 90% less, at least 95% less, or more than 95% less, than the binding affinity of an IL-2 polypeptide comprising the aa sequence set forth in SEQ ID NO:115 for the α, β, and/or γ chains of IL-2R (e.g., an IL-2R comprising polypeptides comprising the aa sequence set forth in SEQ ID NOs:116-118), when assayed under the same conditions.

Human IL-2Rα:
(SEQ ID NO: 116)
ELCDDDPPE IPHATFKAMA YKEGTMLNCE CKRGFRRIKS
GSLYMLCTGN SSHSSWDNQC QCTSSATRNT TKQVTPQPEE
QKERKTTEMQ SPMQPVDQAS LPGHCREPPP WENEATERIY
HFVVGQMVYY QCVQGYRALH RGPAESVCKM THGKTRWTQP
QLICTGEMET SQFPGEEKPQ ASPEGRPESE TSCLVTTTDF
QIQTEMAATM ETSIFTTEYQ VAVAGCVFLL ISVLLLSGLT
WQRRQRKSRR TI.
Human IL-2Rβ:
(SEQ ID NO: 117)
VNG TSQFTCFYNS RANISCVWSQ DGALQDTSCQ
VHAWPDRRRW NQTCELLPVS QASWACNLIL GAPDSQKLTT
VDIVTLRVLC REGVRWRVMA IQDFKPFENL RLMAPISLQV
VHVETHRCNI SWEISQASHY FERHLEFEAR TLSPGHTWEE
APLLTLKQKQ EWICLETLTP DTQYEFQVRV KPLQGEFTTW
SPWSQPLAFR TKPAALGKDT IPWLGHLLVG LSGAFGFIIL
VYLLINCRNT GPWLKKVLKC NTPDPSKFFS QLSSEHGGDV
QKWLSSPFPS SSFSPGGLAP EISPLEVLER DKVTQLLLQQ
DKVPEPASLS SNHSLTSCFT NQGYFFFHLP DALEIEACQV
YFTYDPYSEE DPDEGVAGAP TGSSPQPLQP LSGEDDAYCT
FPSRDDLLLF SPSLLGGPSP PSTAPGGSGA GEERMPPSLQ
ERVPRDWDPQ PLGPPTPGVP DLVDFQPPPE LVLREAGEEV
PDAGPREGVS FPWSRPPGQG EFRALNARLP LNTDAYLSLQ
ELQGQDPTHL V.
Human IL-2Rγ:
(SEQ ID NO: 118)
LNTTILTP NGNEDTTADF FLTTMPTDSL SVSTLPLPEV
QCFVFNVEYM NCTWNSSSEP QPTNLTLHYW YKNSDNDKVQ
KCSHYLFSEE ITSGCQLQKK EIHLYQTFVV QLQDPREPRR
QATQMLKLQN LVIPWAPENL TLHKLSESQL ELNWNNRFLN
HCLEHLVQYR TDWDHSWTEQ SVDYRHKFSL PSVDGQKRYT
FRVRSRFNPL CGSAQHWSEW SHPIHWGSNT SKENPFLFAL
EAVVISVGSM GLIISLLCVY FWLERTMPRI PTLKNLEDLV
TEYHGNFSAW SGVSKGLAES LQPDYSERLC LVSEIPPKGG
ALGEGPGASP CNQHSPYWAP PCYTLKPET.

For example, IL-2 variants with a substitution of phenylalanine at position 42 (e.g., with an alanine), exhibit substantially reduced binding to the IL-2Rα chain, in which case the variant may reduce the activation of Tregs. IL-2 variants with a substitution of histidine at position I6 (e.g., with an alanine) exhibit reduced binding to the IL2Rβ chain, thereby reducing the likelihood of a TMAPP binding to non-target T cells by virtue of off-target binding of the IL-2 MOD. Some IL-2 variants, e.g., those with substitutions of the F42 and H16 amino acids, exhibit substantially reduced binding to the IL-2Rα chain and also reduced binding to the IL2Rβ chain. See, e.g., Quayle, et al., Clin Cancer Res; 26(8) Apr. 15, 2020.

In some cases, a variant IL-2 polypeptide has a single as substitution compared to the IL-2 as sequence set forth in SEQ ID NO:115. In some cases, a variant IL-2 polypeptide has from 2 to 10 as substitutions compared to the IL-2 aa sequence set forth in SEQ ID NO: 115. In some cases, a variant IL-2 polypeptide has 2, 3, 4, 5, 6, 7, 8, 9 or 10 aa substitutions compared to the IL-2 aa sequence set forth in SEQ ID NO:115. In some cases, a variant IL-2 polypeptide has 2 or 3 aa substitutions compared to the IL-2 aa sequence set forth in SEQ ID NO:115.

Suitable variant IL-2 polypeptide sequences include polypeptide sequences comprising an aa sequence having at least 80% (e.g., at least 85%, at least 90%, at least 95%, at least 98%, at least 99%, or 100%) aa sequence identity to at least 80 (e.g., 90, 100, 110, 120, 130 or 133) contiguous aas of SEQ ID NO:115. Potential amino acids where substitutions may be introduced include one or more of the following positions:
(i) position I5, where the as is other than E (e.g., A);
(ii) position I6, where the as is other than H (e.g., A, T, N, C, Q, M, V or W);
(iii) position 20 is an aa other than D (e.g., A);
(iv) position 42, where the as is other than F (e.g., A, M, P, S, T, Y, V or H);
(v) position 45, where the as is other than Y (e.g., A);
(vi) position 88, where the as is other than N (e.g., A or R);
(vii) position I26, where the as is other than Q (e.g., A);
   Combinations of the above substitutions include (H16X, F42X), (D20X, F42X), (E15X, D20X, F42X), (an H16X, D20X, F42X), (H16X, F42X, R88X), (H16X, F42X, Q126X), (D20X, F42X, Q126X), (D20X, F42X, and Y4X), (H16X, D20X, F42X, and Y45X), (D20X, F42X, Y45X, Q126X), (H16X, D20X, F42X, Y45X, Q126X), where X is the substituted aa, optionally chosen from the following: positions 15, 20, 45, 126—A; position I6—A or T, or also N, C, Q, M, V or W; position 42—A, or also M, P, S, T, Y, V or H; position 88—A or R.

IL-2 variants include polypeptides having at least 90% (e.g., at least 95%, 98%, or 99%) aa sequence identity to at least 80 (e.g., at least 90, 100, 110, 120, or 130) contiguous aas of SEQ ID NO:115, wherein the as at position I6 is an aa other than H. In one case, the position of H16 is substituted by Asn, Cys, Gln, Met, Val, or Trp. In one case, the position of H16 is substituted by Ala. In another case, the position of H16 is substituted by Thr. Additionally, or alternatively, IL-2 variants include polypeptides having at least 90% (e.g., at least 95%, 98%, or 99%) as sequence identity to at least 80 (e.g., at least 90, 100, 110, 120, or 130) contiguous aas of SEQ ID NO:115, wherein the aa at position 42 is an aa other than F. In one case, the position of F42 is substituted by Met, Pro, Ser, Thr, Trp, Tyr, Val, or His. In one case, the position of F42 is substituted by Ala.

IL-2 variants include polypeptides comprising an as sequence comprising all or part of human IL-2 polypeptide having a substitution at position H16 and/or F42 (e.g., H16A and/or F42A substitutions).

IL-2 variants include polypeptides having at least 90% (e.g., at least 95%, 98%, or 99%) as sequence identity to at least 80 (e.g., at least 100, 110, 120, or 130) contiguous aas of SEQ ID NO:115, wherein the as at position I6 is an as other than H and the as at position 42 is other than F. In one case, the position of H16 is substituted by Ala or Thr and the position of F42 is substituted by Ala or Thr. In one case, the position of H16 is substituted by Ala and the position of F42 is substituted by Ala (an H16A and F42A variant). In a second case, the position of H16 is substituted by Thr and the position of F42 is substituted by Ala (an H16T and F42A variant). In a third case, the position of H16 is substituted by Ala and the position of F42 is substituted by Thr (an H16A and F42T variant). In a fourth case, the position of H16 is substituted by Thr and the position of F42 is substituted Thr Ala (an H16T and F42T variant). As noted above, such variants will exhibit reduced binding to both the human IL-2Rα chain and IL2Rβ chain.

In any of the wild-type or variant IL-2 sequences provided herein, the cysteine at position I25 may be substituted with an as other than cystine, such as alanine (a C125A substitution). In addition to any stability provided by the substitution, it may be employed where, for example, an epitope containing peptide or additional peptide is to be conjugated to a cysteine residue elsewhere in a TMAPP, thereby avoiding competition from the C125 of the IL-2 MOD sequence.

### 3. PD-L1 and its Variants

As one non-limiting example, a MOD or variant MOD present in a TMAPP is a PD-L1 or variant PD-L1 polypeptide. Wild-type PD-L1 binds to PD1.

A wild-type human PD-L1 polypeptide can comprise the following aa sequence: MRIFAVFIFM TYWHLLNAFT VTVPKDLYVV EYGSNMTIEC KFPVEKQLDL AALIVYWEME DKNIIQFVHG EEDLKVQHSS YRQRARLLKD QLSLGNAALQ ITDVKLQDAG VYRCMISYGG ADYKRITVKV NAPYNKINQR ILVVDPVTSE HELTCQAEGY PKAEVIWTSS DHQVLSGKTT TTNSKREEKL FNVTSTLRIN TITNEIFYCT FRRLDPEENH TAELVIPGNI LNVSIKICLT LSPST (SEQ ID NO:119); where aas 1-18 form the signal sequence, aas 19-127 form the Ig-like V-type or IgV domain, and 133-225 for the Ig-like C2 type domain.

A wild-type human PD-L1 ectodomain aa sequence can comprise the following aa sequence: FT VTVPKDLYVV EYGSNMTIEC KFPVEKQLDL AALIVYWEME DKNIIQFVHG EEDLKVQHSS YRQRARLLKD QLSLGNAALQ ITDVKLQDAG VYRCMISYGG ADYKRITVKV NAPYNKINQR ILVVDPVTSE HELTCQAEGY PKAEVIWTSS DHQVLSGKTT TTNSKREEKL FNVTSTLRIN TITNEIFYCT FRRLDPEENH TAELVIPGNI LNVSIKI (SEQ ID NO:120); where aas 1-109 form the Ig-like V-type or “IgV” domain, and aas 115-207 for the Ig-like C2 type domain.

A wild-type human PD-L1 ectodomain aa sequence can also comprise the following aa sequence: FT VTVPKDLYVV EYGSNMTIEC KFPVEKQLDL AALIVYWEME DKNIIQFVHG EEDLKVQHSS YRQRARLLKD QLSLGNAALQ ITDVKLQDAG VYRCMISYGG ADYKRITVKV NAPYNKINQR ILVVDPVTSE HELTCQAEGY PKAEVIWTSS DHQVLSGKTT TTNSKREEKL FNVTSTLRIN TTNEIFYCT FRRLDPEENH TAELVIPELP LAHPPNER LNVSIKI (SEQ ID NO:121); where aas 1-109 form the Ig-like V-type or “IgV” domain, and aas 115-207 for the Ig-like C2 type domain. See e.g., NCBI Accession and version 3BIK_A, which includes an N-terminal alanine as its first aa.

A wild-type PD-L1 IgV domain, suitable for use as a MOD may comprise aa 18 and aas IgV aas 19-127 of SEQ ID NO:119, and a carboxyl terminal stabilization sequences, such as for instance the last seven aas (bolded and italicized) of the sequence: A FTVTVPKDLY VVEYGSNMTI ECKFPVEKQL DLAALIVYWE MEDKNIIQFV HGEEDLKTQH SSYRQRARLL KDQLSLGNAA [symbol] ITDVKLQD AGVYRCMISY GGADYKRITV KVNAPY [symbol] (SEQ ID NO:122). Where the carboxyl stabilizing sequence comprises a histidine (e.g., a histidine approximately 5 residues to the C-terminal side of the Tyr (Y) appearing as as 117 of SEQ ID NO:122) to about as 122, the histidine may form a stabilizing electrostatic bond with the backbone amide at aas 82 and 83 (bolded and italicized in SEQ ID NO:119 (Q107 and L106 of SEQ ID NO:119). As an alternative, a stabilizing disulfide bond may be formed by substituting one of aas 82 or 83) (Q107 and L106 of SEQ ID NO:119) and one of as residues 121, 122, or 123 (equivalent to as positions 139-141 of SEQ ID NO:119).

A wild-type PD-1 polypeptide can comprise the following aa sequence: PGWFLDSPDR PWNPPTFSPA LLVVTEGDNA TFTCSFSNTS ESFVLNWYRM SPSNQTDKLA AFPEDRSQPG QDCRFRVTQL PNGRDFHMSV VRARRNDSGT YLCGAISLAP KAQIKESLRA ELRVTERRAE VPTAHPSPSP RPAGQFQTLV VGVVGGLLGS LVLLVWVLAV ICSRAARGTI GARRTGQPLK EDPSAVPVFS VDYGELDFQW REKTPEPPVP CVPEQTEYAT IVFPSGMGTS SPARRGSADG PRSAQPLRPE DGHCSWPL (SEQ ID NO:123).

In some cases, a variant PD-L1 polypeptide (e.g., a variant of SEQ ID NO:120 or PD-Li's IgV domain) exhibits reduced binding affinity to PD-1 (e.g., a PD-1 polypeptide comprising the as sequence set forth in SEQ ID NO:123), compared to the binding affinity of a PD-L1 polypeptide comprising the as sequence set forth in SEQ ID NO:119 or SEQ ID NO:120. For example, in some cases, a variant PD-L1 polypeptide binds PD-1 (e.g., a PD-1 polypeptide comprising the aa sequence set forth in SEQ ID NO:123) with a binding affinity that is at least 10% less, at least 20% less, at least 30% less, at least 40% less, at least 50% less, at least 60% less, at least 70% less, at least 80% less, at least 90% less, at least 95% less, or more than 95% less than the binding affinity of a PD-L1 polypeptide comprising the aa sequence set forth in SEQ ID NO: 119 or SEQ ID NO:120.

### 4. 4-1BBL and its Variants

In some cases, a wild-type and/or a variant 4-1BBL MOD polypeptide sequence is present as a MOD in a TMAPP. Wild-type 4-1BBL binds to 4-1BB (CD137).

A wild-type 4-1BBL as sequence can be as follows: MEYASDASLD PEAPWPPAPR ARACRVLPWA LVAGLLLLLL LAAACAVFLA CPWAVSGARA SPGSAASPRL REGPELSPDD PAGLLDLRQG MFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:124). NCBI Reference Sequence: NP_003802.1, where aas 29-49 are a transmembrane region.

In some cases, a variant 4-1BBL polypeptide is a variant of the tumor necrosis factor (TNF) homology domain (THD) of human 4-1BBL. A wild-type as sequence of the THD of human 4-1BBL can comprise, e.g., one of SEQ ID NOs:125-127, as follows:

(SEQ ID NO: 125)
PAGLLDLRQG MFAQLVAQNV LLIDGPLSWY SDPGLAGVSL
TGGLSYKEDT KELVVAKAGV YYVFFQLELR RVVAGEGSGS
VSLALHLQPL RSAAGAAALA LTVDLPPASS EARNSAFGFQ
GRLLHLSAGQ RLGVHLHTEA RARHAWQLTQ GATVLGLFRV
TPEIPAGLPS PRSE;
(SEQ ID NO: 126)
D PAGLLDLRQG MFAQLVAQNV LLIDGPLSWY
SDPGLAGVSL TGGLSYKEDT KELVVAKAGV YYVFFQLELR
RVVAGEGSGS VSLALHLQPL RSAAGAAALA LTVDLPPASS
EARNSAFGFQ GRLLHLSAGQ RLGVHLHTEA RARHAWQLTQ
GATVLGLFRV TPEIPAGLPS PRSE;
and
(SEQ ID NO: 127)
D PAGLLDLRQG MFAQLVAQNV LLIDGPLSWY SDPGLAGVSL
TGGLSYKEDT KELVVAKAGV YYVFFQLELR RVVAGEGSGS
VSLALHLQPL RSAAGAAALA LTVDLPPASS EARNSAFGFQ
GRLLHLSAGQ RLGVHLHTEA RARHAWQLTQ GATVLGLFRV
TPEIPA.

A wild-type 4-1BB as sequence can be as follows: MGNSCYNIVA TLLLVLNFER TRSLQDPCSN CPAGTFCDNN RNQICSPCPP NSFSSAGGQR TCDICRQCKG VFRTRKECSS TSNAECDCTP GFHCLGAGCS MCEQDCKQGQ ELTKKGCKDC CFGTFNDQKR GICRPWTNCS LDGKSVLVNG TKERDVVCGP SPADLSPGAS SVTPPAPARE PGHSPQIISF FLALTSTALL FLLFFLTLRF SVVKRGRKKL LYIFKQPFMR PVQTTQEEDG CSCRFPEEEE GGCEL (SEQ ID NO:128).

A variant 4-1BBL polypeptide exhibits reduced binding affinity to 4-1BB, compared to the binding affinity of a 4-1BBL polypeptide comprising the aa sequence set forth in one of SEQ ID NOs:125-127. For example, a variant 4-1BBL polypeptide may bind 4-1BB with a binding affinity that is at least 10% less, at least 20% less, at least 30% less, at least 40% less, at least 50% less, at least 60% less, at least 70% less, at least 80% less, at least 90% less, at least 95% less, or more than 95% less, than the binding affinity of a 4-1BBL polypeptide comprising the aa sequence set forth in one of SEQ ID NOs:125-127 for a 4-1BB polypeptide (e.g., a 4-1BB polypeptide comprising the aa sequence set forth in SEQ ID NO:128), when assayed under the same conditions.

4-1BBL variants suitable for use as a MOD in a TMAPP include those polypeptides with at least one aa substitution having at least 90%, at least 95%, at least 98%, or at least 99% aa sequence identity to one of SEQ ID NOs:125, 126 or 127.

4-1BBL variants suitable for inclusion in a TMAPP include those with at least one aa substitution (e.g., two, three, or four substitutions) include those having at least 90%, at least 95%, at least 98%, or at least 99% aa sequence identity to at least 140 (e.g., at least 160, 175, 180, or 181) contiguous aas of SEQ ID NO:125.

### V.A(vi). Linkers

As noted above, a TMAPP can include a linker sequence (aa, peptide, or polypeptide linker sequence) or “linker” interposed between any two elements of a TMAPP, e.g., an epitope and an MHC polypeptide; between an MHC polypeptide and an Ig Fc polypeptide; between a first MHC polypeptide and a second MHC polypeptide; etc. Although termed “linkers,” sequences employed for linkers may also be placed at the N- and/or C-terminus of a TMAPP polypeptide to, for example, stabilize the TMAPP polypeptide or protect it from proteolytic degradation.

Suitable polypeptide linkers (also referred to as “spacers”) are known in the art and can be readily selected and can be of any of a number of suitable lengths, e.g., from 2 to 50 aa in length, e.g., from 2 aa to 10 aa, from 10aa to 20 aa, 20 aa to 30 aa, from 30 aa to 40aa, from 40aa to 50aa, or longer than 50aa. In embodiments, a suitable linker can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 aa in length. Linkers can be generally classified into three groups, i.e., flexible, rigid and cleavable. See, e.g., Chen et al. (2013) Adv. Drug Deliv. Rev. 65:1357; and Klein et al. (2014) Protein Engineering, Design & Selection 27:325. Unless stated otherwise, the linkers employed in the TMAPPs of this disclosure are not the cleavable linkers generally known in the art.

Polypeptide linkers in the TMAPP may include, for example, polypeptides that comprise, consist essentially of, or consists of: i) Gly and Ser; ii) Ala and Ser; iii) Gly, Ala, and Ser; iv) Gly, Ser, and Cys (e.g., a single Cys residue); v) Ala, Ser, and Cys (e.g., a single Cys residue); and vi) Gly, Ala, Ser, and Cys (e.g., a single Cys residue). Exemplary linkers may comprise glycine polymers, glycine-serine polymers, glycine-alanine polymers; alanine-serine polymers (including, for example polymers comprising the sequences GSGGS (SEQ ID NO:129) or GGGS (SEQ ID NO:130), any of which may be repeated from 1 to 10 times (e.g., repeated 1, 2, 3, 4, 5, 6, 7, 8, 9, or 10 times); and other flexible linkers known in the art. Glycine and glycine-serine polymers can both be used; both Gly and Ser are relatively unstructured and therefore can serve as a neutral tether between components. Glycine polymers access significantly more phi-psi space than even alanine, and are much less restricted than residues with longer side chains (see Scheraga, Rev. Computational Chem. 11173-142 (1992)). Exemplary linkers may also comprise an aa sequence comprising, but not limited to, GGSG (SEQ ID NO:131), GGSGG (SEQ ID NO:132), GSGSG (SEQ ID NO:133), GSGGG (SEQ ID NO:134), GGGSG (SEQ ID NO:135), GSSSG (SEQ ID NO:136), any which may be repeated from 1 to 10 times (e.g., repeated 1, 2, 3, 4, 5, 6, 7, 8, 9, or 10 times), or combinations thereof, and the like. Linkers can also comprise the sequence Gly(Ser)₄ (SEQ ID NO:137) or (Gly)₄Ser (SEQ ID NO:82), either of which may be repeated from 1 to 10 times (e.g., 1, 2, 3, 4, 5, 6, 7, 8, 9, or 10 times). In one embodiment the linker comprises the aa sequence AAAGG (SEQ ID NO:138), which may be repeated from 1 to 10 times.

Rigid polypeptide linkers comprise a sequence of amino acids that effectively separates protein domains by maintaining a substantially fixed distance/spatial separation between the domains, thereby reducing or substantially eliminating unfavorable interactions between such domains. Rigid polypeptide linkers thus may be employed where it is desired to minimize the interaction between the domains of the TMAPP. Rigid peptide linkers include peptide linkers rich in proline, and peptide linkers having an inflexible helical structure, such as an α-helical structure. Examples of rigid peptide linkers include, e.g., (EAAAK)n (SEQ ID NO:139), A(EAAAK)nA (SEQ ID NO:140), A(EAAAK)nALEA(EAAAK)nA (SEQ ID NO:141), (Lys-Pro)n, (Glu-Pro)n, (Thr-Pro-Arg)n, and (Ala-Pro)n where n is an integer from 1 to 20 (e.g., n is 1, 2, 3, 4, 5, 6, 7, 8, 9, 10, 11, 12, 13, 14, 15, 16, 17, 18, 19, or 20). Non-limiting examples of suitable rigid linkers comprising EAAAK (SEQ ID NO:142) include EAAAK (SEQ ID NO:142), (EAAAK): (SEQ ID NO:143), (EAAAK)₃ (SEQ ID NO:144), A(EAAAK)₄ALEA(EAAAK)₄A (SEQ ID NO:145), and AEAAAKEAAAKA (SEQ ID NO:146). Non-limiting examples of suitable rigid linkers comprising (AP)n include PAPAP (SEQ ID NO:147; also referred to herein as “(AP)2”); APAPAPAP (SEQ ID NO:148; also referred to herein as “(AP)4”); APAPAPAPAPAP (SEQ ID NO:149; also referred to herein as “(AP)6”); APAPAPAPAPAPAPAP (SEQ ID NO:150; also referred to herein as “(AP)8”); and APAPAPAPAPAPAPAPAPAP (SEQ ID NO:151; also referred to herein as “(AP)10”). Non-limiting examples of suitable rigid linkers comprising (KP)n include KPKP (SEQ ID NO:152; also referred to herein as “(KP)2”); KPKPKPKP (SEQ ID NO:153; also referred to herein as “(KP)4”); KPKPKPKPKPKP (SEQ ID NO:154; also referred to herein as “(KP)6”); KPKPKPKPKPKPKPKP (SEQ ID NO:155; also referred to herein as “(KP)8”); and KPKPKPKPKPKPKPKPKPKP (SEQ ID NO:156; also referred to herein as “(KP)10”). Non-limiting examples of suitable rigid linkers comprising (EP)n include EPEP (SEQ ID NO:157; also referred to herein as “(EP)2”); EPEPEPEP (SEQ ID NO:158; also referred to herein as “(EP)4”); EPEPEPEPEPEP (SEQ ID NO:159; also referred to herein as “(EP)6”); EPEPEPEPEPEPEPEP (SEQ ID NO:160; also referred to herein as “(EP)8”); and EPEPEPEPEPEPEPEPEPEP (SEQ ID NO:161; also referred to herein as “(EP)10”).

In some cases, a linker polypeptide, present in a polypeptide of a TMAPP includes a cysteine residue that can form a disulfide bond with a cysteine residue present in another polypeptide of the TMAPP. In some cases, for example, the linker comprises an aa sequence selected from (CGGGS), (GCGGS), (GGCGS), (GGGCS), and (GGGGC) with the rest of the linker comprised of Gly and Ser residues (e.g., GGGGS units that may be repeated from 1 to 10 times, such as 1, 2, 3, 4, 5, 6, 7, 8, 9, or 10 times). Cysteine containing linkers may also be selected from the sequences GCGASGGGGSGGGGS (SEQ ID NO:162), C [symbol] GGSGGGGSGGGGSGGGGS (SEQ ID NO:83), and GCGGSGGGGSGGGGS (SEQ ID NO:163).

Accordingly, the linker to which an epitope is attached may be from about 5 to about 50 aas in length. The linker to which an epitope may be attached may, for example be from about 5 to about 50 aas in length and comprise more than 50% Gly and Ser residues with one cysteine residue. The linker to which an epitope may be attached may be from about 5 to about 50 aas in length and comprise more than 50% (Gly)₄S repeats with one optional cysteine residue. The linker to which an epitope may be attached may be a (Gly)₄S sequence repeated from 3 to 8 (e.g., 3 to 7) times, optionally having one aa replaced by a cysteine residue.

### V.A(vii). Epitopes

A variety of peptide epitopes (also referred to herein as “epitopes”) may be present in a TMAPP or higher order complexes of TMAPPs (such as duplex TMAPPs), and presentable to a TCR on the surface of a T cell.

A peptide epitope present in a TMAPP (e.g., a duplex TMAPP) is designed to be specifically bound by a target T cell that has a T cell receptor (“TCR”) that is specific for the epitope and which specifically binds the peptide epitope of the TMAPP. An epitope-specific T cell thus binds a peptide epitope having a reference aa sequence, but substantially does not bind an epitope that differs from the reference aa sequence.

Among the epitopes that may be bound and presented to a TCR by a TMAPP with Class II MHC presenting sequences or Class II MHC presenting complexes are TiD-associated peptide epitopes derived from of self antigens associated with T1D. A Type 1 Diabetes-associated epitope (also referred to herein as a “T1D peptide epitope” or “T1D epitope”) present in a TMAPP presents a T1D-associated peptide epitope to a TCR on the surface of a T cell.

A T1D peptide epitope can have a length of from about 4 aas to about 25 aas (aa), e.g., the epitope can have a length of from 5 aa to 10 aa, from 10 aa to 15 aa, from 10 aa to 20 aa, or from 20 aa to 25 aa. For example, a T1D epitope present in a TMAPP can have a length of 4 aa, 5 aa, 6 aa, 7 aa, 8 aa, 9 aa, 10 aa, 11 aa, 12 aa, 13 aa, 14 aa, 15 aa, 16 aa, 17 aa, 18 aa, 19 aa, 20 aa, 21 aa, 22 aa, 23 aa, 24 aa, or 25 aa. In some cases, a T1D peptide epitope present in a TMAPP has a length of from 10 aa to 20 aa, e.g., 10 aa, 11 aa, 12 aa, 13 aa, 14 aa, 15 aa, 16 aa, 17 aa, 18 aa, 19 aa and 20 aa.

Antigens associated with type 1 diabetes (T1D) include, e.g., preproinsulin, proinsulin, insulin, insulin B chain, insulin A chain, proinsulin C peptide, 65 kilodaltons (kDa) isoform of glutamic acid decarboxylase (GAD65), 67 kDa isoform of glutamic acid decarboxylase (GAD67), tyrosine phosphatase (IA-2), heat-shock protein HSP65, islet-specific glucose6-phosphatase catalytic subunit related protein (IGRP), islet antigen 2 (IA2), and zinc transporter (ZnT8). See, e.g., Mallone et al. (2011) Clin. Dev. Immunol. 2011:513210; and U.S. Patent Publication No. 2017/0045529. An antigen “associated with” a particular autoimmune disorder is an antigen that is a target of autoantibodies and/or autoreactive T cells present in individuals with that autoimmune disorder, T1D in this instance, where such autoantibodies and/or autoreactive T cells mediate a pathological state associated with the autoimmune disorder. A suitable T1D peptide epitope for inclusion in a TMAPP can be a peptide epitope of from 4 aas to about 25 aas in length of any one of the aforementioned T1D-associated antigens.

As one non-limiting example, a TiD peptide epitope is proinsulin 73-90 (GAGSLQPLALEGSLQKR; SEQ ID NO:164). As another non-limiting example, a T1D peptide epitope is the following insulin (InsA (1-15) peptide: GIVDQCCTSICSLYQ (SEQ ID NO:165). As another non-limiting example, a T1D peptide epitope is the following insulin (InsA(1-15; D4E) peptide: GIVEQCCTSICSLYQ (SEQ ID NO:166). As another non-limiting example, a T1D peptide epitope is the following GAD65 (555-567) peptide: NFFRMVISNPAAT (SEQ ID NO:167). As another non-limiting example, a T1D peptide epitope is the following GAD65 (555-567; F557I) peptide: NFIRMVISNPAAT (SEQ ID NO:168). As another non-limiting example, a T1D peptide epitope is the following islet antigen 2 (IA2) peptide: SFYLKNVQTQETRTLTQFHF (SEQ ID NO:169). As another non-limiting example, a T1D peptide epitope is the following proinsulin peptide: SLQPLALEGSLQSRG (SEQ ID NO:170). As another non-limiting example, a T1D peptide epitope is the following proinsulin peptide GSLQPLALEGSLQSRGIV (SEQ ID NO:171; proIns 75-92(K88S)).

In some cases, a suitable T1D peptide epitope comprises from 4 to 25 contiguous aas of an aa sequence having at least 90%, at least 95%, at least 98%, at least 99%, or 100%, aa sequence identity to aas 25-110 of the following human preproinsulin aa sequence (wherein italicized aas 1-24 form the signal peptide): MALWMRLLPL LALLALWGPD PAAA FVNQHL CGSHLVEALY LVCGERGFFY TPKTRREAED LQVGQVELGG GPGAGSLQPL ALEGSLQKRG IVEQCCTSIC SLYQLENYCN (SEQ ID NO:172); where the T1D peptide epitope has a length of 4 aas, 5 aas, 6 aas, 7, aas, 8 aas, 9 aas, 10 aas, 11 aas, 12 aas, 13 aas, 14 aas, 15 aas, 16 aas, 17 aas, 18 aas, 19 aas, 20 aas, 21 aas, 22 aas, 23 aas, 24 aas, or 25 aas. In some cases, the T1D peptide epitope has the an sequence: GAGSLQPLALEGSLQKRG (SEQ ID NO:173) (proIns 73-90). In some cases, the T1D peptide epitope has the aa sequence: SLQPLALEGSLQKRG (SEQ ID NO:174) (proIns 76-90). In some cases, the T1D peptide epitope has the an sequence: SLQPLALEGSLQSRG (SEQ ID NO:175) (proIns 76-90; K88S). In some cases, the TiD peptide epitope has the aa sequence: QPLALEGSLQKRG (SEQ ID NO:176). In some cases, the T1D peptide epitope has the an sequence: QPLALEGSLQSRG (SEQ ID NO:177).

### V.A(viii). Additional Polypeptides

A polypeptide chain of a TMAPP may include one or more polypeptides (amino acid sequences) in addition to those described above. Suitable additional polypeptides include affinity tags and affinity domains. The one or more additional polypeptides can be included at the N-terminus of a polypeptide chain of a TMAPP, at the C-terminus of a polypeptide chain of a TMAPP, or within (internal to) a polypeptide chain of a TMAPP of the present disclosure.

### Affinity Tags and Affinity Domains

Suitable affinity tags/polypeptide affinity domains include, but are not limited to, hemagglutinin (HA; e.g., YPYDVPDYA (SEQ ID NO:178); FLAG (e.g., DYKDDDDK (SEQ ID NO:179); c-myc (e.g., EQKLISEEDL; SEQ ID NO:180), and the like.

Affinity tags/domains include peptide sequences that can interact with a binding partner, e.g., such as one immobilized on a solid support, useful for identification or purification. DNA sequences encoding multiple consecutive single aas, such as histidine, when fused to the expressed protein, may be used for one-step purification of the recombinant protein by high affinity binding to a resin column, such as nickel Sepharose. Exemplary affinity tags/domains include HisX5 (HHHHH) (SEQ ID NO:181), HisX6 (HHHHHH) (SEQ ID NO:182), C-myc (EQKLISEEDL) (SEQ ID NO:180), Flag (DYKDDDDK) (SEQ ID NO:179), StrepTag (WSHPQFEK) (SEQ ID NO:183), hemagglutinin, e.g., HA Tag (YPYDVPDYA) (SEQ ID NO:178), glutathione-S-transferase (GST), thioredoxin, cellulose binding domains, RYIRS (SEQ ID NO:184), FHHT (SEQ ID NO:185), chitin binding domains, S-peptide, T7 peptide, SH2 domains, C-end RNA tag, WEAAAREACCRECCARA (SEQ ID NO:186), metal binding domains, e.g., zinc binding domains or calcium binding domains such as those from calcium-binding proteins, e.g., calmodulin, troponin C, calcineurin B, myosin light chain, recoverin, S-modulin, visinin, VILIP, neurocalcin, hippocalcin, frequenin, caltractin, calpain large-subunit, S100 proteins, parvalbumin, calbindin D9K, calbindin D28K, calretinin, inteins, biotin, streptavidin, MyoD, Id, leucine zipper sequences, and maltose binding protein.

V.a(ix). Chemical Conjugation Sites and Chemical Conjugation

The term “chemical conjugation site” means any suitable site of a TMAPP that permits the selective formation of a direct or indirect (through an intervening linker or spacer) covalent linkage between the TMAPP and an epitope-containing or payload-containing molecule. Chemical conjugation sites of unconjugated TMAPPs may be (i) active, i.e., capable of forming a direct or indirect (through an intervening linker or spacer) covalent linkage between the TMAPP and an epitope or payload without an additional chemical reaction or transformation of the chemical conjugation site (e.g., a solvent-accessible cysteine sulfhydryl), or (ii) nascent, i.e., requiring a further chemical reaction or enzymatic transformation of the chemical conjugation site to become an active chemical conjugation site (e.g., a sulfatase sequence not yet activated by an fGly enzyme).

The term “selectively formation” means that when an epitope- or payload-containing molecule bearing a moiety that is reactive with an active chemical conjugation site of a TMAPP, the epitope- or payload-containing molecule will be covalently bound to the chemical conjugation site in an amount higher than to any other site in the TMAPP.

Chemical conjugation sites may be introduced into a TMAPP using protein engineering techniques (e.g., by use of an appropriate nucleic acid sequence) to achieve a TMAPP having a desired aa sequence. Chemical conjugation sites can be individual aas (e.g., a cysteine or lysine) or aa sequences (e.g., sulfatase, sortase or transglutaminase sequences) in a protein or polypeptide sequence of the TMAPP.

Where the protein or polypeptide sequence of the TMAPP is derived from a naturally occurring protein (e.g., the MHC domains or an IgG scaffold), the chemical conjugation site may be a site not appearing in the naturally occurring sequence, such as a site resulting from amino acid substitutions (e.g., cysteine substitutions), insertions, and or deletions. The chemical conjugation site may also be a sequence, or part of a sequence, that is not derived from a naturally occurring protein, such as a linker sequence.

In some embodiments, there is only one chemical conjugation site (e.g., one chemical conjugation site added by protein engineering) in each unconjugated TMAPP polypeptide that permits an epitope to be covalently attached such that it can be located in the MHC polypeptide binding groove/cleft and presented to a TCR. Each individual unconjugated TMAPP may comprise more than one chemical conjugation sites, each of which are selected independently to be either the same or different types of chemical conjugation sites, thereby permitting the same or different molecules (e.g., an epitope and one or more payloads) to be selectively conjugated to each of the chemical conjugation sites. Accordingly, each individual or duplexed unconjugated TMAPP may comprise one or more chemical conjugations sites that are selected to be either the same or different types of chemical conjugation sites, thereby permitting the same or different molecules to be selectively conjugated to each of the chemical conjugation sites. The chemical conjugations sites (e.g., for the conjugation of epitope) generally will be the same (e.g., of the same type) so that epitope presenting molecules can be covalently attached to all of the desired sites in, for example, a duplex unconjugated TMAPP, using a single reaction. TMAPP's may contain chemical conjugation sites in addition to those for the conjugation to an epitope, including conjugation sites for the incorporation of, for example, targeting sequences and/or payloads such as labels.

Chemical conjugation sites used to incorporate molecules other than epitopes presenting molecules will, in most instances, be of a different type (e.g., utilize different chemical reactions) and in different locations than the sites used to incorporate epitopes, thereby permitting different molecules to be selectively conjugated to each of the conjugation sites. Where a TMAPP is to comprise a targeting sequence and/or one or more payload molecules, the unconjugated TMAPP may comprise more than one copy of a chemical conjugation site (e.g., chemical conjugation sites added by protein engineering) to permit attachment and of multiple molecules of targeting sequence and/or payload.

Chemical conjugation sites that may be incorporated into unconjugated TMAPP, include, but are not limited to:
a) peptide sequences that acts as an enzyme modification sequence (e.g., sulfatase, sortase, and/or transglutaminase sequences);
b) non-natural aas and/or selenocysteines;
c) chemical conjugation sites comprising individual amino acids;
d) carbohydrate or oligosaccharide moieties; and
e) IgG nucleotide binding sites.

Chemical conjugation sites for the conjugation of epitopes are typically located in a presentation sequence or complex (e.g., MHC α1, α2, β1 or β2 domain sequences, or in a linker attached directly to at least one of those domain sequences). Where aa motifs that permit chemical conjugation (e.g., motifs containing a chemical conjugation site or nascent chemical conjugation site) are to be incorporated into a TMAPP, the may be inserted in, for example, the presenting sequence(s) or presenting complex(es), the scaffold sequence(s) if present, or any of the linkers joining or attached to an element of a TMAPP. Again, where such motifs are to be used for epitope coupling, they will typically be located in in a presentation sequence or complex.

Motifs for chemical conjugation including, but not limited to, sulfatase, transglutaminase, carbohydrate, and nucleotide binding sites may be incorporated into any desired location of a TMAPP. In an embodiment such motifs for chemical conjugation may be excluded from the amino or carboxyl terminal 10 or 20 amino acids. In an embodiment, motifs for chemical conjugation may be added in (e.g., at or near the terminus) of any TMAPP element, including the MHC α chain or β chain polypeptide sequences (e.g., α1, α2, β1, or β2 domain sequences) or any linker sequence joining them. Motifs for chemical conjugation also be added to the scaffold polypeptide (e.g., the Ig Fc) or any of the linkers present in the TMAPP.

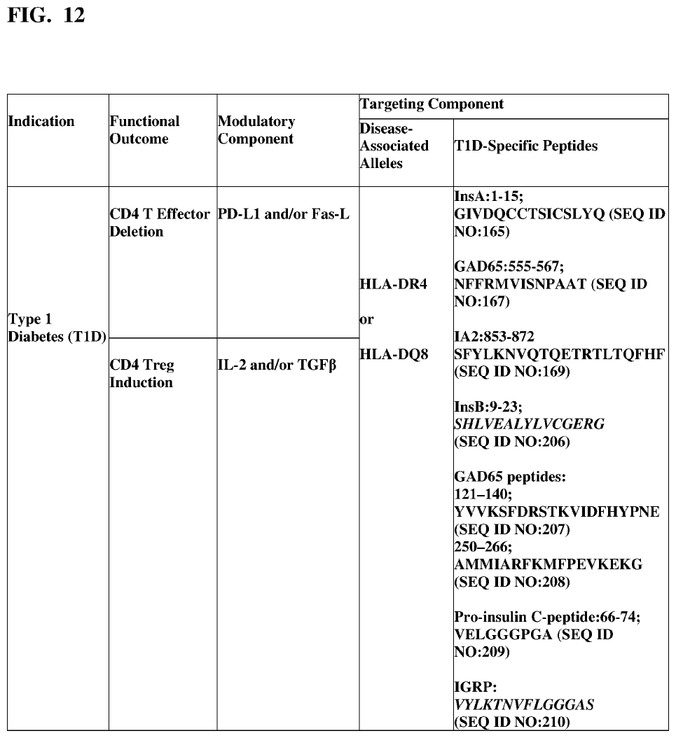

A motifs for chemical conjugation may be incorporated into, or attached to (e.g., via a peptide linker) a TMAPP polypeptide aa sequence having at least 90% (e.g., at least 95%, or at least 98%), or even 100% as sequence identity to at least 70 (e.g., at least 80, or at least 85) or all contiguous aas of an MHC Class II α1, α2, β1, or β2 domain sequence provided in any of FIGS. 4 to 18B. Sequence identity may be determined relative to the corresponding portion of the MHC domain sequences without consideration of the added sulfatase motif or any linker or other sequences present.

A motifs for chemical conjugation may be incorporated into, or attached to (e.g., via a peptide linker) a TMAPP polypeptide as sequence having at least 90% (e.g., at least 95%, or at least 98%) or even 100% as sequence identity to at least 70 (e.g., at least 80, or at least 85) or all contiguous aas of an MHC Class II α1 or α2 domain sequence (e.g., provided in any of FIGS. 4 to 18B).

A motifs for chemical conjugation may be incorporated into, or attached to (e.g., via a peptide linker) a TMAPP polypeptide aa sequence having at least 90% (e.g., at least 95%, 98% or 99%), or even 100% aa sequence identity to at least 70 (e.g., at least 80, or at least 85) or all contiguous aas of an MHC Class II β1 or β2 domain sequence (e.g., provided in any of FIGS. 4 to 18B).

A motifs for chemical conjugation may be incorporated into, or attached to (e.g., via a peptide linker) a-polypeptide in a TMAPP with a sequence having at least 90% (e.g., at least 95%, 98% or 99%), or even 100%) aa sequence identity to at least 70 (e.g., at least 80, or at least 85) or all contiguous aas of an MHC Class II DRA α1 or α2 domain sequence, or an MHC Class II DRB1, DRB3, DRB4 or DRB5 β1, or β2 domain sequence provided in any of FIGS. 4 to 8.

As with motifs for chemical conjugation, chemical conjugation sites (e.g., naturally occurring amino acids, selenocysteines, and non-natural amino acids) may be incorporated into any desired location of a TMAPP. In an embodiment such motifs for chemical conjugation may be excluded from the amino or carboxyl terminal 10 or 20 amino acids. In an embodiment, motifs for chemical conjugation may be added in (e.g., at or near the terminus) of any TMAPP element, including the MHC α chain or β chain polypeptide sequences (e.g., α1, α2, β1, or β2 domain sequences) or any linker sequence directly joined to at least one MHC α chain or β chain polypeptide sequence. Motifs for chemical conjugation also be added to the scaffold polypeptide (e.g., the Ig Fc) or any of the linkers present in the TMAPP.

Chemical conjugation sites may be incorporated into, or attached to (e.g., via a peptide linker) a TMAPP polypeptide aa sequence having at least 90% (e.g., at least 95%, or at least 98%), or even 100% aa sequence identity to at least 70 (e.g., at least 80, or at least 85) or all contiguous aas of an MHC Class II α1, α2, β01, or β2 domain sequence provided in any of FIGS. 4 to 18B. Sequence identity may be determined relative to the corresponding portion of the MHC domain sequences without consideration of the added sulfatase motif or any linker or other sequences present.

A chemical conjugation site may be incorporated into, or attached to (e.g., via a peptide linker) a TMAPP polypeptide aa sequence having at least 90% (e.g., at least 95%, or at least 98%) or even 100% aa sequence identity to at least 70 (e.g., at least 80, or at least 85) or all contiguous aas of an MHC Class II α1 or α2 domain sequence provided in any of FIGS. 4 to 18B.

A chemical conjugation site may be incorporated into, or attached to (e.g., via a peptide linker) a TMAPP polypeptide aa sequence having at least 90% (e.g., at least 95%, 98% or 99%), or even 100% as sequence identity to at least 70 (e.g., at least 80, or at least 85) or all contiguous aas of an MHC Class II β1, or β2 domain sequence provided in any of FIGS. 4 to 18B.

A chemical conjugation site may be incorporated into, or attached to (e.g., via a peptide linker) a TMAPP polypeptide aa sequence having at least 90% (e.g., at least 95%, 98% or 99%), or even 100%) as sequence identity to at least 70 (e.g., at least 80, or at least 85) or all contiguous aas of an MHC Class II DRA α1 or α2 domain sequence, or an MHC Class II DRB1, DRB3, DRB4 or DRB5 β1, or β2 domain sequence provided in any of FIGS. 4 to 8.

A chemical conjugation site may be located in a Class II MHC α chain chemical at several specific positions. Chemical conjugation sites for epitope conjugation may, for example, be located at a chain aa positions 2-5, such as at aas 3 or 4. Chemical conjugation sites for epitope conjugation may, for example be located at α chain aa positions 11-13, such as at aa 12. Chemical conjugation sites for epitope conjugation may, for example be located at α chain an positions 27-30, such as at aas 28 or 29. Chemical conjugation sites for epitope conjugation may, for example be located at α chain aa positions 71-76, such as at aas 72 or 75. Chemical conjugation sites for epitope conjugation may, for example be located at α chain an positions 79-83, such as at aas 80, 81, or 82. Chemical conjugation sites for epitope conjugation may, for example be located at α chain aa positions 92-96, such as at aas 93, 94, or 95. Positions are given based on the mature MHC α chain lacking its signal sequence.

As with the Class II MHC α chain, chemical conjugation sites in the Class II MHC β chain may be located at several specific positions. Chemical conjugation sites for epitope conjugation may, for example be located at β chain an positions 4-11, such as at aas 5, 7 or 10. Chemical conjugation sites for epitope conjugation may, for example be located at β chain an positions 32-34, such as at an 33. Chemical conjugation sites for epitope conjugation may, for example be located at β chain an positions 119-121, such as at aa 120. Chemical conjugation sites for epitope conjugation may, for example be located at β chain an positions 152-158, such as at aas 153 or 156. Where an MHC Class II β2 domain is located at the carboxy terminus of a presenting sequence or presenting complex, a chemical conjugation site (e.g., a cystine residue) may be located in, for example, the last three amino acids of the β2 domain, or in a linker attached to the β2 domain. Positions are given based on the mature MHC β chain lacking its signal sequence.

Chemical conjugation sites and motifs for the conjugation of payload molecules, when present, are typically located on scaffold sequences.

### 1. Sulfatase Motifs

In those embodiments where enzymatic modification is chosen as the means of chemical conjugation, the chemical conjugation site(s) may comprise a sulfatase motif. Sulfatase motifs are usually 5 or 6 aas in length, and are described, for example, in U.S. Pat. No. 9,540,438 and U.S. Pat. Pub. No. 2017/0166639 A1, which are incorporated by reference for the discussion of sulfatase motifs. Insertion of the motif results in the formation of a protein or polypeptide that is sometimes referred to as aldehyde tagged or having an aldehyde tag. The motif may be acted on by formylglycine generating enzyme(s) (“FGE” or “FGEs”) to convert a cysteine or serine in the motif to a formylglycine residue (“fGly” although sometimes denoted “FGly”), which is an aldehyde containing aa, sometimes referred to as oxoalanine, that may be utilized for selective (e.g., site specific) chemical conjugation reactions. Where the term sulfatase motif is utilized in the context of an an sequence, both the nascent chemical conjugation sequence (e.g., a polypeptide containing the unconverted motif) as well as its fGly containing the active chemical conjugation site counterpart are disclosed. Once present in a polypeptide (e.g., of a TMAPP), a fGly residue may be reacted with molecules (e.g., peptide epitopes with or without an intervening linker) comprising a variety of reactive groups including, but not limited to, thiosemicarbazide, aminooxy, hydrazide, and hydrazino groups to form a TMAPP-epitope conjugate having a covalent bond between the TMAPP polypeptide and the now conjugated epitope via the fGly residue. Sulfatase motifs may be used to incorporate not only epitopes (e.g., peptide epitopes), but also to incorporate targeting sequences (e.g., for use in vitro or in vivo) and/or payloads (e.g., in the formation of conjugates with drugs and diagnostic molecules).

In embodiments, the sulfatase motif is at least 5 or 6 aa residues, but can be, for example, from 5 to 16 (e.g., 6-16, 5-14, 6-14, 5-12, 6-12, 5-10, 6-10, 5-8, or 6-8) aas in length. The sulfatase motif may be limited to a length less than 16, 14, 12, 10, or 8 aa residues.

In an embodiment, the sulfatase motif comprises the sequence of in Formula (I):

X1Z1X2Z2X3Z3 (SEQ ID NO:187),

where
Z1 is cysteine or serine;
Z2 is either a proline or alanine residue (which can also be represented by “P/A”);
Z3 is a basic aa (arginine, lysine, or histidine, usually lysine), or an aliphatic aa (alanine, glycine, leucine, valine, isoleucine, or proline, usually A, G, L, V, or I);
X1 is present or absent and, when present, can be any aa, though usually an aliphatic aa, a sulfur-containing aa, or a polar uncharged as (e.g., other than an aromatic as or a charged aa), usually L, M, V, S or T, more usually L, M, S or V, with the proviso that, when the sulfatase motif is at the N-terminus of the target polypeptide, X1 is present; and
X2 and X3 independently can be any aa, though usually an aliphatic aa, a polar, uncharged aa, or a sulfur containing as (e.g., other than an aromatic as or a charged aa), usually S, T, A, V, G or C, more usually S, T, A, V or G.

As indicated above, a sulfatase motif is at least 5 or 6 as residues, but can be, for example, from 5 to 16 aas in length. The motif can contain additional residues at one or both of the N- and C-termini, such that the complete motif includes both a sulfatase motif and an “auxiliary motif.” In an embodiment, the sulfatase motif includes a C-terminal auxiliary motif (i.e., following the Z3 position of the motif).

A variety of FGEs may be employed for the conversion (oxidation) of cysteine or serine in a sulfatase motif to fGly. As used herein, the term formylglycine generating enzyme, or FGE, refers to fGly-generating enzymes that catalyze the conversion of a cysteine or serine of a sulfatase motif to fGly. As discussed in U.S. Pat. No. 9,540,438, the literature often uses the term formylglycine-generating enzymes for those enzymes that convert a cysteine of the motif to fGly, whereas enzymes that convert a serine in a sulfatase motif to fGly are referred to as Ats-B-like.

Sulfatase motifs of Formula (I) amenable to conversion by a prokaryotic FGE often contain a cysteine or serine at Z1 and a proline at Z2 that may be modified either by the “SUMP I-type” FGE or the “AtsB-like” FGE, respectively. Prokaryotic FGE enzymes that may be employed include the enzymes from Clostridium perfringens (a cysteine type enzyme), Klebsiella pneumoniae (a Serine-type enzyme) or the FGE of Mycobacterium tuberculosis. Where peptides containing a sulfatase motif are being prepared for conversion into fGly-containing peptides by a eukaryotic FGE, for example by expression and conversion of the peptide in a eukaryotic cell or cell-free system using a eukaryotic FGE, sulfatase motifs amenable to conversion by a eukaryotic FGE may advantageously be employed.

Host cells for production of polypeptides with unconverted sulfatase motifs, or where the cell expresses a suitable FGE for converting fGly-containing polypeptide sequences, include those of a prokaryotic and eukaryotic organism. Non-limiting examples include Escherichia coli strains, Bacillus spp. (e.g., B. subtilis, and the like), yeast or fungi (e.g., S. cerevisiae, Pichia spp., and the like). Examples of other host cells, including those derived from a higher organism such as insects and vertebrates, particularly mammals, include, but are not limited to, CHO cells, HEK cells, and the like (e.g., American Type Culture Collection (ATCC) No. CCL-2), CHO cells (e.g., ATCC Nos. CRL9618 and CRL9096), CHO DG44 cells, CHO-Ki cells (ATCC CCL-61), 293 cells (e.g., ATCC No. CRL-1573), Vero cells, NIH 3T3 cells (e.g., ATCC No. CRL-1658), Hnh-7 cells, BHK cells (e.g., ATCC No. CCLlO), PC12 cells (ATCC No. CRL1721), COS cells, COS-7 cells (ATCC No. CRL1651), RAT1 cells, mouse L cells (ATCC No. CCLL3), human embryonic kidney (HEK) cells (ATCC No. CRL1573), HLHepG2 cells, and the like.

In an embodiment, the added sulfatase motif is located within the 10 N-terminal aas of a TMAPP polypeptide (e.g., a β1 domain sequence at the N-terminus of a TMAPP polypeptide) or, if present, attached to or within a linker located at the N- or C-terminus of a TMAPP presenting sequence or presenting complex.

U.S. Pat. No. 9,540,438 discusses the incorporation of sulfatase motifs into the various immunoglobulin sequences, including Fc region polypeptides, and is herein incorporated by reference for its teachings on sulfatase motifs and modification of Fc polypeptides and other polypeptides. That patent is also incorporated by reference for its guidance on FGE enzymes, and their use in forming fGly residues, as well as the chemistry related to the coupling of molecules such as epitopes and payloads to fGly residues.

The incorporation of a sulfatase motif may be accomplished by incorporating a nucleic acid sequence encoding the motif at the desired location in a nucleic acid encoding a TMAPP. As discussed below, the nucleic acid sequence may be placed under the control of a transcriptional regulatory sequence(s) (a promoter) and provided with regulatory elements that direct its expression. The expressed protein may be treated with one or more FGEs after expression and partial or complete purification. Alternatively, expression of the nucleic acid in cells that express a FGE that recognizes the sulfatase motif results in the conversion of the cysteine or serine of the motif to fGly.

In view of the foregoing, this disclosure provides for TMAPPs comprising one or more fGly residues incorporated into a TMAPP polypeptide chain as discussed above. The fGly residues may, for example, be in the context of the sequence X1(fGly)X2Z2X3Z3, where: fGly is the formylglycine residue; and Z2, Z3, X1, X2 and X3 are as defined in Formula (I) above. Epitopes and/or payloads may be conjugated either directly or indirectly to the reactive formyl glycine of the sulfatase motif directly or through a peptide or chemical linker. After chemical conjugation the TMAPPs comprise one or more fGly′ residues incorporated in the context of the sequence X1(fGly′)X2Z2X3Z3, where the fGly′ residue is formylglycine that has undergone a chemical reaction and now has a covalently attached epitope or payload.

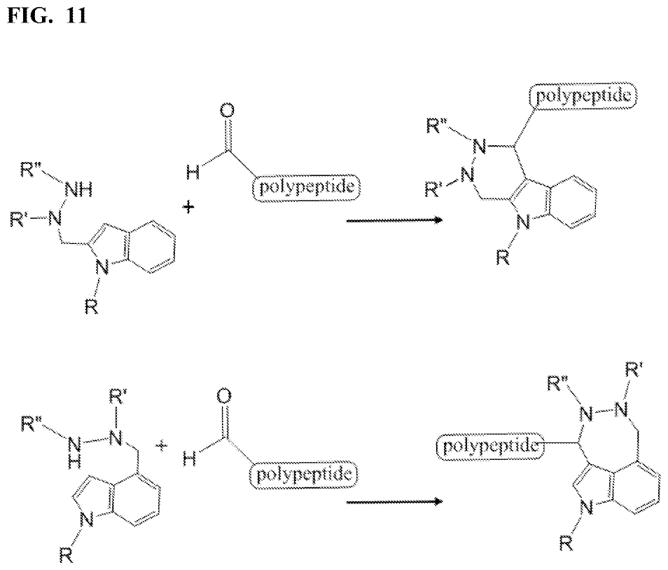

Several chemistries and commercially available reagents can be utilized to conjugate a molecule (e.g., an epitope or payload) to a fGly residue, including, but not limited to, the use of thiosemicarbazide, aminooxy, hydrazide, or hydrazino derivatives of the molecules to be coupled at a fGly-containing chemical conjugation site. For example, epitopes (e.g., peptide epitopes) and/or payloads bearing thiosemicarbazide, aminooxy, hydrazide, hydrazino or hydrazinyl functional groups (e.g., attached directly to an aa of a peptide or via a linker such as a PEG) can be reacted with fGly-containing TMAPP polypeptides to form a covalently linked epitope. See, e.g., FIG. 11. Similarly, targeting sequences and/or payloads such as drugs and therapeutics can be incorporated using, for example, biotin hydrazide as a linking agent.

The disclosure provides for methods of preparing conjugated TMAPPs including TMAPP-epitope conjugates and/or TMAPP-payload conjugates comprising:
a) incorporating a nucleotide sequence encoding a sulfatase motif including a serine or cysteine (e.g., a sulfatase motif of Formula (I) or (II) such as X1CX2PX3Z3 (SEQ ID NO:188); CX1PX2Z3 (SEQ ID NO:189) discussed above) into a nucleic acid encoding an unconjugated TMAPP;
b) expressing the sulfatase motif-containing unconjugated TMAPP polypeptide in a cell that
   i) expresses a FGE and converts the serine or cysteine of the sulfatase motif to a fGly and partially or completely purifying the fGly-containing unconjugated TMAPP, or
   ii) does not express a FGE that converts a serine or cysteine of the sulfatase motif to a fGly, and purifying or partially purifying the TMAPP containing the sulfatase motif and contacting the purified or partially purified TMAPP with a FGE that converts the serine or cysteine of the sulfatase motif into a fGly residue; and
c) contacting the fGly-containing polypeptides with an epitope and/or payload that has been functionalized with a group that forms a covalent bond between the aldehyde of the fGly and epitope and/or payload; thereby forming a TMAPP-epitope conjugate and/or TMAPP payload conjugate.

In such methods of preparing a conjugated TMAPP the epitope (epitope containing molecule) and/or payload may be functionalized by any suitable function group that reacts selectively with an aldehyde group. Such groups may, for example, be selected from the group consisting of thiosemicarbazide, aminooxy, hydrazide, and hydrazino.

### 2. Transglutaminase Enzyme Sites

Transglutaminases catalyze the formation of a covalent bond between the amide group on the side chain of a glutamine residue and a primary amine donor (e.g., a primary alkyl amine, such as is found on the side chain of a lysine residue in a polypeptide). Transglutaminases may be employed to conjugate epitopes and payloads to TMAPPs, either directly through a free amine, or indirectly via a linker comprising a free amine. As such, glutamine residues added to a TMAPP in the context of a transglutaminase site may be considered as chemical conjugation sites when they can be accessed by enzymes such as Streptoverticillium mobaraense transglutaminase. That enzyme (EC 2.3.2.13) is a stable, calcium-independent enzyme catalyzing the 7-acyl transfer of glutamine to the ε-amino group of lysine. Glutamine residues appearing in a sequence are, however, not always accessible for enzymatic modification. The limited accessibility can be advantageous as it limits the number of locations where modification may occur. For example, bacterial transglutaminases are generally unable to modify glutamine residues in native IgG1s; however, Schibli and co-workers (Jeger, S., et al. Angew Chem (Int Engl). 2010; 49:99957 and Dennler P. et al. Bioconjug Chem. 2014; 25(3):569-78) found that deglycosylating IgG1s at N297 rendered glutamine residue Q295 accessible and permitted enzymatic ligation to create an antibody drug conjugate. Further, by producing a N297 to Q297 IgG1 mutant, they introduce two sites for enzymatic labeling by transglutaminase. Modification at N297 also offer the potential to reduce the interaction of the IgG Fc reaction with complement Clq protein.

Where a TMAPP does not contain a glutamine that may be employed as a chemical conjugation site (e.g., it is not accessible to a transglutaminase or not placed in the desired location), a glutamine residue may be added to a sequence to form a transglutaminase site, or a sequence comprising a transglutaminase accessible glutamine (sometimes referred to as a “glutamine tag” or a “Q-tag”), may be incorporated through protein engineering into the polypeptide. The added glutamine or Q-tag may act as a chemical conjugation site for epitopes or payloads. US Pat. Pub. No. 2017/0043033 A 1 describes the incorporation of glutamine residues and Q-tags and the use of transglutaminase for modifying polypeptides and is incorporated herein for those teachings.

Incorporation of glutamine residues and Q-tags may be accomplished chemically where the peptide is synthesized, or by modifying a nucleic acid that encodes the polypeptide and expressing the modified nucleic acid in a cell or cell-free system. In embodiments, the glutamine-containing Q-tag comprises an aa sequence selected from the group consisting of LQG, LLQGG (SEQ ID NO:190), LLQG (SEQ ID NO:191), LSLSQG (SEQ ID NO:192), and LLQLQG (SEQ ID NO:193) (numerous others are available).

As discussed above, glutamine residues and Q-tags may be incorporated into any desired location of a TMAPP. In an embodiment, a glutamine residue or Q-tag may be added in (e.g., at or near the terminus) of any TMAPP element, including the MHC polypeptide sequences or any linker sequence joining them. Glutamine residues and Q-tags may also be added to the scaffold polypeptide (e.g., the Ig Fc) or any of the linkers present in the TMAPP. In an embodiment, the added glutamine residue or Q-tag is attached to the N- or C-terminus of a TMAPP or, if present, attached to or within a linker located at the N- or C-terminus of the TMAPP.

Payloads and epitopes that contain, or have been modified to contain, a primary amine group may be used as the amine donor in a transglutaminase-catalyzed reaction forming a covalent bond between a glutamine residue (e.g., a glutamine residue in a Q-tag) and the epitope or payload.

Where an epitope or payload does not comprise a suitable primary amine to permit it to act as the amine donor, the epitope or payload may be chemically modified to incorporate an amine group (e.g., modified to incorporate a primary amine by linkage to a lysine, aminocaproic acid, cadaverine etc.). Where an epitope or payload comprises a peptide and requires a primary amine to act as the amine donor, a lysine or another primary amine that a transglutaminase can act on may be incorporated into the peptide. Other amine containing compounds that may provide a primary amine group and that may be incorporated into, or at the end of, an alpha amino acid chain include, but are not limited to, homolysine, 2,7-diaminoheptanoic acid, and aminoheptanoic acid. Alternatively, the epitope or payload may be attached to a peptide or non-peptide linker that comprises a suitable amine group. Examples of suitable non-peptide linkers include an alkyl linker and a PEG (polyethylene glycol) linker.

Transglutaminase can be obtained from a variety of sources, including enzymes from: mammalian liver (e.g., guinea pig liver); fungi (e.g., Oomycetes, Actinomycetes, Saccharomyces, Candida, Cryptococcus, Monascus, or Rhizopus transglutaminases); myxomycetes (e.g., Physarum polycephalum transglutaminase); and/or bacteria including a variety of Streptoverticillium, Streptomyces, Actinomadura sp., Bacillus, and the like.

Q-tags may be created by inserting a glutamine or by modifying the aa sequence around existing glutamine residues appearing in a presenting sequence or presenting complex MHC domain and used as a chemical conjugation site for the direct or indirect (through a linker) addition of an epitope or payload. Similarly, Q-tags may be incorporated into a scaffold (e.g., an Ig Fc) or a linker as chemical conjugation sites for the direct or indirect (through a linker) addition of an epitope and/or payload.

### 3. Sortase A Enzyme Sites

Epitopes and payloads may be attached at the N- and/or C-termini TMAPP by incorporating sites for Sortase A conjugation at those locations.

Sortase A recognizes a C-terminal pentapeptide sequence LP(X5)TG/A (SEQ ID NO:194, with X5 being any single amino acid, and G/A being a glycine or alanine), and creates an amide bond between the threonine within the sequence and glycine or alanine in the N-terminus of the conjugation partner.

For attachment of epitopes or payloads to the C-terminal portion of a TMAPP polypeptide an LP(X5)TG/A is provided in the carboxy terminal portion of the desired polypeptide(s). An exposed stretch of glycines or alanines (e.g., (G)₃₋₅ (SEQ ID NOs: 195 and 196 when using Sortase A from Staphylococcus aureus or alanines (A)₃₋₅, SEQ ID NOs: 197 and 198 when using Sortase A from Streptococcus pyogenes) is provided at the N-terminus of a peptide that comprises an epitope (e.g., in a linker attached to the epitope), a peptide payload (or a linker attached thereto), or a peptide covalently attached to a non-peptide epitope or payload.

For attachment of epitopes or payloads to the amino terminus of a TMAPP polypeptide an aa sequence comprising an exposed stretch of glycines (e.g., (G)_{2, 3, 4, or 5}) or alanines (e.g., (A)_{2, 3, 4, or 5}) is provided at the N-terminus, and a LP(X5)TG/A is provided in the carboxy terminal portion of a peptide that comprises an epitope (or a linker attached thereto), a peptide payload (or a linker attached thereto), or a peptide covalently attached to a non-peptide epitope or payload.

Combining Sortase A with the amino and carboxy modified peptides described above results in a cleavage between the Thr and Gly/Ala residues in the LP(X5)TG/A sequence and formation of a covalently coupled complex of the form: carboxy-modified polypeptide-LP(X5)T*G/A-amino-modified polypeptide, where the “*” represents the bond formed between the threonine of the LP(X5)TG/A motif and the glycine or alanine of the N-terminal modified peptide.

In place of LP(X5)TG/A (SEQ ID NO:194), a LPETGG (SEQ ID NO:199) peptide may be used for S. aureus Sortase A coupling, or a LPETAA (SEQ ID NO:200) peptide may be used for S. pyogenes Sortase A coupling. The conjugation reaction still occurs between the threonine and the amino terminal oligoglycine or oligoalanine peptide to yield a carboxy-modified polypeptide-LP(X5)T*G/A-amino-modified polypeptide, where the “*” represents the bond formed between the threonine and the glycine or alanine of the N-terminal modified peptide.

### 4. Selenocysteine and Non-Natural Amino Acids as Chemical Conjugation Sites

One strategy for providing site-specific chemical conjugation sites into a TMAPP polypeptide employs the insertion of aas with reactivity distinct from the naturally occurring proteinogenic L-amino acids aas present in the polypeptide. Such aas include, but are not limited to, the, selenocysteine (Sec), and the non-natural aas: acetylphenylalanine (p-acetyl-L-phenylalanine, pAcPhe); parazido phenylalanine; and propynyl-tyrosine. Thanos et al. in US Pat. Publication No. 20140051836 A1 discuss some other non-natural aas including O-methyl-L-tyrosine, O-4-allyl-L-tyrosine, tri-O-acetyl-GlcNAcβ-serine, isopropyl-L-phenylalanine, p-benzoyl-L-phenylalanine, L-phosphoserine, and a p-propargyloxy-phenylalanine. Other non-natural aas include reactive groups such as, for example, amino, carboxy, acetyl, hydrazino, hydrazido, semicarbazido, sulfanyl, azido and alkynyl. See, e.g., US Pat. Publication No. 20140046030 A1.

In addition to directly synthesizing polypeptides in the laboratory, two methods utilizing stop codons have been developed to incorporate non-natural aas into proteins and polypeptides utilizing transcription-translation systems. The first incorporates selenocysteine (Sec) by pairing the opal stop codon, UGA, with a Sec insertion sequence. The second incorporates non-natural aas into a polypeptide generally through the use of amber, ochre, or opal stop codons. The use of other types of codons such as a unique codon, a rare codon, an unnatural codon, a five-base codon, and a four-base codon, and the use of nonsense and frameshift suppression have also been reported. See, e.g., US Pat. Publication No. 20140046030 A1 and Rodriguez et al., PNAS 103(23)8650-8655(2006). By way of example, the non-natural amino acid acetylphenylalanine may be incorporated at an amber codon using a tRNA/aminoacyl tRNA synthetase pair in an in vivo or cell-free transcription-translation system.

Incorporation of both selenocysteine and non-natural aas requires engineering the necessary stop codon(s) into the nucleic acid coding sequence of a TMAPP polypeptide at the desired location(s), after which the coding sequence is used to express the TMAPP in an in vivo or cell-free transcription-translation system.

In vivo systems generally rely on engineered cell-lines to incorporate non-natural aas that act as bio-orthogonal chemical conjugation sites into polypeptides and proteins. See, e.g., International Published Application No. 2002/085923 entitled “In vivo incorporation of unnatural amino acids.” In vivo non-natural as incorporation relies on a tRNA and an aminoacyl tRNA synthetase pair that is orthogonal to all the endogenous tRNAs and synthetases in the host cell. The non-natural as of choice is supplemented to the media during cell culture or fermentation, making cell-permeability and stability important considerations.

Various cell-free synthesis systems provided with the charged tRNA may also be utilized to incorporate non-natural aas. Such systems include those described in US Pat. Publication No. 20160115487Δ1; Gubens et al., RNA. 2010 August; 16(8): 1660-1672; Kim, D. M. and Swartz, J. R. Biotechnol. Bioeng. 66:180-8 (1999); Kim, D. M. and Swartz, J. R. Biotechnol. Prog. 16:385-90 (2000); Kim, D. M. and Swartz, J. R. Biotechnol. Bioeng. 74:309-16 (2001); Swartz et al, Methods Mol. Biol. 267:169-82 (2004); Kim, D. M. and Swartz, J. R. Biotechnol. Bioeng. 85:122-29 (2004); Jewett, M. C. and Swartz, J. R., Biotechnol. Bioeng. 86:19-26 (2004); Yin, G. and Swartz, J. R., Biotechnol. Bioeng. 86:188-95 (2004); Jewett, M. C. and Swartz, J. R., Biotechnol. Bioeng. 87:465-72 (2004); Voloshin, A. M. and Swartz, J. R., Biotechnol. Bioeng. 91:516-21 (2005).

Once incorporated into the TMAPP, epitopes and/or payload bearing groups reactive with the incorporated selenocysteine or non-natural aa are brought into contact with the TMAPP under suitable conditions to form a covalent bond. By way of example, the keto group of the pAcPhe is reactive towards alkoxyamines, and via oxime coupling can be conjugated directly to alkoxyamine containing epitopes and/or payloads or indirectly to epitopes and payloads via an alkoxyamine containing linker. Selenocysteine reacts with, for example, primary alkyl iodides (e.g., iodoacetamide which can be used as a linker), maleimides, and methylsulfone phenyloxadiazole groups. Accordingly, epitopes and/or payloads bearing those groups or bound to linkers bearing those groups can be covalently bound to polypeptide chains bearing selenocysteines.

As discussed above for other chemical conjugation sites, selenocysteines and/or non-natural aas may be incorporated into any desired location in the TMAPP for use as a chemical conjugation site. The site will preferably be solvent accessible. In an embodiment, mature α chain positions 72 and 75 (e.g., 172 and K75 of a mature DRA polypeptide respectively) are used for the direct or indirect (though a linker) conjugation of an epitope presenting molecule, such as a peptide epitope.

### 5. Amino Acid Chemical Conjugation Sites

Any of the variety of functionalities (e.g., —SH, —NH₃, —OH, —COOH and the like) present in the side chains of naturally occurring amino acids, or at the termini of polypeptides, can be used as chemical conjugation sites. This includes the side chains of lysine and cysteine, which are readily modifiable by reagents including N-hydroxysuccinimide and maleimide functionalities, respectively. The main disadvantages of utilizing such amino acid residues is the potential variability and heterogeneity of the products. For example, an IgG has over 80 lysines, with over 20 at solvent-accessible sites. See, e.g., McComb and Owen, AAPS J. 117(2): 339-351. Cysteines tend to be less widely distributed; they tend to be engaged in disulfide bonds, and may be inaccessible (e.g., not accessible by solvent or to molecules used to modify the cysteines, and not located where it is desirable to place a chemical conjugation site. It is, however, possible to selectively modify TMAPP polypeptides to provide naturally occurring and, as discussed above, non-naturally occurring amino acids at the desired locations for placement of a chemical conjugation site. Modification may take the form of direct chemical synthesis of the polypeptides (e.g., by coupling appropriately blocked amino acids) and/or by modifying the sequence of a nucleic acid encoding the polypeptide followed expression in a cell or cell-free system. Accordingly, this disclosure includes and provides for the preparation of the TMAPP polypeptides by transcription/translation systems capable of incorporating a non-natural aa or natural aa to be used as a chemical conjugation site for epitope or payload conjugation.

This disclosure includes and provides for the preparation of a portion of a TMAPP by transcription/translation systems and joining to its C- or N-terminus a polypeptide bearing a non-natural aa or natural aa prepared by, for example, chemical synthesis. The polypeptide, which may include a linker, may be joined by any suitable method including the use of a sortase as described above for peptide epitopes. In an embodiment, the polypeptide may comprise a sequence of 2, 3, 4, or 5 alanines or glycines that may serve for sortase conjugation and/or as part of a linker sequence.

As discussed above for other chemical conjugation sites, naturally occurring aas may be incorporated into any desired location in the TMAPP for use as a chemical conjugation site. The site will preferably be solvent accessible. In an embodiment, mature α chain positions 72 and 75 (e.g., 172 and K75 of a mature DRA polypeptide respectively) are used for the direct or indirect (though a linker) conjugation of an epitope presenting molecule, such as a peptide epitope.

In any of the embodiments mentioned above where a naturally occurring aa is provided, e.g., via protein engineering, in a polypeptide, the an may be selected from the group consisting of arginine, lysine, cysteine, serine, threonine, glutamic acid, glutamine, aspartic acid, and asparagine. Alternatively, the aa provided as a conjugation site is selected from the group consisting of lysine, cysteine, serine, threonine, and glutamine. The an provided as a conjugation site may also be selected from the group consisting of lysine, glutamine, and cysteine. In one instance, the provided aa is cysteine. In another instance, the provided an is lysine. In still another instance, the provided an is glutamine.

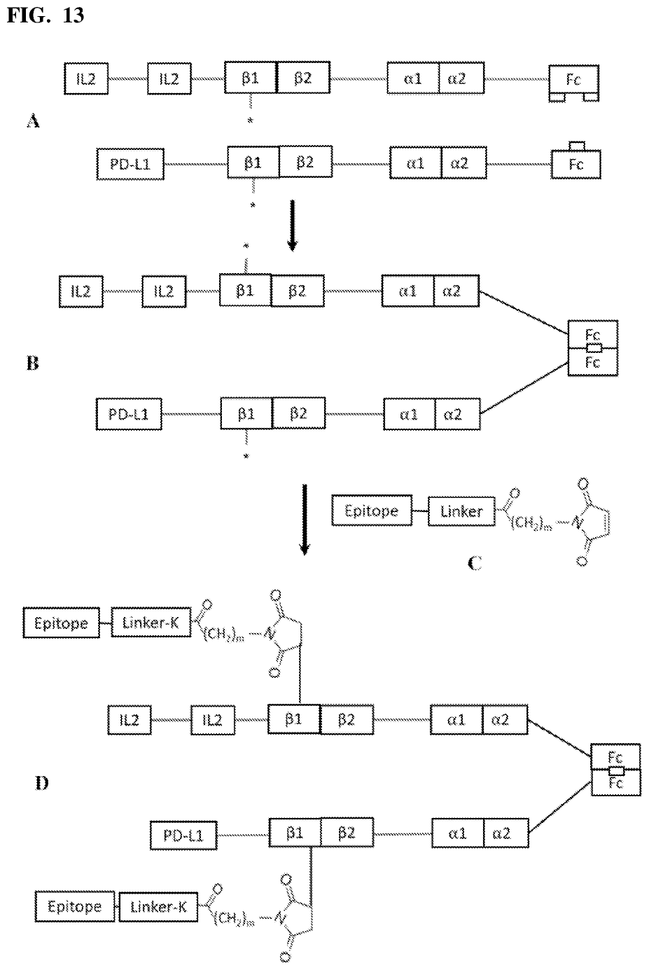

Any method known in the art may be used to couple payloads or epitopes to amino acids provided in an unconjugated TMAPP. By way of example, maleimides may be utilized to couple to sulfhydryls, N-hydroxysuccinimide may be utilized to couple to amine groups, acid anhydrides or chlorides may be used to couple to alcohols or amines, and dehydrating agents may be used to couple alcohols or amines to carboxylic acid groups. Accordingly, using such chemistry an epitope or payload may be coupled directly, or indirectly through a linker (e.g., a homo- or hetero-bifunctional crosslinker), to a location on an TMAPP polypeptide. A number of bifunctional crosslinkers may be utilized, including, but not limited to, those described for linking a payload to a TMAPP described herein below. For example, a peptide epitope (or a peptide-containing payload) including a maleimide group attached by way of a homo- or hetero-bifunctional linker (see, e.g., FIG. 13) or a maleimide amino acid can be conjugated to a sulfhydryl of a chemical conjugation site (e.g., a cysteine residue) that is naturally occurring or provided in a TMAPP.

Maleimido amino acids can be incorporated directly into peptides (e.g., peptide epitopes) using a Diels-Alder/retro-Diels-Alder protecting scheme as part of a solid phase peptide synthesis. See, e.g., Koehler, Kenneth Christopher (2012), “Development and Implementation of Clickable Amino Acids,” Chemical & Biological Engineering Graduate Theses & Dissertations, 31, https://scholar.colorado.edu/chbe_gradetds/31.

A maleimide group may also be appended to an epitope (e.g., a peptide epitope) using a homo- or hetero-bifunctional linker (sometimes referred to as a crosslinker) that attaches a maleimide directly or indirectly (e.g., through an intervening linker that may comprise additional aas bound to the epitope) to the epitope (e.g., peptide epitope). For example, a heterobifunctional N-hydroxysuccinimide-maleimide crosslinker can attach maleimide to an amine group of, a peptide lysine. Some specific cross linkers include molecules with a maleimide functionality and either a N-hydroxysuccinimide ester (NHS) or N-succinimidyl group that can attach a maleimide to an amine (e.g., an epsilon amino group of lysine). Examples of such crosslinkers include, but are not limited to, NHS-PEG4-maleimide, γ-maleimide butyric acid N-succinimidyl ester (GMBS); ε-maleimidocaproic acid N-hydroxysuccinimide ester (EMCS); m-maleimide benzoyl-N-hydroxysuccinimide ester (MBS); and N-(α-maleimidoacetoxy)-succinimide ester (AMAS), which offer different lengths and properties for peptide immobilization. Another amine reactive crosslinker that can incorporate a maleimide group includes N-succinimidyl 4-(2-pyridyldithio)butanoate (SPDB). Additional crosslinkers (bifunctional agents) are recited below. In an embodiment the epitopes coupled to the TMAPP have a maleimido alkyl carboxylic acid coupled to the peptide by an optional linker (see, e.g., FIG. 13), for example, by an amide formed with the epsilon amino group of a lysine. The maleimido carboxylic acid can be, for example, a maleimido ethanoic, propanoic, butanoic, pentanoic, hexanoic, heptanoic, or octanoic acid.

A peptide epitope may be coupled to a naturally occurring cysteine present or provided in (e.g., engineered into) for example, the binding pocket of a TMAPP through a bifunctional linker comprising a maleimide or a maleimide amino acid incorporated into the peptide, thereby forming a TMAPP-epitope conjugate.

As discussed above, a peptide epitope may be conjugated a presenting sequence or presenting complex having cysteine residues that have been substituted into the MHC α chain or β chain aa sequences. By way of example, a peptide epitope comprising maleimido amino acids or bearing a maleimide group as part of a linker attached to the peptide epitope may be covalently attached to a chemical conjugation site (e.g., a cysteine) located at any one of aa positions 4-11 (e.g., at aa 5, 7 or 10) of the mature β chain. Alternatively, a chemical conjugation site (e.g., a cysteine) located at any one of aa positions 79-83 (e.g., at as 80, 81, or 82).

Where conjugation of an epitope, targeting sequences and/or, payload is to be conducted through a cysteine chemical conjugation site present in an unconjugated TMAPP (e.g., using a maleimide modified epitope or payload) a variety of process conditions may affect the conjugation efficiency and the quality (e.g., the amount/fraction of unaggregated duplex TMAPP-epitope conjugate resulting from the reaction) resulting from the conjugation reaction. Conjugation process conditions that may be individually optimized including, but not limited to, (i) prior to conjugation unblocking of cysteine sulfhydryls (e.g., potential blocking groups may be present and removed), (ii) the ratio of the TMAPP to the epitope or payload, reaction pH, (iii) the buffer employed, (iv) additives present in the reaction, (v) the reaction temperature, and (vi) the reaction time.

Prior to conjugation TMAPPs may be treated with a disulfide reducing agent such as dithiothreitol (DTI), mercaptoethanol, or tris(2-carboxyethyl)phosphine (TCEP) to reduce and free cysteines sulfhydryls that may be blocked. Treatment may conducted using relatively low amounts of reducing agent, for example from about 0.5 to 2.0 reducing equivalents per cysteine conjugation site for relatively short periods, and the cysteine chemical conjugation site of the unconjugated TMAPP may be available as a reactive nucleophile for conjugation from about 10 minutes to about 1 hour, or from about 1 hour to 5 hours.

The ratio of the unconjugated TMAPP to the epitope or payload being conjugated may be varied from about 1:2 to about 1:100, such as from about 1:2 to about 1:3, from about 1:3 to about 1:10, from about 1:10 to about 1:20, from about 1:20 to about 1:40, or from about 1:40 to about 1:100. The use of sequential additions of the reactive epitope or payload may be made to drive the coupling reaction to completion (e.g., multiple does of maleimide or N-hydroxy succinimide modified epitopes may be added to react with the TMAPP).

As previously indicated, conjugation reaction may be affected by the buffer, its pH, and additives that may be present. For maleimide coupling to reactive cysteines present in a TMAPP the reactions are typically carried out from about pH 6.5 to about pH 8.0 (e.g., from about pH 6.5 to about pH 7.0, from about pH 7.0 to about pH 7.5, from about pH 7.5 to about pH 8.0, or from about pH 8.0 to about pH 8.5. Any suitable buffer not containing active nucleophiles (e.g., reactive thiols) and preferably degassed to avoid reoxidation of the sulfhydryl may be employed for the reaction. Some suitable traditional buffers include phosphate buffered saline (PBS), Tris-HCl, and (4-(2-hydroxyethyl)-1-piperazineethanesulfonic acid) HEPES. As an alternative traditional buffers, maleimide conjugation reactions may be conducted in buffers/reaction mixtures comprising amino acids such as arginine, glycine, lysine, or histidine. The use of high concentrations of amino acids, e.g., from about 0.1 M (molar) to about 1.5 M (e.g., from about 0.1 to about 0.25, from about 0.25 to about 0.5 from about 0.3 to about 0.6, from about 0.4 to about 0.7, from about 0.5 to about 0.75, from about 0.75 to about 1.0, from about 1.0 to about 1.25 M, or from about 1.25 to about 1.5 M may stabilize the unconjugated and/or unconjugated TMAPP.

Additives useful for maleimide and other conjugation reactions include, but are not limited to: protease inhibitors; metal chelator (e.g., EDTA) that can block unwanted side reactions and inhibit metal dependent proteases if they are present, detergents; detergents (e.g., polysorbate 80 sold as TWEEN 80®, or nonylphenoxypolyethoxyethanol sold under the names NP40 and Tergitol™ NP); and polyols such a sucrose or glycerol that can add to protein stability.

Conjugation of TMAPPs with epitopes, targeting sequences and/or payloads, and particularly conjugation at cysteines using maleimide chemistry, can be conducted over a range of temperatures, such as 0° to 40° C. For example, conjugation reactions, including cysteine-maleimide reactions, can be conducted from about 0° to about 10° C., from about 10° to about 20° C., from about 20° to about 30° C., from about 25° to about 37° C., or from about 30° to about 40° C. (e.g., at about 20° C., at about ° C. or at about 37° C.).

Where a pair of sulfhydryl groups are present, they may be employed simultaneously for chemical conjugation to a TMAPP. In such an embodiment, an unconjugated TMAPP that has a disulfide bond, or that has two cysteines (or selenocysteines) provided at locations proximate to each other, may be utilized as a chemical conjugation site by incorporation of bis-thiol linkers. Bis-thiol linkers, described by Godwin and co-workers, avoid the instability associated with reducing a disulfide bond by forming a bridging group in its place and at the same time permit the incorporation of another molecule, which can be an epitope or payload. See, e.g., Badescu G, et al., (2014), Bioconjug Chem., 25(6):1124-36, entitled Bridging disulfides for stable and defined antibody drug conjugates, describing the use of bis-sulfone reagents, which incorporate a hydrophilic linker (e.g., PEG (polyethylene glycol) linker).

Generally, stoichiometric or near stoichiometric amounts of dithiol reducing agents (e.g., dithiothreitol) are employed to reduce the disulfide bond and allow the bis-thiol linker to react with both cysteine and/or selenocysteine residues. Where multiple disulfide bonds are present, the use of stoichiometric or near stoichiometric amounts of reducing agents may allow for selective modification at one site. See, e.g., Brocchini, et al., Adv. Drug. Delivery Rev. (2008) 60:3-12. Where a TMAPP or duplexed TMAPP does not comprise a pair of cysteines and/or selenocysteines (e.g., a selenocysteine and a cysteine), they may be provided in the polypeptide (by introducing one or both of the cysteines or selenocysteines) to provide a pair of residues that can interact with a bis-thiol linker. The cysteines and/or selenocysteines should be located such that a bis-thiol linker can bridge them (e.g., at a location where two cysteines could form a disulfide bond). Any combination of cysteines and selenocysteines may be employed (i.e. two cysteines, two selenocysteines, or a selenocysteine and a cysteine). The cysteines and/or selenocysteines may both be present on a TMAPP. Alternatively, in a duplex TMAPP the first cysteine and/or selenocysteine is present in the first TMAPP of the duplex and a second cysteine and/or selenocysteine is present in the second TMAPP of the duplex, with the bis-thiol linker acting as a covalent bridge between the duplexed TMAPPs. In another embodiment, a pair of cysteines and/or selenocysteines is incorporated into an MHC polypeptide sequence of a TMAPP as a chemical conjugation site. In an embodiment, a pair of cysteines and/or selenocysteines is incorporated into a polypeptide comprising a sequence having at least 85% (e.g., at least 90%, 95%, 98% or 99%, or even 100%) aa sequence identity to a sequence having at least 150, 175, 200, or 225 contiguous aas of an MHC sequence shown in any of FIGS. 4-18B before the addition of a pair of cysteines or selenocysteines, or into a peptide linker attached to one of those sequences. In one such embodiment the pair of cysteines and/or selenocysteines may be utilized as a bis-thiol linker coupling site for the conjugation of an epitope and/or payload through a peptide or chemical linker attached to the bis-thiol linker. Where the MHC sequence includes a Y84C and Δ139C substitutions the bis-thiol linker may be used to form a covalent bridge between those sites for the covalent coupling of an epitope (e.g., a peptide epitope).

In another embodiment, a pair of cysteines and/or selenocysteines is incorporated into an Ig Fc sequence of a TMAPP to provide a chemical conjugation site. In an embodiment a pair of cysteines and/or selenocysteines is incorporated into a polypeptide comprising an Ig Fc sequence having at least 90% (e.g., at least 95% or at least 99%) or even 100% an sequence identity to a sequence shown in any of the Fc sequences of FIGS. 2A-2G before the addition of the pair of cysteines or selenocysteines. In one such embodiment the pair of cysteines and/or selenocysteines is utilized as a bis-thiol linker coupling site for the conjugation of an epitope and/or payload through a peptide or chemical linker attached to the bis-thiol group. The bis-thiol linker may be used to form a covalent bridge between scaffold polypeptides of a duplex TMAPP. In such a case the cysteines of the lower hinge region that form interchain disulfide bonds, if present in the Ig Fc scaffold polypeptide sequence, may be used to insert the bis-thiol linker.

### 6. Other Chemical Conjugation Sites

a. Carbohydrate Chemical Conjugation Sites

Many proteins prepared by cellular expression contain added carbohydrates (e.g., oligosaccharides of the type added to antibodies expressed in mammalian cells). Accordingly, where sc- or m-TMAPPs are prepared by cellular expression of their polypeptides, carbohydrates may be present and available as site selective chemical conjugation sites in glycol-conjugation reactions. McCombs and Owen, AAPS Journal, (2015) 17(2): 339-351, and references cited therein describe the use of carbohydrate residues for glycol-conjugation of molecules to antibodies.

The addition and modification of carbohydrate residues may also be conducted through the use of chemicals that alter the carbohydrates (e.g., periodate, which introduces aldehyde groups), or by the action of enzymes (e.g., fucosyltransferases) that can incorporate chemically reactive carbohydrates or carbohydrate analogs for use as chemical conjugation sites.

In an embodiment, the incorporation of an IgFc scaffold with known glycosylation sites may be used to introduce site specific chemical conjugation sites into a sc- or m-TMAPP.

This disclosure includes and provides for TMAPPs having carbohydrates as chemical conjugation (glycol-conjugation) sites. The disclosure also includes and provides for the use of such molecules in forming conjugates with epitopes and with other molecules such as drugs and diagnostic agents, and the use of those molecule in methods of treatment and diagnosis.

b. Nucleotide Binding Sites

Nucleotide binding sites offer site-specific functionalization through the use of a UV-reactive moiety that can covalently link to the binding site. Bilgicer et al., Bioconjug Chem. 2014; 25(7):1198-202, reported the use of an indole-3-butyric acid (IBA) moiety can be covalently linked to an IgG at a nucleotide binding site. By incorporation of the sequences required to form a nucleotide binding site, chemical conjugates of any TMAPP with suitably modified epitopes and/or other molecules (e.g., drugs or diagnostic agents) bearing a reactive nucleotide may be employed to prepare TMAPP-epitope conjugates.

This disclosure includes and provides for sc- or m-TMAPPs having nucleotide binding sites as chemical conjugation sites. The disclosure also includes and provides for the use of such molecules in forming conjugates with epitopes and with other molecules such as drugs and diagnostic agents, and the use of those molecule in methods of treatment and diagnosis.

### V.A(x). Bifunctional Linkers and Epitope and Non-Epitope Conjugates

A broad variety of molecules, sometimes called “payloads,” in addition to epitopes may be conjugated to any TMAPP comprising a chemical conjugation site using homobifunctional or heterobifunctional linkers. Furthermore, where TMAPPs multimerize to form higher order species, it may be possible to incorporate monomers conjugated with more than one type of payload molecule in a multimer. Accordingly, in addition to the epitopes, it is possible to introduce one or more types of non-epitope molecules selected from the group consisting of: therapeutic agents, chemotherapeutic agents, diagnostic agents, labels and the like. It will be apparent that some molecules may fall into more than one category (e.g., a radio label may be useful as a diagnostic and as a therapeutic for selectively irradiating a specific tissue or cell type).

As noted above, various polypeptides of any TMAPP (e.g., a scaffold or Fc polypeptide) can be modified at chemical conjugation sites to incorporate payload molecules in addition to epitope peptides. In addition to the specific chemistries discussed above for modification of chemical conjugation sites, crosslinking reagents may be employed to attach epitope and non-epitope “other molecules” to sites in any TMAPP. Bifunctional agents, including those with maleimide and iodo (e.g., iodoacetate) groups react with nucleophiles (e.g., cysteine nucleophiles), and N-hydroxysuccinimide esters react with amines (e.g., primary amines such as those on lysine). Bifunctional agents (also called crosslinking agents) include, but are not limited to, succinimidyl 4-(N-maleimidomethyl)-cyclohexane-1-carboxylate (SMCC), sulfo-SMCC, maleimidobenzoyl-N-hydroxysuccinimide ester (MBS), sulfo-MBS and succinimidyl-iodoacetate.

Some bifunctional linkers for introducing molecules, particularly payloads, into any TMAPP include cleavable linkers and non-cleavable linkers. In some cases, the linker is a proteolytically cleavable linker. Some suitable proteolytically cleavable linkers comprise an amino acid sequence selected from the group consisting of: a) LEVLFQGP (SEQ ID NO:201); b) ENLYTQS (SEQ ID NO:202); c) DDDDK (SEQ ID NO:203); d) LVPR (SEQ ID NO:204); and e) GSGATNFSLLKQAGDVEENPGP (SEQ ID NO:205). Suitable linkers, particularly for payloads (sometimes called “payload linkers”) include, e.g., peptides (e.g., from 2 to 10 amino acids in length; e.g., 2, 3, 4, 5, 6, 7, 8, 9, or 10 amino acids in length), alkyl chains, poly(ethylene glycol), disulfide groups, thioether groups, acid labile groups, photolabile groups, peptidase labile groups, and esterase labile groups.

In addition to the bifunctional agents listed above, non-limiting examples of suitable bifunctional agents, which can also serve as linkers include: N-succinimidyl-[(N-maleimidopropionamido)-tetraethyleneglycol]ester (NHS-PEG4-maleimide); N-succinimidyl 4-(2-pyridyldithio)butanoate (SPDB); disuccinimidyl suberate (DSS); disuccinimidyl glutarate (DGS); dimethyl adipimidate (DMA); N-succinimidyl 4-(2-pyridyldithio)2-sulfobutanoate (sulfo-SPDB); N-succinimidyl 4-(2-pyridyldithio) pentanoate (SPP); N-succinimidyl-4-(N-maleimidomethyl)-cyclohexane-1-carboxy-(6-amidocaproate) (LC-SMCC); K-maleimidoundecanoic acid N-succinimidyl ester (KMUA); γ-maleimide butyric acid N-succinimidyl ester (GMBS); ε-maleimidocaproic acid N-hydroxysuccinimide ester (EMCS); m-maleimide benzoyl-N-hydroxysuccinimide ester (MBS); N-(α-maleimidoacetoxy)-succinimide ester (AMAS); succinimidyl-6-(β-maleimidopropionamide)hexanoate (SMPH); N-succinimidyl 4-(p-maleimidophenyl)butyrate (SMPB); N-(p-maleimidophenyl)isocyanate (PMPI); N-succinimidyl 4(2-pyridylthio)pentanoate (SPP); N-succinimidyl(4-iodo-acetyl)aminobenzoate (SIAB); 6-maleimidocaproyl (MC); maleimidopropanoyl (MP); p-aminobenzyloxycarbonyl (PAB); N-succinimidyl 4-(maleimidomethyl)cyclohexanecarboxylate (SMCC); succinimidyl 3-(2-pyridyldithio)propionate (SPDP); PEG4-SPDP (PEGylated, long-chain SPDP crosslinker); BS(PEG)₅ (PEGylated bis(sulfosuccinimidyl)suberate); BS(PEG)₉ (PEGylated bis(sulfosuccinimidyl)suberate); maleimide-PEG₆-succinimidyl ester; maleimide-PEG₈-succinimidyl ester; maleimide-PEG₁₂-succinimidyl ester; PEG₄-SPDP (PEGylated, long-chain SPDP crosslinker); PEG₁₂-SPDP (PEGylated, long-chain SPDP crosslinker); N-succinimidyl-4-(N-maleimidomethyl)-cyclohexane-1-carboxy-(6-amidocaproate), a “long chain” analog of SMCC (LC-SMCC); 3-maleimidopropanoic acid N-succinimidyl ester (BMPS); N-succinimidyl iodoacetate (SIA); N-succinimidyl bromoacetate (SBA); and N-succinimidyl 3-(bromoacetamido)propionate (SBAP).

Control of the stoichiometry of the reaction may result in some selective modification where engineered sites with chemistry orthogonal to other groups in the TMAPP molecule are not utilized. Reagents that display far more selectivity, such as the bis-thio linkers discussed above tend to permit more precise control of the location and stoichiometry than reagents that react with single lysine, or cysteine residues.

In embodiments where a TMAPP of the present disclosure comprises an Fc polypeptide, the Fc polypeptide can comprise one or more covalently attached molecules of payload attached directly or indirectly through a functional linker. By way of example, where a sc- or a m-TMAPP) comprises a Fc polypeptide, the polypeptide chain comprising the Fc polypeptide can be of the formula (A)-(L)-(C), where (A) is the polypeptide chain comprising the Fc polypeptide; where (L), if present, is a linker; and where (C) is a payload (e.g., a cytotoxic agent). (L), if present, links (A) to (C). In some cases, the polypeptide chain comprising the Fc polypeptide can comprise more than one molecule of payload (e.g., cytotoxic agent), for example 2, 3, 4, 5, or more than 5 molecules of payload). In an embodiment, payload drugs that may be conjugated to a TMAPP (e.g., to the Fc peptide) include sulfasalazine, azathioprine, cyclophosphamide, antimalarials, D-penicillamine, cyclosporine, non-steroidal anti-inflammatory drugs, glucocorticoids, leflunomide, methotrexate, and the like.

In an embodiment, a polypeptide (e.g., an Fc polypeptide) of a TMAPP can be modified with crosslinking reagents such as succinimidyl 4-(N-maleimidomethyl)-cyclohexane-1-carboxylate (SMCC), sulfo-SMCC, maleimidobenzoyl-N-hydroxysuccinimide ester (MBS), sulfo-MBS or succinimidyl-iodoacetate, as described in the literature, to introduce 1-10 reactive groups. The modified Fc polypeptide is then reacted with a thiol-containing agent to produce a conjugate.

In an embodiment, the non-epitope molecules (e.g., a payload) conjugated to any TMAPP are selected from the group consisting of: biologically active agents or drugs, diagnostic agents or labels, nucleotide or nucleoside analogs, nucleic acids or synthetic nucleic acids (e.g., antisense nucleic acids, small interfering RNA, double stranded (ds)DNA, single stranded (ss)DNA, ssRNA, dsRNA), toxins, liposomes (e.g., incorporating a chemotherapeutic such as 5-fluorodeoxyuridine), nanoparticles (e.g., gold or other metal bearing nucleic acids or other molecules, lipids, particle bearing nucleic acids or other molecules), and combinations thereof. Such molecules may be considered and used as drug conjugates, diagnostic agents, and labels.

In an embodiment, the non-epitope molecules conjugated to any TMAPP are selected from the group consisting of: biologically active agents or drugs selected independently from the group consisting of: therapeutic agents (e.g., drug or prodrug), chemotherapeutic agents, cytotoxic agents, antibiotics, antivirals, cell cycle synchronizing agents, ligands for cell surface receptor(s), immunomodulatory agents (e.g., immunosuppressants such as cyclosporine), pro-apoptotic agents, anti-angiogenic agents, cytokines, chemokines, growth factors, proteins or polypeptides, antibodies or an antigen binding fragment thereof, enzymes, proenzymes, hormones and combinations thereof.

In an embodiment the non-epitope molecules conjugated to any TMAPP may be therapeutic agents or chemotherapeutic agents, diagnostic agents, or labels selected independently from the group consisting of photodetectable labels (e.g., dyes, fluorescent labels, phosphorescent labels, luminescent labels), contrast agents (e.g., iodine or barium containing materials), radiolabels, imaging agents, paramagnetic labels/imaging agents (gadolinium containing magnetic resonance imaging labels), ultrasound labels and combinations thereof.

### V.B. Nucleic Acids

The present disclosure provides a nucleic acid comprising a nucleotide sequence encoding one or more polypeptides of a TMAPP that may be conjugated to a T1D-associated epitope. In some cases, the nucleic acid is a recombinant expression vector; thus, the present disclosure provides a recombinant expression vector comprising a nucleotide sequence encoding a.

V.B(i). Nucleic Acids Encoding a TMAPP or TMAPP Forming a Higher Order Complex, Such as a duplex TMAPP,

The present disclosure provides a nucleic acid comprising a nucleotide sequence encoding one or more polypeptides of a TMAPP. In some cases, the nucleic acid is a recombinant expression vector; thus, the present disclosure provides a recombinant expression vector comprising a nucleotide sequence encoding a. In some instances the TMAPP encoded by the nucleotide sequence comprises a scaffold that can associate to form duplexes or higher order complexes, accordingly causing molecules of TMAPP to form duplexes and higher order complexes. Where the TMAPP or duplex TMAPP comprises more than one type of polypeptide, such as where it comprises a trans masked TGF-β MOD and a pair interspecific scaffolds sequences, and/or where the TMAPP comprises a presenting complex, the nucleic acid sequences encoding the different peptides may be located on one or more nucleic acid molecules. The nucleotide sequence(s) comprising any of the TMAPP polypeptides can be operably linked to a transcription control element(s), e.g., a promoter. Accordingly, individual polypeptides of a TMAPP may be encoded on a single nucleic acid (e.g., under the control of separate promoters), or alternatively, may be located on two or more separate nucleic acids (e.g., plasmids).

### V.B(ii). Recombinant Expression Vectors

The present disclosure provides recombinant expression vectors comprising nucleic acids encoding one or more polypeptides of a TMAPP or its higher order complexes. In some cases, the recombinant expression vector is a non-viral vector. In some cases, the recombinant expression vector is a viral construct, such as a recombinant adeno-associated virus construct (see, e.g., U.S. Pat. No. 7,078,387), a recombinant adenoviral construct, a recombinant lentiviral construct, a recombinant retroviral construct, a non-integrating viral vector, etc.

Suitable expression vectors include, but are not limited to, viral vectors (e.g., viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655); adeno-associated virus (see, e.g., Ali et al., Hum Gene Ther 9:81 86, 1998, Flannery et al., PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10.641 648, 1999; Ali et al., Hum Mol Genet 5:591 594, 1996; Srivastava in WO 93/09239, Samulski et al., J. Vir. (1989) 63:3822-3828; Mendelson et al., Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol. 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like. Numerous suitable expression vectors are known to those of skill in the art, and many are commercially available.

Depending on the host/vector system utilized, any of a number of suitable transcription and translation control elements, including constitutive and inducible promoters, transcription enhancer elements, transcription terminators, etc. may be used in the expression vector (see, e.g., Bitter et al. (1987) Methods in Enzymology, 153:516-544).

In some cases, a nucleotide sequence encoding one or more polypeptides of a TMAPP is operably linked to a control element, e.g., a transcriptional control element, such as a promoter. The transcriptional control element may be functional in either a eukaryotic cell, e.g., a mammalian cell such as a human, hamster, or mouse cell; or a prokaryotic cell (e.g., bacterial). In some cases, a nucleotide sequence encoding a DNA-targeting RNA and/or a site-directed modifying polypeptide is operably linked to multiple control elements that allow expression of the nucleotide sequence encoding a DNA-targeting RNA and/or a site-directed modifying polypeptide in both prokaryotic and eukaryotic cells.

Non-limiting examples of suitable eukaryotic promoters (promoters functional in a eukaryotic cell) include the cytomegalovirus (CMV) immediate early, herpes simplex virus (HSV) thymidine kinase, early and late SV40, long terminal repeats (LTRs) from retrovirus, and mouse metallothionein-I. Selection of the appropriate vector and promoter is well within the level of ordinary skill in the art. The expression vector may also contain a ribosome binding site for translation initiation and a transcription terminator. The expression vector may also include appropriate sequences for amplifying expression.

### V.C. Genetically Modified Host Cells

The present disclosure provides a genetically modified host cell, where the host cell is genetically modified with a nucleic acid(s) that encode, or encode and express, TMAPP proteins or higher order complexes of TMAPPs (e.g., duplex TMAPPs).

Suitable host cells include eukaryotic cells, such as yeast cells, insect cells, and mammalian cells. In some cases, the host cell is a cell of a mammalian cell line. Suitable mammalian cell lines include human cell lines, non-human primate cell lines, rodent (e.g., mouse, rat) cell lines, and the like. Suitable mammalian cell lines include, but are not limited to, HeLa cells (e.g., American Type Culture Collection (ATCC) No. CCL-2),™), CHO cells (e.g., ATCC Nos. CRL9618, CCL61,CRL-9618™, CCL-61™, CRL9096), 293 cells (e.g., ATCC No. CRL-1573),™), Vero cells, NIH 3T3 cells (e.g., ATCC No. CRL-1658), Huh-7 cells, BHK cells (e.g., ATCC No. CCL10),CCL-10™), PC12 cells (ATCC No. CRL1721),CRL-1721™), COS cells, COS-7 cells (ATCC No. CRL1651), RAT1 cells, mouse L cells (ATCC No. CCLL3), human embryonic kidney (HEK) cells (ATCC No. CRL1573), HLHepG2 cells, and the like. The host cell may be a mammalian cell that has been genetically modified such that it does not synthesize endogenous MHC Class II heavy chains (MHC).

Genetically modified host cells can be used to produce a TMAPP and higher order complexes of TMAPPs (e.g., a duplex TMAPP). For example, an expression vector comprising nucleotide sequences encoding the TMAPP polypeptide(s) is/are introduced into a host cell, generating a genetically modified host cell, which genetically modified host cell produces the polypeptide(s) (e.g., as an excreted soluble protein).

### V.D. Methods of Producing TMAPPs

The present disclosure provides methods of producing unconjugated TMAPPs (e.g., duplex TMAPPs) with at least one masked TGF-β MOD that may be conjugated to and present T1D-associated peptide epitope. The methods generally involve culturing, in a culture medium, a host cell that is genetically modified with a recombinant expression vector(s) comprising a nucleotide sequence(s) encoding the TMAPP (e.g., a genetically modified host cell of the present disclosure); and isolating the TMAPP from the genetically modified host cell and/or the culture medium. As noted above, in some cases, the individual polypeptide chains of a TMAPP are encoded in separate nucleic acids (e.g., recombinant expression vectors). In some cases, all polypeptide chains of a TMAPP are encoded in a single recombinant expression vector.

Isolation of the TMAPP from the host cell employed for expression (e.g., from a lysate of the expression host cell) and/or the culture medium in which the host cell is cultured, can be carried out using standard methods of protein purification. For example, a lysate of the host cell may be prepared, and the TMAPP purified from the lysate using high performance liquid chromatography (HPLC), exclusion chromatography (e.g., size exclusion chromatography), gel electrophoresis, affinity chromatography, or other purification technique. Alternatively, where the TMAPP is secreted from the expression host cell into the culture medium, the TMAPP can be purified from the culture medium using HPLC, exclusion chromatography, gel electrophoresis, affinity chromatography, or other purification technique. In some cases, the TMAPP is purified, e.g., a composition is generated that comprises at least 80% by weight, at least about 85% by weight, at least about 95% by weight, or at least about 99.5% by weight, of the TMAPP in relation to contaminants related to the method of preparation of the product and its purification. The percentages can be based upon total protein.

In some cases, e.g., where the expressed TMAPP comprises an affinity tag or affinity domain, the TMAPP can be purified using an immobilized binding partner of the affinity tag. For example, where a TMAPP comprises an Ig Fc polypeptide, the TMAPP can be isolated from genetically modified mammalian host cell and/or from culture medium comprising the TMAPP by affinity chromatography, e.g., on a Protein A column, a Protein G column, or the like. An example of a suitable mammalian cell is a CHO cell; e.g., an Expi-CHO—S™ cell (e.g., ThermoFisher Scientific, Catalog #A29127).

Where the polypeptides of the TMAPP comprise suitable scaffold sequences they will self-assemble into dimers, and where applicable, spontaneously form disulfide bonds between, for example, Ig Fc scaffold polypeptide sequences. Similarly, the first and second presenting sequences of TMAPP presenting complexes will self-assemble, and where suitable cysteines are present, form disulfide bonds between the presenting complex peptides.

Following, their production, the TMAPPs are subject to conjugation with a T1D-associated epitope presenting molecule to form a TMAPP-epitope conjugate.

### V.E. Compositions

The present disclosure provides compositions, including pharmaceutical compositions, comprising a TMAPP-epitope conjugate and/or higher order complexes of TMAPP-epitope conjugates (e.g., duplex TMAPP-epitope conjugates). Pharmaceutical composition can comprise, in addition to a TMAPP-epitope conjugate, one or more known carriers, excipients, diluents, buffers, salts, surfactants (e.g., non-ionic surfactants), amino acids (e.g., arginine), etc., a variety of which are known in the art and need not be discussed in detail herein. For example, see “Remington: The Science and Practice of Pharmacy”, 19^{th} Ed. (1995), or latest edition, Mack Publishing Co.

In some cases, a subject pharmaceutical composition will be suitable for administration to a subject, e.g., will be sterile and/or substantially free of pyrogens. For example, in some embodiments, a subject pharmaceutical composition will be suitable for administration to a human subject, e.g., where the composition is sterile and is substantially free of detectable pyrogens and/or other toxins, or such detectable pyrogens and/or other toxins are below a permissible limit.

The compositions may, for example, be in the form of aqueous or other solutions, powders, granules, tablets, pills, suppositories, capsules, suspensions, sprays, and the like. The composition may be formulated according to the various routes of administration described below.

Where a TMAPP-epitope conjugate or higher order TMAPP-epitope conjugate complex (e.g., duplex TMAPP-epitope conjugate) is administered as an injectable (e.g., subcutaneously, intraperitoneally, intramuscularly, intralymphatically, and/or intravenously) directly into a tissue, a formulation can be provided as a ready-to-use dosage form, or as non-aqueous form (e.g., a reconstitutable storage-stable powder) or an aqueous form, such as liquid composed of pharmaceutically acceptable carriers and excipients. TMAPP-epitope conjugates may also be provided so as to enhance serum half-life of the subject protein following administration. For example, the protein may be provided in a liposome formulation, prepared as a colloid, or other conventional techniques for extending serum half-life. A variety of methods are available for preparing liposomes, as described in, e.g., Szoka et al. 1980 Ann. Rev. Biophys. Bioeng. 9:467, U.S. Pat. Nos. 4,235,871, 4,501,728 and 4,837,028. The preparations may also be provided in controlled release or slow-release forms.

In some cases, a TMAPP-epitope conjugate composition comprises: a) a TMAPP-epitope conjugate or higher order complex (e.g., a duplex TMAPP-epitope conjugate); and b) saline (e.g., 0.9% NaCl). In some cases, the composition is sterile and/or substantially pyrogen free, or the amount of detectable pyrogens and/or other toxins are below a permissible limit. In some cases, the composition is suitable for administration to a human subject, e.g., where the composition is sterile and is free of detectable pyrogens and/or other toxins, or the amount of detectable pyrogens and/or other toxins are below a permissible limit. Thus, the present disclosure provides a composition comprising: a) a TMAPP-epitope conjugate or higher order TMAPP-epitope conjugate complex (e.g., duplex TMAPP-epitope conjugate); and b) saline (e.g., 0.9% NaCl), where the composition is sterile and is substantially free of detectable pyrogens and/or other toxins, or such detectable pyrogens and/or other toxins are below a permissible limit.

Other examples of components suitable for inclusion in formulations suitable for parenteral administration include isotonic sterile injection solutions, anti-oxidants, bacteriostats, and solutes that render the formulation isotonic with the blood of the intended recipient, suspending agents, solubilizers, thickening agents, stabilizers, and preservatives. A pharmaceutical composition can be present in a container, e.g., a sterile container, such as a syringe. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets.

The concentration of a TMAPP-epitope conjugate in a formulation can vary widely. For example, a TMAPP-epitope conjugate or higher order TMAPP-epitope conjugate complex (e.g., duplex TMAPP-epitope conjugate) may be present from less than about 0.1% (usually at least about 2%) to as much as 20% to 50% or more by weight (e.g., from 1% to 10%, 5% to 15%, 10% to 20% by weight, or 20-50% by weight) by weight. The concentration will usually be selected primarily based on fluid volumes, viscosities, and patient-based factors in accordance with the particular mode of administration selected and the patient's needs.

The present disclosure provides a container comprising a composition, e.g., a liquid composition. The container can be, e.g., a syringe, an ampoule, and the like. In some cases, the container is sterile. In some cases, both the container and the composition are sterile and substantially free of detectable pyrogens and/or other toxins, or such detectable pyrogens and/or other toxins are below a permissible limit. A pharmaceutical composition or a container comprising a composition (e.g., pharmaceutical composition) set forth herein may be packaged as a kit. The kit may comprise, for example, the composition or the container comprising a composition along with instructions for use of those materials. Materials packaged as a kit may be sterile and/or substantially free of detectable pyrogens and/or other toxins, or such detectable pyrogens and/or other toxins are below a permissible limit.

### V.F. Methods of Utilizing TMAPP-Epitope Conjugates

TMAPP-epitope conjugates and higher order TMAPP-epitope conjugate complexes (e.g., duplex TMAPP-epitope conjugate) are useful for modulating an activity of a T cell. Thus, the present disclosure provides methods of modulating an activity of a T cell, the methods generally involving contacting a target T cell with a TMAPP-epitope conjugate or a higher order TMAPP-epitope conjugate complex (e.g., duplex TMAPP-epitope conjugate).

### V.F(i). Methods of Modulating T Cell Activity

The present disclosure provides a method of selectively modulating the activity of an epitope-specific T cell, the method comprising contacting the T cell with a TMAPP-epitope conjugate comprising a T1D peptide epitope, where contacting the T cell with a TMAPP-epitope conjugate selectively modulates the activity of the epitope-specific T cell. In some cases, the contacting occurs in vivo (e.g., in a mammal such as a human, rat, mouse, dog, cat, pig, horse, or primate). In some cases, the contacting occurs in vitro. In some cases, the contacting occurs in vivo.

In some cases, a TMAPP-epitope conjugate reduces activity of an autoreactive T cell and/or an autoreactive B cell. In some cases, a TMAPP-epitope conjugate increases the number and/or activity of a regulator T cell (Treg), resulting in reduced activity of an autoreactive T cell and/or an autoreactive B cell.

In some cases, a TMAPP-epitope conjugate (e.g., duplex TMAPP-epitope conjugate) is contacted with an epitope-specific CD4⁺ T cell. In some cases, the epitope-specific T cell is a CD4⁺ CD8⁺ (double positive) T cell (see e.g., Boher et al Front. Immunol., 29 Mar. 2019 on the www at: doi.org/10.3389/fimmu.2019.00622 and Matsuzaki et al. J. Immuno. Therapy of Cancer 7: Article number: 7 (2019)). In some cases, the epitope-specific T cell is a NK-T cell (see, e.g., Nakamura et al. J, Immunol. 2003 Aug. 1; 171(3):1266-71). In some cases, the epitope-specific T cell is a T (Treg). The contacting may result in modulating the activity of a T cell, which can result in, but is not limited to proliferation and/or maintenance of regulatory T cells (e.g., when IL-2 MOD polypeptides are present, the effect of which may be amplified by the presence of retinoic acids such as all trans retinoic acid).

In some cases, a TMAPP-epitope conjugate is contacted with an epitope-specific CD4⁺ T cell. In some cases, the CD4⁺ T cell is a Th1 that produces, among other things, interferon gamma, and which may be a target for inhibition in autoimmunity. In some cases, the CD4+ T cell is a Th2 cell that produces, among other things, IL-4. Th2 cells may be inhibited to suppress autoimmune diseases such T1D. In some cases, the CD4⁺ T cell is a Th17 cell that produces, among other things, IL-17, and which may be inhibited to suppress autoimmune diseases such as T1D. In some cases, the CD4⁺ T cell is a Th9 cell that produces, among other things, IL-9, and which may be inhibited to suppress its actions in autoimmune conditions such as TiD. In some cases, the CD4⁺ T cell is a Tfh cell that produces, among other things, IL-21 and IL-4, and which may be inhibited to suppress autoimmune diseases such as T1D.

In some cases, the T cell being contacted with a TMAPP-epitope conjugate is a regulatory T cell (Treg) that is CD4⁺, FOXP3⁺, and CD25⁺. Tregs can suppress autoreactive T cells.

The present disclosure provides a method of increasing proliferation of Tregs, the method comprising contacting Tregs with a TMAPP-epitope conjugate, where the contacting increases proliferation of Tregs specific/selective for epitope presented by the TMAPP-epitope conjugate. The present disclosure provides a method of increasing the number of epitope specific Tregs in an individual, the method comprising administering to the individual a TMAPP-epitope conjugate, where the administering results in an increase in the number of Tregs specific to the epitope presented by the TMAPP-epitope conjugate in the individual. For example, the number of Tregs can be increased by at least 5%, at least 10%, at least 15%, at least 20%, at least 25%, at least 30%, at least 40%, at least 50%, at least 60%, at least 70%, at least 80%, at least 90%, at least 2-fold, at least 2.5-fold, at least 5-fold, at least 10-fold, or more than 10-fold.

In some cases, the cell being contacted with a TMAPP-epitope conjugate is a helper T cell, where contacting the helper T cell with a TMAPP-epitope conjugate inhibits or blocks the proliferation and/or differentiation of Th1 and/or Th2 cells specific/selective for the epitope presented by the TMAPP-epitope conjugate by, for example, inhibiting the expression of the transcription factors T-bet and/or GATA3. The suppression of Th1 and/or Th2 cells results in the decreased activity and/or number effector cells such as CD8′ cytotoxic T cells specific to the epitope.

In some cases a TMAPP-epitope conjugate interacts with T cells that are subject to IL-2 receptor activation provided either by an IL-2 MOD of the TMAPP-epitope conjugate or IL-2 in the T cell environment resulting in: (i) activation, proliferation, or maintenance of T reg cells specific for the epitope presented by the TMAPP-epitope conjugate; and/or (ii) suppression of epitope specific Th1 cell development; and/or (iii) suppression of epitope specific Th2 cell development; and/or (iv) suppression of epitope specific cytotoxic T lymphocyte (CTL) development. The addition of retinoic acid (e.g., all trans retinoic acid) may potentiate the action of the TGF-β-bearing TMAPP-epitope conjugates described herein a TMAPP-epitope conjugate in any of those functions, particularly activation, proliferation, or maintenance of T reg cells where the TMAPP-epitope conjugate bears one or more IL-2 MODs. Where the epitope is an TD1-associated epitope the TMAPP-epitope conjugate can be utilized to suppress responses to the epitope and effect treatment of T1D.

TMAPP-epitope conjugates may interact with T cells in the presence of IL-2 and PD1 receptor agonist, either or both of which may be provided by IL-2 or PD-L1 MODs of the TMAPP-epitope conjugate and/or IL-2 or PD-L1 present in the T cell's environment during the interaction. Under such conditions the TMAPP-epitope conjugate along with agonist of the IL-2 and PD1 receptors may regulate the development, maintenance, and function of Treg cells (e.g., induced regulatory T cells) specific for the epitope presented by the TMAPP-epitope conjugate. See, e.g., Franciso et al., J Exp Med., 206(13):3015-3029 (2009). Accordingly, masked TGF-β MOD-bearing TMAPP-epitope conjugates along with agonist of the IL-2 receptor and PD1 receptor (e.g., a TMAPP-epitope conjugate bearing one or more masked TGF-β MODs and additionally one or more IL-2 MODs and one or more PD-L1 MODs) may be employed to suppress immune responses to epitopes of autoantigens associate with T1D.

### V.F(ii). Treatment Methods

The present disclosure provides treatment methods, the methods comprising administering to the individual composition comprising an amount of a TMAPP-epitope conjugate or higher order TMAPP-epitope conjugate, such as duplex TMAPP-epitope conjugate, effective to selectively modulate the activity of a T1D epitope-specific T cell in an individual and to treat the individual. In some cases, a treatment method comprises administering to an individual in need thereof a pharmaceutical composition comprising an effective amount of a TMAPP-epitope conjugate or higher order TMAPP-epitope conjugate such as duplex TMAPP-epitope conjugate useful for treating type 1 diabetes (T1D) occurring in human patients and in experimental animal models (e.g., non-obese diabetic (NOD) mouse and the Biobreeding (BB) rat).

The present disclosure provides a method of selectively modulating the activity of a T1D epitope-specific T cell in an individual, the method comprising administering to the individual a pharmaceutical composition comprising an effective amount of a TMAPP-epitope conjugate or higher order TMAPP-epitope conjugate, such as duplex TMAPP-epitope conjugate, where the TMAPP-epitope conjugate selectively modulates the activity of the epitope-specific T cell in the individual. Selectively modulating the activity of an epitope-specific T cell can treat T1D in the individual. Thus, the present disclosure provides a treatment method comprising administering to an individual in need thereof a pharmaceutical composition comprising an effective amount of a TMAPP-epitope conjugate or higher order TMAPP-epitope conjugate such as duplex TMAPP-epitope conjugate for the purpose of treating T1D.

The present disclosure provides a method of treating T1D in an individual, the method comprising administering to the individual a pharmaceutical composition comprising an effective amount of a TMAPP-epitope conjugate or higher order TMAPP-epitope conjugate, such as a duplex TMAPP-epitope conjugate, where the TMAPP-epitope conjugate comprises a T1D peptide epitope (as described above), and where the TMAPP-epitope conjugate comprises PD-L1. In some cases, an “effective amount” of a TMAPP-epitope conjugate or higher order TMAPP-epitope conjugate is an amount that, when administered in one or more doses to an individual in need thereof, reduces the number of self-reactive (i.e., reactive with aT1D-associated antigen) CD4+ and/or CD8+ T cells by, for example, at least 10%, at least 15%, at least 20%, at least 25%, at least 30%, at least 40%, at least 50%, at least 60%, at least 70%, at least 80%, at least 90%, or at least 95% (e.g., from 10% to 50%, or from 50% to 95%) compared to number of self-reactive T cells in the individual before administration of the TMAPP-epitope conjugate, or in the absence of administration with the TMAPP-epitope conjugate. In some cases, an “effective amount” of a TMAPP-epitope conjugate is an amount that, when administered in one or more doses to an individual in need thereof, reduces production of Th1 cytokines (e.g., IL-2, IL-10, and TNF-alpha/beta) in the individual. In some cases, an “effective amount” of a TMAPP-epitope conjugate is an amount that, when administered in one or more doses to an individual in need thereof, reduces production of Th2 cytokines (e.g., IL-4, IL-5, and/or IL-13) in the individual. In some cases, an “effective amount” of a TMAPP-epitope conjugate is an amount that, when administered in one or more doses to an individual in need thereof, reduces production of Th17 cytokines (e.g., IL-17A, IL-17F, and/or IL-22) in the individual. In some cases, an “effective amount” of a TMAPP-epitope conjugate is an amount that, when administered in one or more doses to an individual in need thereof, ameliorates one or more symptoms associated with T1D in the individual. In some instances, the TMAPP-epitope conjugate reduces the number of CD4+ self-reactive T cells (i.e., the number of CD4+ T cells reactive with a T1D-associated antigen), which in turn leads to a reduction in CD8+ self-reactive T cells. In some instances, the TMAPP-epitope conjugate increases the number or activity (e.g., IL-10 and/or TGF-β production) of CD4+ Tregs specific for a TiD peptide epitope presented by the TMAPP-epitope conjugate, which in turn may reduce the number or activity of CD4+ self-reactive T cells, B cells, and/or CD8+T self-reactive T cells specific for that epitope.

The present disclosure provides a method of reducing elevated blood sugar (e.g., glucose) in an individual (e.g., a mammal such as a human) having or suspected of having T1D, the method comprising administering to the individual an effective amount of a TMAPP-epitope conjugate, or one or more nucleic acids comprising nucleotide sequences encoding the TMAPP-epitope conjugate, where the TMAPP-epitope conjugate comprises a T1D peptide epitope (as described above). The individual may be a human having a fasting blood sugar in excess of 130 or about 140 mg/dL, or postprandial blood sugar in excess of 180 or about 200 mg/dL, and wherein the treatment reduces fasting blood sugar (e.g., such as to a level below 130 mg/dL) or post prandial blood sugar (e.g., to less than 180 mg/dL) in the individual relative to the level prior to receiving the TMAPP-epitope conjugate. The reduction in blood sugar may be maintained for a period of at least about a week, at least about two weeks, at least about a month (30 days), or more than one month.

The present disclosure provides a method of reducing prediabetic glycosylated hemoglobin referred to as hemoglobin A1C (also referred to as hemoglobin A1c or HbA1c) levels (in the range of 5.7% to 6.4%) or diabetic hemoglobin Δ1C levels (above 6.4%) in an individual (e.g., a mammal such as a human) having or suspected of having T1D, the method comprising administering to the individual an effective amount of a TMAPP-epitope conjugate, or one or more nucleic acids comprising nucleotide sequences encoding the TMAPP-epitope conjugate, where the TMAPP-epitope conjugate comprises a T1D peptide epitope (as described above). The treatment reduces diabetic hemoglobin Δ1C (e.g., to less than 6.4%, and preferably to less than 5.7%), or prediabetic A1C (e.g., such as to less than 5.7% and into the normal range) in the individual relative to the level prior to receiving the TMAPP-epitope conjugate. The reduction in hemoglobin A1C may be maintained for a period of at least about a week, at least about two weeks, at least about a month (30 days), or more than one month.

### V.F(iii). Methods of Selectively Delivering a MOD

The present disclosure provides a method of delivering TGF-β either alone or in combination with a MOD polypeptide such as IL-2, 4-1BBL, PD-L1, or a reduced-affinity variant of any thereof (e.g., PD-L1 and/or an IL-2 variant disclosed herein) to a selected T cell or a selected T cell population, e.g., in a manner such that a TCR specific for a given T1D-assocaited epitope. As used herein, the phrases “selectively delivers” and “selectively provides” means that the majority of T cells for which the TMAPP-epitope conjugate provides detectable TGF-β modulation comprise a TCR that specifically or preferentially binds the epitope of the TMAPP-epitope conjugate.

The present disclosure thus provides a method of delivering TGF-β (masked TGF-β) and a MOD polypeptide such as a PD-L1 polypeptide, or a reduced-affinity variant of a naturally occurring MOD polypeptide such as a PD-L1 variant, selectively to a target T cell bearing a TCR specific for the T1D-associated epitope presented by a TMAPP-epitope conjugate (e.g., duplex TMAPP-epitope conjugate). The present disclosure provides a method of delivering a TGF-β and an IL-2 MOD polypeptide sequence, or a reduced-affinity variant of IL-2, selectively to a target T cell bearing a TCR specific for the T1D epitope presented by a TMAPP-epitope conjugate (e.g., duplex TMAPP-epitope conjugate). The method comprises contacting a population of T cells with a TMAPP-epitope conjugate (e.g., duplex TMAPP-epitope conjugate). The population of T cells can be a mixed population that comprises: i) the target T cell with a TCR specific to a target epitope; and ii) non-target T cells that are not specific for the target epitope presented by the TMAPP-epitope conjugate-associated peptide epitope (e.g., T cells that are specific for an epitope(s) other than the epitope to which the epitope-specific T cell binds). Epitope-specific T cells specific for the peptide epitope present in the TMAPP-epitope conjugate (e.g., duplex TMAPP-epitope conjugate) bind to the peptide MHC complex provided by the TMAPP-epitope conjugate thereby delivering the TGF-β and any other additional MOD polypeptide in the TMAPP-epitope conjugate ((e.g., PD-L1 or a reduced-affinity variant of PD-L1) selectively to the bound T cells.

Thus, the present disclosure provides a method of delivering TGF-β and an IL-2 MOD, PD-L1 MOD, and/or a reduced-affinity variant of IL-2 and/or PD-L1, to T cell selective for the T1D epitope presented by the TMAPP-epitope conjugate. Similarly, the disclosure provides a method of delivering TGF-β, and an IL-2, MOD polypeptide and/or a reduced-affinity variant of a naturally occurring IL-2 MOD polypeptide to a target T cell that is selective for the T1D epitope presented by the TMAPP-epitope conjugate. In some cases, the IL-2 MOD bears a substitution at position H16 and/or F42 (e.g., H16 and F42 such as H16A and F42A) (see supra SEQ ID NO:103).

For example, a TMAPP-epitope conjugate or higher order TMAPP-epitope conjugate complex (e.g., duplex TMAPP-epitope conjugate) is contacted with a population of T cells comprising: i) target T cells that are specific for the epitope present in the TMAPP-epitope conjugate or a higher order TMAPP-epitope conjugate complex; and ii) non-target T cells, e.g., a T cells that are specific for a second epitope(s) that is not the epitope present in the TMAPP-epitope conjugate or a higher order TMAPP-epitope conjugate complex. Contacting the population results in substantially selective delivery of the TGF-β and any other MOD polypeptide(s) present in the TMAPP-epitope conjugate (e.g., naturally-occurring or variant MOD polypeptide(s)) to the target T cell. Less than 50%, less than 40%, less than 30%, less than 25%, less than 20%, less than 15%, less than 10%, less than 5%, or less than 4%, 3%, 2% or 1%, of the TMAPP-epitope conjugate or higher order TMAPP-epitope conjugate complex (e.g., duplex TMAPP-epitope conjugate) may bind to non-target T cells and, as a result, the MOD polypeptide (e.g., PD-L1 or PD-L1 variant) is selectively delivered to target T cell (and accordingly, not effectively delivered to the non-target T cells).

The population of T cells to which a MOD and/or variant MOD is selectively delivered may be in vivo. In some cases, the population of T cells to which a MOD and/or variant MOD is selectively delivered is in vitro.

In some cases, the population of T cells to which a MOD and/or variant MOD is selectively delivered may be in vivo. In some cases, the population of T cells is in vitro. For example, a mixed population of T cells is obtained from an individual, and is contacted with a TMAPP-epitope conjugate (e.g., duplex TMAPP-epitope conjugate) in vitro. Such contacting, which can comprise single or multiple exposures of the T cells to one or more defined doses and/or exposure schedules in the context of in vitro cell culture, can be used to determine whether the mixed population of T cells includes T cells that are specific for the epitope presented by the TMAPP-epitope conjugate. The presence of T cells that are specific for the epitope presented by the TMAPP-epitope conjugate can be determined by assaying a sample comprising a mixed population of T cells, which population of T cells comprises T cells that are not specific for the epitope (non-target T cells) and may comprise T cells that are specific for the epitope (target T cells). Known assays can be used to detect the desired modulation of the target T cells, thereby providing an in vitro assay that can determine whether a particular TMAPP-epitope conjugate (e.g., duplex TMAPP-epitope conjugate) possesses an epitope that binds to T cells present in the individual, and thus whether the TMAPP-epitope conjugate has potential use as a therapeutic composition for that individual. Suitable known assays for detection of the desired modulation (e.g., activation/proliferation or inhibition/suppression) of target T cells include, e.g., flow cytometric characterization of T cell phenotype, numbers, and/or antigen specificity. Such an assay to detect the presence of epitope-specific T cells, e.g., a companion diagnostic, can further include additional assays (e.g., effector cytokine ELISpot assays) and/or appropriate controls (e.g., antigen-specific and antigen-nonspecific multimeric peptide-HLA staining reagents) to determine whether the TMAPP-epitope conjugate or higher order TMAPP-epitope conjugate complex is selectively binding, modulating (activating or inhibiting), and/or expanding the target T cells. Thus, for example, the present disclosure provides a method of detecting, in a mixed population of T cells obtained from an individual, the presence of a target T cell that binds an epitope of interest, the method comprising: a) contacting in vitro the mixed population of T cells with a TMAPP-epitope conjugate (e.g., duplex TMAPP-epitope conjugate) comprising an epitope; and b) detecting modulation (activation or inhibition) and/or proliferation of T cells in response to said contacting, wherein modulation of and/or proliferation of T cells indicates the presence of the target T cell. Alternatively, and/or in addition, if activation and/or expansion (proliferation) of the desired T cell population (e.g., Tregs) is obtained using a TMAPP-epitope conjugate (e.g., a duplex TMAPP-epitope conjugate), then all or a portion of the population of T cells comprising the activated/expanded T cells can be administered back to the individual as a therapy.

The population of T cells to be targeted by a TMAPP-epitope conjugate may be in vivo in an individual. In such instances, a method for selectively delivering TGF-β an any other MOD polypeptide (e.g., wt. or variant IL-2 polypeptides) to an epitope-specific T cell comprises administering the TMAPP-epitope conjugate (e.g., duplex TMAPP-epitope conjugate) to the individual.

In some instances, the epitope-specific T cell to which TGF-β and any other MOD polypeptide sequence present in the TMAPP-epitope conjugate (e.g., a wild-type or reduced affinity IL-2 and/or PD-L1 MOD) is being selectively delivered is referred to herein is a target regulatory T cell (Treg) that may inhibit or suppresses activity of an autoreactive T cell.

### V.G. Dosages

A suitable dosage can be determined by an attending physician or other qualified medical personnel, based on various clinical factors. As is well known in the medical arts, dosages for any one patient depend upon many factors, including the patient's size, body surface area, age, the particular polypeptide or nucleic acid to be administered, sex of the patient, time, and route of administration, general health, and other drugs being administered concurrently. A TMAPP-epitope conjugate (whether as a single TMAPP or as a higher order complex such as a duplex TMAPP-epitope conjugate) may be administered in amounts between 1 ng/kg body weight and 20 mg/kg body weight per dose; for example from 0.1 μg/kg body weight to 1.0 mg/kg body weight, from 0.1 mg/kg body weight to 0.5 mg/kg body weight, from 0.5 mg/kg body weight to 1 mg/kg body weight, from 1.0 mg/kg body weight to 5 mg/kg body weight, from 5 mg/kg body weight to 10 mg/kg body weight, from 10 mg/kg body weight to 15 mg/kg body weight, and from 15 mg/kg body weight to 20 mg/kg body weight. Doses below 0.1 mg/kg body weight or above 20 mg/kg are envisioned, especially considering the aforementioned factors. Amounts thus include from about 0.1 mg/kg body weight to about 0.5 mg/kg body weight, from about 0.5 mg/kg body weight to about 1 mg/kg body weight, from about 1.0 mg/kg body weight to about 5 mg/kg body weight, from about 5 mg/kg body weight to about 10 mg/kg body weight, from about 10 mg/kg body weight to about 15 mg/kg body weight, from about 15 mg/kg body weight to about 20 mg/kg body weight, and above about 20 mg/kg body weight.

Those of skill will readily appreciate that dose levels can vary as a function of the TMAPP-epitope conjugate or higher order TMAPP-epitope conjugate complex (e.g., duplex TMAPP-epitope conjugate), the severity of the symptoms and the susceptibility of the subject to side effects. Preferred dosages for a given compound are readily determinable by those of skill in the art by a variety of means.

In some cases, multiple doses of a TMAPP-epitope conjugate or higher order TMAPP-epitope conjugate complex (e.g., duplex TMAPP-epitope conjugate) are administered. The frequency of administration of a TMAPP-epitope conjugate or higher order TMAPP-epitope conjugate complex (e.g., duplex TMAPP-epitope conjugate) can vary depending on any of a variety of factors, e.g., severity of the symptoms, patient response, etc. For example, in some cases, a TMAPP-epitope conjugate or higher order TMAPP-epitope conjugate (e.g., duplex TMAPP-epitope conjugate) is administered less frequently than once per month, e.g., once every two, three, four, six or more months, once per year, or once per month or more frequently, e.g., twice per month, three times per month, every other week (qow), one every three weeks, once every four weeks, once per week (qw), twice per week (biw), three times per week (tiw), four times per week, five times per week, six times per week, every other day (qod), daily (qd), twice a day (qid), or three times a day (tid).

The duration of administration of a TMAPP-epitope conjugate, e.g., the period of time over which a TMAPP-epitope conjugate is administered, can vary, depending on any of a variety of factors, e.g., patient response, etc. For example, a TMAPP-epitope conjugate can be administered over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more, including continued administration for the patient's life.

Where treatment is of a finite duration, following successful treatment, it may be desirable to have the patient undergo periodic maintenance therapy to prevent the recurrence of the T1D disease state, wherein a TMAPP-epitope conjugate is administered in maintenance doses, ranging from those recited above, i.e., 0.1 mg/kg body weight to about 0.5 mg/kg body weight, from about 0.5 mg/kg body weight to about 1 mg/kg body weight, from about 1.0 mg/kg body weight to about 5 mg/kg body weight, from about 5 mg/kg body weight to about 10 mg/kg body weight, from about 10 mg/kg body weight to about 15 mg/kg body weight, from about 15 mg/kg body weight to about 20 mg/kg body weight, and above about 20 mg/kg body weight. The periodic maintenance therapy can be once per month, once every two months, once every three months, once every four months, once every five months, once every six months, or less frequently than once every six months.

### V.H. Routes of Administration

A TMAPP-epitope conjugate or higher order TMAPP-epitope conjugate (e.g., a duplex TMAPP-epitope conjugate) is administered to an individual using any available method and route suitable for drug delivery, including in vivo and in vitro methods, as well as systemic and localized routes of administration. A TMAPP-epitope conjugate or higher order TMAPP-epitope conjugate complex can be administered to a host using any available conventional methods and routes suitable for delivery of conventional drugs, including systemic or localized routes. In general, routes of administration contemplated for use in a method include, but are not necessarily limited to, enteral, parenteral, and inhalational routes.

Conventional and pharmaceutically acceptable routes of administration include intramuscular, intratracheal, subcutaneous, intradermal, topical application, intravenous, intraarterial, rectal, nasal, oral, and other enteral and parenteral routes of administration. Of these, intravenous, intramuscular and subcutaneous may be more commonly employed. Routes of administration may be combined, if desired, or adjusted depending upon, for example, the TMAPP-epitope conjugate or higher order TMAPP-epitope conjugate complex (e.g., duplex TMAPP-epitope conjugate) and/or the desired effect. A TMAPP-epitope conjugate or higher order TMAPP-epitope conjugate complex can be administered in a single dose or in multiple doses.

### V.I. Subjects Suitable for Treatment

Subjects suitable for treatment with a method include individuals who have T1D, including individuals who have been diagnosed as having T1D, and individuals who have been treated for T1D but who failed to respond to the treatment. Suitable subjects also may include individuals who have been diagnosed as being likely to develop T1D or who have symptoms indicating the imminent onset of T1D.

Subjects suitable for treatment include those with T1D or a genetic disposition to develop T1D including a family history of T1D (e.g., a grandparent, parent, or sibling with T1D). As discussed above, a number of serotypes have been associated with a substantial risk of developing T1D (e.g., DR3, DR4, DQ2.5 and DQ8.1), and others with a moderate risk of developing T1D (e.g., DR1, DR8, DR9 and DQ5.

Subjects suitable for treatment include those who have T1D, including individuals who have been diagnosed as having T1D, and individuals who have been treated for T1D but who failed to respond to the treatment who have fasting blood sugars (blood sugar after not eating or drinking for 8 hours) in excess of about 130 mg/dL (or about 140 mg dL), and/or a blood sugar higher than about 180 mg/dL (or about 200 m/dL) 2 hours after a meal (postprandial hyperglycemia). This includes individuals with (i) a genetic disposition to T1D such as a family history of T1D and/or a serotype associated with T1D (e.g., DR4), and (ii) either an elevated fasting blood sugar in excess of 130 or about 140 mg/dL, or postprandial blood sugar in excess of 180 or about 200 mg/dL. See, e.g., www.cdc.gov/diabetes/managing/managing-blood-sugar/bloodglucosemonitoring.html.

Subjects suitable for treatment include those who have T1D, including individuals who have been diagnosed as having T1D, and individuals who have been treated for T1D but who failed to respond to the treatment who have hemoglobin Δ1C levels from 5.7 to 6.4% (prediabetic levels) or hemoglobin A1C levels above 6.4% (diabetic levels). See, e.g., www.cdc.gov/diabetes/managing/managing-blood-sugar/a1c.html. This includes individuals with both prediabetic or diabetic A1C levels and a genetic disposition to T1D such as a family history of T1D and/or a serotype associated with T1D (e.g., DR4)

### VI. THE CERTAIN ASPECTS

Certain aspects, including embodiments/aspects of the present subject matter described above, may be beneficial alone or in combination, with one or more other aspects recited hereinbelow. In addition, while the present subject matter has been disclosed with reference to certain aspects recited below and in the claims, numerous modifications, alterations, and changes to the described aspects/embodiments are possible without departing from the sphere and scope of the present disclosure. Accordingly, it is intended that the present disclosure not be limited to the described embodiments, aspects, and claims, but that it has the full scope defined by the language of this disclosure and equivalents thereof.
1. A TMAPP-epitope conjugate comprising:
   (i) a presenting sequence or a presenting complex,
   (ii) optionally at least one scaffold polypeptide sequence, and
   (iii) a TGF-β sequence, a masking sequence that binds to a TGF-β sequence reversibly masking it, or at least one masked TGF-β MOD; wherein
   (a) each presenting sequence comprises MHC Class II α1, α2, β1, and β2 domain polypeptide sequences;
   (b) each presenting complex comprises a presenting complex first sequence and a presenting complex second sequence, wherein
      the presenting complex first sequence and presenting complex second sequence comprises at least one of the α1, α2, β1, and β2 polypeptide sequences, and the presenting complex first sequence and presenting complex second sequence together
      comprise the MHC Class II α1, α2, β1, and β2 domain polypeptide sequences, and
   (c) each masked TGF-β MOD comprises a masking sequence and TGF-β sequence;
   wherein the TMAPP-epitope conjugate optionally comprises one or more independently selected additional MODs (wt. and/or variant) or pairs of additional MODs (e.g., in tandem, both wt, both variant, or one wt. and one variant) (e.g., located at the N-terminus of a presenting sequence, presenting complex first and/or second sequences);
   wherein a the TMAPP-epitope conjugate comprises a chemical conjugation site for conjugation of an epitope presenting molecule, and optionally comprises an additional chemical conjugation site for the conjugation of a payload, and wherein a T1D-associated epitope has been conjugated to the chemical conjugation site; and
   wherein the TMAPP-epitope conjugate optionally comprises one or more linker sequences that are selected independently. (see, e.g., FIGS. 1A to 1H and FIGS. 10A to 10D).
It is understood that the amino acid sequence of any component (e.g., MHC-Class II polypeptide sequences) does not include an amino acid sequence that will anchor the TMAPP in a mammalian cell (e.g., a COS cell) membrane (e.g., the TMAPP does not comprise an MHC transmembrane domain, or a portion thereof, that will anchor the TMAPP in a cell membrane. The above components may or may not be arranged in the stated order from N-terminus to C-terminus in the TMAPP.
2. The TMAPP-epitope conjugate of aspect 1, comprising from N-terminus to C-terminus:
   (i) a presenting sequence or a presenting complex,
   (ii) optionally at least one scaffold polypeptide sequence, and
   (iii) a TGF-β sequence, a masking sequence that binds to a TGF-β sequence reversibly masking it, or at least one masked TGF-β MOD.
3. The TMAPP-epitope conjugate of aspects 1 or 2, wherein the TMAPP comprises a presenting sequence (see e.g., FIG. 9E to 9J).
4. The TMAPP-epitope conjugate of aspect 3, wherein the presenting sequence comprises, ordered from N-terminus to C-terminus, the β1, β2, α1, and α2 domain polypeptide sequences.
5. The TMAPP-epitope conjugate of aspect 3, wherein the presenting sequence comprises, ordered from N-terminus to C-terminus, the β1, α1, α2, and β2 domain polypeptide sequences.
6. The TMAPP-epitope conjugate of aspect 3, wherein the presenting sequence comprises, ordered from N-terminus to C-terminus, the at, α2, β1, and β2 domain polypeptide sequences.
7. The TMAPP-epitope conjugate of aspects 1 or 2, wherein the TMAPP comprises a presenting complex (see e.g., FIG. 9A to 9D).
8. The TMAPP-epitope conjugate of aspect 7, wherein: the presenting complex first sequence comprises (e.g., ordered from N-terminus to C terminus) the β1 and β2 domain polypeptide sequences; and the presenting complex second sequence comprises the al, and α2 domain polypeptide sequences.
9. The TMAPP-epitope conjugate of aspect 7, wherein: the presenting complex first sequence comprises (e.g., ordered from N-terminus to C terminus) the al, and α2 domain polypeptide sequences; and the presenting complex second sequence comprises the β31 and β2 domain polypeptide sequences.
10. The TMAPP-epitope conjugate of aspect 7, wherein: the presenting complex first sequence comprises (e.g., ordered from N-terminus to C terminus) the β1 al, and α2 domain polypeptide sequences; and the presenting complex second sequence comprises the β2 domain polypeptide sequence.
11. The TMAPP-epitope conjugate of aspect 7, wherein: the presenting complex first sequence comprises the β2 domain polypeptide sequence; and the presenting complex second sequence comprises (e.g., ordered from N-terminus to C terminus) the β1, al, and α2 domain polypeptide sequences.
12. The TMAPP-epitope conjugate of any preceding aspect, wherein the presenting sequence or a presenting complex comprises a disulfide bond formed between one of MHC α1 or α2 domain polypeptide sequence and one of the 01 or β2 domain polypeptide sequences.
13. The TMAPP-epitope conjugate of any preceding aspect, comprising a disulfide bond formed between cysteines positioned at:
   α chain position 3 and β chain position I9 or 20,
   α chain position 4 and β chain position I9 or 20,
   α chain position 28 and β chain position I51, 152, or 153,
   α chain position 29 and β chain position I51, 152, or 153,
   α chain position 80, 81, or 82 and β chain position 33,
   α chain position 93 and β chain position I53 of 156,
   α chain position 94 and β chain position I20 or 156, or
   α chain position 95 and β chain position I20 or 156;
wherein the α chain comprises the mature α1 and α2 domains, and the β chain comprises the mature β1 and β2 domains.
14. The TMAPP-epitope conjugate of any preceding aspect, comprising a disulfide bond formed between cysteines positioned at:
   α chain position I2 and β chain position 7 or 10,
   α chain position 80 and β chain position 5 or 7,
   α chain position 81 and β chain position 5 or 7, or
   α chain position 82 and β chain position 5 or 7;
wherein the α chain comprises the mature α1 and α2 domains, and the 0 chain comprises the mature β1 and β2 domains.
15. The TMAPP-epitope conjugate of aspect 14, comprising at least one presenting sequence or a presenting complex that comprises a disulfide bond formed between cysteines positioned at:
   α chain position 80 and β chain position 5 or 7; or
   α chain position 81 and β chain position 5 or 7.
16. The TMAPP-epitope conjugate of any preceding aspect comprises at least one linker attached to the: (i) the presenting sequence or the presenting complex; (ii) the scaffold polypeptide sequence if present; (iii) the TGF-β sequence or masking sequence; or (iv) the one or more additional MODs if present.
17. The TMAPP-epitope conjugate of any preceding aspect comprises at least one linker attached to one or two or the α1, α2, β1, and β2 domain peptide sequences.
18. The TMAPP-epitope conjugate of any preceding aspect, wherein the TMAPP-epitope conjugate comprises at least one linker comprising:
   (i) Gly (polyG or polyglycine), Gly and Ala (e.g., GA or AG), Ala and Ser (e.g., AS or SA), Gly and Ser (e.g., GS, GSGGS, GGGS, GGSG, GGSGG, GSGSG, GSGGG, GGGSG, GSSSG, GGGGS), or Ala and Gly (e.g., AAAGG), any of which may be repeated 1, 2, 3, 4, 5, 6, 7, 8, 9, or 10 times; or
   (ii) a cysteine-containing linker sequence selected from CGGGS, GCGGS, GGCGS, GGGCS, and GGGGC, with the remainder of the linker comprised of Gly and Ser residues (e.g., GGGGS units that may be repeated from 1 to 10 times.
19. The TMAPP-epitope conjugate of any preceding aspect, wherein the TMAPP-epitope conjugate comprises at least one rigid peptide linker.
20. The TMAPP-epitope conjugate of any preceding aspect, wherein the TMAPP-epitope conjugate comprise at least one linker aa sequence independently selected from GCGASGGGGSGGGGS, GCGGSGGGGSGGGGSGGGGS, GCGGSGGGGSGGGGS, and GCGGS(G₄S) where the G₄S unit may be repeated from 1 to 10 times (e.g., repeated 1, 2, 3, 4, 5, 6, 7, 8, 9, or 10 times); wherein the linker cysteine residue optionally forms a disulfide bond (e.g., with another peptide sequence of the unconjugated TMAPP).
21. The TMAPP-epitope conjugate of any preceding aspect, wherein the MHC class II α1 and α2 domain polypeptide sequences comprise human class II α1 and α2 domain polypeptide having at least 90% or at least 95% (e.g., at least 98% or 100%) sequence identity to an HLA DR alpha (DRA), DQ alpha 1 (DQA1), or DQ alpha 2 (DQA2) α1 and α2 domain polypeptide sequences.
22. The TMAPP-epitope conjugate of any preceding aspect, wherein the MHC class II α1 and α2 domain polypeptide sequences comprise human class II α1 and α2 domain polypeptide having at least 98% or 100% sequence identity to an HLA DR alpha (DRA), DQ alpha 1 (DQA1), or DQ alpha 2 (DQA2) α1 and α2 domain polypeptide sequences.
23. The TMAPP-epitope conjugate of any preceding aspect, wherein the MHC class II β1 and β2 domain polypeptide sequences comprises human class β1 and β2 domain polypeptide sequences having at least 90% or at least 95% (e.g., at least 98% or 100%) sequence identity to an HLA DR beta 1 (DRB1), DR beta 3 (DRB3), DR beta 4 (DRB4), DR beta 5 (DRB5), DQ beta 1 (DQB1), or DQ beta 2 (DQB2) β1 and β2 domain polypeptide sequences.
24. The TMAPP-epitope conjugate of any preceding aspect, wherein the MHC class II β1 and β2 domain polypeptide sequences comprises human class β1 and β2 domain polypeptide sequences having at least 98% or 100% sequence identity to an HLA DR beta 1 (DRB1), DR beta 3 (DRB3), DR beta 4 (DRB4), DR beta 5 (DRB5), DQ beta 1 (DQB1), or DQ beta 2 (DQB2) β1 and β2 domain polypeptide sequences.
25. The TMAPP-epitope conjugate of any preceding aspect, wherein:
   a. the α1 and α2 domain polypeptide sequences each having at least 90% or at least 95% (e.g., at least 98% or 100%) sequence identity to all or at least about 50 (e.g., at least about 60, 70, 80, 85, or 90) contiguous aas of the HLA DR alpha (DRA) α1 or α2 domain polypeptide sequence provided in FIG. 4; and
   b. the 0l and β2 domain polypeptide sequences each having at least 90% or at least 95% (e.g., at least 98% or 100%) sequence identity to all or at least about 50 (e.g., at least about 60, 70, 80, 85, or 90) contiguous aas of an HLA DR beta 1 (DRB1), DR beta 3 (DRB3), DR beta 4 (DRB4), or DR beta 5 (DRB5) β1 or β2 domain polypeptide sequence provided in any one of FIG. 5, 6, 7, or 8.
26. The TMAPP-epitope conjugate of any preceding aspect, wherein:
   a. the α1 and α2 domain polypeptide sequences each having at least 98% or 100% sequence identity to all or at least about 50 (e.g., at least about 60, 70, 80, 85, or 90) contiguous aas of the HLA DR alpha (DRA) α1 or α2 domain polypeptide sequence provided in FIG. 4; and
   b. the β1 and β2 domain polypeptide sequences each having at least 98% or 100% sequence identity to all or at least about 50 (e.g., at least about 60, 70, 80, 85, or 90) contiguous aas of an HLA DR beta 1 (DRB1), DR beta 3 (DRB3), DR beta 4 (DRB4), or DR beta 5 (DRB5) β1 or β2 domain polypeptide sequence provided in any one of FIG. 5, 6, 7, or 8.
27. The TMAPP-epitope conjugate of any of aspects 1-26, wherein:
   the β1 and β2 domain polypeptide sequences each have at least 90% or at least 95% (e.g., at least 98% or 100%) sequence identity to all or at least about 50 (e.g., at least about 60, 70, 80, 85, or 90) contiguous aas of an HLA DRB1*0301, DRB1*0401, DRB1*0402, DRB1*0405, DRB1*0801, or DRB1*0901 β31 or β2 domain polypeptide sequence provided in FIG. 5.
28. The TMAPP-epitope conjugate of aspect 27, wherein:
   the β1 and β2 domain polypeptide sequences each have at least 98% or 100% sequence identity to all or at least about 50 (e.g., at least about 60, 70, 80, 85, or 90) contiguous aas of an HLA DRB1*0301, DRB1*0401, DRB1*0402, DRB1*0405, DRB1*0801, or DRB1*0901 β31 or β2 domain polypeptide sequence provided in FIG. 5.
29. The TMAPP-epitope conjugate of any of aspects 1-26, wherein:
   the β1 and β2 domain polypeptide sequences each have at least 90% or at least 95% (e.g., at least 98% or 100%) sequence identity to all or at least about 50 (e.g., at least about 60, 70, 80, 85, or 90) contiguous aas of an HLA DRB1*0301, DRB1*0401, DRB1*0402, or DRB1*0405 β1 or β2 domain polypeptide sequence provided in FIG. 5.
30. The TMAPP-epitope conjugate of aspect 29, wherein:
   the β1 and β2 domain polypeptide sequences each have at least 98% or 100% sequence identity to all or at least about 50 (e.g., at least about 60, 70, 80, 85, or 90) contiguous aas of an HLA DRB1*0301, DRB1*0401, DRB1*0402, or DRB1*0405 β1 or β2 domain polypeptide sequence provided in FIG. 5.
31. The TMAPP-epitope conjugate of any of aspects 1-24, wherein:
   a. the α1 and α2 domain polypeptide sequences each having at least 90% or at least 95% (e.g., at least 98% or 100%) sequence identity to all or at least about 50 (e.g., at least about 60, 70, 80, 85, or 90) contiguous aas of the HLA DQ alpha 1 or DQ alpha 2 (DQA1 or DQA2) α1 or α2 domain polypeptide sequence; and
   b. the β1 and β2 domain polypeptide sequences each having at least 90% or at least 95% (e.g., at least 98% or 100%) sequence identity to all or at least about 50 (e.g., at least about 60, 70, 80, 85, or 90) contiguous aas of an HLA DQ beta 1 or DQ beta 2 (DQB1 or DQB2) β1 or β2 domain polypeptide sequence.
32. The TMAPP-epitope conjugate of any of aspects 1-24, wherein:
   a. the α1 and α2 domain polypeptide sequences each having at least at least 98% or 100% sequence identity to all or at least about 50 (e.g., at least about 60, 70, 80, 85, or 90) contiguous aas of the HLA DQ alpha 1 or DQ alpha 2 (DQA1 or DQA2) α1 or α2 domain polypeptide sequence; and
   b. the β1 and β2 domain polypeptide sequences each having at least 98% or 100% sequence identity to all or at least about 50 (e.g., at least about 60, 70, 80, 85, or 90) contiguous aas of an HLA DQ beta 1 or DQ beta 2 (DQB1 or DQB2) β1 or β2 domain polypeptide sequence.
33. The TMAPP-epitope conjugate of any of aspects 1-24, wherein:
   a. the α1 and α2 domain polypeptide sequences each having at least 90% or at least 95% (e.g., at least 98% or 100%) sequence identity to all or at least about 60 (e.g., at least about 70, 80, 85, or 90) contiguous aas of an HLA DQ alpha 1 (DQA1) α1 or α2 domain polypeptide sequence; and
b. the β1 and β2 domain polypeptide sequences each having at least 90% or at least 95% (e.g., at least 98% or 100%) sequence identity to all or at least about 60 (e.g., at least about 70, 80, 85, or 90) contiguous aas of an HLA DQ beta 1 (DQB1) β1 or β2 domain polypeptide sequence.
34. The TMAPP-epitope conjugate of aspect 33, wherein:
   a. the α1 and α2 domain polypeptide sequences each having at least 90% or at least 95% (e.g., at least 98% or 100%) sequence identity to all or at least about 60 (e.g., at least about 70, 80, 85, or 90) contiguous aas of an HLA DQ alpha 1 (DQA1) α1 or α2 domain polypeptide sequence; and
   b. the β1 and β2 domain polypeptide sequences each having at least 98% or 100% sequence identity to all or at least about 60 (e.g., at least about 70, 80, 85, or 90) contiguous aas of an HLA DQ beta 1 (DQB1) β1 or β2 domain polypeptide sequence.
35. The TMAPP-epitope conjugate of any of aspects 1-24, wherein:
   a. the α1 and α2 domain polypeptide sequences each having at least 90% or at least 95% (e.g., at least 98% or 100%) sequence identity to all or at least about 60 (e.g., at least about 70, 80, 85, or 90) contiguous as of an HLA DQ alpha 2 (DQA2) α1 or α2 domain polypeptide sequence; and
   b. the β1 and β2 domain polypeptide sequences each having at least 90% or at least 95% (e.g., at least 98% or 100%) sequence identity to all or at least about 60 (e.g., at least about 70, 80, 85, or 90) contiguous as of an HLA DQ beta 2 (DQB2) β1 or β2 domain polypeptide sequence.
36. The TMAPP-epitope conjugate of aspect 35, wherein:
   a. the α1 and α2 domain polypeptide sequences each having at least 98% or 100% sequence identity to all or at least about 60 (e.g., at least about 70, 80, 85, or 90) contiguous aas of an HLA DQ alpha 2 (DQA2) α1 or α2 domain polypeptide sequence; and
   b. the β1 and β2 domain polypeptide sequences each having at least 98% or 100% sequence identity to all or at least about 60 (e.g., at least about 70, 80, 85, or 90) contiguous aas of an HLA DQ beta 2 (DQB2) β1 or β2 domain polypeptide sequence.
37. The TMAPP-epitope conjugate of any of aspects 1-24, wherein:
   a. the α1 and α2 domain polypeptide sequences each having at least 90% or at least 95% (e.g., at least 98% or 100%) sequence identity to all or at least about 60 (e.g., at least about 70, 80, 85, or 90) contiguous aas of an HLA DQA1*0501 α1 or α2 domain polypeptide sequence; and
b. the β1 and β2 domain polypeptide sequences each having at least 90% or at least 95% (e.g., at least 98% or 100%) sequence identity to all or at least about 60 (e.g., at least about 70, 80, 85, or 90) contiguous aas of an HLA DQB1*0201 β31 or β2 domain polypeptide sequence.
38. The TMAPP-epitope conjugate of aspect 37, wherein:
   a. the α1 and α2 domain polypeptide sequences each having at least 98% or 100% sequence identity to all or at least about 60 (e.g., at least about 70, 80, 85, or 90) contiguous aas of an HLA DQA1*0501 α1 or α2 domain polypeptide sequence; and
   b. the β1 and β2 domain polypeptide sequences each having at least 98% or 100% sequence identity to all or at least about 60 (e.g., at least about 70, 80, 85, or 90) contiguous aas of an HLA DQB1*0201 β1 or β2 domain polypeptide sequence.
39. The TMAPP-epitope conjugate of any of aspects 1-24, wherein:
   a. the α1 and α2 domain polypeptide sequences each having at least 90% or at least 95% (e.g., at least 98% or 100%) sequence identity to all or at least about 60 (e.g., at least about 70, 80, 85, or 90) contiguous aas of an HLA DQA1*0301 α1 or α2 domain polypeptide sequence; and
   b. the 0l and β2 domain polypeptide sequences each having at least 90% or at least 95% (e.g., at least 98% or 100%) sequence identity to all or at least about 60 (e.g., at least about 70, 80, 85, or 90) contiguous aas of an HLA DQB1*0302 β1 or β2 domain polypeptide sequence.
40. The TMAPP-epitope conjugate of aspect 39, wherein:
   a. the α1 and α2 domain polypeptide sequences each having at least 98% or 100% sequence identity to all or at least about 60 (e.g., at least about 70, 80, 85, or 90) contiguous aas of an DQA1*0301 α1 or α2 domain polypeptide sequence; and
   b. the β1 and β2 domain polypeptide sequences each having at least 98% or 100% sequence identity to all or at least about 60 (e.g., at least about 70, 80, 85, or 90) contiguous aas of an DQB1*0302 β1 or β2 domain polypeptide sequence.
41. The TMAPP-epitope conjugate of any preceding aspect, wherein the TMAPP does not comprise a scaffold or does not comprise an Ig Fc scaffold.
42. The TMAPP-epitope conjugate of any preceding aspect, wherein the TMAPP-epitope conjugate does not comprise a scaffold polypeptide sequence (e.g., that is capable of binding to the scaffold polypeptide sequence of another TMAPP).
43. The TMAPP-epitope conjugate of any of aspects 1 to 40, wherein the TMAPP-epitope conjugate comprises a scaffold polypeptide sequence.
44. The TMAPP-epitope conjugate of aspect 43, wherein the scaffold polypeptide sequence is a non-interspecific sequence or interspecific sequence.
45. The TMAPP-epitope conjugate of aspect 44, wherein the interspecific and non-interspecific sequence are selected from the group consisting of: immunoglobulin heavy chain constant regions (Ig Fc e.g., Ig CH2-CH3); collectin polypeptides, coiled-coil domains, leucine-zipper domains; Fos polypeptides; Jun polypeptides; Ig CH1; Ig C_{L} κ; Ig C_{L} λ; knob-in-hole without disulfide (“KiH”); knob-in hole with a stabilizing disulfide bond (“KiHs-s”); HA-TF; ZW-1; 7.8.60; DD-KK; EW-RVT; EW-RVTs-s; and A107 sequences.
46. The TMAPP-epitope conjugate of any of aspects 43 to 45 complexed to form a duplex TMAPP-epitope conjugate or higher order TMAPP-epitope conjugate comprising at least a first TMAPP-epitope conjugate and a second TMAPP-epitope conjugate:
   (i) the first TMAPP-epitope conjugate comprises a first scaffold polypeptide sequence; and
   (ii) the second TMAPP-epitope conjugate comprises a second scaffold polypeptide sequence;
wherein the first TMAPP-epitope conjugate and the second TMAPP-epitope conjugate are associated by binding interactions between the first scaffold polypeptide sequence and second scaffold polypeptide sequence, and wherein the interactions optionally including one or more interchain covalent bonds (e.g., one or two disulfide bonds); and
wherein the duplex or higher order unconjugated TMAPP comprises at least one masked TGF-β MOD wherein the masking sequence and the TGF-β sequence are present in cis or in trans.
47. The TMAPP-epitope conjugate or duplex TMAPP-epitope conjugate of aspect 46, wherein the first scaffold polypeptide sequence and the second scaffold polypeptide sequence are interspecific sequences.
48. The TMAPP-epitope conjugate or duplex TMAPP-epitope conjugate of aspect 47, wherein the first scaffold polypeptide sequence and the second scaffold polypeptide sequence are selected from the group consisting of: a Fos and Jun polypeptide pair; Ig CH1 and Ig CL κ or λ constant region polypeptide pair; and interspecific immunoglobulin heavy chain constant regions pairs (e.g., Ig Fc pairs including, but not limited to, a KiH pair, a KiHs-s pair, a HA-TF polypeptide pair, a ZW-1 polypeptide pair, a 7.8.60 polypeptide pair, a DD-KK polypeptide pair, an EW-RVT polypeptide pair, an EW-RVTs-s polypeptide pair, or an Δ107 polypeptide pair).
49. The TMAPP-epitope conjugate or duplex TMAPP-epitope conjugate of aspect 47, wherein the first scaffold polypeptide sequence and the second scaffold polypeptide sequence are an interspecific Ig Fc pair.
50. The TMAPP-epitope conjugate or duplex TMAPP-epitope conjugate of aspect 47, wherein the first scaffold polypeptide sequence and the second scaffold polypeptide sequence are selected from the group consisting of: a KiH pair; a KiHs-s pair; a HA-TF polypeptide pair; a ZW-1 polypeptide pair; a 7.8.60 polypeptide pair; a DD-KK polypeptide pair; an EW-RVT polypeptide pair; an EW-RVTs-s polypeptide pair; and an Δ107 polypeptide pair.
51. The TMAPP-epitope conjugate or duplex TMAPP-epitope conjugate of aspect 48, wherein the first scaffold polypeptide sequence and the second scaffold polypeptide sequence are a KiH pair or a KiHs-s pair.
52. The TMAPP-epitope conjugate or duplex TMAPP-epitope conjugate of any of aspects 1 to 46, wherein the first scaffold polypeptide sequence and the second scaffold polypeptide sequence wherein the first scaffold polypeptide sequence and second scaffold polypeptide sequence are non-interspecific sequences.
53. The TMAPP-epitope conjugate or duplex TMAPP-epitope conjugate of aspect 52, wherein the first scaffold polypeptide sequence and second scaffold polypeptide sequence are identical.
54. The TMAPP-epitope conjugate or duplex TMAPP-epitope conjugate of aspect 52, wherein the first scaffold polypeptide sequence and second scaffold polypeptide sequence are not identical.
55. The TMAPP-epitope conjugate or duplex TMAPP-epitope conjugate of any of aspects 52 to 54, wherein the first scaffold polypeptide sequence and second scaffold polypeptide sequence are selected from the group consisting of: immunoglobulin heavy chain constant region sequences (Ig Fc sequences or Ig Fc CH2-CH3 region sequences); collectin family dimerization sequences; coiled-coil domain sequences; and leucine-zipper domain sequences.
56. The TMAPP-epitope conjugate or duplex TMAPP-epitope conjugate of any of aspects 52 to 55, wherein the first scaffold polypeptide sequence and second scaffold polypeptide sequence are Ig Fc sequences.
57. The TMAPP-epitope conjugate or duplex TMAPP-epitope conjugate of any of aspects 55 to 56, wherein the Ig Fc sequences are selected from the group consisting of IgA, IgD, IgE, IgG and IgM Ig Fc sequences.
58. The TMAPP-epitope conjugate or duplex TMAPP-epitope conjugate of any of aspects 56 to 57, wherein the Ig Fc sequences are selected from IgG1, IgG2, IgG3, and IgG4 CH2-CH3 domain sequences
59. The TMAPP-epitope conjugate or duplex TMAPP-epitope conjugate of any of aspects 55 to 56, wherein the Ig Fc sequences comprise a CH2 and/or CH3 domain having at least about 90% (e.g., at least about 95% or at least about 98%) or 100% as sequence identity to an as sequence of the CH2 and/or CH3 domains of an Ig Fc region of an IgG1, IgG2, IgG3, or IgG4 sequence set forth in any of FIGS. 2D to 2G.
60. The TMAPP-epitope conjugate or duplex TMAPP-epitope conjugate of any of aspects 55 to 56, wherein the Ig Fc sequences comprise a CH2 and/or CH3 domain having at least about 90% (e.g., at least about 95% or at least about 98%) or 100% as sequence identity to aIgG1 as sequence of FIG. 2D.
61. The duplex TMAPP-epitope conjugate of any of aspects 55 to 60, wherein the first scaffold polypeptide sequence and the second scaffold polypeptide sequence are covalently linked.
62. The duplex TMAPP-epitope conjugate of aspect 61, wherein the first scaffold polypeptide sequence and the second scaffold polypeptide sequence are covalently linked by one or two disulfide bonds.
63. The TMAPP-epitope conjugate or duplex TMAPP-epitope conjugate of any of aspects 55 to 62, wherein when the scaffold polypeptide sequences comprise one or more IgFc sequences the IgFc sequences comprise one or more IgFc sequence substitutions that reduce or substantially eliminate IgFc mediated antibody-dependent cellular cytotoxicity (ADCC) and/or complement-dependent cytotoxicity (CDC) functions relative to level of ADCC and/or CDC observed in the absence of the IgFc sequence substitutions.
For example, the IgFc sequences, may comprise one or more substitutions at L234, L235, G236, G237, P238, S239, D270, N297, K322, P329, and/or P331 (respectively, aas L14, L15, G16, G17, P18, S19, N77, D50, K102, P109, and P111 of the wt. IgG1 aa sequence SEQ ID NO:4 provided in FIG. 2D).
64. The TMAPP-epitope conjugate or duplex TMAPP-epitope conjugate of any preceding aspect, comprising a masked TGF-β MOD in cis.
65. The TMAPP-epitope conjugate or duplex TMAPP-epitope conjugate of aspect 64, wherein the masked TGF-β MOD comprises from N-terminus to C-terminus the masking sequence and the TGF-β sequence.
66. The TMAPP-epitope conjugate or duplex TMAPP-epitope conjugate of aspect 64, wherein the masked TGF-β MOD comprises from N-terminus to C-terminus the TGF-β sequence and the masking sequence.
67. The TMAPP-epitope conjugate or duplex TMAPP-epitope conjugate of any of aspects 64 to 66, comprising a masked TGF-β MOD located at the N-terminus of one or more of: a presenting complex; a presenting complex first sequence; or a presenting complex second sequence.
68. The TMAPP-epitope conjugate or duplex TMAPP-epitope conjugate of any of aspects 64 to 67, comprising a masked TGF-β MOD located at the C-terminus of one or more of: scaffold polypeptide sequence; or a presenting complex second sequence.
69. The TMAPP-epitope conjugate or duplex TMAPP-epitope conjugate of any of aspects 1 to 68, comprising a masked TGF-β MOD in trans.
70. The TMAPP-epitope conjugate or duplex TMAPP-epitope conjugate of aspect 69, wherein the masking sequence is located at the N-terminus of the presenting complex first sequence or the presenting complex second sequence, and the TGF-β sequence is located at the other of the presenting complex first sequence or the presenting complex second sequence.
71. The duplex TMAPP-epitope conjugate of any of aspects 46 to 70, wherein the masking sequence is located at the C-terminus of the first scaffold polypeptide sequence, and the TGF-β sequence is located at the C-terminus of the second scaffold polypeptide sequence.
72. The TMAPP-epitope conjugate or duplex TMAPP-epitope conjugate of any preceding aspect comprising one or more independently selected additional MODs (wt. and/or variant) or pairs of additional MODs in tandem (both wt, both variant, or one wt. and one variant).
73. The TMAPP-epitope conjugate or duplex TMAPP-epitope conjugate of aspect 72, comprising an addition MOD or pair of additional MODs in tandem located at the N-terminus of a presenting sequence, presenting complex first sequence and/or presenting complex second sequence.
74. The TMAPP-epitope conjugate or duplex TMAPP-epitope conjugate of aspect 72, comprising an addition MOD or pair of additional MODs in tandem located at the C-terminus of a presenting sequence, presenting complex first sequence, presenting complex second sequence, or scaffold polypeptide sequence.
75. The TMAPP-epitope conjugate or duplex TMAPP-epitope conjugate of any of aspects 71 to 74, wherein the additional MOD is located on a masking sequence or TGF-β sequence (e.g., located on a TGF-β MOD creating a TGF-β MOD-additional MOD in tandem).
76. The TMAPP-epitope conjugate or duplex TMAPP-epitope conjugate of any preceding aspect wherein at least one TGF-β sequence is a TGF-ββ1 polypeptide optionally comprising a substitution of C77.
77. The TMAPP-epitope conjugate or duplex TMAPP-epitope conjugate of aspect 76, wherein the TGF-β1 polypeptide comprises an amino acid sequence having at least 90% (e.g., at least 95% or at least 98%) or 100% aa sequence identity to at least 70, at least 80, at least 90, at least 100, at least 110, or 112 aas of the TGF-β1 as sequence set forth in FIG. 14 (SEQ ID NO:211), and optionally comprising a substitution of C77.
78. The TMAPP-epitope conjugate or duplex TMAPP-epitope conjugate of aspect 76, wherein the TGF-β1 polypeptide comprises an amino acid sequence having at least at least 90% (e.g., at least 95% or at least 98%) or 100% aa sequence identity to at least 70, at least 80, at least 90, at least 100, at least 110, or contiguous 112 aas of the TGF-β1 amino acid sequence AL DTNYCFSSTE KNCCVRQLYI DF [symbol] KDLGWKW IHEPKGYHAN FCLGPCPYIW SLDTQYSKVL ALYNQHNPGA SAAPCCVPQA LEPLPIVYY [symbol] G [symbol] KPKVEQLS NMIVRSCKCS (SEQ ID NO:101), and optionally comprising a substitution of C77. For example, the TGF-β1 as sequence may have at least 90%, or at least 95% (e.g., at least 98% or 100%) as sequence identity to the TGF-β1 amino acid sequence of SEQ ID NO:101) optionally comprising a substitution of C77.
79. The TMAPP-epitope conjugate or duplex TMAPP-epitope conjugate of any of aspects 76 to 78, wherein the TGF-β1 as sequence comprises a C77S substitution.
80. The TMAPP-epitope conjugate or duplex TMAPP-epitope conjugate of any of aspects 1 to 75, wherein at least one TGF-β sequence is a TGF-β2 polypeptide optionally comprising a substitution of C77.
81. The TMAPP-epitope conjugate or duplex TMAPP-epitope conjugate of aspect 80, wherein the TGF-β2 polypeptide comprises an amino acid sequence having at least at least 90% (e.g., at least 95% or at least 98%) or 100% as sequence identity to at least 70, at least 80, at least 90, at least 100, at least 110, or 112 aas of the mature TGF-β2 as sequence set forth in FIG. 14 (SEQ ID NO:212), and optionally comprising a substitution of C77.
82. The TMAPP-epitope conjugate or duplex TMAPP-epitope conjugate of aspect 80, wherein the TGF-β2 polypeptide comprises an amino acid sequence having a at least 90% (e.g., at least 95% or at least 98%) or 100% as sequence identity to at least 70, at least 80, at least 90, at least 100, at least 110, or 112 contiguous aas of the TGF-β2 amino acid sequence ALDAAYCFRN VQDNCCLRPL YIDF [symbol] RDLG WKWIHEPKGY NANFCAGACP YLWSSDTQHS RVLSLYNTIN PEASASPCCV SQDLEPLTIL YY [symbol] G [symbol] TPKIE QLSNMIVKSC KCS (SEQ ID NO:103), and optionally comprising a substitution of C77. For example, the TGF-β2 as sequence may have at least 90%, or at least 95%, (e.g., at least 98% or 100%) as sequence identity to the TGF-β2 amino acid sequence of SEQ ID NO:103) optionally comprising a substitution of C77.
83. The TMAPP-epitope conjugate or duplex TMAPP-epitope conjugate of any of aspects 80 to 82, wherein the TGF-β2 as sequence comprises a C77S substitution.
84. The TMAPP-epitope conjugate or duplex TMAPP-epitope conjugate of any of aspects 1-75, wherein at least one TGF-β sequence is a TGF-β3 polypeptide optionally comprising a substitution of C77.
85. The TMAPP-epitope conjugate or duplex TMAPP-epitope conjugate of aspect 84, wherein the TGF-β3 polypeptide comprises an amino acid sequence having at least 90% (e.g., at least 95% or at least 98%) or 100% aa sequence identity to at least 70, at least 80, at least 90, at least 100, at least 110, or 112 aas of the TGF-β3 aa sequence set forth in FIG. 14 (SEQ ID NO:213), and optionally comprising a substitution of C77.
86. The TMAPP-epitope conjugate or duplex TMAPP-epitope conjugate of aspect 84, wherein the TGF-β3 polypeptide comprises an amino acid sequence having at least 90% (e.g., at least 95% or at least 98%) or 100% aa sequence identity to at least 70, at least 80, at least 90, at least 100, at least 110, or 11? ˜s aas of the TGF-β3 amino acid sequence ALDTNYCFRN LEENCCVRPL YIDF [symbol] QDLGW KWVHEPKGYY ANFCSGPCPY LRSADTTHST VLGLYNTLNP EASASPCCVP QDLEPLTILY Y [symbol] G [symbol] RTPKVEQ LSNMVVKSCK CS (SEQ ID NO:105), and optionally comprising a substitution of C77. For example, the TGF-β3 aa sequence may have at least 90%, or at least 95%, (e.g., at least 98% or 100%) aa sequence identity to the TGF-β3 amino acid sequence of SEQ ID NO:105) optionally comprising a substitution of C77.
87. The TMAPP-epitope conjugate or duplex TMAPP-epitope conjugate of any of aspects 84 to 86, wherein the TGF-β3 an sequence comprises a C77S substitution.
88. The TMAPP-epitope conjugate or duplex TMAPP-epitope conjugate of any of aspects 76 to 87, wherein the TGF-β sequence comprising a substitution at one or more of position 25, 92, and/or 94.
89. The TMAPP-epitope conjugate or duplex TMAPP-epitope conjugate of any preceding aspect, wherein at least one masking sequence is a TGF-β receptor (TβR, e.g., a TβRI, TβRII, or TβRIII an sequence) polypeptide, anti-TGF-β antibody (e.g., anti-TGF-β1, anti-TGF-β2, and/or anti-TGF-β3) or antibody-related polypeptide/aa sequence (e.g., antigen binding fragment, Fab, Fab′, single chain antibody, scFv, peptide aptamer, or nanobody an sequence).
90. The TMAPP-epitope conjugate or duplex TMAPP-epitope conjugate of aspect 89, wherein the masking sequence comprises all or part of a TβRI ectodomain.
91. The TMAPP-epitope conjugate or duplex TMAPP-epitope conjugate of aspect 89, wherein the masking sequence comprise an amino acid sequence having at least 90% (e.g., at least 95% or at least 98%) or 100% aa sequence identity to at least 70, at least 80, at least 90, at least 100, or 103 aas of the following TβRI ectodomain an sequence: LQCFCHL CTKDNFTCVT DGLCFVSVTE TTDKVIHNSM CIAEIDLIPR DRPFVCAPSS KTGSVTITYC CNQDHCNKIE LPTTVKSSPG LGPVEL (SEQ ID NO:107) See e.g., FIG. 16A). For example, the masking sequence may have at least 90%, or at least 95% (e.g., at least 98% or 100%) an sequence identity to the TβRI ectodomain an sequence of SEQ ID NO:107).
92. The TMAPP-epitope conjugate or duplex TMAPP-epitope conjugate of aspect 89, wherein the masking sequence comprises all or part of a TβRII ectodomain (See e.g., FIG. 16B).
93. The TMAPP-epitope conjugate or duplex TMAPP-epitope conjugate of aspect 89, wherein the masking sequence comprise an amino acid sequence having at least 90% (e.g., at least 95% or at least 98%) or 100% aa sequence identity to at least 70, at least 80, at least 90, at least 100, at least
110, at least 120, at least 130, at least 140, at least 150 or at least 154 aas of T3RII isoform A ectodomain as sequence: IPPHVQK SDVEMEAQKD EIICPSCNRT AHPLRHINND MIVTDNNGAV KFPQLCKFCD VRFSTCDNQK SCMSNCSITS ICEKPQEVCV AVWRKNDENI TLETVCHDPK LPYHDFILED AASPKCIMKE KKKPGETFFM CSCSSDECND NIIFSEE (SEQ ID NO:108) optionally comprising a substitution at any one or more of F55, D57, S77, E80, and D143, which correspond to F30, D32, S52, E55 and D118 of the mature isoform B. For example, the TβRII isoform A ectodomain as sequence may have at least 90%, or at least 95% (e.g., at least 98% or 100%) aa sequence identity to the TDRII isoform A ectodomain as sequence of SEQ ID NO:108), and optionally comprise a substitution at any one or more of F55, D57, S77, E80, and D143.
94. The TMAPP-epitope conjugate or duplex TMAPP-epitope conjugate of aspect 89, wherein the masking sequence comprise an amino acid sequence having at least 90% (e.g., at least 95% or at least 98%) or 100% aa sequence identity to at least 70, at least 80, at least 90, at least 100, at least 110, at least 120, at least 130, at least 140 or 143 aas of T3RII isoform B ectodomain aa sequence: IPPHVQKSVN NDMIVTDNNG AVKFPQLCK [symbol] CDVRFSTCDN QKSCMSNCSI T [symbol] IC [symbol] KPQEV CVAVWRKNDE NITLETVCHD PKLPYHDFIL EDAASPKCIM KEKKKPGETF FMCSCSS [symbol] EC NDNIIFSEEY NTSNPDLLLV IFQ (SEQ ID NO:109), optionally comprising a substitution at any one or more of F30, D32, 552, E55 and D118 of the mature isoform B. For example, the TβRII isoform B ectodomain as sequence may have at least 90%, or at least 95% (e.g., at least 98% or 100%) as sequence identity to the TβRII isoform B ectodomain as sequence of SEQ ID NO:109).
95. The TMAPP-epitope conjugate or duplex TMAPP-epitope conjugate of aspect 89, wherein the masking sequence comprise an amino acid sequence having at least 90% (e.g., at least 95% or at least 98%) or 100% aa sequence identity to at least 70, at least 80, at least 90, at least 100, at least 110, at least 120, at least 130, at least 140, or 142 aas of TβRII isoform B ectodomain as sequence: IPPHVQKSVN NDMIVTDNNG AVKFPQLCK [symbol] C [symbol] VRFSTCDN QKSCMSNCSI T [symbol] IC [symbol] KPQEV VAVWRKNDE NITLETVCHD PKLPYHDFIL EDAASPKCIM KEKKKPGETF FMCSCSSDEC NDNIIFSEE (SEQ ID NO:1 10), optionally comprising a substitution at any one or more of F30, D32, S52, E55 and D118 of the mature isoform B. For example, the TβRII isoform B ectodomain as sequence may have at least 90%, or at least 95% (e.g., at least 98% or 100%) as sequence identity to the TβRII isoform B ectodomain as sequence of SEQ ID NO:110).
96. The TMAPP-epitope conjugate or duplex TMAPP-epitope conjugate of aspect 89, wherein the masking sequence comprise an amino acid sequence having at least 90% (e.g., at least 95% or at least 98%) or 100% aa sequence identity to at least 70, at least 80, at least 90, at least 100, at least
110, or 114 aas of TβRII isoform B Δ14 ectodomain as sequence: VTDNNG AVKFPQLCK [symbol] C [symbol] VRFSTCDN QKSCMSNCSI TSIC [symbol] KPQEV CVAVWRKNDE NITLETVCHD PKLPYHDFIL EDAASPKCIM KEKKKPGETF FMCSCSS [symbol] EC NDNIIFSEE (SEQ ID NO:111), optionally comprising a substitution at any one or more of F30, D32, S52, E55 and D118 of the mature isoform B. For example, the TβRII isoform B Δ14 ectodomain aa sequence may have at least 90%, or at least 95% (e.g., at least 98% or 100%) aa sequence identity to the TpIRII isoform B Δ14 ectodomain as sequence of SEQ ID NO:111).
97. The TMAPP-epitope conjugate or duplex TMAPP-epitope conjugate of aspect 89, wherein the masking sequence comprise an amino acid sequence having at least 90% (e.g., at least 95% or at least 98%) or 100% aa sequence identity to at least 70, at least 80, at least 90, at least 100, or 104 aas of TβRII isform B Δ25 ectodomain a sequence: QLCK [symbol] C [symbol] VRESTCDN QKSCMSNCSI TSIC [symbol] KPQEV CVAVWRKNDE NITLETVCHD PKLPYHDFIL EDAASPKCIM KEKKKPGETF FMCSCSS [symbol] EC NDNIIFSEE (SEQ ID NO: 112), optionally comprising a substitution at any one or more of F55, D57, S77, E80, and D143, which correspond to F30, D32, 552, E55 and D118 of the mature isoform B. For example, the TβRII isoform B Δ25 ectodomain as sequence may have at least 90%, or at least 95% (e.g., at least 98% or 100%) aa sequence identity to the TpIRII isoform B Δ25 ectodomain as sequence of SEQ ID NO:112).
98. The TMAPP-epitope conjugate or duplex TMAPP-epitope conjugate of aspect 89, wherein the masking sequence comprise an amino acid sequence having at least 90% (e.g., at least 95% or at least 98%) or 100% aa sequence identity to at least 70, at least 80, at least 90, at least 100, at least 110, or 111 aas of a TβRII isoform B Δ25 ectodomain containing as sequence: QLCKE C [symbol] VRFSTCDN QKSCMSNCSI T [symbol] ICEKPQEV CVAVWRKNDE NITLETVCHD PKLPYHDFIL EDAASPKCIM KEKKKPGETF FMCSCSS [symbol] EC NDNIIFSEEY NTSNPD (SEQ ID NO:217), optionally comprising a substitution at any one or F30, D32, S52, and E55 of the mature isoform B. For example, the TβRII isoform B Δ25 ectodomain as sequence may have at least 90%, or at least 95% (e.g., at least 98% or 100%) as sequence identity to the TβRII isoform B Δ25 ectodomain as sequence of SEQ ID NO:217).
99. The TMAPP-epitope conjugate or duplex TMAPP-epitope conjugate of aspect 93 comprising a substitution in the TβRII isoform A sequence of at least one as (e.g., at least two aas) selected from the groups consisting of L52, F55, D57, S74, 175, T76, S77, 178, E80, V102, D143, and E144 for isoform A.
100. The TMAPP-epitope conjugate or duplex TMAPP-epitope conjugate of aspect 99, wherein the at least one as is (e.g., at least two aas are) selected from the group consisting of L52A, F55A, D57A, D57N, S74A, I75A, T76A, S77A, S77L, I78A, E80A, V102A, D143A, D143R, E144A, and/or E144Q.
101. The TMAPP-epitope conjugate or duplex TMAPP-epitope conjugate of any of aspects 94 to 98, comprising a substitution in the TβRII isoform B sequence of at least one as (e.g., at least two aas) selected from the groups consisting of substitutions at L27, F30, D32, S49, 150, T51, S52, 153, E55, V77, D118, and/or E119.
102. The TMAPP-epitope conjugate or duplex TMAPP-epitope conjugate of aspect 101, comprising a substitution in the TβRII isoform B sequence of at least one aa is (e.g., at least two aas are) selected from the groups consisting L27A, F30A, D32A, D32N, S49A, I50A, T51A, S52A, S52L, I53A, E55A, V77A, D118A, D118R, E119A, and/or E119Q.
103. The TMAPP-epitope conjugate or duplex TMAPP-epitope conjugate of aspect 101 or 102 comprising, a D118A or D118R substitution in the TβRII isoform B sequence.
104. The TMAPP-epitope conjugate or duplex TMAPP-epitope conjugate of aspect 103, comprising more of a F30A, D32N, S52L and/or E55A substitution in the TβRII isoform B sequence.
105. The TMAPP-epitope conjugate or duplex TMAPP-epitope conjugate of any of aspects 94 to 104, comprising an N-terminal deletion in the TβRII receptor mature polypeptide aa sequence.
106. The TMAPP-epitope conjugate or duplex TMAPP-epitope conjugate of aspect 105, wherein from 1 to 14, or 1 to 25 amino acids of the mature TβRII polypeptide sequence have been deleted from the N-terminus of the mature polypeptide (see. e.g., FIG. 16B SEQ ID NOs: 111, 112, and 217).
107. The TMAPP-epitope conjugate or duplex TMAPP-epitope conjugate of aspect 89, wherein the masking sequence comprises all or part of a TβRIII ectodomain (See e.g., FIG. 16C).
108. The TMAPP-epitope conjugate or duplex TMAPP-epitope conjugate of aspect 89, wherein the masking sequence comprise an amino acid sequence having at least 90% (e.g., at least 95% or at least 98%) or 100% aa sequence identity to at least 70, at least 80, at least 90, at least 100, or 120 aas of a TβRIII A isoform or B isoform ectodomain sequences (e.g., provided in FIG. 16C as SEQ ID NO:218 or SEQ ID NO:219). For example, the TβRIII A isoform or B isoform ectodomain aa sequence may have at least 90%, or at least 95% (e.g., at least 98% or 100%) aa sequence identity to the TβRIII isoform A or B ectodomain as sequence of SEQ ID NO:218 or 219).
109. The TMAPP-epitope conjugate or duplex TMAPP-epitope conjugate of aspect 89, wherein the masking sequence comprise an anti-TGF-β antibody or antibody-related polypeptide/aa sequence.
110. The TMAPP-epitope conjugate or duplex TMAPP-epitope conjugate of any preceding aspect, wherein at least one chemical conjugation site or additional chemical conjugation site is selected from the group consisting of:
   a) an amino acid chemical conjugation site (e.g., at an amino acid side chain such as at the thiol group of a cysteine, particularly one that is solvent accessible and exposed on the surface of the TMAPP and not part of a disulfide bond);
   b) non-natural amino acids and/or selenocysteines;
   c) a peptide sequence that acts as an enzyme modification sequence (e.g., sulfatase, transglutaminase or sortase sites);
   d) carbohydrate or oligosaccharide; and
   e) IgG nucleotide binding sites.
111. The TMAPP-epitope conjugate or duplex TMAPP-epitope conjugate of aspect 110, wherein at least one of the chemical conjugation sites or additional chemical conjugation site is an amino acid chemical conjugation site (e.g., provided in an aa sequence of The TMAPP-epitope conjugate using the techniques of molecular biology and protein engineering).
112. The TMAPP-epitope conjugate or duplex TMAPP-epitope conjugate of aspect 111, wherein the amino acid chemical conjugation site is a cysteine.
113. The TMAPP-epitope conjugate or duplex TMAPP-epitope conjugate of aspect 111, wherein the cysteine is provided in an as sequence of the TMAPP-epitope conjugate or duplex TMAPP-epitope conjugate using the techniques of molecular biology and protein engineering (e.g., the cysteine is substituted for another aa).
114. The TMAPP-epitope conjugate or duplex TMAPP-epitope conjugate of aspect 110, wherein at least one of the chemical conjugation sites is a non-natural amino acid or a selenocysteine.
115. The TMAPP-epitope conjugate or duplex TMAPP-epitope conjugate of aspect 110, wherein at least one of the chemical conjugation sites is an enzyme modification sequence selected from the group consisting of: a sulfatase motif; a Sortase A enzyme site; and a transglutaminase site.
116. The TMAPP-epitope conjugate or duplex TMAPP-epitope conjugate of aspect 110, wherein at least one of the chemical conjugation sites is a sulfatase motif; a Sortase A enzyme site; and a transglutaminase site.
117. The TMAPP-epitope conjugate or duplex TMAPP-epitope conjugate of aspect 110, wherein at least one of the chemical conjugation sites is a carbohydrate or oligosaccharide.
118. The TMAPP-epitope conjugate or duplex TMAPP-epitope conjugate of aspect 110, wherein at least one of the chemical conjugation sites is an IgG nucleotide binding sites.
119. The TMAPP-epitope conjugate or duplex TMAPP-epitope conjugate of any preceding aspect, wherein the chemical conjugation site is located in a presenting sequence, presenting complex first sequence, or presenting complex second sequence.
120. The TMAPP-epitope conjugate or duplex TMAPP-epitope conjugate of aspect 119, wherein the chemical conjugation site, and optionally an additional chemical conjugation site, is located in: an MHC Class II α1, α2, β1, or β2 domain polypeptide sequence; a polypeptide linker directly attached to at least one of the α1, α2, β1, or β2 domain polypeptide sequences; a scaffold polypeptide sequence; or a polypeptide linker directly attached to a scaffold
121. The TMAPP-epitope conjugate or duplex TMAPP-epitope conjugate of aspect 120, wherein the chemical conjugation site is located in a α1, α2, β1, or β2 domain polypeptide sequence having at least 90% (e.g., at least 95%, or at least 98%), or even 100% as sequence identity to an MHC Class II α1, α2, β1, or β2 domain sequence provided in any of FIGS. 4 to 18B.
122. The TMAPP-epitope conjugate or duplex TMAPP-epitope conjugate of aspect 120, wherein the chemical conjugation site is located in a α1, α2, β1, or $2 domain polypeptide sequence having at least 90% (e.g., at least 95%, or at least 98%), or even 100% as sequence identity to an MHC Class II α1 or α2 domain sequence provided in any of FIGS. 4 to 18B.
123. The TMAPP-epitope conjugate or duplex TMAPP-epitope conjugate of aspect 120, wherein the chemical conjugation site is located in a α1, α2, β11, or $2 domain polypeptide sequence having at least 90% (e.g., at least 95%, or at least 98%), or even 100% as sequence identity to an MHC Class II β1 or $2 domain sequence provided in any of FIGS. 4 to 18B.
124. The TMAPP-epitope conjugate or duplex TMAPP-epitope conjugate of aspect 120, wherein the chemical conjugation site is located in a α1, α2, β1, or β2 domain polypeptide sequence having at least 90% (e.g., at least 95%, or at least 98%), or even 100% as sequence identity to an MHC Class II DRA α1 or α2 domain sequence, or an MHC Class II DRB1, DRB3, DRB4 or DRB5 β1, or 02 domain sequence provided in any of FIGS. 4 to 8.
125. The TMAPP-epitope conjugate or duplex TMAPP-epitope conjugate of any of aspects 1 to 124, wherein the additional chemical conjugation site is located in a scaffold polypeptide sequence or in a polypeptide linker directly attached to a scaffold polypeptide sequence.
126. The TMAPP-epitope conjugate or duplex TMAPP-epitope conjugate of any of aspects 1 to 125, wherein the chemical conjugation site is located in a Class II MHC α α chain α1 or α2 domain polypeptide sequence position selected from:
   (i) α chain as positions 2-5, such as at aas 3 or 4;
   (ii) α chain aa positions 11-13, such as at as 12;
   (iii) α chain aa positions 27-30, such as at aas 28 or 29;
   (iv) α chain as positions 71-76, such as at aas 72 or 75;
   (v) α chain as positions 79-83, such as at aas 80, 81; or
   (vi) α chain as positions 92-96, such as at aas 93, 94, or 95.
127. The TMAPP-epitope conjugate or duplex TMAPP-epitope conjugate of any of aspects 1 to 125, wherein the chemical conjugation site is located in a Class II MHC α β chain β1 or β2 domain polypeptide sequence position selected from:
   (i) β chain aa positions 4-11, such as at aas 5, 7 or 10;
   (ii) β chain as positions 119-121, such as at as 120; or
   (iii) 0 chain as positions 152-158, such as at aas 153 or 156.
128. The TMAPP-epitope conjugate or duplex TMAPP-epitope conjugate of any of aspects 1 to 125, wherein when an MHC Class 11 β2 domain is located at the carboxy terminus of a presenting sequence or presenting complex, a chemical conjugation site (e.g., a cystine residue) may be located in the last three aas of the β2 domain (the three carboxy most aas), or in a linker attached to the carboxy terminus of the β2 domain.
129. The TMAPP-epitope conjugate or duplex TMAPP-epitope conjugate of any of aspects 119 to 128, wherein at least one of the chemical conjugation sites or additional chemical conjugation site is an amino acid chemical conjugation site (e.g., provided in an aa sequence of the TMAPP-epitope conjugate using the techniques of molecular biology and protein engineering).
130. The TMAPP-epitope conjugate or duplex TMAPP-epitope conjugate of aspect 129, wherein the amino acid chemical conjugation site is a cysteine.
131. The TMAPP-epitope conjugate or duplex TMAPP-epitope conjugate of any preceding aspect comprising at least one additional MOD (wt. or variant), or pair of additional MODs in tandem (both wt, both variant, or one wt. and one variant).
132. The TMAPP-epitope conjugate or duplex TMAPP-epitope conjugate of any preceding aspect, wherein the additional MOD (wt. or variant) or the additional pair of MODs (wt. or variant) are selected independently from the group consisting of: IL-1, IL-2, IL-4, IL-6, IL-7, IL-10, IL-12, IL-15, IL-17, IL-21, IL-23, CD7, CD30L, CD40, CD70, CD80, (B7-1), CD83, CD86 (B7-2), HVEM (CD270), ILT3 (immunoglobulin-like transcript 3), ILT4(immunoglobulin-like transcript 4), Fas ligand (FasL), ICAM (intercellular adhesion molecule), ICOS-L (inducible costimulatory ligand), JAG1 (CD339), lymphotoxin beta receptor, 3/TR6, OX40L (CD252), PD-L1, PD-L2, TGF-β1, TGF-β2, TGF-β3, and 4-1BBL polypeptide sequences.
133. The TMAPP-epitope conjugate or duplex TMAPP-epitope conjugate of any preceding aspect, wherein the additional MOD (wt. or variant) or the additional pair of MODs (wt. or variant) are selected independently from the group consisting of: 4-1BBL, PD-L1, IL-2, OX40L (CD252), ICOS-L, ICAM, CD30L, CD40, CD83, HVEM (CD270), JAG1 (CD339), CD70, CD80, and CD86, polypeptide sequences.
134. The TMAPP-epitope conjugate or duplex TMAPP-epitope conjugate of any preceding aspect, wherein the additional MOD (wt. or variant) or the additional pair of MODs (wt. or variant) are selected independently from the group consisting of IL-2, PD-L1, 4-1BBL polypeptide sequences and variants of any thereof. For example, The TMAPP-epitope conjugate or duplex TMAPP-epitope conjugate may comprise at least one IL-2 MOD (wt. or variant) and/or at least one PD-L1 (wt. or variant) polypeptide sequence(s).
135. The TMAPP-epitope conjugate or duplex TMAPP-epitope conjugate of any preceding aspect, wherein the additional MOD (wt. or variant) or the additional pair of MODs (wt. or variant) comprise at least one IL-2 MOD (wt. or variant) polypeptide sequence, or at least one pair of IL-2 MOD (wt. or variant) polypeptide sequences in tandem.
136. The TMAPP-epitope conjugate or duplex TMAPP-epitope conjugate of any preceding aspect, wherein the additional MOD (wt. or variant) or the additional pair of MODs (wt. or variant) comprise at least one PD-L1 MOD (wt. or variant) polypeptide sequence.
137. The TMAPP-epitope conjugate or duplex TMAPP-epitope conjugate of any preceding aspect, wherein the additional MOD (wt. or variant) or the additional pair of MODs (wt. or variant) comprise at least one 4-1BBL MOD (wt. or variant) polypeptide sequence.
138. The TMAPP-epitope conjugate or duplex TMAPP-epitope conjugate of any preceding aspect comprising an additional polypeptide, or a payload.
139. The TMAPP-epitope conjugate or duplex TMAPP-epitope conjugate of aspect 138, wherein the additional polypeptide is an affinity tag or a targeting sequence.
140. The TMAPP-epitope conjugate or duplex TMAPP-epitope conjugate of aspect 139, wherein the additional peptide is a targeting sequence selected from the group consisting of: antibody or antigen binding fragment/portion thereof (e.g., an scFv or a nanobody such as a heavy chain nanobody or a light chain nanobody).
141. The TMAPP-epitope conjugate or duplex TMAPP-epitope conjugate of aspect 139 or 140, wherein the targeting sequence is directed to a tissue affected by an autoimmune disease, an autoantigen, or allergen.
142. The TMAPP-epitope conjugate or duplex TMAPP-epitope conjugate of any preceding aspect, wherein the T1D-associated epitope is covalently bound through a linker to the chemical conjugation site.
143. The TMAPP-epitope conjugate or duplex TMAPP-epitope conjugate of any preceding aspect, wherein the T1D-associated peptide epitope is selected from an epitope of preproinsulin, proinsulin, insulin, insulin B chain, insulin A chain, 65 kDa isoform of glutamic acid decarboxylase (GAD65), 67 kDa isoform of glutamic acid decarboxylase (GAD67), tyrosine phosphatase (IA-2), heat-shock protein HSP65, islet-specific glucose6-phosphatase catalytic subunit related protein (IGRP), islet antigen 2 (IA2), and zinc transporter (ZnT8).
144. The TMAPP-epitope conjugate or duplex TMAPP-epitope conjugate of any of aspects 1-142, wherein the T1D-associated peptide epitope is selected from the group consisting of proinsulin 73-90 peptide GAGSLQPLALEGSLQKR SEQ ID NO:164), insulin (InsA (1-15) peptide GIVDQCCTSICSLYQ (SEQ ID NO:165), insulin (InsA(1-15; D4E) peptide GIVEQCCTSICSLYQ (SEQ ID NO:166), GAD65 (555-567) peptide NFFRMVISNPAAT (SEQ ID NO:167), GAD65 (555-567; F5571) peptide NFIRMVISNPAAT (SEQ ID NO:168), islet antigen 2 (IA2) peptide SFYLKNVQTQETRTLTQFHF (SEQ ID NO:169), proinsulin peptide SLQPLALEGSLQSRG (SEQ ID NO:170), InsB (9-23) peptide SHLVEALYLVCGERG (SEQ ID NO:206), GAD65 (121-140) peptide YVVKSFDRSTKVIDFHYPNE (SEQ ID NO:207), GAD65 (250-266) peptide AMMIARFKMFPEVKEKG (SEQ ID NO:208), Pro-insulin C-peptide (66-74) VELGGGPGA (SEQ ID NO:209), IGRP peptide VYLKTNVFLGGGAS (SEQ ID NO:210), and proinsulin peptide GSLQPLALEGSLQSRGIV (SEQ ID NO:171; proIns 75-92(K88S)).
145. The TMAPP, or higher order TMAPP (e.g., duplex TMAPP) of any of aspects 1-142, wherein the T1D-associated peptide comprises from 4 to 25 contiguous aas of an aa sequence having at least 90%, at least 95%, at least 98%, at least 99%, or 100%, aa sequence identity to aas 25-110 of the human preproinsulin aa sequence (wherein italicized aas 1-24 form the signal peptide):

(SEQ ID NO: 172)
MALWMRLLPL LALLALWGPD PAAA FVNQHL CGSHLVEALY
LVCGERGFFY TPKTRREAED LQVGQVELGG GPGAGSLQPL
ALEGSLQKRG IVEQCCTSIC SLYQLENYCN.

146. The TMAPP, or higher order TMAPP (e.g., duplex TMAPP) of any of aspects 1 to 142, wherein the T1D-associated peptide epitope has the aa sequence: GAGSLQPLALEGSLQKRG (SEQ ID NO:173) (proIns 73-90), SLQPLALEGSLQKRG (SEQ ID NO:174) (proIns 76-90), SLQPLALEGSLQSRG (SEQ ID NO:175) (proIns 76-90; K88S), QPLALEGSLQKRG (SEQ ID NO:176), or the sequence QPLALEGSLQSRG (SEQ ID NO:177).
147. The TMAPP-epitope conjugate or duplex TMAPP-epitope conjugate of any preceding aspect, wherein the peptide epitope is from about 4 aas (aa) to about 25 as (e.g., the epitope can have a length of from 4 aa to 10 aa, from about 6 aa to about 12 aa, from 8 aa to 20 aa, from 10 aa to 15 aa, from 10 as to 20 aa, from 15 aa to 20 aa, or from 20 aa to 25 aa).
148. The TMAPP-epitope conjugate or duplex TMAPP-epitope conjugate of any preceding aspect, wherein the peptide epitope is from about 8 aa to about 20 aa.
149. The TMAPP-epitope conjugate or duplex TMAPP-epitope conjugate of any of aspect 142-148, wherein the T1D-associated epitope and linker are covalently attached to a cysteine that has reacted with a maleimide of the linker. (See, e.g., FIG. 13.)
150. The TMAPP-epitope conjugate or duplex TMAPP-epitope conjugate of aspect 149, wherein the T1D-associated epitope and linker comprises the structure {epitope-(conjugation site)-aa1-aa2-aa3-aa4-aa5-[remainder of linker if present]}, wherein at least one of aa1 to aa5 is a linker cysteine, and wherein the linker cysteine forms a disulfide bond with a cysteine located in the α1 or α2, domain polypeptide sequences; and optionally, wherein the remainder of aa1 to aa5 are selected independently from Gly and Ser.
151. The TMAPP-epitope conjugate or duplex TMAPP-epitope conjugate of aspect 150, wherein the cysteine is located in the α1 domain polypeptide sequences.
152. The TMAPP-epitope conjugate or duplex TMAPP-epitope conjugate of aspect 151, wherein the cysteine is located in the α1 domain polypeptide sequence at one of positions 71 to 76.
153. The TMAPP-epitope conjugate or duplex TMAPP-epitope conjugate of aspect 151, wherein the cysteine is located in the α1 domain polypeptide sequence at one of positions 72 or 75.
154. The TMAPP-epitope conjugate or duplex TMAPP-epitope conjugate of any of aspects 150-153, wherein aa3 is the linker cysteine.
155. The TMAPP-epitope conjugate or duplex TMAPP-epitope conjugate of any of aspects 151 to 154, wherein when the presenting sequence comprises polypeptide having greater than 90% or greater than 95% sequence identity to aas 26-203 of the mature DRA polypeptide (see FIG. 4), the cysteine is located in the α1 domain polypeptide sequences is located at position 72 or 75 as the result of I72C or K75C substitution.
156. A pharmaceutical composition comprising one or more TMAPP-epitope conjugates, or duplex TMAPP-epitope conjugates of any preceding aspect.
157. A pharmaceutical composition comprising one or more duplex TMAPP-epitope conjugates of any one of aspects 1 to 155.
158. A method of treatment or prophylaxis of a patient or subject having Type 1 Diabetes (“T1D”), comprising administering to a patient or subject (e.g., a diabetic or prediabetic patient in need thereof) a pharmaceutical composition comprising an effective amount of one or more TMAPP-epitope conjugates, duplex T-MAPP-epitope conjugates, or higher order TMAPP-epitope conjugate complexes of any of aspects 1 to 155.
159. The method of aspect 158, wherein the treatment reduces hemoglobin AlC or blood sugar (e.g., glucose) levels in a diabetic or prediabetic patient or subject relative to the hemoglobin A1C or blood sugar levels prior to administration of the one or more TMAPP-epitope conjugates, duplex T-MAPP-epitope conjugates, or higher order TMAPP-epitope conjugate complexes.
160. The method of any of aspects 158 to 159, wherein the patient or subject is a mammalian patient or subject.
161. The method of any of aspects 158 to 160, wherein the patient or subject is human.
162. The method of any of aspects 158 to 160, wherein the patient or subject is non-human (e.g., rodent, lagomorph, bovine, canine, feline, rodent, murine, caprine, simian, ovine, equine, lapine, porcine, etc.).
163. A method of selectively delivering one or more MOD (wt. and/or variant) polypeptide sequences to a cell, tissue, patient or subject, the method comprising:
   (i) contacting (e.g., administering) a cell, tissue, patient or subject (e.g., a patient in need thereof) an effective amount of one or more TMAPP-epitope conjugate, duplex TMAPP-epitope conjugates, or higher order TMAPP-epitope conjugates of any of aspects 1 to 155, or a pharmaceutical composition comprising of aspect 156 or 157; or
   (ii) contacting a cell or tissue, either in vitro or in vivo, with one or more TMAPP-epitope conjugate, duplex TMAPP-epitope conjugates, or higher order TMAPP-epitope conjugates of any of aspects 1 to 155, or a pharmaceutical composition comprising of aspect 156 or 157, and optionally administering the cell, tissue, or progeny thereof to the patient/subject.

1. A TMAPP-epitope conjugate comprising:
(i) a presenting sequence or a presenting complex,
(ii) optionally at least one scaffold polypeptide sequence, and
(iii) a TGF-β sequence, a masking sequence that binds to a TGF-β sequence reversibly masking it, or at least one masked TGF-β MOD; wherein
(a) each presenting sequence comprises MHC Class II α1, α2, β1, and β2 domain polypeptide sequences;
(b) each presenting complex comprises a presenting complex first sequence and a presenting complex second sequence, wherein
the presenting complex first sequence and presenting complex second sequence comprises at least one of the α1, α2, β1, and β2 polypeptide sequences, and
the presenting complex first sequence and presenting complex second sequence together comprise the MHC Class II α1, α2, β1, and β2 domain polypeptide sequences, and
(c) each masked TGF-β MOD comprises a masking sequence and TGF-β sequence;
wherein the TMAPP-epitope conjugate optionally comprises one or more independently selected additional MODs (wt. and/or variant) or pairs of additional MODs (e.g., in tandem, both wt, both variant, or one wt. and one variant) (e.g., located at the N-terminus of a presenting sequence, presenting complex first and/or second sequences);
wherein a the TMAPP-epitope conjugate comprises a chemical conjugation site for conjugation of an epitope presenting molecule, and optionally comprises an additional chemical conjugation site for the conjugation of a payload, and wherein a T1D-associated epitope has been conjugated to the chemical conjugation site; and
wherein the TMAPP-epitope conjugate optionally comprises one or more linker sequences that are selected independently. (see, e.g., FIGS. 1A to 1H and FIGS. 10A to 10D).
It is understood that the amino acid sequence of any component (e.g., MHC-Class II polypeptide sequences) does not include an amino acid sequence that will anchor the TMAPP in a mammalian cell (e.g., a COS cell) membrane (e.g., the TMAPP does not comprise an MHC transmembrane domain, or a portion thereof, that will anchor the TMAPP in a cell membrane. The above components may or may not be arranged in the stated order from N-terminus to C-terminus in the TMAPP.

2. The TMAPP-epitope conjugate of claim 1, comprising from N-terminus to C-terminus:
(i) a presenting sequence or a presenting complex,
(ii) optionally at least one scaffold polypeptide sequence, and
(iii) a TGF-β sequence, a masking sequence that binds to a TGF-β sequence reversibly masking it, or at least one masked TGF-β MOD.

3. The TMAPP-epitope conjugate of claim 1 or 2, comprising:
(A) a presenting sequence that comprises, ordered from N-terminus to C-terminus
(i) the β1, β2, α1, and α2 domain polypeptide sequences,
(ii) the β1, α1, α2, and β2 domain polypeptide sequences, or
(iii) α1, α2, β1, and β2 domain polypeptide sequences; or
(B) a presenting complex wherein
(i): the presenting complex first sequence comprises, ordered from N-terminus to C terminus, the β1 and β2 domain polypeptide sequences; and the presenting complex second sequence comprises the α1, and α2 domain polypeptide sequences,
(ii) the presenting complex first sequence comprises, ordered from N-terminus to C terminus, the α1, and α2 domain polypeptide sequences; and the presenting complex second sequence comprises the β1 and β2 domain polypeptide sequences,
(iii) the presenting complex first sequence comprises, ordered from N-terminus to C terminus, the β1 α1, and α2 domain polypeptide sequences; and the presenting complex second sequence comprises the β2 domain polypeptide sequence,
(iv) the presenting complex first sequence comprises the β2 domain polypeptide sequence; and the presenting complex second sequence comprises, ordered from N-terminus to C terminus, the β1, α1, and α2 domain polypeptide sequences, or
(v) the presenting sequence or a presenting complex comprising a disulfide bond formed between one of MHC α1 or α2 domain polypeptide sequence and one of the β1 or β2 domain polypeptide sequences.

4. The TMAPP-epitope conjugate of any preceding claim, wherein the presenting sequence or a presenting complex comprises a disulfide bond formed between one of MHC α1 or α2 domain polypeptide sequence and one of the β1 or β2 domain polypeptide sequences.

5. The TMAPP-epitope conjugate of any preceding claim, wherein:
(i). the MHC class II α1 and α2 domain polypeptide sequences comprise human class II α1 and α2 domain polypeptide having at least 90% or at least 95% (e.g., at least 98% or 100%) sequence identity to an HLA DR alpha (DRA), DQ alpha 1 (DQA1), or DQ alpha 2 (DQA2) α1 and α2 domain polypeptide sequences; and
(ii). the MHC class II β1 and β2 domain polypeptide sequences comprises human class β1 and β2 domain polypeptide sequences having at least 90% or at least 95% (e.g., at least 98% or 100%) sequence identity to an HLA DR beta 1 (DRB1), DR beta 3 (DRB3), DR beta 4 (DRB4), DR beta 5 (DRB5), DQ beta 1 (DQB1), or DQ beta 2 (DQB2) β1 and β2 domain polypeptide sequences.

6. The TMAPP-epitope conjugate of any preceding claim, wherein:
(i) the α1 and α2 domain polypeptide sequences each have at least 90% or at least 95% (e.g., at least 98% or 100%) sequence identity to all or at least about 50 (e.g., at least about 60, 70, 80, 85, or 90) contiguous aas of the HLA DR alpha (DRA) α1 or α2 domain polypeptide sequence provided in FIG. 4; and
(ii) the β1 and β2 domain polypeptide sequences each have at least 90% or at least 95% (e.g., at least 98% or 100%) sequence identity to all or at least about 50 (e.g., at least about 60, 70, 80, 85, or 90) contiguous aas of an HLA DR beta 1 (DRB1), DR beta 3 (DRB3), DR beta 4 (DRB4), or DR beta 5 (DRB5) 01 or β2 domain polypeptide sequence provided in any one of FIG. 5, 6, 7, or 8.

7. The TMAPP-epitope conjugate of any preceding claim, wherein:
(i) the α1 and α2 domain polypeptide sequences each have at least 90% or at least 95% (e.g., at least 98% or 100%) sequence identity to all or at least about 50 contiguous aas of a HLA DRA*0101 or HLA DRA*0102 polypeptide sequence; and
(ii) the β1 and β2 domain polypeptide sequences each have at least 90% or at least 95% (e.g., at least 98% or 100%) sequence identity to at least about 60 contiguous aas of an DRB1*0301, DRB1*0401, DRB1*0402, DRB1*0405, DRB1*0801, or DRB1*0901 polypeptide sequence.

8. The TMAPP-epitope conjugate of any of claims 1-5, wherein:
(i) the α1 and α2 domain polypeptide sequences each have at least 90% or at least 95% (e.g., at least 98% or 100%) sequence identity to at least 60 contiguous aas of a HLA DQ alpha 1 or DQ alpha 2 (DQA1 or DQA2) polypeptide sequence; and
(ii) the β1 and β2 domain polypeptide sequences each have at least 90% or at least 95% (e.g., at least 98% or 100%) sequence identity to at least 60 contiguous aas of an HLA DQ beta 1 or DQ beta 2 (DQB1 or DQB2) polypeptide sequence.

9. The TMAPP-epitope conjugate of claim 5 wherein:
(i) the α1 and α2 domain polypeptide sequences each have at least 90% or at least 95% (e.g., at least 98% or 100%) sequence identity to at least 60 contiguous aas of a DQA1*0501 polypeptide sequence, and
β1 and β2 domain polypeptide sequences each have at least 90% or at least 95% (e.g., at least 98% or 100%) sequence identity to at least 60 contiguous aas of a DQB1*0201 polypeptide sequence; or
(ii) α1 and α2 domain polypeptide sequences each have at least 90% or at least 95% (e.g., at least 98% or 100%) sequence identity to at least 60 contiguous aas of a DQA1*0301 polypeptide sequence, and
β1 and β2 domain polypeptide sequences each have at least 90% or at least 95% (e.g., at least 98% or 100%) sequence identity to at least 60 contiguous aas of a DQB1*0302 polypeptide sequence.

10. The TMAPP-epitope conjugate of any preceding claim, wherein the presenting sequence or the presenting complex comprises a disulfide bond formed between one of MHC α1 or α2 domain polypeptide sequence and one of the β1 or β2 domain polypeptide sequences.

11. The TMAPP-epitope conjugate of any preceding claim, comprising a scaffold polypeptide sequence that is a non-interspecific sequence or interspecific sequence.

12. The TMAPP-epitope conjugate of claim 11, wherein the interspecific and non-interspecific sequence are selected from the group consisting of: immunoglobulin heavy chain constant regions (Ig Fc e.g., Ig CH2-CH3); collectin polypeptides, coiled-coil domains, leucine-zipper domains; Fos polypeptides; Jun polypeptides; Ig CH1; Ig C_{L} κ; Ig C_{L} λ; knob-in-hole without disulfide (“KiH”); knob-in hole with a stabilizing disulfide bond (“KiHs-s”); HA-TF; ZW-1; 7.8.60; DD-KK; EW-RVT; EW-RVTs-s; and Δ107 sequences.

13. The TMAPP-epitope conjugate of any of claims 11 to 12 complexed to form a duplex TMAPP-epitope conjugate or higher order TMAPP-epitope conjugate comprising at least a first TMAPP-epitope conjugate and a second TMAPP-epitope conjugate:
(i) the first TMAPP-epitope conjugate comprises a first scaffold polypeptide sequence; and
(ii) the second TMAPP-epitope conjugate comprises a second scaffold polypeptide sequence;
wherein the first TMAPP-epitope conjugate and the second TMAPP-epitope conjugate are associated by binding interactions between the first scaffold polypeptide sequence and second scaffold polypeptide sequence, and wherein the interactions optionally including one or more interchain covalent bonds (e.g., one or two disulfide bonds); and
wherein the duplex or higher order unconjugated TMAPP comprises at least one masked TGF-β MOD wherein the masking sequence and the TGF-β sequence are present in cis or in trans.

14. The TMAPP-epitope conjugate or duplex TMAPP-epitope conjugate of claim 46, wherein the first scaffold polypeptide sequence and the second scaffold polypeptide sequence are interspecific sequences.

15. The TMAPP-epitope conjugate or duplex TMAPP-epitope conjugate of any of claims 1 to 14, comprising a masked TGF-β MOD in trans, wherein the masking sequence is located at the N-terminus of the presenting complex frst sequence or the presenting complex second sequence, and the TGF-β sequence is located at the other of the presenting complex first sequence or the presenting complex second sequence.

16. The TMAPP-epitope conjugate or duplex TMAPP-epitope conjugate of any of claims 1 to 14, comprising a masked TGF-β MOD in trans, wherein the masking sequence is located at the C-terminus of the first scaffold polypeptide sequence, and the TGF-β sequence is located at the C-terminus of the second scaffold polypeptide sequence.

17. The TMAPP-epitope conjugate or duplex TMAPP-epitope conjugate of any preceding claim, comprising a masked TGF-β MOD in cis.

18. The TMAPP-epitope conjugate of any preceding claim, wherein the TGF-β sequence is:
(i) a TGF-β 1 polypeptide optionally comprising a substitution of C77;
(ii) a TGF-β2 polypeptide optionally comprising a substitution of C77; or
(iii) is a TGF-β3 polypeptide optionally comprising a substitution of C77.

19. The TMAPP-epitope conjugate or duplex TMAPP-epitope conjugate of any preceding claim, wherein the TGF-β sequence is a TGF-β3 polypeptide optionally comprising a substitution of C77.

20. The TMAPP-epitope conjugate or duplex TMAPP-epitope conjugate of any preceding claim, wherein at least one masking sequence is a TGF-β receptor (TβR) polypeptide, anti-TGF-β antibody or antibody-related polypeptide/aa sequence.

21. The TMAPP-epitope conjugate or duplex TMAPP-epitope conjugate of claim 20, wherein the TBR polypeptide comprises a TβRI, TβRII, or TβRIII aa sequence.

22. The TMAPP-epitope conjugate or duplex TMAPP-epitope conjugate of claim 21, wherein the TBR polypeptide comprises:
(i) a TβRII isoform A ectodomain aa sequence;
(ii) a TβRII isoform B ectodomain aa sequence;
(iii) a Tβ3RII isoform B Δ14 (14 aa N-terminal deletion) ectodomain aa sequence; or
(iv) a TβRII isoform B Δ25 (25 aa N-terminal deletion) ectodomain an sequence.

23. The TMAPP-epitope conjugate or duplex TMAPP-epitope conjugate of any preceding claim, wherein the chemical conjugation site or the additional chemical conjugation site is selected from the group consisting of:
a) an amino acid chemical conjugation site;
b) non-natural amino acids and/or selenocysteines;
c) a peptide sequence that acts as an enzyme modification sequence (e.g., sulfatase, transglutaminase or sortase sites);
d) carbohydrate or oligosaccharide; and
e) IgG nucleotide binding sites.

24. The TMAPP-epitope conjugate or duplex TMAPP-epitope conjugate of claim 23, wherein the amino acid chemical conjugation site is a cysteine.

25. The TMAPP-epitope conjugate or duplex TMAPP-epitope conjugate of any preceding claim, wherein the additional MOD (wt. or variant) or the additional pair of MODs (wt. or variant) are selected independently from the group consisting of IL-2, PD-L1, 4-1BBL polypeptide sequences and variants of any thereof. For example, the unconjugated TMAPP or unconjugated duplex TMAPP may comprise at least one IL-2 MOD (wt. or variant) and/or at least one PD-L1 (wt. or variant) polypeptide sequence(s).

26. The TMAPP-epitope conjugate or duplex TMAPP-epitope conjugate of any preceding claim, wherein the additional MOD (wt. or variant) or the additional pair of MODs (wt. or variant) comprise at least one IL-2 MOD (wt. or variant) polypeptide sequence, or at least one pair of IL-2 MOD (wt. or variant) polypeptide sequences in tandem.

27. The TMAPP-epitope conjugate or duplex TMAPP-epitope conjugate of any preceding claim, wherein the additional MOD (wt. or variant) or the additional pair of MODs (wt. or variant) comprise at least one PD-L1 MOD (wt. or variant) polypeptide sequence.

28. The TMAPP-epitope conjugate or duplex TMAPP-epitope conjugate of any preceding claim wherein the additional MOD (wt. or variant) or the additional pair of MODs (wt. or variant) comprise at least one 4-1BBL MOD (wt. or variant) polypeptide sequence.

29. The TMAPP-epitope conjugate or duplex TMAPP-epitope conjugate of any preceding claim, wherein the peptide epitope is from about 8 aa to about 20 aa.

30. The TMAPP-epitope conjugate or duplex TMAPP-epitope conjugate of claim 29, comprising an epitope covalently bound directly, or indirectly through a linker, to the chemical conjugation site to form a TMAPP-epitope conjugate or duplex TMAPP-epitope conjugate.

31. The TMAPP-epitope conjugate or duplex TMAPP-epitope conjugate of any preceding claim, wherein the T1D-associated peptide epitope is selected from an epitope of preproinsulin, proinsulin, insulin, insulin B chain, insulin A chain, 65 kDa isoform of glutamic acid decarboxylase (GAD65), 67 kDa isoform of glutamic acid decarboxylase (GAD67), tyrosine phosphatase (IA-2), heat-shock protein HSP65, islet-specific glucose6-phosphatase catalytic subunit related protein (IGRP), islet antigen 2 (IA2), and zinc transporter (ZnT8).

32. The TMAPP-epitope conjugate or duplex TMAPP-epitope conjugate of any of claims 1 to 30, wherein the T1D-associated peptide epitope is selected from the group consisting of proinsulin 73-90 peptide GAGSLQPLALEGSLQKR SEQ ID NO:164), insulin (InsA (1-15) peptide GIVDQCCTSICSLYQ (SEQ ID NO:165), insulin (InsA(I-15; D4E) peptide GIVEQCCTSICSLYQ (SEQ ID NO:166), GAD65 (555-567) peptide NFFRMVISNPAAT (SEQ ID NO:167), GAD65 (555-567; F557I) peptide NFIRMVISNPAAT (SEQ ID NO:168), islet antigen 2 (IA2) peptide SFYLKNVQTQETRTLTQFHF (SEQ ID NO:169), proinsulin peptide SLQPLALEGSLQSRG (SEQ ID NO:170), InsB (9-23) peptide SHLVEALYLVCGERG (SEQ ID NO:206), GAD65 (121-140) peptide YVVKSFDRSTKVIDFHYPNE (SEQ ID NO:207), GAD65 (250-266) peptide AMMIARFKMFPEVKEKG (SEQ ID NO:208), Pro-insulin C-peptide (66-74) VELGGGPGA (SEQ ID NO:209), IGRP peptide VYLKTNVFLGGGAS (SEQ ID NO:210), and proinsulin peptide GSLQPLALEGSLQSRGIV (SEQ ID NO:171; proIns 75-92(K88S)).

33. The TMAPP, or higher order TMAPP (e.g., duplex TMAPP) of any of claims 1 to 30, wherein the T1D-associated peptide comprises from 4 to 25 contiguous aas of an aa sequence having at least 90%, at least 95%, at least 98%, at least 99%, or 100%, an sequence identity to aas 25-110 of the human preproinsulin an sequence (wherein italicized aas 1-24 form the signal peptide):
(SEQ ID NO: 172)
MALWMRLLPL LALLALWGPD PAAA FVNQHL CGSHLVEALY
LVCGERGFFY TPKTRREAED LQVGQVELGG GPGAGSLQPL
ALEGSLQKRG IVEQCCTSIC SLYQLENYCN.

34. The TMAPP, or higher order TMAPP (e.g., duplex TMAPP) of any of claims 1 to 30, wherein the T1D-associated peptide epitope has the an sequence: GAGSLQPLALEGSLQKRG (SEQ ID NO:173) (proIns 73-90), SLQPLALEGSLQKRG (SEQ ID NO:174) (proIns 76-90), SLQPLALEGSLQSRG (SEQ ID NO:175) (proIns 76-90; K88S), QPLALEGSLQKRG (SEQ ID NO:176), or the sequence QPLALEGSLQSRG (SEQ ID NO:177).

35. A pharmaceutical composition comprising one or more TMAPP-epitope conjugates, or duplex TMAPP-epitope conjugates of any one of claims 1 to 34.

36. A method of treatment or prophylaxis of a patient or subject having Type 1 Diabetes (“T1D”), comprising administering to a patient or subject (e.g., a diabetic or prediabetic patient in need thereof) a pharmaceutical composition comprising an effective amount of one or more TMAPP-epitope conjugates, duplex T-MAPP-epitope conjugates, or higher order TMAPP-epitope conjugate complexes of any of claims 1 to 34.

37. The method of claim 36, wherein the treatment reduces hemoglobin A1C or blood sugar (e.g., glucose) levels in a diabetic or prediabetic patient or subject relative to the hemoglobin A1C or blood sugar levels prior to administration of the one or more TMAPP-epitope conjugates, duplex T-MAPP-epitope conjugates, or higher order TMAPP-epitope conjugate complexes.

38. The method of any of claims 36 to 37, wherein the patient or subject is a mammalian patient or subject.

39. The method of any of claims 36 to 38, wherein the patient or subject is human.

40. A method of selectively delivering one or more MOD (wt. and/or variant) polypeptide sequences to a cell, tissue, patient or subject, the method comprising:
(i) contacting (e.g., administering) a cell, tissue, patient or subject (e.g., a patient in need thereof) an effective amount of one or more TMAPP-epitope conjugate, duplex TMAPP-epitope conjugates, or higher order TMAPP-epitope conjugates of any of claims 1 to 34, or a pharmaceutical composition of claim 35; or
(ii) contacting a cell or tissue, either in vitro or in vivo, with one or more TMAPP-epitope conjugate, duplex TMAPP-epitope conjugates, or higher order TMAPP-epitope conjugates of any of claims 1 to 34, or a pharmaceutical composition comprising of claim 35, and optionally administering the cell, tissue, or progeny thereof to the patient/subject.


SEQUENCE LISTING (220 sequences)

SEQ ID NO: 1 - protein, 353 aa, Homo sapiens
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 2 - protein, 383 aa, Homo sapiens
PTKAPDVFPIISGCRHPKDNSPVVLACLITGYHPTSVTVTWYMGTQSQPQRTFPEIQRRDSYYMTSSQLSTPLQQWRQGEYKCVVQHTASKSKKEIFRWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPRSTTFWAWSVLRVPAPPSPQPATYTCVVSHEDSRTLLNASRSLEVSYVTDHGPMK

SEQ ID NO: 3 - protein, 222 aa, Homo sapiens
ADPCDSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGK

SEQ ID NO: 4 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 227 aa, Homo sapiens
DKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 227 aa, Homo sapiens
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 325 aa, Homo sapiens
STKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 10 - protein, 246 aa, Homo sapiens
HKPSNTKVDKRVELKTPLGDTTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 223 aa, Homo sapiens
PPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPG

SEQ ID NO: 13 - protein, 453 aa, Homo sapiens
GSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 14 - protein, 112 aa, Homo sapiens
FTVRETASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKT

SEQ ID NO: 15 - protein, 106 aa, Homo sapiens
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 106 aa, Homo sapiens
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 17 - protein, 254 aa, Homo sapiens
MAISGVPVLGFFIIAVLMSAQESWAIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETTENVVCALGLTVGLVGIIIGTIFIIKGLRKSNAAERRGPL

SEQ ID NO: 18 - protein, 266 aa, Homo sapiens
MVCLKLPGGSCMTALTVTLMVLSSPLALAGDTRPRFLWQLKFECHFFNGTERVRLLERCIYNQEESVRFDSDVGEYRAVTELGRPDAEYWNSQKDLLEQRRAAVDTYCRHNYGVGESFTVQRRVEPKVTVYPSKTQPLQHHNLLVCSVSGFYPGSIEVRWFRNGQEEKAGVVSTGLIQNGDWTFQTLVMLETVPRSGEVYTCQVEHPSVTSPLTVEWRARSESAQSKMLSGVGGFVLGLLFLGAGLFIYFRNQKGHSGLQPTGFLS

SEQ ID NO: 19 - protein, 266 aa, Homo sapiens [sequence omitted]

SEQ ID NO: 20 - protein, 266 aa, Homo sapiens [sequence omitted]

SEQ ID NO: 21 - protein, 266 aa, Homo sapiens
MVCLRLPGGSCMAVLTVTLMVLSSPLALAGDTRPRFLEYSTSECHFFNGTERVRYLDRYFHNQEENVRFDSDVGEFRAVTELGRPDAEYWNSQKDLLEQKRGRVDNYCRHNYGVVESFTVQRRVHPKVTVYPSKTQPLQHHNLLVCSVSGFYPGSIEVRWFRNGQEEKTGVVSTGLIHNGDWTFQTLVMLETVPRSGEVYTCQVEHPSVTSPLTVEWRARSESAQSKMLSGVGGFVLGLLFLGAGLFIYFRNQKGHSGLQPRGFLS

SEQ ID NO: 22 - protein, 266 aa, Homo sapiens [sequence omitted]

SEQ ID NO: 23 - protein, 266 aa, Homo sapiens [sequence omitted]

SEQ ID NO: 24 - protein, 266 aa, Homo sapiens [sequence omitted]

SEQ ID NO: 25 - protein, 266 aa, Homo sapiens [sequence omitted]

SEQ ID NO: 26 - protein, 266 aa, Homo sapiens
MVCLKFPGGSCMAALTVTLMVLSSPLALAGDTRPRFLEQVKHECHFFNGTERVRFLDRYFYHQEEYVRFDSDVGEYRAVTELGRPDAEYWNSQKDLLEQRRAEVDTYCRHNYGVVESFTVQRRVYPEVTVYPAKTQPLQHHNLLVCSVNGFYPGSIEVRWFRNGQEEKTGVVSTGLIQNGDWTFQTLVMLETVPRSGEVYTCQVEHPSLTSPLTVEWRARSESAQSKMLSGVGGFVLGLLFLGAGLFIYFRNQKGHSGLQPTGFLS

SEQ ID NO: 27 - protein, 266 aa, Homo sapiens [sequence omitted]

SEQ ID NO: 28 - protein, 266 aa, Homo sapiens [sequence omitted]

SEQ ID NO: 29 - protein, 266 aa, Homo sapiens [sequence omitted]

SEQ ID NO: 30 - protein, 266 aa, Homo sapiens [sequence omitted]

SEQ ID NO: 31 - protein, 266 aa, Homo sapiens
MVCLKLPGGSCMAALTVTLMVLSSPLALAGDTQPRFLWQGKYKCHFFNGTERVQFLERLFYNQEEFVRFDSDVGEYRAVTELGRPVAESWNSQKDILEDRRGQVDTVCRHNYGVGESFTVQRRVHPEVTVYPAKTQPLQHHNLLVCSVSGFYPGSIEVRWFRNGQEEKAGVVSTGLIQNGDWTFQTLVMLETVPRSGEVYTCQVEHPSVMSPLTVEWRARSESAQSKMLSGVGGFVLGLLFLGAGLFIYFRNQKGHSGLQPTGFLS

SEQ ID NO: 32 - protein, 266 aa, Homo sapiens [sequence omitted]

SEQ ID NO: 33 - protein, 266 aa, Homo sapiens [sequence omitted]

SEQ ID NO: 34 - protein, 266 aa, Homo sapiens [sequence omitted]

SEQ ID NO: 35 - protein, 266 aa, Homo sapiens [sequence omitted]

SEQ ID NO: 36 - protein, 266 aa, Homo sapiens [sequence omitted]

SEQ ID NO: 37 - protein, 266 aa, Homo sapiens [sequence omitted]

SEQ ID NO: 38 - protein, 266 aa, Homo sapiens
MVCLRLPGGSCMAVLTVTLMVLSSPLALAGDTRPRFLEYSTSECHFFNGTERVRFLDRYFYNQEEYVRFDSDVGEFRAVTELGRPDEEYWNSQKDFLEDERAAVDTYCRHNYGVVESFTVQRRVHPKVTVYPSKTQPLQHHNLLVCSVSGFYPGSIEVRWFRNGQEEKTGVVSTGLIHNGDWTFQTLVMLETVPRSGEVYTCQVEHPSVTSPLTVEWRARSESAQSKMLSGVGGFVLGLLFLGAGLFIYFRNQKGHSGLQPRGFLS

SEQ ID NO: 39 - protein, 266 aa, Homo sapiens [sequence omitted]

SEQ ID NO: 40 - protein, 266 aa, Homo sapiens [sequence omitted]

SEQ ID NO: 41 - protein, 266 aa, Homo sapiens [sequence omitted]

SEQ ID NO: 42 - protein, 266 aa, Homo sapiens [sequence omitted]

SEQ ID NO: 43 - protein, 266 aa, Homo sapiens [sequence omitted]

SEQ ID NO: 44 - protein, 266 aa, Homo sapiens [sequence omitted]

SEQ ID NO: 45 - protein, 266 aa, Homo sapiens [sequence omitted]

SEQ ID NO: 46 - protein, 266 aa, Homo sapiens [sequence omitted]

SEQ ID NO: 47 - protein, 266 aa, Homo sapiens
MVCLKLPGGSCMTALTVTLMVLSSPLALSGDTRPRFLWQPKRECHFFNGTERVRFLDRYFYNQEESVRFDSDVGEFRAVTELGRPDAEYWNSQKDILEQARAAVDTYCRHNYGVVESFTVQRRVQPKVTVYPSKTQPLQHHNLLVCSVSGFYPGSIEVRWFLNGQEEKAGMVSTGLIQNGDWTFQTLVMLETVPRSGEVYTCQVEHPSVTSPLTVEWRARSESAQSKMLSGVGGFVLGLLFLGAGLFIYFRNQKGHSGLQPTGFLS

SEQ ID NO: 48 - protein, 266 aa, Homo sapiens [sequence omitted]

SEQ ID NO: 49 - protein, 266 aa, Homo sapiens [sequence omitted]

SEQ ID NO: 50 - protein, 266 aa, Homo sapiens [sequence omitted]

SEQ ID NO: 51 - protein, 266 aa, Homo sapiens; identical to SEQ ID NO: 47

SEQ ID NO: 52 - protein, 266 aa, Homo sapiens; identical to SEQ ID NO: 47

SEQ ID NO: 53 - protein, 266 aa, Homo sapiens [sequence omitted]

SEQ ID NO: 54 - protein, 266 aa, Homo sapiens [sequence omitted]

SEQ ID NO: 55 - protein, 266 aa, Homo sapiens
MVCLKLPGGSSLAALTVTLMVLSSRLAFAGDTRPRFLELRKSECHFFNGTERVRYLDRYFHNQEEFLRFDSDVGEYRAVTELGRPVAESWNSQKDLLEQKRGRVDNYCRHNYGVGESFTVQRRVHPQVTVYPAKTQPLQHHNLLVCSVSGFYPGSIEVRWFRNGQEEKAGVVSTGLIQNGDWTFQTLVMLETVPRSGEVYTCQVEHPSVTSALTVEWRARSESAQSKMLSGVGGFVLGLLFLGAGLFIYFRNQKGHSGLQPTGFLS

SEQ ID NO: 56 - protein, 266 aa, Homo sapiens [sequence omitted]

SEQ ID NO: 57 - protein, 266 aa, Homo sapiens
MVCLKLPGGSSLAALTVTLMVLSSRLAFAGDTRPRFLELLKSECHFFNGTERVRFLERHFHNQEEYARFDSDVGEYRAVRELGRPDAEYWNSQKDLLEQKRGQVDNYCRHNYGVVESFTVQRRVHPQVTVYPAKTQPLQHHNLLVCSVSGFYPGSIEVRWFRNGQEEKAGVVSTGLIQNGDWTFQTLVMLETFPRSGEVYTCQVEHPSVTSPLTVEWSARSESAQSKMLSGVGGFVLGLLFLGAGLFIYFRNQKGHSGLQPTGFLS

SEQ ID NO: 58 - protein, 266 aa, Homo sapiens [sequence omitted]

SEQ ID NO: 59 - protein, 266 aa, Homo sapiens
MVCLKLPGGSCMAALTVTLTVLSSPLALAGDTQPRFLEQAKCECHFLNGTERVWNLIRYIYNQEEYARYNSDLGEYQAVTELGRPDAEYWNSQKDLLERRRAEVDTYCRYNYGVVESFTVQRRVQPKVTVYPSKTQPLQHHNLLVCSVNGFYPGSIEVRWFRNSQEEKAGVVSTGLIQNGDWTFQTLVMLETVPRSGEVYTCQVEHPSMMSPLTVQWSARSESAQSKMLSGVGGFVLGLLFLGTGLFIYFRNQKGHSGLQPTGLLS

SEQ ID NO: 60 - protein, 266 aa, Homo sapiens
MVCLKLPGGSCMAALTVTLTVLSSPLALAGDTQPRFLEQAKCECHFLNGTERVWNLIRYIYNQEEYARYNSDLGEYQAVTELGRPDAEYWNSQKDLLERRRAEVDTYCRYNYGVVESFTVQRRVQPKVTVYPSKTQPLQHHNLLVCSVNGFYPGSIEVRWFRNGQEEKAGVVSTGLIQNGDWTFQTLVMLETVPRSGEVYTCQVEHPSMMSPLTVQWSARSESAQSKMLSGVGGFVLGLLFLGTGLFIYFRNQKGHSGLQPTGLLS

SEQ ID NO: 61 - protein, 266 aa, Homo sapiens
MVCLKLPGGSYMAKLTVTLMVLSSPLALAGDTRPRFLQQDKYECHFFNGTERVRFLHRDIYNQEEDLRFDSDVGEYRAVTELGRPDAEYWNSQKDFLEDRRAAVDTYCRHNYGVGESFTVQRRVEPKVTVYPARTQTLQHHNLLVCSVNGFYPGSIEVRWFRNSQEEKAGVVSTGLIQNGDWTFQTLVMLETVPRSGEVYTCQVEHPSVTSPLTVEWRAQSESAQSKMLSGVGGFVLGLLFLGAGLFIYFKNQKGHSGLHPTGLVS

SEQ ID NO: 62 - protein, 159 aa, Homo sapiens
MKNHLLFWGVLAVFIKAVHVKAQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 63 - protein, 178 aa, Homo sapiens
IKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHW

SEQ ID NO: 64 - protein, 79 aa, Homo sapiens
VIIQAEFYLNPDQSGEFMFDFDGDEIFHVDMAKKETVWRLEEFGRFASFEAQGALANIAVDKANLEIMTKRSNYTPITN

SEQ ID NO: 65 - protein, 94 aa, Homo sapiens
VPPEVTVLTNSPVELREPNVLICFIDKFTPPVVNVTWLRNGKPVTTGVSETVFLPREDHLFRKFHYLPFLPSTEDVYDCRVEHWGLDEPLLKHW

SEQ ID NO: 66 - protein, 199 aa, Homo sapiens
GDTRPRFLEQVKHECHFFNGTERVRFLDRYFYHQEEYVRFDSDVGEYRAVTELGRPDAEYWNSQKDLLEQKRAAVDTYCRHNYGVGESFTVQRRVYPEVTVYPAKTQPLQHHNLLVCSVNGFYPASIEVRWFRNGQEEKTGVVSTGLIQNGDWTFQTLVMLETVPRSGEVYTCQVEHPSLTSPLTVEWRARSESAQSKM

SEQ ID NO: 67 - protein, 199 aa, Homo sapiens; identical to SEQ ID NO: 66

SEQ ID NO: 68 - protein, 94 aa, Homo sapiens
DTRPRFLEQVKHECHFFNGTERVRFLDRYFYHQEEYVRFDSDVGEYRAVTELGRPDAEYWNSQKDLLEQKRAAVDTYCRHNYGVGESFTVQRRV

SEQ ID NO: 69 - protein, 95 aa, Homo sapiens
GDTRCRFLEQVKHECHFFNGTERVRFLDRYFYHQEEYVRFDSDVGEYRAVTELGRPDAEYWNSQKDLLEQKRAAVDTYCRHNYGVGESFTVQRRV

SEQ ID NO: 70 - protein, 103 aa, Homo sapiens
YPEVTVYPAKTQPLQHHNLLVCSVNGFYPGSIEVRWFRNGQEEKTGVVSTGLIQNGDWTFQTLVMLETVPRSGEVYTCQVEHPSLTSPLTVEWRARSESAQSK

SEQ ID NO: 71 - protein, 104 aa, Homo sapiens
YPEVTVYPAKTQPLQHHNLLVCSVNGFYPASIEVRWFRNGQEEKTGVVSTGLIQNGDCTFQTLVMLETVPRSGEVYTCQVEHPSLTSPLTVEWRARSESAQSKM

SEQ ID NO: 72 - protein, 197 aa, Homo sapiens
DTRPRFLELRKSECHFFNGTERVRYLDRYFHNQEEFLRFDSDVGEYRAVTELGRPVAESWNSQKDLLEQKRGRVDNYCRHNYGVGESFTVQRRVHPQVTVYPAKTQPLQHHNLLVCSVSGFYPGSIEVRWFRNGQEEKAGVVSTGLIQNGDWTFQTLVMLETVPRSGEVYTCQVEHPSVTSALTVEWRARSESAQSK

SEQ ID NO: 73 - protein, 29 aa, Homo sapiens
MVCLKLPGGSSLAALTVTLMVLSSRLAFA

SEQ ID NO: 74 - protein, 94 aa, Homo sapiens
DTRPRFLELRKSECHFFNGTERVRYLDRYFHNQEEFLRFDSDVGEYRAVTELGRPVAESWNSQKDLLEQKRGRVDNYCRHNYGVGESFTVQRRV

SEQ ID NO: 75 - protein, 103 aa, Homo sapiens
HPQVTVYPAKTQPLQHHNLLVCSVSGFYPGSIEVRWFRNGQEEKAGVVSTGLIQNGDWTFQTLVMLETVPRSGEVYTCQVEHPSVTSALTVEWRARSESAQSK

SEQ ID NO: 76 - protein, 19 aa, Homo sapiens
MVCLKLPGGSCMAALTVTL

SEQ ID NO: 77 - protein, 105 aa, Homo sapiens
TVLSSPLALAGDTQPRFLEQAKCECHFLNGTERVWNLIRYIYNQEEYARYNSDLGEYQAVTELGRPDAEYWNSQKDLLERRRAEVDTYCRYNYGVVESFTVQRRV

SEQ ID NO: 78 - protein, 103 aa, Homo sapiens
QPKVTVYPSKTQPLQHHNLLVCSVNGFYPGSIEVRWFRNGQEEKAGVVSTGLIQNGDWTFQTLVMLETVPRSGEVYTCQVEHPSMMSPLTVQWSARSESAQSK

SEQ ID NO: 79 - protein, 105 aa, Homo sapiens
MVLSSPLALAGDTRPRFLQQDKYECHFFNGTERVRFLHRDIYNQEEDLRFDSDVGEYRAVTELGRPDAEYWNSQKDFLEDRRAAVDTYCRHNYGVGESFTVQRRV

SEQ ID NO: 80 - protein, 102 aa, Homo sapiens
EPKVTVYPARTQTLQHHNLLVCSVNGFYPGSIEVRWFRNSQEEKAGVVSTGLIQNGDWTFQTLVMLETVPRSGEVYTCQVEHPSVTSPLTVEWRAQSESAQS

SEQ ID NO: 81 - protein, 19 aa, Homo sapiens
MVCLKLPGGSYMAKLTVTL

SEQ ID NO: 82 - protein, 5 aa, Artificial Sequence
  Peptide suitable for use as a linker.
GGGGS

SEQ ID NO: 83 - protein, 20 aa, Artificial Sequence
  Peptide suitable for use as a linker.
GCGGSGGGGSGGGGSGGGGS

SEQ ID NO: 84 - protein, 33 aa, Homo sapiens
RMKQIEDKIEEILSKIYHIENEIARIKKLIGER

SEQ ID NO: 85 - protein, 32 aa, Homo sapiens
LSSIEKKQEEQTSWLIWISNELTLIRNELAQS

SEQ ID NO: 86 - protein, 31 aa, Homo sapiens
LSSIEKKLEEITSQLIQISNELTLIRNELAQ

SEQ ID NO: 87 - protein, 31 aa, Homo sapiens
LSSIEKKLEEITSQLIQIRNELTLIRNELAQ

SEQ ID NO: 88 - protein, 31 aa, Homo sapiens
LSSIEKKLEEITSQLQQIRNELTLIRNELAQ

SEQ ID NO: 89 - protein, 31 aa, Homo sapiens
LSSLEKKLEELTSQLIQLRNELTLLRNELAQ

SEQ ID NO: 90 - protein, 31 aa, Homo sapiens
ISSLEKKIEELTSQIQQLRNEITLLRNEIAQ

SEQ ID NO: 91 - protein, 47 aa, Homo sapiens
LEIEAAFLERENTALETRVAELRQRVQRLRNRVSQYRTRYGPLGGGK

SEQ ID NO: 92 - protein, 31 aa, Homo sapiens
LKSVENRLAVVENQLKTVIEELKTVKDLLSN

SEQ ID NO: 93 - protein, 31 aa, Homo sapiens
LARIEEKLKTIKAQLSEIASTLNMIREQLAQ

SEQ ID NO: 94 - protein, 31 aa, Homo sapiens
VSRLEEKVKTLKSQVTELASTVSLLREQVAQ

SEQ ID NO: 95 - protein, 30 aa, Homo sapiens
IQSEKKIEDISSLIGQIQSEITLIRNEIAQ

SEQ ID NO: 96 - protein, 31 aa, Homo sapiens
LMSLEKKLEELTQTLMQLQNELSMLKNELAQ

SEQ ID NO: 97 - protein, 18 aa, Homo sapiens
VDLEGSTSNGRQCAGIRL

SEQ ID NO: 98 - protein, 27 aa, Homo sapiens
EDDVTTTEELAPALVPPPKGTCAGWMA

SEQ ID NO: 99 - protein, 27 aa, Homo sapiens
GHDQETTTQGPGVLLPLPKGACTGQMA

SEQ ID NO: 100 - protein, 64 aa, Homo sapiens
VTAFSNMDDMLQKAHLVIEGTFIYLRDSTEFFIRVRDGWKKLQLGELIPIPADSPPPPALSSNP

SEQ ID NO: 101 - protein, 112 aa, Homo sapiens
ALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 102 - protein, 112 aa, Homo sapiens
ALDTNYCFSSTEKNCCVRQLYIDFRKDLGWKWIHEPKGYHANFCLGPCPYIWSLDTQYSKVLALYNQHNPGASAAPSCVPQALEPLPIVYYVGRKPKVEQLSNMIVRSCKCS

SEQ ID NO: 103 - protein, 112 aa, Homo sapiens
ALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 104 - protein, 112 aa, Homo sapiens
ALDAAYCFRNVQDNCCLRPLYIDFKRDLGWKWIHEPKGYNANFCAGACPYLWSSDTQHSRVLSLYNTINPEASASPSCVSQDLEPLTILYYIGKTPKIEQLSNMIVKSCKCS

SEQ ID NO: 105 - protein, 112 aa, Homo sapiens [sequence omitted]

SEQ ID NO: 106 - protein, 112 aa, Homo sapiens
ALDTNYCFRNLEENCCVRPLYIDFRQDLGWKWVHEPKGYYANFCSGPCPYLRSADTTHSTVLGLYNTLNPEASASPSCVPQDLEPLTILYYVGRTPKVEQLSNMVVKSCKCS

SEQ ID NO: 107 - protein, 93 aa, Homo sapiens
LQCFCHLCTKDNFTCVTDGLCFVSVTETTDKVIHNSMCIAEIDLIPRDRPFVCAPSSKTGSVTTTYCCNQDHCNKIELPTTVKSSPGLGPVEL

SEQ ID NO: 108 - protein, 154 aa, Homo sapiens
IPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEE

SEQ ID NO: 109 - protein, 143 aa, Homo sapiens
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQ

SEQ ID NO: 110 - protein, 129 aa, Homo sapiens
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEE

SEQ ID NO: 111 - protein, 114 aa, Homo sapiens
TDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEE

SEQ ID NO: 112 - protein, 104 aa, Homo sapiens
QLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEE

SEQ ID NO: 113 - protein, 112 aa, Homo sapiens
SQLCKFCDVRFSTCDNQKSCMSNCSITSICEKPQEVCVAVWRKNDENITLETVCHDPKLPYHDFILEDAASPKCIMKEKKKPGETFFMCSCSSAECNDNIIFSEEYNTSNPD

SEQ ID NO: 114 - protein, 112 aa, Homo sapiens; identical to SEQ ID NO: 106

SEQ ID NO: 115 - protein, 133 aa, Homo sapiens [sequence omitted]

SEQ ID NO: 116 - protein, 251 aa, Homo sapiens
ELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQVAVAGCVFLLISVLLLSGLTWQRRQRKSRRTI

SEQ ID NO: 117 - protein, 524 aa, Homo sapiens
VNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 118 - protein, 347 aa, Homo sapiens
LNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 119 - protein, 245 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPGNILNVSIKICLTLSPST

SEQ ID NO: 120 - protein, 219 aa, Homo sapiens
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPGNILNVSIKI

SEQ ID NO: 121 - protein, 227 aa, Homo sapiens
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERLNVSIKI

SEQ ID NO: 122 - protein, 124 aa, Homo sapiens
AFTVTVPKDLYVVEYGSNMTIECKFPVEKQLDLAALIVYWEMEDKNIIQFVHGEEDLKTQHSSYRQRARLLKDQLSLGNAALQITDVKLQDAGVYRCMISYGGADYKRITVKVNAPYAAALHEH

SEQ ID NO: 123 - protein, 268 aa, Homo sapiens
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 124 - protein, 254 aa, Homo sapiens
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 125 - protein, 174 aa, Homo sapiens
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 126 - protein, 175 aa, Homo sapiens
DPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 127 - protein, 167 aa, Homo sapiens
DPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPA

SEQ ID NO: 128 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 129 - protein, 5 aa, Artificial Sequence
  Peptide suitable for use as a linker.
GSGGS

SEQ ID NO: 130 - protein, 4 aa, Artificial Sequence
  Peptide suitable for use as a linker.
GGGS

SEQ ID NO: 131 - protein, 4 aa, Artificial Sequence
  Peptide suitable for use as a linker.
GGSG

SEQ ID NO: 132 - protein, 5 aa, Artificial Sequence
  Peptide suitable for use as a linker.
GGSGG

SEQ ID NO: 133 - protein, 5 aa, Artificial Sequence
  Peptide suitable for use as a linker.
GSGSG

SEQ ID NO: 134 - protein, 5 aa, Artificial Sequence
  Peptide suitable for use as a linker.
GSGGG

SEQ ID NO: 135 - protein, 5 aa, Artificial Sequence
  Peptide suitable for use as a linker.
GGGSG

SEQ ID NO: 136 - protein, 5 aa, Artificial Sequence
  Peptide suitable for use as a linker.
GSSSG

SEQ ID NO: 137 - protein, 5 aa, Artificial Sequence
  Peptide suitable for use as a linker.
GSSSS

SEQ ID NO: 138 - protein, 5 aa, Artificial Sequence
  Peptide suitable for use as a linker.
AAAGG

SEQ ID NO: 139 - protein, 5 aa, Artificial Sequence
  Peptide suitable for use as a linker. The EAAAK may be repeated 2 to 20 times.
EAAAK

SEQ ID NO: 140 - protein, 7 aa, Artificial Sequence
  Peptide suitable for use as a linker. The EAAAK subset may be repeated 2 to 20 times.
AEAAAKA

SEQ ID NO: 141 - protein, 16 aa, Artificial Sequence
  Peptide suitable for use as a linker. The EAAAK subset may be repeated independently 2 to 20 times.
AEAAAKALEAEAAAKA

SEQ ID NO: 142 - protein, 5 aa, Artificial Sequence
  Peptide suitable for use as a linker
EAAAK

SEQ ID NO: 143 - protein, 10 aa, Artificial Sequence
  Peptide suitable for use as a linker.
EAAAKEAAAK

SEQ ID NO: 144 - protein, 15 aa, Artificial Sequence
  Peptide suitable for use as a linker.
EAAAKEAAAKEAAAK

SEQ ID NO: 145 - protein, 46 aa, Artificial Sequence
  Peptide suitable for use as a linker.
AEAAAKEAAAKEAAAKEAAAKALEAEAAAKEAAAKEAAAKEAAAKA

SEQ ID NO: 146 - protein, 12 aa, Artificial Sequence
  Peptide suitable for use as a linker.
AEAAAKEAAAKA

SEQ ID NO: 147 - protein, 5 aa, Artificial Sequence
  Peptide suitable for use as a linker.
PAPAP

SEQ ID NO: 148 - protein, 8 aa, Artificial Sequence
  Peptide suitable for use as a linker.
APAPAPAP

SEQ ID NO: 149 - protein, 12 aa, Artificial Sequence
  Peptide suitable for use as a linker.
APAPAPAPAPAP

SEQ ID NO: 150 - protein, 16 aa, Artificial Sequence
  Peptide suitable for use as a linker.
APAPAPAPAPAPAPAP

SEQ ID NO: 151 - protein, 20 aa, Artificial Sequence
  Peptide suitable for use as a linker.
APAPAPAPAPAPAPAPAPAP

SEQ ID NO: 152 - protein, 4 aa, Artificial Sequence
  Peptide suitable for use as a linker.
KPKP

SEQ ID NO: 153 - protein, 8 aa, Artificial Sequence
  Peptide suitable for use as a linker.
KPKPKPKP

SEQ ID NO: 154 - protein, 12 aa, Artificial Sequence
  Peptide suitable for use as a linker.
KPKPKPKPKPKP

SEQ ID NO: 155 - protein, 16 aa, Artificial Sequence
  Peptide suitable for use as a linker.
KPKPKPKPKPKPKPKP

SEQ ID NO: 156 - protein, 20 aa, Artificial Sequence
  Peptide suitable for use as a linker.
KPKPKPKPKPKPKPKPKPKP

SEQ ID NO: 157 - protein, 4 aa, Artificial Sequence
  Peptide suitable for use as a linker.
EPEP

SEQ ID NO: 158 - protein, 8 aa, Artificial Sequence
  Peptide suitable for use as a linker.
EPEPEPEP

SEQ ID NO: 159 - protein, 12 aa, Artificial Sequence
  Peptide suitable for use as a linker.
EPEPEPEPEPEP

SEQ ID NO: 160 - protein, 16 aa, Artificial Sequence
  Peptide suitable for use as a linker.
EPEPEPEPEPEPEPEP

SEQ ID NO: 161 - protein, 20 aa, Artificial Sequence
  Peptide suitable for use as a linker.
EPEPEPEPEPEPEPEPEPEP

SEQ ID NO: 162 - protein, 15 aa, Artificial Sequence
  Linker
GCGASGGGGSGGGGS

SEQ ID NO: 163 - protein, 15 aa, Artificial Sequence
  Peptide suitable for use as a linker.
GCGGSGGGGSGGGGS

SEQ ID NO: 164 - protein, 17 aa, Homo sapiens
GAGSLQPLALEGSLQKR

SEQ ID NO: 165 - protein, 15 aa, Homo sapiens
GIVDQCCTSICSLYQ

SEQ ID NO: 166 - protein, 15 aa, Homo sapiens
GIVEQCCTSICSLYQ

SEQ ID NO: 167 - protein, 13 aa, Homo sapiens
NFFRMVISNPAAT

SEQ ID NO: 168 - protein, 13 aa, Homo sapiens
NFIRMVISNPAAT

SEQ ID NO: 169 - protein, 20 aa, Homo sapiens
SFYLKNVQTQETRTLTQFHF

SEQ ID NO: 170 - protein, 15 aa, Homo sapiens
SLQPLALEGSLQSRG

SEQ ID NO: 171 - protein, 18 aa, Homo sapiens
GSLQPLALEGSLQSRGIV

SEQ ID NO: 172 - protein, 110 aa, Homo sapiens
MALWMRLLPLLALLALWGPDPAAAFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 173 - protein, 18 aa, Homo sapiens
GAGSLQPLALEGSLQKRG

SEQ ID NO: 174 - protein, 15 aa, Homo sapiens
SLQPLALEGSLQKRG

SEQ ID NO: 175 - protein, 15 aa, Homo sapiens
SLQPLALEGSLQSRG

SEQ ID NO: 176 - protein, 13 aa, Homo sapiens
QPLALEGSLQKRG

SEQ ID NO: 177 - protein, 13 aa, Homo sapiens
QPLALEGSLQSRG

SEQ ID NO: 178 - protein, 9 aa, Artificial Sequence
  Epitope tag hemagglutinin
YPYDVPDYA

SEQ ID NO: 179 - protein, 8 aa, Artificial Sequence
  Epitope tag FLAG
DYKDDDDK

SEQ ID NO: 180 - protein, 10 aa, Artificial Sequence
  Epitope tag c-myc
EQKLISEEDL

SEQ ID NO: 181 - protein, 5 aa, Homo sapiens
HHHHH

SEQ ID NO: 182 - protein, 6 aa, Homo sapiens
HHHHHH

SEQ ID NO: 183 - protein, 8 aa, Artificial Sequence
  Strep Tag
WSHPQFEK

SEQ ID NO: 184 - protein, 5 aa, Artificial Sequence
  Affinity Tag
RYIRS

SEQ ID NO: 185 - protein, 4 aa, Artificial Sequence
  Affinity Tag
FHHT

SEQ ID NO: 186 - protein, 17 aa, Artificial Sequence
  Affinity Tag
WEAAAREACCRECCARA

SEQ ID NO: 187 - protein, 6 aa, Artificial Sequence
  sulfatase motif
  VARIANT (1)..(1): Position 1 (X1) is present or absent and, when present, can be any amino acid, usually L, M, V, S or T, more usually L, M, S or V, with the proviso that, when the sulfatase motif is at the N-terminus of the target polypeptide, X1 is present.
  VARIANT (2)..(2): Position 2 (Z1) is cysteine or serine
  VARIANT (3)..(3): Position 3 (X2) can be any amino acid, though usually an aliphatic aa, a polar, uncharged aa, or a sulfur containing amino acid (e.g., other than an aromatic amino acid or a charged amino acid), usually S, T, A, V, G or C, more usually S, T, A, V or G.
  VARIANT (4)..(4): Position 4 (Z2) is either a proline or alanine residue (which can also be represented by “P/A”)
  VARIANT (5)..(5): Position 5 (X3) can be any amino acid, though usually an aliphatic aa, a polar, uncharged aa, or a sulfur containing amino acid (e.g., other than an aromatic amino acid or a charged amino acid), usually S, T, A, V, G or C, more usually S, T, A, V or G.
  VARIANT (6)..(6): Position (Z3) is a basic amino acid (arginine, lysine, or histidine, usually lysine), or an aliphatic aa (alanine, glycine, leucine, valine, isoleucine, or proline, usually A, G, L, V, or I).
XXXXXX

SEQ ID NO: 188 - protein, 6 aa, Artificial Sequence
  sulfatase motif
  VARIANT (1)..(1): Position 1 (X1) is present or absent and, when present, can be any aa, usually L, M, V, S or T, more usually L, M, S or V, with the proviso that, when the sulfatase motif is at the N-terminus of the target polypeptide, X1 is present.
  VARIANT (3)..(3): Position (X2) can be any amino acid, though usually an aliphatic aa, a polar, uncharged aa, or a sulfur containing amino acid (e.g., other than an aromatic amino acid or a charged amino acid), usually S, T, A, V, G or C, more usually S, T, A, V or G.
  VARIANT (5)..(5): Position 5 (X3) can be any amino acid, though usually an aliphatic aa, a polar, uncharged aa, or a sulfur containing amino acid (e.g., other than an aromatic amino acid or a charged amino acid), usually S, T, A, V, G or C, more usually S, T, A, V or G.
  VARIANT (6)..(6): Position 6 (Z3) is a basic amino acid (arginine, lysine, or histidine, usually lysine), or an aliphatic aa (alanine, glycine, leucine, valine, isoleucine, or proline, usually A, G, L, V, or I).
XCXPXX

SEQ ID NO: 189 - protein, 5 aa, Artificial Sequence
  Sulfatase motif
  VARIANT (2)..(2): Position 2 (X1) is present or absent and, when present, can be any aa, usually L, M, V, S or T, more usually L, M, S or V, with the proviso that, when the sulfatase motif is at the N-terminus of the target polypeptide, X1 is present.
  VARIANT (4)..(4): Position 4 (X2) can be any amino acid, though usually an aliphatic aa, a polar, uncharged aa, or a sulfur containing amino acid (e.g., other than an aromatic amino acid or a charged amino acid), usually S, T, A, V, G or C, more usually S, T, A, V or G.
  VARIANT (5)..(5): Position 5 (Z3) is a basic amino acid (arginine, lysine, or histidine, usually lysine), or an aliphatic aa (alanine, glycine, leucine, valine, isoleucine, or proline, usually A, G, L, V, or I).
CXPXX

SEQ ID NO: 190 - protein, 5 aa, Artificial Sequence
  Transglutaminase Enzyme Site
LLQGG

SEQ ID NO: 191 - protein, 4 aa, Artificial Sequence
  Transglutaminase Enzyme Site
LLQG

SEQ ID NO: 192 - protein, 6 aa, Artificial Sequence
  Transglutaminase Enzyme Site
LSLSQG

SEQ ID NO: 193 - protein, 6 aa, Artificial Sequence
  Transglutaminase Enzyme Site
LLQLQG

SEQ ID NO: 194 - protein, 5 aa, Artificial Sequence
  Sortase A Enzyme Site
  SITE (3)..(3): X5 can be any single amino acid.
  SITE (5)..(5): X can be a glycine or alanine.
LPXTX

SEQ ID NO: 195 - protein, 5 aa, Artificial Sequence
  Sortase enzyme conjugation partner sequence
GGGGG

SEQ ID NO: 196 - protein, 4 aa, Artificial Sequence
  Sortase enzyme conjugation partner sequence
GGGG

SEQ ID NO: 197 - protein, 5 aa, Artificial Sequence
  Sortase enzyme conjugation partner sequence
AAAAA

SEQ ID NO: 198 - protein, 4 aa, Artificial Sequence
  Sortase enzyme conjugation partner sequence
AAAA

SEQ ID NO: 199 - protein, 6 aa, Artificial Sequence
  Sortase A Enzyme Site
LPETGG

SEQ ID NO: 200 - protein, 6 aa, Artificial Sequence
  Sortase A Enzyme Site
LPETAA

SEQ ID NO: 201 - protein, 8 aa, Artificial Sequence
  Peptide suitable for use as a proteolytically cleavable linker.
LEVLFQGP

SEQ ID NO: 202 - protein, 7 aa, Artificial Sequence
  Peptide suitable for use as a proteolytically cleavable linker.
ENLYTQS

SEQ ID NO: 203 - protein, 5 aa, Artificial Sequence
  Peptide suitable for use as a proteolytically cleavable linker.
DDDDK

SEQ ID NO: 204 - protein, 4 aa, Artificial Sequence
  Peptide suitable for use as a proteolytically cleavable linker.
LVPR

SEQ ID NO: 205 - protein, 22 aa, Artificial Sequence
  Peptide suitable for use as a proteolytically cleavable linker.
GSGATNFSLLKQAGDVEENPGP

SEQ ID NO: 206 - protein, 15 aa, Homo sapiens
SHLVEALYLVCGERG

SEQ ID NO: 207 - protein, 20 aa, Homo sapiens
YVVKSFDRSTKVIDFHYPNE

SEQ ID NO: 208 - protein, 17 aa, Homo sapiens
AMMIARFKMFPEVKEKG

SEQ ID NO: 209 - protein, 9 aa, Homo sapiens
VELGGGPGA

SEQ ID NO: 210 - protein, 14 aa, Homo sapiens
VYLKTNVFLGGGAS

SEQ ID NO: 211 - protein, 390 aa, Homo sapiens
MPPSGLRLLPLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 212 - protein, 413 aa, Homo sapiens
MHYCVLSAFLILHLVTVALSLSTCSTLDMDQFMRKRIEAIRGQILSKLKLTSPPEDYPEPEEVPPEVISIYNSTRDLLQEKASRRAAACERERSDEEYYAKEVYKIDMPPFFPSEAIPPTFYRPYFRIVRFDVSAMEKNASNLVKAEFRVFRLQNPKARVPEQRIELYQILKSKDLTSPTQRYIDSKVVKTRAEGEWLSFDVTDAVHEWLHHKDRNLGFKISLHCPCCTFVPSNNYIIPNKSEELEARFAGIDGTSTYTSGDQKTIKSTRKKNSGKTPHLLLMLLPSYRLESQQTNRRKKRALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 213 - protein, 412 aa, Homo sapiens
MKMHLQRALVVLALLNFATVSLSLSTCTTLDFGHIKKKRVEAIRGQILSKLRLTSPPEPTVMTHVPYQVLALYNSTRELLEEMHGEREEGCTQENTESEYYAKEIHKFDMIQGLAEHNELAVCPKGITSKVFRFNVSSVEKNRTNLFRAEFRVLRVPNPSSKRNEQRIELFQILRPDEHIAKQRYIGGKNLPTRGTAEWLSFDVTDTVREWLLRRESNLGLEISIHCPCHTFQPNGDILENIHEVMEIKFKGVDNEDDHGRGDLGRLKKQKDHHNPHLILMMIPPHRLDNPGQGGQRKKRALDTNYCFRNLEENCCVRPLYIDFRQDLGWKWVHEPKGYYANFCSGPCPYLRSADTTHSTVLGLYNTLNPEASASPCCVPQDLEPLTILYYVGRTPKVEQLSNMVVKSCKCS

SEQ ID NO: 214 - protein, 503 aa, Homo sapiens
MEAAVAAPRPRLLLLVLAAAAAAAAALLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVELAAVIAGPVCFVCISLMLMVYICHNRTVIHHRVPNEEDPSLDRPFISEGTTLKDLIYDMTTSGSGSGLPLLVQRTIARTIVLQESIGKGRFGEVWRGKWRGEEVAVKIFSSREERSWFREAEIYQTVMLRHENILGFIAADNKDNGTWTQLWLVSDYHEHGSLFDYLNRYTVTVEGMIKLALSTASGLAHLHMEIVGTQGKPAIAHRDLKSKNILVKKNGTCCIADLGLAVRHDSATDTIDIAPNHRVGTKRYMAPEVLDDSINMKHFESFKRADIYAMGLVFWEIARRCSIGGIHEDYQLPYYDLVPSDPSVEEMRKVVCEQKLRPNIPNRWQSCEALRVMAKIMRECWYANGAARLTALRIKKTLSQLSQQEGIKM

SEQ ID NO: 215 - protein, 592 aa, Homo sapiens
MGRGLLRGLWPLHIVLWTRIASTIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 216 - protein, 567 aa, Homo sapiens
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 217 - protein, 111 aa, Homo sapiens
QLCKFCDVRFSTCDNQKSCMSNCSITSICEKPQEVCVAVWRKNDENITLETVCHDPKLPYHDFILEDAASPKCIMKEKKKPGETFFMCSCSSAECNDNIIFSEEYNTSNPD

SEQ ID NO: 218 - protein, 851 aa, Homo sapiens
MTSHYVIAIFALMSSCLATAGPEPGALCELSPVSASHPVQALMESFTVLSGCASRGTTGLPQEVHVLNLRTAGQGPGQLQREVTLHLNPISSVHIHHKSVVFLLNSPHPLVWHLKTERLATGVSRLFLVSEGSVVQFSSANFSLTAETEERNFPHGNEHLLNWARKEYGAVTSFTELKIARNIYIKVGEDQVFPPKCNIGKNFLSLNYLAEYLQPKAAEGCVMSSQPQNEEVHIIELITPNSNPYSAFQVDITIDIRPSQEDLEVVKNLILILKCKKSVNWVIKSFDVKGSLKIIAPNSIGFGKESERSMTMTKSIRDDIPSTQGNLVKWALDNGYSPITSYTMAPVANRFHLRLENNAEEMGDEEVHTIPPELRILLDPGALPALQNPPIRGGEGQNGGLPFPFPDISRRVWNEEGEDGLPRPKDPVIPSIQLFPGLREPEEVQGSVDIALSVKCDNEKMIVAVEKDSFQASGYSGMDVTLLDPTCKAKMNGTHFVLESPLNGCGTRPRWSALDGVVYYNSIVIQVPALGDSSGWPDGYEDLESGDNGFPGDMDEGDASLFTRPEIVVFNCSLQQVRNPSSFQEQPHGNITFNMELYNTDLFLVPSQGVFSVPENGHVYVEVSVTKAEQELGFAIQTCFISPYSNPDRMSHYTIIENICPKDESVKFYSPKRVHFPIPQADMDKKRFSFVFKPVFNTSLLFLQCELTLCTKMEKHPQKLPKCVPPDEACTSLDASIIWAMMQNKKTFTKPLAVIHHEAESKEKGPSMKEPNPISPPIFHGLDTLTVMGIAFAAFVIGALLTGALWYIYSHTGETAGRQQVPTSPPASENSSAAHSIGSTQSTPCSSSSTA

SEQ ID NO: 219 - protein, 850 aa, Homo sapiens
MTSHYVIAIFALMSSCLATAGPEPGALCELSPVSASHPVQALMESFTVLSGCASRGTTGLPQEVHVLNLRTAGQGPGQLQREVTLHLNPISSVHIHHKSVVFLLNSPHPLVWHLKTERLATGVSRLFLVSEGSVVQFSSANFSLTAETEERNFPHGNEHLLNWARKEYGAVTSFTELKIARNIYIKVGEDQVFPPKCNIGKNFLSLNYLAEYLQPKAAEGCVMSSQPQNEEVHIIELITPNSNPYSAFQVDITIDIRPSQEDLEVVKNLILILKCKKSVNWVIKSFDVKGSLKIIAPNSIGFGKESERSMTMTKSIRDDIPSTQGNLVKWALDNGYSPITSYTMAPVANRFHLRLENNEEMGDEEVHTIPPELRILLDPGALPALQNPPIRGGEGQNGGLPFPFPDISRRVWNEEGEDGLPRPKDPVIPSIQLFPGLREPEEVQGSVDIALSVKCDNEKMIVAVEKDSFQASGYSGMDVTLLDPTCKAKMNGTHFVLESPLNGCGTRPRWSALDGVVYYNSIVIQVPALGDSSGWPDGYEDLESGDNGFPGDMDEGDASLFTRPEIVVFNCSLQQVRNPSSFQEQPHGNITFNMELYNTDLFLVPSQGVFSVPENGHVYVEVSVTKAEQELGFAIQTCFISPYSNPDRMSHYTIIENICPKDESVKFYSPKRVHFPIPQADMDKKRFSFVFKPVFNTSLLFLQCELTLCTKMEKHPQKLPKCVPPDEACTSLDASIIWAMMQNKKTFTKPLAVIHHEAESKEKGPSMKEPNPISPPIFHGLDTLTVMGIAFAAFVIGALLTGALWYIYSHTGETAGRQQVPTSPPASENSSAAHSIGSTQSTPCSSSSTA

SEQ ID NO: 220 - protein, 248 aa, Homo sapiens
QLCKFCDVRFSTCDNQKSCMSNCSITSICEKPQEVCVAVWRKNDENITLETVCHDPKLPYHDFILEDAASPKCIMKEKKKPGETFFMCSCSSAECNDNIIFSEEYNTSNPDGGGGSGGGGSGGGGSGGGGSGGGGSALDTNYCFRNLEENCCVRPLYIDFRQDLGWKWVHEPKGYYANFCSGPCPYLRSADTTHSTVLGLYNTLNPEASASPSCVPQDLEPLTILYYVGRTPKVEQLSNMVVKSCKCS